(19) United States
(12) Patent Application Publication
(10) Pub. No.: US 2015/0218620 A1
(43) Pub. Date: Aug. 6, 2015

(54) METHODS TO CAPTURE AND/OR REMOVE HIGHLY ABUNDANT RNAS FROM A HETEROGENOUS RNA SAMPLE

(71) Applicant: Integrated DNA Technologies, Inc., Coralville, IA (US)

(72) Inventors: Mark Aaron Behlke, Coralville, IA (US); Rami Zahr, San Francisco, CA (US)

(21) Appl. No.: 14/613,344

(22) Filed: Feb. 3, 2015

Related U.S. Application Data
(60) Provisional application No. 61/935,436, filed on Feb. 4, 2014.
(60) Provisional application No. 61/935,184, filed on Feb. 3, 2014.

(51) Int. Cl. C12Q 1/68

(52) U.S. Cl. CPC C12Q 1/6806; C12Q 1/6874

(57) ABSTRACT

The invention is directed to a method of using DNA oligonucleotides as baits to capture and selectively remove highly abundant RNAs from a heterogeneous RNA sample for improved enrichment of other RNAs that are unrelated to the highly abundant RNAs.

### CROSS-REFERENCE TO RELATED APPLICATIONS

This application claims benefit of priority under 35 U.S.C. 119 to U.S. provisional patent application Ser. Nos. 61/935,184 and 61/935,436, filed Feb. 3, 2014 and Feb. 4, 2014, respectively and entitled “METHODS TO CAPTURE AND/OR REMOVE HIGHLY ABUNDANT RNAS FROM A HETEROGENEOUS RNA SAMPLE,” the contents of which are herein incorporated by reference in their entirety.

### SEQUENCE LISTING

The instant application contains a Sequence Listing that has been submitted in ASCII format via EFS-Web and is hereby incorporated by reference in its entirety. Said ASCII copy, created on ______, is named IDT01-006-______ST25.txt, and is ______ bytes in size.

### FIELD OF THE INVENTION

This invention relates to methods for ribonucleic acid (RNA) selection, removal and enrichment. In particular, the invention pertains to DNA oligonucleotides as hybridization baits to capture and/or remove highly abundant RNAs from a heterogeneous RNA sample for improved enrichment of other RNAs that are unrelated to the highly abundant RNAs. The oligonucleotide compositions and reagents find robust applications for preparing cDNA libraries and cDNA nucleic acid templates for next generation sequencing applications.

### BACKGROUND OF THE INVENTION

Nucleic acid hybridization has a significant role in biotechnology applications pertaining to identification, selection, and sequencing of nucleic acids. Sequencing applications with genomic nucleic acids as the target materials demand one to select nucleic acid targets of interest from a highly complex mixture. The quality of the sequencing efforts depends on the efficiency of the selection process, which, in turn, relies upon how well nucleic acid targets can be enriched relative to non-target sequences.

A variety of methods have been used to enrich for desired sequences from a complex pool of nucleic acids, such as genomic DNA or cDNA. These methods include the polymerase chain reaction (PCR), molecular inversion probes (MIPs), or sequence capture by hybrid formation (“hybrid capture;” See, for example, Mamanova, L., Coffey, A. J., Scott, C. E., Kozarewa, I., Turner, E. H., Kumar, A., Howard, E., Shendure, J. and Turner, D. J. (2010) “Target-enrichment strategies for next-generation sequencing,” Nat. Methods 7:111-118.). Hybrid capture offers advantages over other methods in that this method requires fewer enzymatic amplification or manipulation procedures of the nucleic acid target as compared to the other methods. The hybrid capture method introduces fewer errors into the final sequencing library as a result. For this reason, the hybrid capture method is a preferred method for enriching for desired sequences from a complex pool of nucleic acids and is ideal for preparing templates in next generation sequencing (NGS) applications, where single molecular detection events occur and users may intend to identify rare mutations present in a mixed sequence population where errors introduced by polymerase action cannot easily be distinguished from natural variation.

The NGS applications usually involve randomly breaking long genomic DNA, RNA, or cDNA into smaller fragment sizes having a size distribution of 100-3,000 bp in length, depending upon the NGS platform used. The DNA termini are enzymatically treated to facilitate ligation and universal DNA adaptors are ligated to the ends to provide the resultant NGS templates. The terminal adaptor sequences provide a universal site for primer hybridization so that clonal expansion of the desired DNA targets can be achieved and introduced into the automated sequencing processes used in NGS applications. The hybrid capture method is intended to reduce the complexity of the pool of random DNA fragments from, for example, from 3×10⁹ bases (the human genome) to much smaller subsets of 10³ to 10⁸ bases that are enriched for specific sequences of interest. The efficiency of this process directly relates to the quality of capture and enrichment achieved for desired DNA sequences from the starting complex pool.

The NGS applications typically use the hybrid capture method of enrichment in the following manner. A prepared pool of NGS templates is heat denatured and mixed with a pool of capture probe oligonucleotides (“baits”). The baits are designed to hybridize to the regions of interest within the target genome and are usually 60-200 bases in length and further are modified to contain a ligand that permits subsequent capture of these probes. One common capture method incorporates a biotin group (or groups) on the baits. Other capture ligands can be used. After hybridization is complete to form the DNA template:bait hybrids, capture is performed with a component having affinity for only the bait. For example, streptavidin-magnetic beads can be used to bind the biotin moiety of biotinylated-baits that are hybridized to the desired DNA targets from the pool of NGS templates. Washing removes unbound nucleic acids, reducing the complexity of the retained material. The retained material is then eluted from the magnetic beads and introduced into automated sequencing processes, providing for ‘capture enrichment’, where the captured nucleic acids are retained as an enriched pool for subsequent study.

Another strategy is to use hybrid capture to remove sequences homologous to those of the capture probes or baits, thereby enriching the remaining complex nucleic acid sample for desired sequence content by clearing or removing undesired content which is homologous to the capture probes. This strategy is generally of little use when the nucleic acid sample is genomic DNA, where removal of a minority of undesired sequences does not appreciably enrich the remaining sample for desired sequences. However, this approach can have significant benefit when applied to a sample of total cellular RNA. Typically sequencing of RNA (RNA-Seq) by NGS methods involves conversion of RNA to cDNA (before or after fragmentation), ligation of cDNA fragments to linkers, library preparation, and sequencing similar to what is done for genomic DNA (see: Cloonan, N. et al. (2008) Stem cell transcriptome profiling via massive-scale mRNA sequencing. Nat. Methods 5, 613-619; Mortazavi, A., Williams, B. A., McCue, K., Schaeffer, L. & Wold, B. (2008) Mapping and quantifying mammalian transcriptomes by RNA-seq. Nat. Methods 5, 621-628; Guttman, M. et al. (2010) Ab initio reconstruction of cell type-specific transcriptomes in mouse reveals the conserved multi-exonic structure of lincRNAs. Nat. Biotechnol. 28, 503-510.). A major problem with RNA-Seq, however, is that ˜80-95% of the total RNA sample is ribosomal RNA (rRNA). RNA-Seq is typically performed to study the mRNA, long-non-coding RNAs, and other unique RNAs, which are generally present at low frequencies. Having 80-95% of the sequence space consumed by sequencing unwanted rRNA increases cost and decreases throughput. Methods that remove rRNA prior to sequencing greatly improve the amount of useful sequence information obtained from an RNA-Seq NGS run.

This same strategy could be useful, for example, to remove any overexpressed RNA from a total RNA sample, not just rRNA. One such example is encountered in sequencing reticulocyte RNA, which contains an overabundance of hemoglobin mRNA. Removal of hemoglobin mRNA improves the ability to study non-hemoglobin RNAs present in reticulocytes. One method described which could be applied to removal of hemoglobin mRNA, rRNA, or any other overabundant species was described by Ambion in US patent application US2006/0257902 (Mendoza, L. G., Moturi, S., Setterquist, R., and Whitley, J. P., METHOD AND COMPOSITIONS FOR DEPLETING ABUNDANT RNA TRANSCRIPTS). In this application, methods are disclosed whereby RNA capture baits are made by in vitro transcription (IVT) from DNA templates. The RNA baits comprise two domains, a universal capture domain and a target binding domain. The target binding domain binds to (e.g., is complementary to and anneals to) the overabundant RNA species that is desired to be depleted. The RNA bait is hybridized to a complex RNA mixture, the baits anneal to their targets, then the bait:target complexes are removed by hybridization to magnetic beads (or other solid phase particles) that bear sequence tags complementary to the universal capture domain on the RNA bait. The captured overabundant species are removed from the complex mixture, which is then used for downstream applications, such as sequencing.

It is also possible to purify mRNA from total RNA by capture using oligo-dT to bind the poly-A tail present on mRNAs. However, this method can still retain a significant amount of rRNA contamination. Methods have been disclosed to improve the results obtained using this approach largely through use of improved buffers and hybridization methods. See US patent U.S. Pat. No. 6,812,341 (Conrad, R. C., HIGH EFFICIENCY MRNA ISOLATION METHODS AND COMPOSITION, Nov. 2, 2004). However, this approach only serves to capture mRNA. It has recently been appreciated that an important fraction of long non-coding RNAs (IncRNAs) and some translated mRNAs (such as those encoding histone proteins) do not have poly-A tails and therefore would not be captured using this approach. Therefore to obtain a comprehensive evaluation of the RNA species present in a cell using RNA-Seq methods, it is more preferable to remove rRNA from the complex mixture than to purify/isolate the poly-A mRNA fraction.

One current method to remove rRNA from total cellular RNA prior to performing RNA-Seq experiments is the “Ribo-Zero rRNA removal kit” sold by Epicentre/Illumina. See: http://www.epibio.com/applications/ma-sequencing/rrna-removal/ribo-zero-gold-kits-(human-mouse-rat). In this method, biotin-tagged RNA baits are made using in vitro transcription (IVT) with biotin-UTP so that the biotin label is present internally in the RNA bait capture probe. See US Patent Application by Sooknanan, R. R., US2011/0040081, METHODS, COMPOSITIONS, AND KITS FOR GENERATING RRNA-DEPLETED SAMPLES OR ISOLATING RRNA FROM SAMPLES.

Biotin-labeled RNA bait capture probes are expensive to prepare owing to the significant cost of biotin-UTP as a starting material. Accordingly, the cost of performing RNA-Seq experiments for NGS applications can be significant depending upon the number of RNA baits required as capture probes.

Another current method to remove rRNA from total cellular RNA prior to performing RNA-Seq experiments is the “GeneRead rRNA depletion kit” sold by QIAGEN (cat. no. 180211). See: http://www.qiagen.com/products/catalog/assay-technologies/next-generation-sequencing/generead-rrna-depletion-kit. This approach employs simpler synthetic DNA baits but relies upon a more complex clearance approach using a antibody that recognizes the RNA:DNA heteroduplex structure formed by the DNA capture bait and the rRNA target, followed by secondary capture/pull-down by a magnetic bead derivatized to bind antibody fragments. See: O'Neil, D., Glowatz, H., and Schlumpberger, M. (2013) Ribosomal RNA depletion for efficient use of RNA-seq capacity. Current Protocols in Molecular Biology 4.19.1-4.19.8 (July 2013). Due to the simple DNA baits employed, this method is less expensive to perform than the biotinylated-RNA bait method from Epicentre, but is still a costly step in RNA-Seq library production. Yet another current method to remove rRNA from total cellular RNA prior to performing RNA-Seq experiments is the “RiboMinus™ Eukaryote Kit for RNA-Seq” sold by Ambion/Life Technologies. See: http://www.lifetechnologies.com/us/en/home/life-science/dna-rna-purification-analysis/rna-ex traction/rna-applications/ribosomal-rna-depletion/ribominustechnologypage.html. This method employs synthetic DNA baits modified with high affinity locked nucleic acid (LNA) residues. This modification enables the baits to be shorter and retain high binding affinity; however, the LNA modification is costly. In this case the baits are modified with a terminal biotin ligand, permitting clearance of the unwanted rRNA:bait complex with streptavidin-magnetic beads. In spite of the simple biotin-SA-bead capture approach, this method remains expensive to perform due to the higher cost of manufacture of LNA-modified capture baits. Another method to remove ribosomal RNA is disclosed in U.S. patent application Ser. No. 12/940,981, “Methods for depleting RNA from nucleic acid samples”, Sinicropi et al. In this method, unmodified DNA oligonucleotides complementary to ribosomal RNA are hybridized to an RNA sample containing ribosomal RNA, and the sample is then treated with RNase H, an enzyme that selectively cleaves the RNA strand in an RNA/DNA heteroduplex. The sample is then treated with DNase to cleave the excess DNA oligonucleotides. Disadvantages of this method are the lengthy and complex temperature gradient required for the oligonucleotide hybridization step and the requirement for 2 nuclease steps. Nuclease treatment runs the risk of degradation of desired RNA, due to either non-specific activity of nucleases for degrading non-target nucleic acids, or to contamination of a specific nuclease (for example RNase H) with other nuclease(s) (for example RNase A) having unwanted activity (for example, activity directed toward degradation of mRNA).

Several of the methods for rRNA depletion described above include a series of steps where the undesired RNA (e.g. rRNA) complexed with biotinylated capture oligonucleotide(s), and also excess biotinylated capture oligonucleotides not complexed with undesired RNA, are removed by linking the complex and the excess capture oligos to streptavidin-modified magnetic particles, and then removing the particles along with the undesired RNA/capture oligonucleotide complex. The step of removing the magnetic particles is typically accomplished by placing the vessel containing the reaction components on a magnetic stand for several minutes to attract the magnetic particles (linked to the undesired RNA/oligo complex) to the side of the vessel and then removing the fluid containing the desired RNA and transferring it to a second vessel. These steps are time-consuming, require the use of additional consumables (the second vessels and pipet tips used for transfer), and run the risk of introducing errors in sample identity during transfer of the fluid with desired RNA (i.e. risk of sample mix-up during transfer). Improved methods for accomplishing the steps of magnetic attraction and sample transfer that avoid these drawbacks would reduce the time and cost required for sample preparation and also minimize the risk of sample mix-up.

There is a need for more economical reagents and improved methods for ribonucleic acid (RNA) selection, removal and enrichment such that highly abundant RNAs can be removed from a heterogeneous RNA sample for improved enrichment of other RNAs that are unrelated to the highly abundant RNAs. Economical approaches for preparing cDNA nucleic acid templates for next generation sequencing applications would dramatically reduce the cost of RNA-Seq experiments for NGS applications.

### BRIEF SUMMARY OF THE INVENTION

In one aspect, the invention relates to a method of selecting an undesired RNA target from a population of RNA molecules. The method includes two steps. The first step includes contacting the population of RNA molecules with one or more DNA oligonucleotides comprising a bait to form a mixture wherein the DNA bait anneals or hybridizes to any complementary RNA species present in the mixture. The second step includes removing the undesired RNA target:bait complex from the mixture.

In a second aspect, the invention relates to a method of performing massively parallel sequencing of RNA from a sample. The method includes four steps. The first step includes contacting the complex population of total RNA with a plurality of DNA oligonucleotides comprising baits to form a mixture. At least one member of the plurality of DNA oligonucleotides comprising baits has substantial sequence complementarity to a sequence within at least one species of an undesired RNA target. The second step includes isolating at least one species of an undesired RNA target from the mixture to form a depleted population of total RNA. The third step includes preparing a cDNA library from the depleted population of total RNA. The fourth step includes sequencing the double-stranded cDNA library generated from the depleted library population of total RNA.

In a third aspect, the invention relates to a kit that includes a capture reagent for use in a selection method of an undesired RNA. The capture reagent includes a plurality of DNA bait oligonucleotides. Each member of the plurality of DNA bait oligonucleotides is prepared individually by a synthetic chemical process.

### BRIEF DESCRIPTION OF THE DRAWINGS

FIG. 1 depicts a strategy for selection and removal of undesired RNA targets from a total RNA mixture without co-selection of desired RNAs. The DNA baits are illustrated as short lines coupled to a terminal bulb (signifying an exemplary 5′-biotin moiety), and the bead coupled to streptavidin (starlet symbol) to capture the biotin-coupled complex.

FIG. 2 shows a gelshift assay demonstrating binding of bait probes to rRNA. Varying amounts of stock DNA bait solution were hybridized to 1 μg of human total genomic RNA (see Example 1), separated on an agarose gel, stained with ethidium bromide, and visualized using UV-induced fluorescence. An inverted gel image is shown. Lane 1: 0.25 μL bait solution, Lane 2: 0.5 μL bait solution; Lane 3: 1.0 μL bait solution; Lane 4: 1.5 jμL bait solution; Lane 5: control with no bait.

FIG. 3 shows removal of rRNA from total RNA using biointylated baits and capture with streptavidin (SA) magnetic beads. Varying amounts of stock DNA bait solution were hybridized to 1 μg of human total genomic RNA (See Example 2). The rRNA:bait complexes were removed using varying amounts of SA-magnetic beads. The remaining nucleic acids present in the samples were separated on an agarose gel, stained with ethidium bromide, and visualized using UV-induced fluorescence. An inverted gel image is shown. Lane 1: 0.25 μL bait solution+20 μL SA-mag beads; Lane 2: 0.25 μL bait solution+30 μL SA-mag beads; Lane 3: 0.5 μL bait solution+20 μL SA-mag beads; Lane 4: 0.5 μL bait solution+30 μL SA-mag beads; Lane 5: 0.25 μL bait solution with no SA-mag bead clearance; Lane 6: 0.5 μL bait solution with no SA-mag bead clearance.

FIG. 4 shows rRNA depletion from RNA-Seq NGS libraries. Total human cellular RNA (1 μg or 3 μg) was depleted of rRNA using the method of the invention. A sample was mock-treated as control. RNAs were converted to cDNA and NGS libraries were prepared and sequencing performed on a MiSEQ instrument. Sequencing reads were mapped to the human genome and the relative percent of total reads mapping to rRNA sequences, human non-rRNA sequences, and unmapped sequences (e.g., primer dimers and other elements of non-human origin) is indicated.

FIG. 5 shows removal of rRNA from total RNA using biointylated baits and capture with streptavidin (SA) magnetic beads using a DNase-free protocol. DNA bait solution was hybridized to 2 μg of human total genomic RNA (See Example 2) and removed using SA-magnetic beads. Samples were separated on an agarose gel, stained with ethidium bromide, and visualized using UV-induced fluorescence. An inverted gel image is shown. Lane 1: 2 μg of human total genomic RNA+1 μL bait solution; Lane 2: mock depletion of 2 μg of human total genomic RNA with no bait solution.

FIG. 6 shows rRNA depletion from total human RNA assayed by RT-PCR. Total human cellular RNA (2 μg) was depleted of rRNA using the method of the invention. A sample was mock-treated as control. RNAs were converted to cDNA and end point PCR was performed using the primers indicated. Samples were separated by agarose electrophoresis and visualized by ethidium bromide fluorescence. An inverted gel image is shown. Lanes 1,2: 12S mitochondrial rRNA; Lanes 3,4: 16S mitochondrial rRNA; Lanes 5,6: 18S cytoplasmic rRNA; Lanes 7,8: 28S cytoplasmic rRNA; Lanes 9,10: GAPDH mRNA. Input RTs as follows: Lanes 1,3,5,7,9,11,13 were from the RNA prep depleted of rRNA by hybridization to bait and Lanes 2,4,6,8,10,12,14 were from mock-hybridized RNA not depleted with bait (control).

### DETAILED DESCRIPTION OF THE INVENTION

Certain terms are first defined. Additional terms are defined throughout the specification.

Terms used herein are intended as “open” terms (for example, the term “including” should be interpreted as “including but not limited to,” the term “having” should be interpreted as “having at least,” the term “includes” should be interpreted as “includes but is not limited to,” etc.).

Furthermore, in those instances where a convention analogous to “at least one of A,B and C, etc.” is used, in general such a construction is intended in the sense of one having ordinary skill in the art would understand the convention (for example, “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “from,” “to,” “up to,” “at least,” “greater than,” “less than,” and the like, include the number recited and refer to ranges which can subsequently be broken down into sub-ranges as discussed above.

A range includes each individual member. Thus, for example, a group having 1-3 members refers to groups having 1, 2, or 3 members. Similarly, a group having 6 members refers to groups having 1, 2, 3, 4, 5, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, the articles “a” and “an” refer to one or to more than one (for example, to at least one) of the grammatical object of the article.

As used herein, “or” is used herein to mean, and is used interchangeably with, the term “and/or”, unless context clearly indicates otherwise. The use of the term “and/or” in some places herein does not mean that uses of the term “or” are not interchangeable with the term “and/or” unless the context clearly indicates otherwise.

“About” and “approximately” shall generally mean an acceptable degree of error for the quantity measured given the nature or precision of the measurements. Exemplary degrees of error are within 25 percent (%), typically, within 10%, and more typically, within 5% of a given value or range of values.

The term “affinity tag” refers to a ligand that permits detection and/or selection of an oligonucleotide sequence to which the ligand is attached. For the purposes of this disclosure, a bait may include an affinity tag. In particular, the affinity tag is positioned typically at either or both the 3′-terminus and/or 5′-terminus of an oligonucleotide through the use of conventional chemical coupling technology. Exemplary affinity tags include biotin, digoxigenin, streptavidin, polyhistidine (for example, (His6),), glutathione-S-transferase (GST), HaloTag®, AviTag, Calmodulin-tag, polyglutamate tag, FLAG-tag, HA-tag, Myc-tag, S-tag, SBP-tag, Softag 3, V5 tag, Xpress tag, a hapten, among others.

“Acquire” or “acquiring” as the terms are used herein, refer to obtaining possession of a physical entity, or a value, for example, a numerical value, by “directly acquiring” or “indirectly acquiring” the physical entity or value. “Directly acquiring” means performing a process (for example, performing a synthetic or analytical method) to obtain the physical entity or value. “Indirectly acquiring” refers to receiving the physical entity or value from another party or source (for example, a third party laboratory that directly acquired the physical entity or value). Directly acquiring a physical entity includes performing a process that includes a physical change in a physical substance, for example, a starting material. Exemplary changes include making a physical entity from two or one starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, for example, performing an analytical process which includes a physical change in a substance, for example, a sample, analyte, or reagent (sometimes referred to herein as “physical analysis”), performing an analytical method, for example, a method which includes one or more of the following: separating or purifying a substance, for example, an analyte, or a fragment or other derivative thereof, from another substance; combining an analyte, or fragment or other derivative thereof, with another substance, for example, a buffer, solvent, or reactant; or changing the structure of an analyte, or a fragment or other derivative thereof, for example, by breaking or forming a covalent or non-covalent bond, between a first and a second atom of the analyte; or by changing the structure of a reagent, or a fragment or other derivative thereof, for example, by breaking or forming a covalent or non-covalent bond, between a first and a second atom of the reagent.

“Acquiring a sequence” or “acquiring a read” as the term is used herein, refers to obtaining possession of a nucleotide sequence or amino acid sequence, by “directly acquiring” or “indirectly acquiring” the sequence or read. “Directly acquiring” a sequence or read means performing a process (for example, performing a synthetic or analytical method) to obtain the sequence, such as performing a sequencing method (for example, a Next Generation Sequencing (NGS) method). “Indirectly acquiring” a sequence or read refers to receiving information or knowledge of, or receiving, the sequence from another party or source (for example, a third party laboratory that directly acquired the sequence). The sequence or read acquired need not be a full sequence, for example, sequencing of at least one nucleotide, or obtaining information or knowledge, that identifies one or more of the alterations disclosed herein as being present in a subject constitutes acquiring a sequence.

Directly acquiring a sequence or read includes performing a process that includes a physical change in a physical substance, for example, a starting material, such as a tissue or cellular sample, for example, a biopsy, or an isolated nucleic acid (for example, DNA or RNA) sample. Exemplary changes include making a physical entity from two or more starting materials, shearing or fragmenting a substance, such as a genomic DNA fragment; separating or purifying a substance (for example, isolating a nucleic acid sample from a tissue); combining two or more separate entities into a mixture, performing a chemical reaction that includes breaking or forming a covalent or non-covalent bond. Directly acquiring a value includes performing a process that includes a physical change in a sample or another substance as described above.

“Acquiring a sample” as the term is used herein, refers to obtaining possession of a sample, for example, a tissue sample or nucleic acid sample, by “directly acquiring” or “indirectly acquiring” the sample. “Directly acquiring a sample” means performing a process (for example, performing a physical method such as a surgery or extraction) to obtain the sample. “Indirectly acquiring a sample” refers to receiving the sample from another party or source (for example, a third party laboratory that directly acquired the sample). Directly acquiring a sample includes performing a process that includes a physical change in a physical substance, for example, a starting material, such as a tissue, for example, a tissue in a human patient or a tissue that has was previously isolated from a patient. Exemplary changes include making a physical entity from a starting material, dissecting or scraping a tissue; separating or purifying a substance (for example, a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, for example, as described above.

“Bait,” as used herein, is type of hybrid capture reagent. A bait can be a nucleic acid molecule, for example, a DNA or RNA molecule, which can hybridize to (for example, be complementary to), and thereby allow capture of a nucleic acid target. In one embodiment, a bait is an RNA molecule (for example, a naturally-occurring or modified RNA molecule); a DNA molecule (for example, a naturally-occurring or modified DNA molecule), or a combination thereof. In other embodiments, the bait includes incorporation of chemical modifiers which increase binding affinity of the bait to the target RNA nucleic acid, such as locked nucleic acid residues (LNAs), 2′-O-methyl RNA residues, or other similar modifiers as are well known to those with skill in the art. In other embodiments, a bait is a peptide nucleic acid (PNA) molecule. In other embodiments, a bait includes a binding entity, for example, an affinity tag, that allows capture and separation, for example, by binding to a binding entity, of a hybrid formed by a bait and a nucleic acid hybridized to the bait. In one embodiment, a bait is suitable for solution phase hybridization. A “DNA bait” refers to a bait composed of DNA residues, and an “RNA bait” refers to a bait composed of RNA residues.

“Bait set,” as used herein, refers to one or a plurality of bait molecules.

“Binding entity” means any molecule to which molecular tags can be directly or indirectly attached that is capable of specifically binding to an analyte. The binding entity can be an affinity tag on each bait sequence. In certain embodiments, the binding entity allows for separation of the bait/member hybrids from the hybridization mixture by binding to a partner, such as an avidin molecule, or an antibody that binds to the hapten or an antigen-binding fragment thereof. Exemplary binding entities include, but are not limited to, an affinity tag, a biotin molecule, a hapten, an antibody, an antibody binding fragment, a peptide, and a protein.

“Complementary” refers to sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region that is antiparallel to the first region if the residue is thymine or uracil. Similarly, it is known that a cytosine residue of a first nucleic acid strand is capable of base pairing with a residue of a second nucleic acid strand that is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an antiparallel fashion, at least about 50%, at least about 75%, at least about 90%, or at least about 95%/o of the nucleotide residues of the first portion are capable of base pairing with nucleotide residues in the second portion. In other embodiments, all nucleotide residues of the first portion are capable of base pairing with nucleotide residues in the second portion.

As used herein, the term “library” refers to a collection of members. In one embodiment, the library includes a collection of nucleic acid members, for example, a collection of whole genomic, subgenomic fragments, cDNA, cDNA fragments, RNA, RNA fragments, or a combination thereof. In one embodiment, a portion or all of the library members comprises a non-target adaptor sequence. The adaptor sequence can be located at one or both ends. The adaptor sequence can be useful, for example, for a sequencing method (for example, an NGS method), for amplification, for reverse transcription, or for cloning into a vector.

The library can comprise a collection of members, for example, a target member (for example, a highly abundant RNA). The members of the library can be from a single individual. In embodiments, a library can comprise members from more than one subject (for example, 2, 3, 4, 5, 6, 7, 8, 9, 10, 20, 30 or more subjects), for example, two or more libraries from different subjects can be combined to from a library having members from more than one subject. In one embodiment, the subject is human having, or at risk of having, a cancer or tumor.

“Library-catch” refers to a subset of a library, for example, a subset enriched for preselected, undesired RNAs, for example, product captured by hybridization with preselected baits.

“Member” or “library member” or other similar term, as used herein, refers to a nucleic acid molecule, for example, a DNA, RNA, or a combination thereof, that is the member of a library. Typically, a member is a DNA molecule, for example, genomic DNA or cDNA. A member can be fragmented, for example, sheared or enzymatically prepared, genomic DNA. Members comprise sequence from a subject and can also comprise sequence not derived from the subject, for example, a non-target sequence such as adaptors sequence, a primer sequence, or other sequences that allow for identification, for example, “barcode” or “index” sequences.

“Next-generation sequencing or NGS or NG sequencing” as used herein, refers to any sequencing method that determines the nucleotide sequence of either individual nucleic acid molecules (for example, in single molecule sequencing) or clonally expanded proxies for individual nucleic acid molecules in a high through-put fashion (for example, greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, for example, in Metzker, M. (2010) Nature Reviews Genetics 11:31-46, incorporated herein by reference.

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

The term “nucleic acid target” refers to the nucleic acid having complementarity with a bait. For the purposes of this disclosure, a nucleic acid target is an undesired RNA sequence in a biological sample. Examples of an undesired RNA sequence include highly abundant RNA such as rRNA, tRNA, and other cellular RNAs that represent a significant fraction, e.g. at least about 5% 10% of the total RNA present in a biological sample. Examples of such other cellular RNAs include globin RNA from red blood cells and immunoglobulin RNA from B cells. Other examples include the mRNAs encoding beta-actin, GAPDH, cyclophilin, and other so-called “housekeeping genes” which are generally present at high levels in eukaryotic total RNA preparations, and which are generally not of interest for quantitative analysis using NGS or other methods.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

“Plurality,” as used herein, means “at least two” or “two or more.”

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (e.g., a DNA polymerase or reverse transcriptase) in an appropriate buffer and at a suitable temperature. Primer extension can also be carried out in the absence of one or more of the nucleoside triphosphates in which case an extension product of limited length is produced. As used herein, the term “primer” is intended to encompass the oligonucleotides used in ligation-mediated reactions, in which one oligonucleotide is “extended” by ligation to a second oligonucleotide which hybridizes at an adjacent position. Thus, the term “primer extension”, as used herein, refers to both the polymerization of individual nucleoside triphosphates using the primer as a point of initiation of DNA synthesis and to the ligation of two oligonucleotides to form an extended product.

A primer is preferably a single-stranded DNA. The appropriate length of a primer depends on the intended use of the primer but typically ranges from 6 to 50 nucleotides, preferably from 15-35 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the nucleic acid target, but which facilitates cloning or detection of the amplified product. The region of the primer that is sufficiently complementary to the template to hybridize is referred to herein as the hybridizing region.

“Residue,” as used herein when referencing “DNA residues” or “RNA residues” in a nucleic acid molecule, refers to an internucleotide monomer comprising at least a nucleobase covalently bonded to a sugar moiety. An RNA molecule or DNA molecule, including modifications thereof, typically comprises a plurality of residues.

“Sample,” “tissue sample,” “patient sample,” “patient cell or tissue sample” or “specimen,” each refers to a collection of similar cells obtained from a tissue, or circulating cells, of a subject or patient. The source of the tissue sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid or interstitial fluid; or cells from any time in gestation or development of the subject. The tissue sample can contain compounds that are not naturally intermixed with the tissue in nature such as preservatives, anticoagulants, buffers, fixatives, nutrients, antibiotics or the like. In one embodiment, the sample is preserved as a frozen sample or as formaldehyde- or paraformaldehyde-fixed paraffin-embedded (FFPE) tissue preparation. For example, the sample can be embedded in a matrix, for example, an FFPE block or a frozen sample.

The term “biological sample” refers to a material obtained from a biological source. Examples of a biological sample include a cell, a tissue, a fluid (for example, blood), an excrement (for examples, feces or urine), a biopsy, a swab, a skin scraping, among others. Biological samples include “Sample,” “tissue sample,” “patient sample,” “patient cell or tissue sample” or “specimen,” as those terms are used herein.

The term “tiling” refers to covering a specific region of a nucleic acid target with one or more baits through hybridization of the bait(s) to the nucleic acid target. The terms “1-fold tiling” or “100% tiling” refer to conditions enabling covering of an entire region, or most (>50%) of an entire region, of a nucleic acid target with a plurality of baits through hybridization of the plurality of baits to the nucleic acid target, wherein the plurality of baits can be aligned end-to-end along the complementary strand of the nucleic acid target and where all members of the plurality of baits can hybridize to the region of a nucleic acid target. The terms “n-fold tiling” or “n-fold redundant tiling” refer to conditions enabling covering of an entire region of a nucleic acid target with a plurality of baits through hybridization of the plurality of baits to the nucleic acid target, wherein the plurality of baits are separated by a spacing distance that is I/n times the average bait length along the complementary strand of the nucleic acid target and wherein at least n members of the plurality of baits have the ability to hybridize completely to the common inter-spacing region of the nucleic acid target. For example, 4-fold tiling using a plurality of baits having an average length of 120 nucleotides results in hybridization of the plurality of baits at a spacing of 30 nucleotides along a given region of the nucleic acid target, wherein at least four bait sequences have the ability to hybridize to the common inter-spacing region of the nucleic acid target. For example, 2-fold tiling using a plurality of baits having an average length of 120 nucleotides results in hybridization of the plurality of baits at a spacing of 60 nucleotides along a given region of the nucleic acid target, wherein at least two bait sequences have the ability to hybridize to the common inter-spacing region of the nucleic acid target. As used herein, when referring to hybridizing baits to a region of a nucleic acid target, “n-fold covering,” “n-fold coverage,” “n×coverage” “n×coverage strategy” and “n-fold tiling” have the same meanings are used interchangeably.

As used herein, “unmarked RNA” refers to a nucleic acid that is not modified or prepared to include a unique tag sequence or label enabling its detection. An example of an unmarked RNA includes an RNA from a biological sample.

As used herein, “marked RNA” refers to a nucleic acid that is modified or prepared to include a unique tag sequence or label enabling its detection. A marked RNA will typically have the same primary sequence of an unmarked RNA except for the inclusion of the unique tag sequence or label. A marked RNA can be obtained in a variety of ways, such as by IVT methods.

A “control nucleic acid sample” or “reference nucleic acid sample” as used herein, refers to nucleic acid molecules from a control or reference sample. Typically, it is DNA, for example, genomic DNA, RNA, or cDNA derived from RNA, not containing the alteration or variation in the gene or gene product. In certain embodiments, the reference or control nucleic acid sample is a wild type or a non-mutated sequence. In certain embodiments, the reference nucleic acid sample is purified or isolated (for example, it is removed from its natural state). In other embodiments, the reference nucleic acid sample is from a non-tumor sample, for example, a blood control, a normal adjacent tumor (NAT), or any other non-cancerous sample from the same or a different subject. In some embodiments, the reference nucleic acid sample can be a marked RNA that permits detection of the efficiency of a method for selecting an unmarked RNA.

“Sequencing” a nucleic acid molecule requires determining the identity of at least 1 nucleotide in the molecule. In embodiments the identity of less than all of the nucleotides in a molecule are determined. In other embodiments, the identity of a majority or all of the nucleotides in the molecule is determined.

Headings, for example, (a), (b), (i) etc., are presented merely for ease of reading the specification and claims. The use of headings in the specification or claims does not require the steps or elements be performed in alphabetical or numerical order or the order in which they are presented.

The present invention employs affinity-tagged DNA baits to remove highly abundant RNA (for example, rRNA) from a total RNA or other complex RNA sample. Methods have been described to employ affinity-tagged DNA baits to enrich DNA sequences from complex mixtures (see, for example, protocols and commercial products relating to xGen® Lockdown® Probes from Integrated DNA Technologies at: http://www.idtdna.com/pages/products/nextgen/target-capture/xgen-lockdown-probes). Also see U.S. patent application Ser. No. 13/935,451 (2013) (Behlke, M. A., Havens, J. R., Jarosz, M., Zwirko, Z., Lipson, D., and Juhn, F. S., TM-ENHANCED BLOCKING OLIGONUCLEOTIDES AND BAITS FOR IMPROVED TARGET ENRICHMENT IN MASSIVELY PARALLEL SEQUENCING EXPERIMENTS, and U.S. Provisional Application No. 61/745,435, filed Dec. 21, 2012, entitled “TM-ENHANCED BLOCKING OLIGONUCLEOTIDES AND BAITS FOR IMPROVED TARGET ENRICHMENT IN MASSIVELY PARALLEL SEQUENCING EXPERIMENTS,” by Behlke et al.).

Existing methods use DNA probes to enrich desired sequences by hybrid capture from DNA samples; the desired species are captured by the DNA baits, eluted, recovered, and used for downstream applications. In contrast, the present invention uses DNA probes to capture and remove unwanted RNA species, such as highly abundant rRNA, from RNA samples. In this case, the desired species are not captured by the DNA baits; instead, the unwanted species are captured by the DNA baits and are removed from the sample by affinity selection of the baits. The remaining material in the sample is thereby enriched for desired sequences by removing the undesired sequences from the complex mixture.

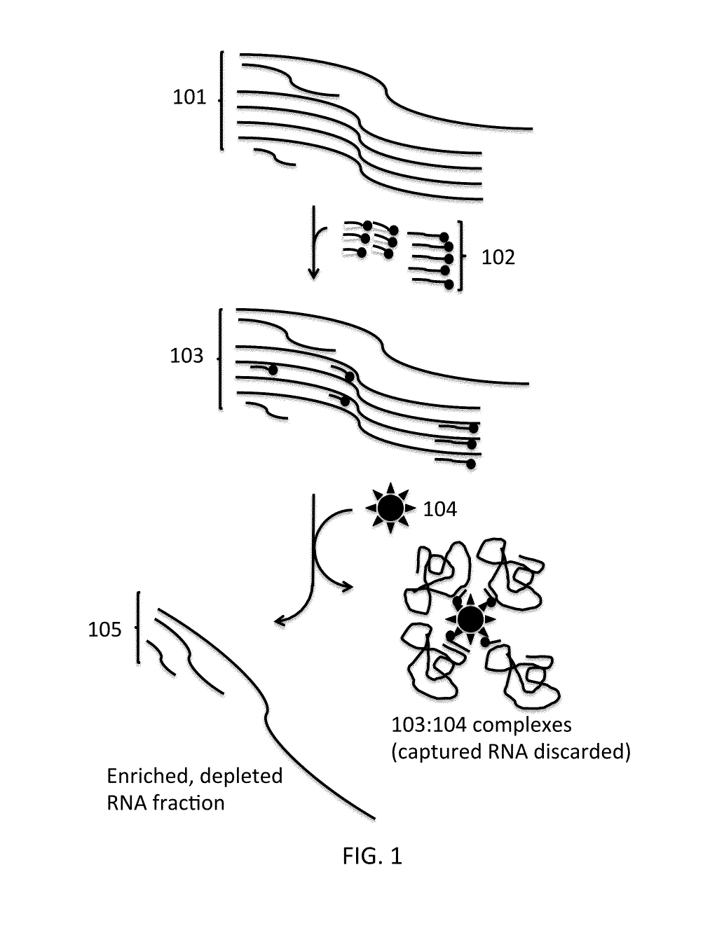

Referring to FIG. 1, the principle of selection and removal of undesired RNA sequences (for example, rRNA) with DNA baits is illustrated for a typical NGS application. Total RNA 101 (10 ng-10 g, typically around 1 μg) and biotinylated DNA oligonucleotide baits 102 are mixed together and briefly (for example, <5 minutes) heat-denatured at 60-95° C. in a suitable buffer mixture adjusted to include a final concentration of sodium chloride (for example, 400 mM) and Tris-CI pH 8 (for example, 10 mM) buffer or similar hybridization buffer (such as Saline Sodium Citrate buffer (SSC), TMAC (tetramethyl ammonium chloride)), with or without formamide, as are well known to those with skill in the art, followed by hybridization at about 50-70° C. for a period of time, then cooled to and maintained at room temperature for a period of time. Optimal hybridization temperature will vary with buffer composition and, for example, will be significantly lower when containing increasing amounts of formamide. The mixture containing DNA bait:rRNA complexes 103 is then incubated with streptavidin-magnetic beads 104 to permit capture of DNA bait:rRNA complexes 103. The remaining rRNA-depleted sample 105 is processed for cDNA synthesis and library preparation as appropriate for the sequence method employed.

Basic methods and protocols for capture can be similar or even identical to those employed for DNA capture as previously taught in the above cited prior art. It may be beneficial to adjust buffer composition or hybridization temperature for working with RNA capture owing to the potential complexity of RNA folding and competing RNA secondary structures that can reduce DNA bait hybridization to nucleic acid targets and subsequent RNA capture; such methods are well known to those with skill in the art. In the present invention, the captured material is discarded and the cleared total RNA sample is retained for future use. In preferred embodiments, the cleared total RNA is further purified and concentrated for future use. An example of method for further purification and concentration is by solid-phase extraction of the cleared RNA onto magnetic beads. Procedural details for magnetic-bead-based purification/concentration of nucleic acids are disclosed in the product literature for Mag-Bind RXNPure® Plus magnetic beads (cat #M 1386, Omega Bio-Tek).

Because captured material is discarded, DNA baits of captured material can be processed and recycled for use in subsequent RNA capture experiments depending upon the application. Thus, DNA baits of the present invention can afford certain additional economical advantages over the use of RNA baits for RNA capture.

DNA baits are typically synthesized with an affinity tag that permits capture of the bait:target complex. A preferred affinity tag includes biotin. Highly preferred DNA baits include biotin at both the 5′-terminus and the 3′ terminus of the oligonucleotide. Including biotin affinity tags at both termini can increase the efficiency with which the baits are captured onto the streptavidin magnetic beads, and also offer the advantage that the modifications at each terminus minimize the ability of excess baits to be ligated into the NGS library, thus reducing contamination of the library with bait sequences. The DNA baits can be made of a variety of lengths, wherein baits having a length from about 30 nucleotides to about 200 nucleotides being routine. DNA baits having a length of about 60-120 nucleotides are generally preferred. DNA baits having a length of about 60 nucleotides are especially preferred because the relatively short size maximizes their removal during the final purification steps used to recover the desired RNA in a pure, concentrated form. DNA baits typically include unmodified canonical nucleobases that are arranged in a primary sequence to enable hybridization to the nucleic acid target. Random “N-domain” region and/or the use of universal bases (for examples, inosine, 3-nitropyrrole, and 5-nitroindole, among others) can be employed to permit baits to hybridize and bind/capture targets bearing sequence polymorphisms (e.g., to make a single set of capture baits which will efficiently remove rRNA from RNA derived from a mixed bacterial population). Other affinity tags can be employed, as are well known to those with skill in the art. Affinity tags can be placed internally within the bait sequence, however it is generally preferred to place the tag modification at the 5′- or the 3′-end of the bait. It is more preferred to place the affinity tag at both the 5′- and 3′-ends.

Tₘ-enhanced oligonucleotides as DNA baits can be used as well; however, the cost of the synthetic Tₘ-enhanced nucleoside reagents necessary for preparing such Tₘ-enhanced DNA baits is more costly than conventional synthetic nucleoside reagents. For this reason, DNA baits prepared with conventional synthetic nucleoside reagents are generally preferred in the method disclosed herein. However, use of Tₘ-enhancing modifications may be beneficial to improve capture efficiency if the baits for are short, for example 20-40 nucleotides. Short baits may be desirable when high specificity of capture is required, for example, if it is desired to remove RNAs derived from one species but not a related species present in a mixed source RNA sample.

### Design of DNA Baits Against Exemplary RNA Targets

To prepare libraries for NGS from human RNA, DNA baits complementary to human cytoplasmic ribosomal 28S, 18S, 5S, and 5.8S RNA species as well as human mitochondrial ribosomal 16S and 12S RNA species preferably should be synthesized and employed; however, the bulk of rRNA sequences present in total RNA represent the human 28S and 18S species. Sequences of these rRNA species are shown in Appendix 1. A similar strategy can be employed to make bait pools for capture of other mammalian species, such as mouse, rat, monkey, etc. or non-mammalian species, such as worms, frog, fish, bird and prokaryotic or archaeal species.

Ribosomal RNAs are long, have subdomains with very high GC content, and naturally form highly complex, folded structures. These features make it difficult to design good capture probes/baits. However, it is not necessary to synthesize baits that span all complex, difficult regions. It is sufficient to synthesize baits which capture unique sequences that flank highly structured regions. Importantly, DNA baits inherently show lower hairpin and secondary structure formation than RNA baits, so DNA baits as described herein will perform better than the same sequences made as RNA baits (by, for example, IVT methods). Even so, the structure present in the rRNA target can render their capture inefficient. In this case, hybridization in buffers which normalize A:T vs. G:C base pair binding strength may be beneficial, such as tetramethyl ammonium chloride (TMAC) based buffer systems. Hybridization can also be driven to favor capture by providing the DNA capture baits at higher concentrations than the rRNA targets.

It is efficient to tile DNA baits end-to-end with no overlap for DNA exon capture purposes. DNA baits were designed using design criteria in place for design of IDT xGen® Lockdown® Probe DNA exon capture products. Appendix 2 shows sequences of the rRNA capture set using this approach and in Appendix 3 an edited set which eliminates domains having GC content >85%. It is expected that the probe set will improved synthesis quality and improved performance if probe GC content is kept at 85% or less. Appendix 4 shows sequences of the rRNA capture set designed using a 2× overlap strategy and in Appendix 5 an edited 2× overlap set which eliminates domains having GC content >85%. The 2× overlap set will likely show slightly higher clearance of rRNA, but it may not be necessary to use the extra probes present in this set.

The capture baits shown in Appendices 2-5 employ 120 nucleotide oligomers with a single 5′-biotin modification. This design has proven to be very effective as a tool for capture enrichment of target DNA sequences for NGS sequencing application; one version of this strategy is currently sold as Lockdown® Probes by Integrated DNA Technologies, Inc. (Coralville, Iowa (US)). For the capture-enrichment sequencing application, achieving high target specificity is highly desired; if target capture is less than 100% or less than 90% or less than 80%, and so on, there is little impact on the quality of NGS sequence data output. For the new rRNA clearance application of the present invention, achieving high capture efficiency is highly desired. Therefore capture rates above 80% are preferred, above 90% are more preferred, and approaching 100% are most preferred. Chemical synthesis of long capture baits is done preferably using a high efficiency synthesis platform, such as the Ultramer® manufacturing system in place at Integrated DNA Technologies, Inc. where coupling efficiency of each sequential base addition averages 99.5% or higher. Even with this very high coupling efficiency, a 120 nucleotide oligomer will be, on average, around 55% full-length product with the remaining 45% comprising all possible truncation products, most of which will be 5′-end capped via synthesis capping chemistry and will therefore not have a 5′-biotin ligand. These truncation failure products can hybridize to target RNA (e.g., rRNA) and can also remain as excess unhybridized oligomers. In either case, the oligomers lacking a biotin ligand will not be captured and cleared and therefore will remain in the RNA pool which is used to make an NGS sequencing library, making capture efficiency lower than desired and/or contributing directly to contamination of the NGS library. Use of purification methods, such as HPLC or PAGE, could be used to increase purity of the bait DNAs, however use of such methods adds to manufacturing time and cost and reduces yield.

As an alternative to purification, employing shorter baits, such as 60 nucleotide length, will result in oligomers having around 75% full-length product, leaving 25% truncated products lacking a 5′-biotin. Therefore making capture baits in this size range may be preferable to the longer 120 nucleotide capture baits for the present capture-removal application used in target enrichment methods. Even shorter baits can be employed, so long as hybridization conditions are adjusted for the lower binding affinity expected from shorter baits. Chemical modifications can be incorporated into the shorter baits to increase binding affinity and allow for use of bait pools having mixed sequence lengths so help normalize Tₘ between baits (e.g., 40 nucleotide baits, 60 nucleotide baits, and 120 nucleotide baits in a single pool, used in a single hybridization mixture under identical conditions).

As a further consideration, it is possible that some DNA baits used in rRNA capture/clearance, especially those baits which are not biotin end-modified, could remain in the RNA sample after SA-magnetic bead capture. Such sequences could become incorporated into the downstream NGS library and contribute to unwanted background sequencing reads. This possibility can be eliminated if the DNA baits are 3′-end blocked, preventing adaptor ligation and subsequent participation in library construction steps. An improved bait design would therefore comprise a “medium length” synthetic oligonucleotide, such as a 60 nucleotide oligomer (within a 40-80 nucleotide range is preferred) having both a 5′- and a 3′-biotin, or other capture ligand. This design provides a 3′-end block (e.g., the 3′-biotin group) and also has double biotin modification, which will ensure that almost all or all bait DNAs will have at least a single capture ligand present, maximizing clearance of bound rRNA molecules while at the same time preventing participation of residual DNA baits in NGS library construction. A set of 60 nucleotide dual-biotin DNA capture baits for rRNA clearance is shown in Appendix 6.

Other exemplary RNA targets that can be selected for removal according to the methods described herein include mRNAs encoding ribosomal RNA proteins (see Appendix 7). Appendix 8 shows sequences of the ribosomal protein mRNA capture set using the method of the present invention. Yet other exemplary RNA targets include highly abundant mRNAs encoding globins found in red blood cells (see Appendix 9). Appendix 10 shows sequences of the globin mRNA capture set using the method of the present invention.

Certain pol III transcripts like tRNA are considered as undesired RNA species owing to their abundance in total RNA. Yet the removal of tRNA from a total RNA population is customarily unnecessary for RNA-seq experiments in NGS applications, likely because the highly modified tRNA sequences are inefficiently copied into NGS libraries. Clearance of tRNA species is nevertheless included in the scope of the present invention, and may be of particular utility if downstream applications include sequencing methods that include short RNA fragments within this size range.

Further advantages are afforded by the use of baits whose structure and activity have been verified according to a standardized product specification with a quality control procedure. Though other procedures are available for preparing baits, it is preferable to prepare as a capture reagent a composition that includes a plurality of baits (that is, a set of discrete bait oligonucleotides), wherein each member of the plurality of baits is prepared individually.

As used in this context, the number of members of the plurality of bait oligonucleotides includes ranges from about 10 to about 100, from about 10 to about 1000, and from 10 to about 10,000. This range naturally varies with the application and the number and size of RNA species targeted for clearance. Even larger size bait sets, such as 10,000 to 100,000 or more, are commonly employed in positive selection methods, where the captured sequences are retained for downstream applications. Smaller bait sets, such as falling within ranges from about 10 to about 100, from about 10 to about 1000, and from 10 to about 10,000 are commonly employed in negative selection methods, where the captured sequences are discarded and the cleared sample is retained for downstream applications.

More preferably, each member of the plurality of baits is individually synthesized by a chemical process, wherein the quality of the product can be monitored during synthesis, after synthesis, and after optional purification. Even more preferably, each member of the plurality is prepared by a synthetic chemical process and purified, wherein both the quality of the synthesis and purification can be independently assessed. Most preferably, each member of the plurality of baits has an independent product specification from other members of the plurality of baits so that the plurality of baits can be obtained, wherein the structure and activity of each member is normalized relative to other members within the plurality of baits. The use of a plurality of baits having normalized activity enables more complete and uniform coverage of a given target of interest, particularly for targets having high GC-content regions. These advantages can be realized for oligonucleotide baits of all types.

Oligonucleotides that serve as baits include at least one modification that enables selection of bait:undesired RNA hybrids from the population of RNA templates 101 during hybrid capture. One example of a preferred modification includes biotin that can be incorporated into the oligonucleotide bait during chemical synthesis and used with solid support media containing or coupled to avidin or streptavidin for hybrid selection. Other capture ligands can be employed, such as digoxigenin or other groups as are well known to those with skill in the art.

### Nucleic Acid Samples

A variety of tissue samples can be the source of the nucleic acid samples used in the present methods. Total RNA can be isolated from a biological sample (for example, a tumor sample, a normal adjacent tissue (NAT), a blood sample, a sample containing circulating tumor cells (CTC) or any normal control)). In certain embodiments, the biological sample can be preserved as a frozen sample or as formaldehyde- or paraformaldehyde-fixed paraffin-embedded (FFPE) tissue preparation. For example, the sample can be embedded in a matrix, for example, an FFPE block or a frozen sample. The isolating step can include flow-sorting of individual chromosomes; and/or micro-dissecting a subject's sample (for example, a tumor sample, a NAT, a blood sample).

Protocols for RNA isolation are disclosed, for example, in the Maxwell® 16 Total RNA Purification Kit Technical Bulletin (Promega Literature #TB351, August 2009) and in the BiooPure RNA Isolation Reagent instruction manual (Bioo Scientific cat #5301). A widely used method for RNA isolation is disclosed in U.S. Pat. No. 4,843,155, Chomczynski P, “Product and process for isolating RNA” (1989).

The isolated nucleic acid samples (for example, total RNA samples) can be fragmented or sheared by practicing routine techniques. For example, genomic DNA can be fragmented by physical shearing methods, enzymatic cleavage methods, chemical cleavage methods, and other methods well known to those skilled in the art. For NGS RNA-Seq applications, typically intact total RNA is employed, optionally treated for enrichment using poly-T selection for poly-A RNA species or rRNA negative selection as taught herein, cDNA is made from the RNA, and shearing is done on the double-stranded cDNA species. Fragmentation may also be carried out on the input RNA prior to cDNA synthesis, for example using chemical fragmentation. The nucleic acid library can contain all or substantially all of the complexity of the transcriptome. The term “substantially all” in this context refers to the possibility that there can in practice be some unwanted loss of transcriptome complexity during the initial steps of the procedure. The methods described herein also are useful in cases where the nucleic acid library is a portion of the transcriptome, that is, where the complexity of the transcriptome is reduced by design. In some embodiments, any selected portion of the transcriptome can be selected for removal and clearance with the methods described herein.

Methods featured in the invention can further include isolating a nucleic acid sample to provide a library (for example, a nucleic acid library as described herein). For example, the nucleic acid sample used to generate the library includes RNA or cDNA derived from RNA. In some embodiments, the RNA includes total cellular RNA. In other embodiments, certain abundant RNA sequences (for example, ribosomal RNAs) have been depleted. In some embodiments, the poly(A)-tailed mRNA fraction in the total RNA preparation has been enriched. In some embodiments, the cDNA is produced by random-primed cDNA synthesis methods. In other embodiments, the cDNA synthesis is initiated at the poly(A) tail of mature mRNAs by priming by oligo(dT)-containing oligonucleotides. Methods for depletion, poly(A) enrichment, and cDNA synthesis are well known to those skilled in the art.

The method can further include amplifying the nucleic acid sample by specific or non-specific nucleic acid amplification methods that are well known to those skilled in the art. In some embodiments, certain embodiments, the nucleic acid sample is amplified, for example, by whole-genome amplification methods such as random-primed strand-displacement amplification.

The nucleic acid sample used to generate the library can also include RNA or cDNA derived from RNA. In some embodiments, the RNA includes total cellular RNA. In other embodiments, certain abundant RNA sequences (for example, ribosomal RNAs) have been depleted. In other embodiments, the poly(A)-tailed mRNA fraction in the total RNA preparation has been enriched. In some embodiments, the cDNA is produced by random-primed cDNA synthesis methods. In other embodiments, the cDNA synthesis is initiated at the poly(A) tail of mature mRNAs by priming by oligo(dT)-containing oligonucleotides. Methods for depletion, poly(A) enrichment, and cDNA synthesis are well known to those skilled in the art.

The method can further include amplifying the nucleic acid sample by specific or non-specific nucleic acid amplification methods that are known to those skilled in the art. The nucleic acid sample can be amplified, for example, by whole-genome amplification methods such as random-primed strand-displacement amplification.

The nucleic acid sample can be fragmented or sheared by physical or enzymatic methods as described herein, and ligated to synthetic adaptors, size-selected (for example, by preparative gel electrophoresis) and amplified (for example, by PCR). The fragmented and adaptor-ligated group of nucleic acids is used without explicit size selection or amplification prior to hybrid selection.

### Hybridization Conditions

The methods featured in the present invention include the step of contacting the target sample (for example, a total RNA sample, an NGS library, or other heterogeneous mixture) with a plurality of baits to first hybridize to unwanted RNA species and then remove unwanted captured RNA species. The contacting step can be effected in solution hybridization. In certain embodiments, the method includes repeating the hybridization step by one or more additional rounds of solution hybridization. In some embodiments, the methods further include subjecting the library hybridization/capture to one or more additional rounds of solution hybridization with the same or different collection of baits.

Variations in efficiency of selection can be adjusted by altering the concentration of the baits and the composition of the hybridization solution. In one embodiment, the efficiency of selection is adjusted by leveling the efficiency of individual baits within a group (for example, a first, second or third plurality of baits) by adjusting the relative abundance of the baits, or the density of the binding entity (for example, the hapten or affinity tag density) in reference to differential sequence capture efficiency observed when using an equimolar mix of baits, and then introducing a differential excess as much of internally-leveled group 1 to the overall bait mix relative to internally-leveled group 2.

In certain embodiments, the methods described herein can achieve high coverage of the sequences targeted for removal. In one embodiment, the percent of target bases complementary to bait probes is about 50%, or about 60%, or about 70%, or about 80%, or about 90%, or about 100%. Regions of a target nucleic acid not directly complementary to bait probes can be depleted so long as said regions are linked (e.g. are an adjacent sequence) to a target sequence complementary to a bait. This feature of the system can assist with capture depletion of targets such as the human 28S rRNA without having to provide 100% coverage of the target in the bait pool. This target has local regions with >85% GC content and these areas are prone to form highly stable secondary structures which are difficult to invade for probe hybridization. Further, these sequences can also be difficult for chemical synthesis. Making a probe pool that excludes these regions can improve quality of the bait set and yet still result in efficient capture of the entire target.

Prior to hybridization, baits can be denatured according to methods well known in the art.

In general, hybridization steps include contacting DNA bait composition under hybridizing conditions with the target sequences to be removed and depleting those sequences after hybridization/binding of the bait composition to the target.

Baits are hybridized or annealed to the target sequences under hybridizing conditions. “Hybridizing conditions” are conditions that facilitate annealing between a bait and a nucleic acid target. Since annealing of different baits will vary depending on probe length, base composition and the like, annealing is facilitated by varying bait concentration, hybridization temperature, salt concentration and other factors well known in the art.

Varying parameters, such as the concentrations, base compositions, complexities, and lengths of the baits, the tiling extents, as well as salt concentrations, temperatures, and length of incubation, facilitates identification of optimal hybridization conditions. For example, hybridizations can be performed in hybridization buffer containing 5×SSPE, 5×Denhardt's solution, 5 mM EDTA and 0.1% SDS and blocking DNA to suppress non-specific hybridization. Alternatively, hybridization can be performed in 5×SSC. In other embodiments, the hybridizations can be performed in a buffer containing tetramethyl ammonium chloride (TMAC), such as are well known to those with skill in the art. One embodiment of the invention involves use of an optimized hybridization buffer that minimizes the Tₘ difference between oligonucleotides of different sequence. In the buffer system described herein, hybridization is regulated more by the length of matched base pairs present between probe and target such that the effects of mismatches are magnified while variations in sequence which do not contribute to mismatch are minimized. In one embodiment, the hybridization buffer is a combination of Tris at a pH around 8; EDTA; Sarkosyl; Ovalbumin; CTAB; Ficoll Type 400; PVP-360; tetramethyl ammonium chloride (TMAC); and blocking DNA; optionally, formamide can be added to adjust optimal hybridization temperature. In another embodiment, the composition of the hybridization buffer is: 37.5 mM Tris pH 8, 3 mM EDTA, 0.25% Sarkosyl, 0.4 mg/mL Ovalbumin, 1 mM CTAB, 0.4 mg/mL Ficoll Type 400, 0.4 mg/mL PVP-360, 2.5M TMAC, 10 μg/mL denatured/sheared salmon sperm DNA; optionally, formamide can be added up to a final concentration of 50%. See also methods disclosed by Goldrick for hybridization and capture buffer compositions and protocols (US Patent Application, Goldrick et al., METHODS AND COMPOSITIONS FOR IMPROVING REMOVAL OF RIBOSOMAL RNA FROM BIOLOGICAL SAMPLES, US 2014/0295418).

In general, hybridization conditions, as described above, include incubation for periods ranging from about 10 minutes to about 30 minutes to about 1 hour to about 4 hours to about 24 hours at temperatures ranging from about 20° C. (for example RT) to about 70° C., more typically about 60° C., depending on the precise composition of the hybridization buffer. Hybridization can optionally be performed in sequential steps where incubation temperature is shifted or ramped between temperatures. For example, a hybridization can be performed for 10 minutes at 60° C. followed by 15 minutes at 37° C.

Marked RNAs can be used to assess the efficiency of selection and removal of undesired RNAs. A marked RNA can be prepared that corresponds to the unmarked RNA species targeted to a DNA bait set. The marked RNA can include a label to enable its detection in the total RNA sample before and after hybridization to the DNA bait set and removal using a suitable capture reagent directed to the DNA bait affinity tag. A total RNA sample can be spiked with a known amount of the marked RNA. The extent of selection and removal of the unmarked RNA can be assessed by quantitating the respective amounts of marked RNA present in the captured RNA fraction as compared to the non-captured RNA fraction (that is collective fraction that includes the supernatant and post-bead wash fractions). Thus, different empirical parameters can be rapidly assessed to identify specific conditions that yield efficient hybrid selection and removal of the undesired RNA species.

The methods described herein are adaptable to standard liquid handling methods and devices. In some embodiments, the method is carried out using automated liquid handling technology as is known in the art, such as devices that handle multiwell plates (see for example, Gnirke, A. et al. (2009) Nat Biotechnol. 27(2):182-189). This can include, but not limited to, automated library construction, and steps of solution hybridization including set-up and post-solution hybridization washes. For example, an apparatus can be used for carrying out such automated methods for the bead-capture step after the solution hybridization reaction. Exemplary apparatus can include, but is not limited to, the following positions: a position for a multi-well plate containing streptavidin-coated magnetic beads, a position for the multiwall plate containing the solution hybrid-selection reactions, I/O controlled heat blocks to preheat reagents and to carry out hybridization and/or washing steps at a user-defined temperature, a position for a rack of pipet tips, a position with magnets laid out in certain configurations that facilitate separation of supernatants from magnet-immobilized beads, a washing station that washes pipet tips and disposed of waste, and positions for other solutions and reagents. In one embodiment, the apparatus is designed to process up to 96 depletions including the bait+RNA hybridization step, the streptavidin bead-capture step, through the final desired RNA clean-up and concentration step in parallel. In another embodiment, one or more positions have a dual function. In yet another embodiment, the user is prompted by the protocol to exchange one plate for another. In such automated systems, the devices are configured to capture the post-streptavidin bead capture supernatant fraction for further collection and processing, as the non-captured RNA includes the desired RNA species of the present method. In yet another embodiment, the automated system is configured to insert a magnet into the vessel containing the solution hybridization reaction and the affinity-coated magnetic beads (for example, streptavidin-coated magnetic beads), for the purpose of attracting said magnetic beads, wherein the magnetic beads are linked through the affinity group to the bait oligonucleotides containing a capture moiety (for example, biotin), and wherein a subset of the bait oligonucleotides are hybridized to nucleic acid targeted for removal (for example, ribosomal RNA).

In one embodiment, the selected subgroup of nucleic acids are amplified (for example, by PCR) prior to being analyzed by sequencing or genotyping. In another embodiment, the subgroup is analyzed without an amplification step, for example, when the selected subgroup is analyzed by sensitive analytical methods that can read single molecules.

### Sequencing

Any method of sequencing can be used. Sequencing of nucleic acids isolated by selection methods are typically carried out using next-generation sequencing (NGS). Next-generation sequencing includes any sequencing method that determines the nucleotide sequence of either individual nucleic acid molecules or clonally expanded proxies for individual nucleic acid molecules in a highly parallel fashion (for example, greater than 10⁵ molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, for example, in Metzker, M. (2010) Nature Reviews Genetics 11:31-46, incorporated herein by reference.

In one embodiment, the next-generation sequencing allows for the determination of the nucleotide sequence of an individual nucleic acid molecule (for example, Helicos BioSciences' HeliScope Gene Sequencing system, and Pacific Biosciences' PacBio RS system). In other embodiments, the sequencing method determines the nucleotide sequence of clonally expanded proxies for individual nucleic acid molecules (for example, the Solexa sequencer, Illumina Inc., San Diego, Calif.; 454 Life Sciences (Branford, Conn.), and Ion Torrent). For example, massively parallel short-read sequencing (for example, the Solexa sequencer, Illumina Inc., San Diego, Calif.), which generates more bases of sequence per sequencing unit than other sequencing methods that generate fewer but longer reads. Other methods or machines for next-generation sequencing include, but not limited to, the sequencers provided by 454 Life Sciences (Branford, Conn.), Applied Biosystems (Foster City, Calif.; SOLiD sequencer), Helicos BioSciences Corporation (Cambridge, Mass.), and emulsion and microfluidic sequencing technology nanodroplets (for example, GnuBio droplets).

Platforms for next-generation sequencing include, but are not limited to, Roche/454's Genome Sequencer (GS) FLX System, Illumina/Solexa's Genome Analyzer (GA), Life/APG's Support Oligonucleotide Ligation Detection (SOLiD) system. Polonator's G.007 system, Helicos BioSciences' HeliScope Gene Sequencing system, and Pacific Biosciences' PacBio RS system.

NGS technologies can include one or more of steps, for example, template preparation, sequencing and imaging, and data analysis.

Additional exemplary sequencing methodologies are known in the art, for example, some of which are described in commonly owned, U.S. Ser. No. 13/339,986 and PCT/US11/67725, both filed on Dec. 29, 2011, the contents of which are incorporated by reference.

### EXAMPLES

The present invention is additionally described by reference to the following Examples, which are offered by way of illustration and are not intended to limit the invention in any manner. Standard techniques well known in the art or techniques specifically described below can be utilized.

### Example 1

### Hybridization of DNA Baits to Total RNA

The present example demonstrates hybridization of the synthetic DNA capture baits to rRNA present in a total RNA sample.

Five samples were prepared, each containing 1 g of total RNA extracted from cultured human cells (HEK293T) using the BiooPure RNA Isolation reagent (Bioo Scientific Corp., Austin, Tex. (US)) modified to allow the RNA to be recovered using solid phase extraction onto magnetic beads. The RNA was hybridized as described in the specification to different amounts of a mixture of human rRNA biotinylated bait oligonucleotides (Appendix 6), each of which was 60 nucleotides in length with biotin modification at both the 3′- and 5′-ends. The final bait pool reagent contained a final concentration of 100 μm oligonucleotide capture baits comprising 0.87 μM of each of the 106 somatic rRNA specific baits and 0.17 μM of each of the 42 mitochondrial rRNA specific baits (Appendix 6). The final amount of pooled oligonucleotide baits was 1.9 μg per μL of bait mixture. The amounts of bait mixture used in each hybridization are shown in Table 1.

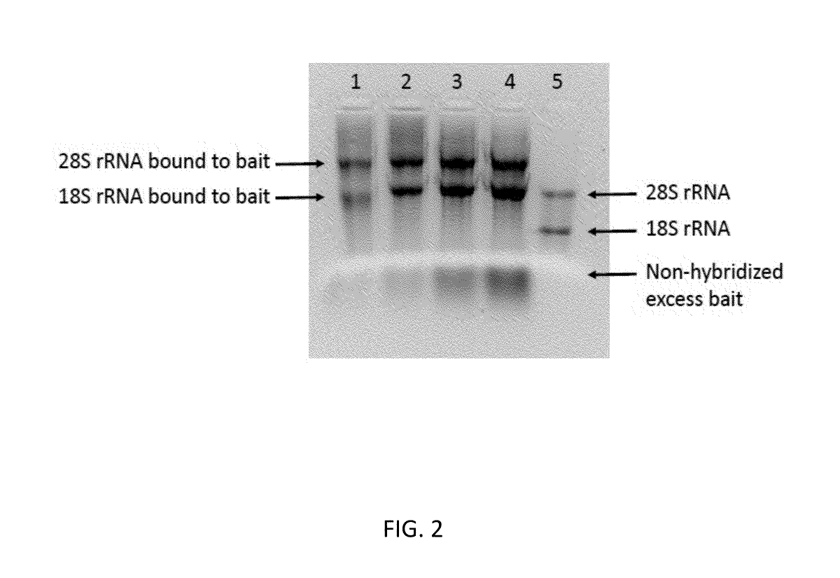

TABLE 1
Amounts of rRNA baits used in capture/gelshift assays

| Total human RNA | Bait mixture | Lane on gel (FIG. 2) |
|---|---|---|
| 1 μg | 0.25 μL | 1 |
| 1 μg | 0.5 μL | 2 |
| 1 μg | 1.0 μL | 3 |
| 1 μg | 1.5 μL | 4 |
| 1 μg | — | 5 |

The RNA samples and bait pool were combined into a total final volume of 30 μL of a hybridization solution containing 10 mM Tris pH 8, 400 mM NaCl and incubated for 10 minutes at 60° C. and then for 15 minutes at 37° C.

Following hybridization, the nucleic acid species were separated on a 2% agarose gel prepared and run in the presence of ethidium bromide to allow staining and detection of the RNA (FIG. 2). The control sample in lane 5 shows the positions of the 18S and 28S ribosomal RNA bands in the absence of hybridization to bait. The remaining lanes show an upward shift in mobility of the 18S and 28S bands after hybridization to the bait pool corresponding to the increase in molecular weight of the rRNA:bait complex compared with native rRNA. The increased intensity of bands in hybridized samples reflects the increased binding of ethidium bromide to double-stranded nucleic acid compared to single-stranded nucleic acid (in this case the rRNA:DNA heteroduplexes). The diffuse low molecular weight material in lanes 3 and 4 comprises excess unhybridized bait. The gel image shows the rRNA bands are maximally up-shifted using 0.5 μL of the bait pool per 1 μg total RNA. As bait concentration is further increased, no additional upward molecular weight shift is observed and excess non-hybridized low molecular weight baits are seen at the bottom of the gel. This example demonstrates efficient hybridization of baits to the rRNA target is achieved under conditions employed and that 0.5 μL of the bait pool is sufficient to fully bind the rRNA present in 1 lag total human RNA.

### Example 2

### Removal of rRNA:bait Complexes Using Streptavidin (SA) Magnetic Beads

The present example demonstrates clearance of rRNA:bait complexes from a total RNA sample using magnetic SA beads.

Six samples were prepared, each containing 1 μg of total RNA extracted from cultured human cells (HEK293T) using the BiooPure RNA Isolation reagent (Bioo Scientific Corp., Austin, Tex. (US)) modified to allow the RNA to be recovered using solid phase extraction onto magnetic beads. The RNA was hybridized as described in the specification to different amounts of a mixture of human rRNA biotinylated bait oligonucleotides (Appendix 6), each of which was 60 nucleotides in length with biotin modification at both the 3′- and 5′-ends. The final bait pool reagent contained a final concentration of 100 μM oligonucleotide capture baits comprising 0.87 μM of each of the 106 somatic rRNA specific baits and 0.17 μM of each of the 42 mitochondrial rRNA specific baits (Appendix 6). The final amount of pooled oligonucleotide baits was 1.9 μg per μL of bait mixture. The amounts of bait mixture used in each hybridization are shown in Table 2.

TABLE 2
Amounts of rRNA baits used in SA-magnetic bead reactions

| | | | Lane |
|---|---|---|---|
| Total human RNA | Bait mixture | SA magnetic beads | on gel (FIG. 2) |
| 1 μg | 0.25 μL | 20 μL | 1 |
| 1 μg | 0.25 μL | 30 μL | 2 |
| 1 μg | 0.5 μL | 20 μL | 3 |
| 1 μg | 0.5 μL | 30 μL | 4 |
| 1 μg | 0.25 μL | — | 5 |
| 1 μg | 0.5 μL | — | 6 |

The RNA samples and bait pool were combined into a total final volume of 30 μL of a hybridization solution containing 10 mM Tris pH 8, 400 mM NaCl and incubated for 10 minutes at 60° C. and then for 15 minutes at 37° C.

Prior to use, streptavidin magnetic beads obtained from Solulink (NanoLink Streptavidin magnetic beads, cat #M-1002) were prepared by adding 20 (or 30) μL of well-mixed beads to 0.5 mL of Bead Wash Solution (150 mM NaCl/5 mM Tris pH 7.5/2 mM EDTA), vortex mixed, then attracted to a magnet by placing the vessel containing the beads and wash solution in contact with said magnet for 1 minute and removing the fluid without disturbing the beads on the vessel wall. The vessel was removed from the magnetic stand and the beads resuspended in 20 μL of Bead Hybridization Solution, said Bead Hybridization Solution having a composition disclosed in U.S. Patent Application Publication US20140295418 to Goldrick et al., “METHODS AND COMPOSITIONS FOR IMPROVING REMOVAL OF RIBOSOMAL RNA FROM BIOLOGICAL SAMPLES.” In a preferred embodiment, the composition of said Bead Hybridization Solution was 300 mM NaCl, 10 mM MgCl₂, 5% Polyethylene Glycol mw 8000. Components of the Bead Hybridization Solution may be obtained from Sigma Chemical Co.

At the end of the hybridization period, 20 tμL of prepared magnetic beads (prepared from 20 μL or 30 μL of Nanolink beads) in Bead Hybridization Solution were added to the vessel containing the 30 μL hybridization reaction and, after vortex mixing, the reaction was incubated at room temperature for 15 minutes to allow binding to occur between the biotin on the hybridized template:bait complexes and the streptavidin on the beads. Following the incubation period, the streptavidin beads along with biotinylated bait oligos and associated nucleic acid hybridized to the bait, were removed by inserting a rod magnet into the reaction vessel. One suitable rod magnet is a neodymium-iron-boron rare-earth magnet that is 25.4 mm in length and having a diameter of 3.2 mm (Magcraft, Vienna, Va.; part #NSNO750/N40). For ease of handling, one end of the rod magnet was connected to a pipet tip by inserting it into the narrow end of a standard P-200 tip. The rod magnet was inserted into the vessel to a level of about 1 mm-2 mm beneath the surface of the reaction components, for a duration of about 5 seconds. This interval is sufficient to allow the magnetic beads and associated reaction components to be attracted to the tip of the rod magnet. The rod magnet was then withdrawn from the vessel, removing the SA-magnetic beads and bound rRNA:bait complexes, leaving the desired RNA not targeted for removal in the vessel. The magnetic beads and associated components were removed from the rod magnet by wiping the tip of the magnet with a tissue (for example a KimWipe), in order to re-use the rod magnet for processing subsequent samples. After wiping the rod magnet to remove the beads, the rod magnet was further cleaned by rinsing in ethanol.

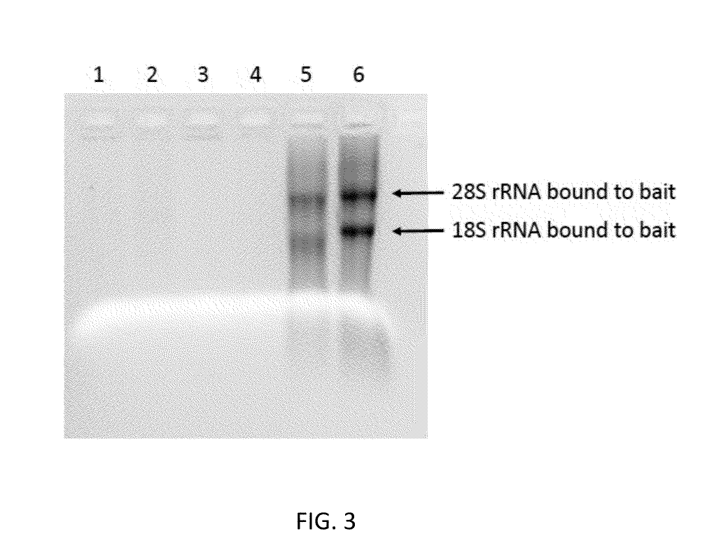

Following removal of the SA-magnetic beads and rRNA:bait complexes, the remaining nucleic acid species were separated on a 1% agarose gel prepared and run in the presence of ethidium bromide to allow staining and detection of the RNA (FIG. 3). The control samples in lanes 5 and 6 did not undergo SA-magnetic bead binding and show the positions of the 18S and 28S ribosomal RNA bands complexed to the biotinylated bait oligonucleotides. Lanes 1-4 show the remaining RNA left after rRNA removal by bait capture. Essentially, no RNA is visible in these lanes, consistent with near total elimination of the rRNA species. The remaining mRNA (spread over a molecular weight range of <500 to >10,000 nucleotides) is not visible when using this detection method when starting with the low input amount of total RNA employed. This example demonstrates efficient removal of the rRNA target is achieved under conditions employed and that 20 μL of the SA-magnetic beads is sufficient to fully bind 0.5 μL of bait pool.

### Example 3

### Depletion of rRNA from RNA-Seq Libraries

The present example demonstrates clearance of rRNA from RNA-Seq libraries using biotinylated baits and SA-magnetic beads.

Total RNA was extracted from cultured human cells (HEK293T) using the BiooPure RNA Isolation reagent (Bioo Scientific Corp., Austin, Tex. (US)). The RNA (1 μg or 3 μg) was hybridized as described in the specification to 3 μL of a mixture of human rRNA biotinylated bait oligonucleotides (Appendix 3), each of which was 120 nucleotides in length biotin with 5′-biotin. The final bait pool reagent contained equimolar amounts of capture oligonucleotides complementary to human cytoplasmic rRNA species at a concentration of 27 μM (approximately 1 mg per mL) and mitochondrial ribosomal RNAs at 1/10 this concentration, 2.7 μM (approximately 0.1 mg per mL). The amounts of bait mixture used in each hybridization were are shown in Table 2. A 3 μg control RNA samples was mock treated, meaning it was processed through the method without the addition of capture baits to the hybridization mixture.

The RNA samples and bait pool were combined into a total final volume of 50 μL of a hybridization solution containing 10 mM Tris pH 8, 400 mM NaCl and incubated for 10 minutes at 60° C. and then for 20 minutes at room temperature.

Streptavidin magnetic beads (Solulink NanoLink Streptavidin magnetic beads, cat #M-1002) were prepared as described in Example 2. Each of the 3 RNA samples were mixed with 35 μL of SA-magnetic beads and incubated for 15 minutes at room temperature. The beads were attracted to a magnet for 4 minutes and liquid was removed to a fresh tube. The fluid from samples that had been hybridized to biointylated baits should be enriched for mRNA and depleted of rRNA while the fluid from the mock-treated sample should contain total RNA, including the undesired rRNA. The samples were then treated with DNase by combining each with 15 μL of 10× DNase buffer (0.2 M Tris pH 8, 20 mM MgCl₂, 10 mM CaCl₂), 48 μL water, and 2 μL DNase 1 (Sigma cat#D5319, ˜5,000 Kunitz units/mg protein) and incubated for 20 minutes at 37° C. The DNase was then inactivated by combining each sample with 8 μL of 0.5 M EDTA and incubating for 5 minutes at 70° C. The treated samples were purified by combining each with 220 μL magnetic beads (Omega Bio-tek Mag-Bind EZ Pure), incubating 10 minutes at room temperature, attracting the beads to magnet and removing fluid, washing the beads twice with 0.5 ml 75% ethanol, and eluting the bound RNA by resuspending the beads in 50 μL of 0.1 mM EDTA, storing for 2 minutes at room temperature, attracting to a magnet for 2 minutes, and transferring the fluid to a fresh tube. The RNA was then used as input for making RNA-Seq libraries using the NEXTflex nondirectional RNASeq kit (Bioo Scientific Corp. cat #5129). The libraries were amplified for 15 cycles of PCR.

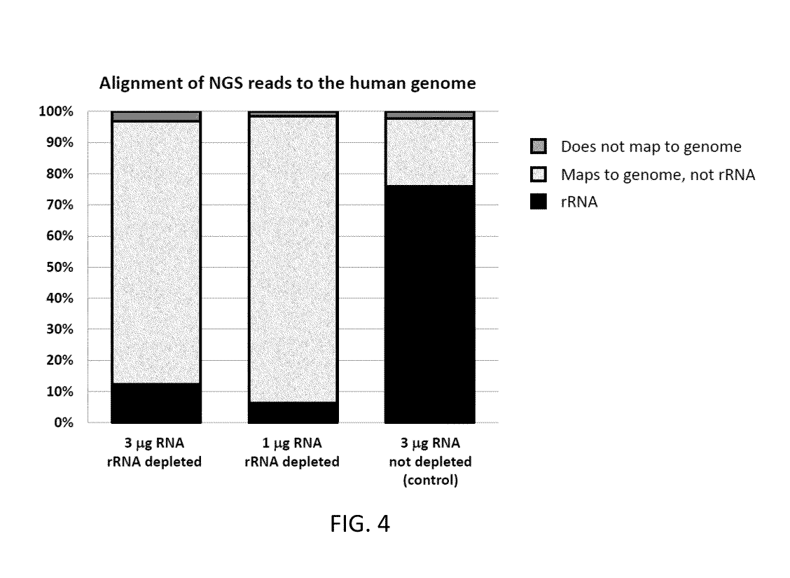

The 3 NGS libraries (3 μg depleted, 1 μg depleted, and 3 μg control non-depleted) were pooled and sequenced on an Illumina MiSEQ instrument using the V2 kit with 75×75 cycles. Sample identity was tracked by bar codes (CTTGTA, ATCACG, and TTAGGC) using established methods. Reads were mapped to the human genome and binned into 3 categories: 1) rRNA sequence, 2) human genome, not rRNA, and 3) does not map to the human genome. Results are shown in Table 3 and are graphically plotted in FIG. 4.

TABLE 3
Results of rRNA clearance from NGS RNA-Seq libraries

| | 3 μg RNA | 1 μg RNA | 3 μg RNA |
|---|---|---|---|
| | depleted | depleted | not depleted |
| rRNA | 12% | 6% | 76% |
| Human, not rRNA | 85% | 92% | 22% |
| unmapped | 3% | 2% | 2% |

Consistent with expectation, RNA-Seq performed on untreated human total RNA showed a large fraction of the sequencing reads mapped to rRNA genes with only 22% of reads representing useful sequence. In contrast, the 1 μg depleted sample showed 92% useful sequencing reads and the 3 g depleted sample showed 85% useful sequencing reads. The higher amount of residual rRNA present in the 3 μg depleted sample relative to the 1 μg depleted sample suggests that the amount of bait employed was insufficient for clearing rRNA sequences from the larger amount of total RNA. Better results would be expected if additional bait was used, in a similar ratio to that employed in the 1 μg depleted sample.

### Example 4

### Depletion of rRNA from Total RNA without DNase Treatment

Many protocols employ a DNase treatment to eliminate residual DNA capture baits and prevent these sequences from contaminating downstream PCR or NGS assays. The 60 nucleotide dual-biotin baits (Appendix 6) should have minimal risk to give false signals in such downstream applications, due to their shorter length and chemical end-blocking groups. The present example demonstrates a DNase-free processing method.

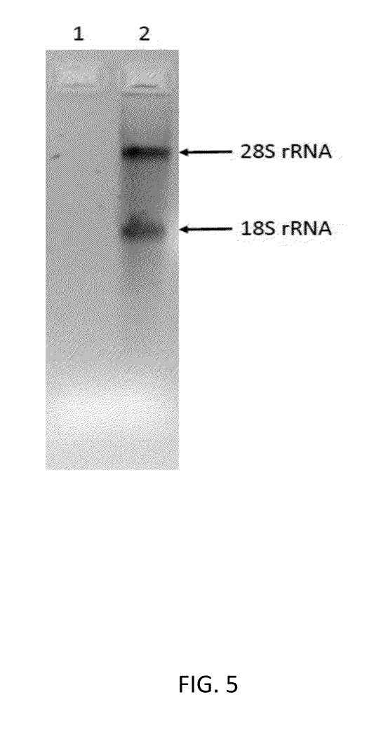

Total human cellular RNA samples (2 μg) were hybridized with 1 μL of the 60-nucleotide dual-biotin bait pool (see Examples 1 and 2, sequences from Appendix 6) in oligo hybridization buffer (400 mM NaCl, 10 mM Tris pH 8) in a final volume of 30 μL for 10 minutes at 60° C. and then for 15 minutes at 37° C. A control mock-hybridized preparation was assembled and treated in the same way, except that bait probes were not added. Each reaction was then individually mixed with 30 μL of prepared streptavidin-conjugated magnetic beads (NanoLink™ beads, Solulink). Beads were prepared by vortexing in 0.5 ml of Bead Wash (150 mM NaCl, 5 mM Tris pH 7.5, 2 mM EDTA), attracting to a magnet for 2 minutes, removing the wash solution, and resuspending the bead pellet in 30 μL of Bead Hyb solution (300 mM NaCl, 16% PEG 8000). The reactions were incubated at room temp for 15 minutes without agitation (no agitation was necessary since the beads remained suspended), then the reactions were placed on a magnetic stand for 3 minutes to concentrate the bead and the fluid removed. Half of each sample was separated on a 2% agarose gel with ethidium bromide and visualized by UV-induced fluorescence. The gel image is shown in FIG. 5. Lane 1 shows the sample which underwent rRNA clearance and no evidence for remaining rRNA is seen. Other cellular RNAs are present (such as mRNAs), but are not visualized using this approach due to the low amount of material present (see detection of GAPDH mRNA in Example 5). Lane 2 shows the mock-treated sample, which shows the rRNA present in total RNA and also demonstrates that the procedure does not degrade the RNA.

### Example 5

### Depletion of rRNA from Total RNA Measured by RT-PCR

The present example demonstrates clearance of rRNA from total RNA assessed using RT-PCR assays for human cytoplasmic and mitochondrial rRNA using the DNase-free processing method.

Total human cellular RNA (2 μg) was hybridized with 1 μL of the 60-nucleotide dual-biotin bait pool (see Examples 1 and 2, sequences from Appendix 6) in oligo hybridization buffer (400 mM NaCl, 10 mM Tris pH 8) in a final volume of 30 μL for 10 minutes at 60° C. and then for 15 minutes at 37° C. A control mock-hybridized preparation was assembled and treated in the same way, except that the bait probes were not added. Each reaction was then individually mixed with 30 j±L of prepared streptavidin-conjugated magnetic beads (NanoLink™ beads, Solulink). Beads were prepared by vortexing in 0.5 ml of Bead Wash (150 mM NaCl, 5 mM Tris pH 7.5, 2 mM EDTA), attracting to a magnet for 2 minutes, removing the wash solution, and resuspending the bead pellet in 30 μL of Bead Hyb solution (300 mM NaCl, 16% PEG 8000). The reactions were incubated at room temp for 15 minutes without agitation (no agitation was necessary since the beads remained suspended), then the reactions were placed on a magnetic stand for 3 minutes to concentrate the bead and the fluid removed.

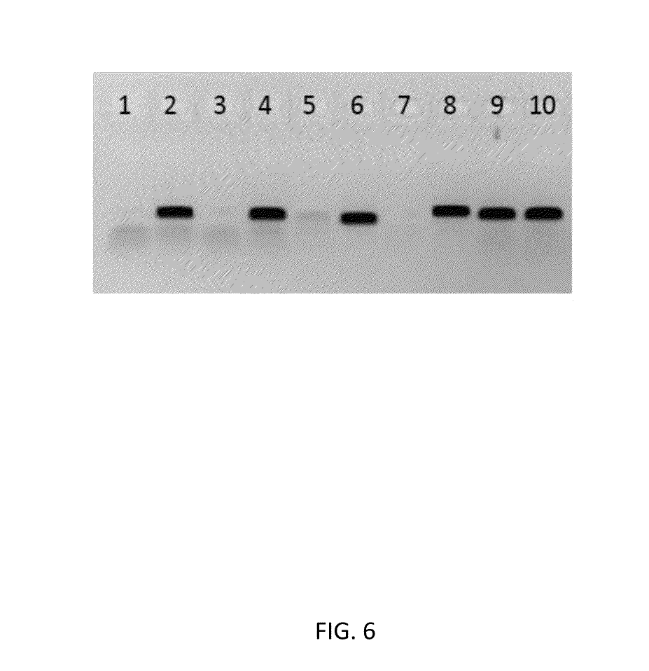

Aliquots (5 μL) of the rRNA-depleted and mock-depleted samples were converted to cDNA by reverse transcription using M-MLV reverse transcriptase in 20 μL reactions according to standard protocols. The reactions were diluted with 90 μL of water; 4 μL of each diluted reverse-transcription reactions was used as template for PCR reactions (20 μL) with primers indicated in Table 4. The reactions that employed rRNA primers were run for 20 cycles. The reactions that employed GAPDH primers were run for 28 cycles. The PCR reactions were separated on a 2% agarose gel in the presence of ethidium bromide and visualized by UV-induced fluorescence. A gel image is shown in FIG. 6. Reactions are summarized, including primer sequences, in Table 4.

TABLE 4
RT-PCR reactions efficiency
of rRNA clearance

| FIG. | | | | | |
|---|---|---|---|---|---|
| 5 | Human | | | | SEQ |
| gel | total | Bait | | Primer | ID |
| lane | RNA | Pool | Target | Seq | No. |
| 1 | 2 μg | 1 μL | Mitochondrial | 12S For: AGACCCA | 1 |
| | | | 12S rRNA | AACTGGGATTAGATAC | |
| 2 | 2 μg | — | | 12s Rev: TTAAGCT | 2 |
| | | | | GTGGCTCGTAGTG | |
| 3 | 2 μg | 1 μL | Mitochondrial | 16S For: AAAGAGC | 3 |
| | | | 16S rRNA | ACACCCGTCTATG | |
| 4 | 2 μg | — | | 16S Rev: TCTTGGA | 4 |
| | | | | CAACCAGCTATCAC | |
| 5 | 2 μg | 1 μL | Human | 18S For: GCGGTAA | 5 |
| | | | 18S rRNA | TTCCAGCTCCAATAG | |
| 6 | 2 μg | — | | 18S Rev: CCGCTCC | 6 |
| | | | | CAAGATCCAACTA | |
| 7 | 2 μg | 1 μL | Human | 28S For: CGTCGTG | 7 |
| | | | 28S rRNA | AGACAGGTTAGTTT | |
| 8 | 2 μg | — | | 28S Rev: CCTCAGC | 8 |
| | | | | CAAGCACATACA | |
| 9 | 2 μg | 1 μL | GAPDH | GAPDH For: ACACC | 9 |
| | | | | CACTCCTCCACCTTT | |
| 10 | 2 μg | — | | GAPDH Rev: TGCTG | 10 |
| | | | | TAGCCAAATTCGTTG | |

Using RT-PCR, it is clear that the rRNA clearance method of the present invention largely removed rRNA from a total RNA sample. The four rRNA-specific RT-PCR assays show little if any detectable residual rRNA amplicon (lanes 1, 3, 5, and 7) compared to the strong amplicon seen with mock-depletion (lanes 2, 4, 6, and 8). In comparison, an RT-PCR assay specific for GAPDH mRNA shows no difference between depleted (lane 9) and mock-depleted (lane 10) samples, consistent with the depletion method removing rRNA with little to no effect on other cellular RNA species.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the world wide web at ncbi.nlm.nih.gov.

The terminology used herein is for the purpose of describing particular embodiments only, and is not intended to be limiting. With respect to the use of substantially, any plural and/or singular terms herein, those having skill in the art can translate from the plural as is appropriate to the context and/or application. The various singular/plural permutations may be expressly set forth herein for the sake of clarity.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiments or examples disclosed, but that the present invention will include all embodiments falling within the scope of the appended claims.

APPENDIX 1
rRNA Sequences to use for capture bait design

Homo sapiens RNA, 28S ribosomal 5 (RNA28S5), ribosomal RNA (SEQ ID NO: 11)
NCBI Reference Sequence: NR_003287.2

| LOCUS | NR_003287 5070 bp rRNA linear PRI 16-JUL-2013 |
|---|---|
| DEFINITION | Homo sapiens RNA, 28S ribosomal 5 (RNA28S5), ribosomal RNA. |
| ACCESSION | NR_003287 |
| VERSION | NR_003287.2 GI: 225637499 |
| KEYWORDS | RefSeq. |
| SOURCE | Homo sapiens (human) |
| 1 | cgcgacctca gatcagacgt ggcgacccgc tgaatttaag catattagtc agcggaggag |
| 61 | aagaaactaa ccaggattcc ctcagtaacg gcgagtgaac agggaagagc ccagcgccga |
| 121 | atccccaccc cgcagcgggg cgcgggacat gtggcatacg gaagacccgc tacccggcac |
| 181 | cgctcgtggg gggcccaagt ccttctgatc gaggcccagc ccatggacgg tgtgaggccg |
| 241 | gtagcagccc ccagcgcgcc gggcccgggt cttcccggag tcgggttgct tgggaatgca |
| 301 | gcccaaagcg ggtggLaaac tccatctaag gctaaatacc ggcacgagac cgatagtcaa |
| 361 | caagtaccgt aagggaaagt tgaaaagaac tttgaagaga gagttcaaga gggcgtgaaa |
| 421 | ccattaagag gtaaacgagt ggggtccgcg cagtccgccc ggaggattca acccagcggc |
| 481 | ggatccggcc gtgtcggcgg cccgacggat ctttcccgcc ccccgttcct cccgacccct |
| 541 | ccacccgccc tcccttcccc cgccgcccct cctcctcctc cccggagggg gcgggctccg |
| 601 | gcgggtgcgg gggtgggcgg gcggggccgg gggtggggtc ggcgggggac cgtcccccga |
| 661 | ccggcgaccg gccgccgccg ggcgcatttc caccgcggcg gtgcgccgcg accggctccg |
| 721 | ggacggctgg gaaagcccgg cggggaagat ggctcagggg gccccgtccg tccgtccgtc |
| 781 | cgtcctcctc ctcccccgtc tccgcccccc ggccccgcgt cctccctcgg gagggcgcgc |
| 841 | gggtcggggc ggcggcggcg gcggcggtgg cggcggcggc ggcggcggcg ggaccgaaac |
| 901 | cccccccgag tgttacagcc cccccggcag cagcactcgc cgaatcccgg ggccgaggga |
| 961 | gcgagacccg tcgccgcgct ctcccccctc ccggcgccca cccccgcggg gaatcccccg |
| 1021 | cgaggggggt ctcccccacg ggggcgcgcc gacgtctcct cgtgggaggg ccggaccacc |
| 1081 | cctcccacgg cgcgaccact ctcccacccc tcctccccgc gcccccaccc cggcaacggg |
| 1141 | gggggtgccg cgcgcgagtc gggaagcggg gcggactgtc cccagtgcgc cccgggcggg |
| 1201 | tcgcgccgtc gggcccgggg gaggttctct cggggccacg cgcgcgtccc ccgaagaggg |
| 1261 | ggacggcgga gcgagcgcac ggggtcggcg gcgacgtcgg ctacccaccc gacccgtctt |
| 1321 | gaaacacgga ccaaggagtc taacacgtac gcgagtcggg ggctcgcacg aaagccgccg |
| 1381 | tggcgcaatg aagatgaagg ccggcgcgct cgccgaccga ggtgggatcc cgaggcctct |
| 1441 | ccagtccgcc gagggcgcac caccggcccg tctcacccgc cgcgccgggg aggtggagca |
| 1501 | cgagcgcacg tgttaggacc cgaaagatgg tgaactatgc ctgggcaggg cgaagccaga |
| 1561 | ggaaactctg gtggaggtcc gtagcggtcc tgacgtgcaa atcggtcgtc cgacctgggt |
| 1621 | ataggggcga aagactaatc gaaccatcta gtagctggtt ccctccaaag tttccctcag |
| 1681 | gatagctggc gctctcgcag acccaacgca cccccgccac gcagttttat ccggtaaagc |
| 1741 | gaatgattag aggtcttggg gccgaaacga tctcaaccta ttctcaaact ttaaatgggt |
| 1801 | aagaagcccg gctcgctggc gtggagccgg gcgtggaatg cgagtgccta gtgggccact |
| 1861 | tttggtaagc agaactggcg ctgcgggatg aaccgaacgc cgggttaagg cgcccgatgc |
| 1921 | cgacgctcat cagaccccag aaaaggtgtt ggttgatata gacagcagga cggtggccat |
| 1981 | ggaagtcgga atccgctaag gagtgtgtaa caactcacct gccgaatcaa ctagccctga |
| 2041 | aaatggatgg cgctggagcg tcgggcccat acccggccgt caccggcagt cgagagtgga |
| 2101 | cgggagcggc gggggcggcg cgcgcgcgcg cgcgtgtggt gtgcgtcgga gggcggcggc |
| 2161 | ggcggcggcg gcgggggtgt ggggtccttc ccccgccccc ccccccacgc ctcctcccct |
| 2221 | cctcccgccc acgccccact ccccaccccc gaagccccgc ggacgctacg ccgcaacgag |
| 2281 | taagagggcc gctgcggtga gccttgaagc ctagggcgca ggcccgagtg gagccgccgc |
| 2341 | aggtgcagat cttggtggta gtagcaaata ttcaaacgag aactttgaag gccgaagtgg |
| 2401 | agaagggttc catgtgaaca gcagttgaac atgggtcagt cggtcctgag agatgggcga |
| 2461 | gcgccgttcc gaagggacgg gcgatggcct ccgttgccct cggccgatcg aaagggagtc |
| 2521 | gggttcagat ccccgaatcc ggagtggcgg agatgagcgc cgcgaggcgt ccagtgcgat |
| 2581 | aacgcgaccg atcccggaga agccggcgag agccccgggg agagttctct tttctttgtg |
| 2641 | aagggcaggg cgccctggaa tgggttcgcc ccgagagagg ggcccgtgcc ttggaaagcg |
| 2701 | tcgcggttcc ggcggcatcc ggtaagctct cgctggccct tgaaaatccg ggggagaggg |
| 2761 | tgtaaatctc gcgccgggcc gtacccatat ccgcagcagg tctccdaggt gaacagcctc |
| 2821 | tggcatgttg gaacaatgta ggtaagggaa gtcggcaagc cggatccgta acttcgggat |
| 2881 | aaggattggc tctaagggct gggtcggtcg ggctggggcg cgaagcgggg ctgggcgcgc |
| 2941 | gccgcaactg gacgaggcgc cgccgccccc cccacgcccg gggcaccccc ctcgcggccc |
| 3001 | tcccccgccc caccccgcgc gcgccgctcg ctccctcccc gccccgcgcc ctctctctct |
| 3061 | ctctctcccc cgctccccgt cctcccccct ccccgaggga gcgccgcgtg ggggcggcgg |
| 3121 | cggggggaga agggtcgggg cggcaggggc cggcggcggc ccgccgcggg gccccggcgg |
| 3181 | cgggggcacg gtcccccgcg aggggggccc gggcacccgg ggggccggcg gcggcggcga |
| 3241 | ctctggacgc gagccgggcc cttcccgtgg atcgccccag ctgcggcggg cgtcgcggcc |
| 3301 | gcccccgggg agcccggcgg gcgccggcgc gccccccccc ccaccccacg tctcgtcgcg |
| 3361 | cgcgcgtccg ctgggggcgg ggagcggtcg ggcggcggcg gtcggcgggc ggcggggcgg |
| 3421 | ggcggttcgt ccccccgccc tacccccccg gccccgtctg ccccccgttc ccccctcctc |
| 3481 | ctcggcacgc ggcagcggcg acggcaggcg gcggaagggc cgcgggccgg tcccccccac |
| 3541 | cgggtccgcc cccagggccg cggttccgcg cggcgcctcg cctcggccgg cgcctagcag |
| 3601 | ccgacttaga actagtgcgg accaggggaa tccgactgtt taattaaaac aaagcatcgc |
| 3661 | gaaggcccgc ggcgggtgtt gacgcgatgt gatttctgcc cagtgctctg aatgtcaaag |
| 3721 | tgaagaaatt caatgaagcg cgggtaaacg gcgggagtaa ctatgactct cttaaggtag |
| 3781 | ccaaatgcct cgtcatctaa ttagtgacgc gcatgaatga atgaacaaga ttcccaetgt |
| 3841 | ccctacctac tatccagcga aaccacagcc aagggaacgg gcttgacgga atcaacgggg |
| 3901 | aaagaagacc ctgttgagct tgactctagt ctggcacggt gaagagacat gagaggtgta |
| 3961 | gaataagtgg gaggcccccg gcgccccccc ggtgtcccca cgaggggccc ggggcggggt |
| 4021 | ccgccggccc tgcgggccgc cggtgaaata ccactactct gatcgttttt tcactgaccc |
| 4081 | ggtgagacgg gggagcgagc cccgagggac tctcgcttct ggcgccaagc acccggccac |
| 4141 | gcgccggccg ggcacgaccc actccgggga cagtgccagg tggggagttt gactggggcg |
| 4201 | gtacacctgt caaacggtaa cgcaggtgtc ctaaggcgag ctcagggagg acagaaacct |
| 4261 | cccgtggagc agaagggcaa aagctcgctt gatcttgatt ttcagtacga atacagaccg |
| 4321 | tgaaagcggg gcctcacgat ccttctgacc ttttgggttt taagcaggag gtgtcagaaa |
| 4381 | agttaccaca gggataactg gcttatggcg gccaagcgtt catagcaacg tcgctttttg |
| 4441 | atccttcgat gtcggctctt cctatcattg tgaagcagaa ttcaccaagc gttggattgt |
| 4501 | tcacccacta atagggaacg tgagctgggt ttagaccgtc gtgagacagg ttagttttac |
| 4561 | cctactgatg atgtgttatt gccatggtaa tcctgctcag tacgagagga accgcaggtt |
| 4621 | cagacatttg gtgtatgtgc ttggctgagg agccaatggg gcgaagctac catctgtggg |
| 4681 | attatgactg aacacctcta agtcagaatc ccgcccaggc ggaacgatac agcagcgccg |
| 4741 | cggagcctcg gttggcctcg gatagccggt cccccgcctg tccccgccgg cgggccgccc |
| 4801 | cccccctcca cgcgccccgc gcgcgcggga gggcgcgtgc cccgccgcgc gccgggaccg |
| 4861 | gggtccagtg cggagtgccc ttcgtcctgg gaaacggggc gcggccggag aggcggccgc |
| 4921 | cccctcgccc gtcacgcacc gcacgttcgt ggggaacctg gcgctaaacc attcgtagac |
| 4981 | gacctgcttc tgggtcgagg tttcatacgt aacagagcaa ctccctcgct gcgatctatt |
| 5041 | gaaagtcagc cctcgacaca agggtttgtc |

Homo sapiens RNA, 18S ribosomal 5 (RNA18S5), ribosomal RNA (SEQ ID NO: 12)
NCBI Refernce Sequence: NP_003286.2

| | |
|---|---|
| LOCUS | NR_003286 1869 bp rRNA linear PRI 16-JUL-2013 |
| DEFINITION | Homo sapiens RNA, 18S ribosomal 5 (RNA18S5), ribosomal RNA. |
| ACCESSION | NR_003286 |
| VERSION | NR_003286.2 GI: 225637497 |
| KEYWORDS | RefSeq. |
| SOURCE | Homo sapiens (human) |
| 1 | tacctggttg atcctgccag tagcatatgc ttgtctcaaa gattaagcca tgcatgtctg |
| 61 | agtacgcacg gccggtacag tgaaactgcg aatggctcat taaatcagtt atggttcctt |
| 121 | tggtcgctcg ctcctctcct acttggataa ctgtggtaat tctagagcta atacatgccg |
| 181 | acgggcactg acccccttcg cgggggggat gcgtgcattt atcagatcaa aaccaacccg |
| 241 | gtcagcccct ctccggcccc ggccgggggg cgggcgccgg cggctttggt gactctagat |
| 301 | aacctcgggc cgatcgcacg ccccccgtgg cggcgacgac ccattcgaac gtctgcccta |
| 361 | tcaactttcg atggtagtcg ccgtgcctac catggtgacc acgggtgacg gggaatcaag |
| 421 | gttcgattcc ggagagggag cctgagaaac ggctaccaca tccaaggaag gcagcaggcg |
| 481 | cgcaaattac ccactcccga cccggggagg tagtgacgcc aaataacaat acaggactct |
| 541 | ttcgaggccc tgtaattgga atgagtccac tttaaatcct ttaacgagga tccattggag |
| 601 | ggcaagtctg gtgccagcag ccgcggtaat tccagctcca atagcgtata ttaaagttac |
| 661 | tgcagttaaa aagctcgtag ttggatcttg ggagcaggcg gacggtccgc cgcgaggcga |
| 721 | gccaccgccc gtccccgccc cttgcctctc ggcgccccct cgatgctctt agctgagtgt |
| 781 | cccgcggggc ccgaagcgtt tactttgaca aaattagagt gttcaaagca ggcccgagcc |
| 841 | gcctggatac cgcagctagg aataatggaa taggaccgcg gttctatttt gttggttttc |
| 901 | ggaactgagg ccatgattaa gaggaacggc caggggcatt cgtattacgc cgctagaggt |
| 961 | gaaattcttg gaccggcgca agacggacca gagcgaaagc atttgccaag aatgttttca |
| 1021 | ttaatcaaga acgaaagtcg gaggttcgaa gacgatcaga taccgtcgta gttccgacca |
| 1081 | taaacgatgc cgaccggcga tgcggcggcg ttattcccat gacccgccgg gcagcttccg |
| 1141 | ggaaaccaaa gtctttgggt tccgggggga gtatggttgc aaagctgaaa cttaaaggaa |
| 1201 | ttgacggaag ggcaccacca agagtggaac ctgcgactta atttgactca acacgggaaa |
| 1261 | cctcacccgg cccggacacg gacaggattg acagattgat agctctttct cgattccgtg |
| 1321 | ggtggtggtg catggccgtt cttagttggt ggagcgattt gtctggttaa ttccgataac |
| 1381 | gaacgagact ctggcatgct aactagttac gcgacccccg agcggtcggc gtccccccac |
| 1441 | ttcttagagg gacaagtggc gttcagccac ccgagattga gcaataacag gtctgtgatg |
| 1501 | cccttagatg tccggggctg cacgcgcgct acactgacta gctcagcgtg tgcctaccct |
| 1561 | acgccggcag gcgcgggtaa cccgttgaac cccattcgtg atggggatcg gggattgcaa |
| 1621 | ttattcccca tgaacgagga attcccagta agtgcgggtc ataagcttgc gttgattaag |
| 1681 | tccctgccct ttgtacacac cgcccgtcgc tactaccgat tggatggttt agtgaggccc |
| 1741 | tcggatcggc cccgccgggg tcggcccacg gccctggcgg agcgctgaga agacggtcga |
| 1801 | acttgactat ctaaaggaag taaaagtcat aacaaagttt ccgtaggtga acctgcggaa |
| 1861 | ggatcatta |

Homo sapiens RNA, 5S ribosomal RNA (SEQ ID NO: 13)
>gi|189571632|ref|NR_023379.1| Homo sapiens RNA, 5S ribosomal 17 (RNA5S17),
ribosomal RNA
GTCTACGGCCATACCACCCTGAACGCGCCCGATCTCGTCTGATCTCGGAAGCTAAGCAGGGTCGGGCCTG
GTTAGTACTTGGATGGGAGACCGCCTGGGAATACCGGGTGCTGTAGGCTTT
Homo sapiens RNA, 5.8S ribosomal RNA (SEQ ID NO: 14)

| | |
|---|---|
| LOCUS | NR_003285 156 bp rRNA linear PRI 16-JUL-2013 |
| DEFINITION | Homo sapiens DNA, 5.8S ribosomal 5 (RNA5-8S5), ribosomal RNA. |
| ACCESSION | NR_003285 |
| VERSION | NR_003285.2 GI: 142372596 |
| KEYWORDS | RefSeq. |
| SOURCE | Homo sapiens (human) |
| 1 | gactcttagc ggtggatcac tcggctcgtg cgtcgatgaa gaacgcagct agctgcgaga |
| 61 | attaatgtga attgcdggac acattgatca tcgacacttc gaacgcactt gcggccccgg |
| 121 | gttcctcccg gggctacgcc tgtctgagcg tcgctt |
| LOCUS | KF899911 16572 bp DNA circular PRI 08-DEC-2013 |
| DEFINITION | Homo sapiens haplogroup Ilala mitochondrion, complete genome. |
| ACCESSION | KF899911 |
| VERSION | KF899911.1 GI: 562889938 |
| SOURCE | mitochondrion Homo sapiens (human) |

Human Mitchondrial 163 rRNA (SEQ ID NO: 15)

| | |
|---|---|
| | gctaaa |
| 1681 | cctagcccca aacccactcc accttactac cagacaacct taaccaaacc atttacccaa |
| 1741 | ataaagtata ggcgatagaa attgaaacct ggcgcaatag atatagtacc gcaagggaaa |
| 1801 | gatgaaaaat tataaccaag cataatatag caaggactaa cccctatacc ttctgcataa |
| 1861 | tgaattaact agaaataact ttgcaaggag agccaaagct aagacccccg aaaccagacg |
| 1921 | agctacctaa gaacagctaa aagagcacac ccgtctatgt agcaaaatag tgggaagatt |
| 1981 | tataggtaga ggcgacaaac ctaccgagcc tggtgatagc tggttgtcca agatagaatc |
| 2041 | ttagttcaac tttaaatttg cccacagaac cctctaaatc cccttgtaaa tttaactgtt |
| 2101 | agtccaaaga ggaacagctc tttggacact aggaaaaaac cttgtagaga gagtaaaaaa |
| 2161 | tttaacaccc atagtaggcc taaaagcagc caccaattaa gaaagcgttc aagctcaaca |
| 2221 | cccactacct aaaaaatccc aaacatataa ctgaactcct cacacccaat tggaccaatc |
| 2281 | tatcacccta tagaagaact aatgttagta taagtaacat gaaaacattc tcctccgcat |
| 2341 | aagcctgcgt cagattaaaa cactgaactg acaattaaca gcccaatatc tacaatcaac |
| 2401 | caacaagtca ttattaccct cactgtcaac ccaacacaga catgctcata aggaaaggtt |
| 2461 | aaaaaaagta aaaggaactc ggcaaatctt accccgcctg tttaccaaaa acatcacctc |
| 2521 | tagcatcacc agtattagag gcaccgcctg cccagtgaca catgtttaac ggccgcggta |
| 2581 | ccctaaccgt gcaaaggtag cataatcact tgttccttaa atagggacct gtatgaatgg |
| 2641 | ctccacgagg gttcagctgt ctcttacttt taaccagtga aattgacctg cccgtgaaga |
| 2701 | ggcgggcatg acacagcaag acgagaagac cctatagagc tttaatttat taatgcaaac |
| 2761 | agtacctaac aaacccacag gtcctaaact accaaacctg cattaaaaat ttcggttggg |
| 2821 | gcgacctcgg agcagaaccc aacctccgag cagtacatgc taagacttca ccagtcaaag |
| 2881 | cgaactacta tactcaattg atccaataac ttgaccaacg gaacaagtta ccctagggat |
| 2941 | aacagcgcaa tcctattcta gagtccatat caacaatagg gtttacgacc tcgatgttgg |
| 3001 | atcaggacat cccgatgatg cagccgctat taaaggttcg tttgttcaac gattaaagtc |
| 3061 | ctacgtgatc tgagttcaga ccggagtaat ccaggttggt ttctatctac ttcaaattcc |
| 3121 | tccctgtacg aaaggacaag agaaataagg cctacttcac aaagcgcctt cccccgtaaa |
| 3181 | tgatatcatc tcaacttagt attataccca cacccaccca agaacagggt tt |

Human Mitchondrial 12S rRNA (SEQ ID NO: 16)

| | |
|---|---|
| | a ataggtttgg |
| 661 | tcctagcctt tctattagct ttcagtaaga ttacacatgc aagcatcccc gttccagtga |
| 721 | gttcaccctc taaatcacca cgatcaaaag ggacaagcat caagcacgca gcaatgcagc |
| 781 | tcaaaacgct tagcctagcc acacccccac gagaaacagc agtgattaac ctttagcaat |
| 841 | aaacgaaagt ttaactaagc tatactaacc ccagggttgg tcaatttcgt gccagccacc |
| 901 | gcggtcacac gattaaccca agtcaataga agccggcgta aagagtgttt tagatcaccc |
| 961 | cctccccaat aaagctaaaa ctcacctgag ttgtaaaaaa ctccagttga cacaaaatag |
| 1021 | actacgaaag tggctttaac atatctgaac acacaatagc taagacccaa actgggatta |
| 1081 | gataccccac tatgcttagc cctaaacctc aacagttaaa tcaacaaaac tgctcgccag |
| 1141 | aacactacga gccacagctt aaaactcaaa ggacctggcg gtgcttcata tccctctaga |
| 1201 | ggagcctgtt ctgtaatcga taaaccccga tcaacctcac cacctcttgc tcagcctata |
| 1261 | taccgccatc ttcagcaaac cctgatgaag gctacaaagt aagcgcaagt acccacgtaa |
| 1321 | agacgttagg tcaaggtgta gcccatgagg tggcaagaaa tgggctacat tttctacccc |
| 1381 | agaaaactac gatagccctt atgaaactta aaggtcgaaa gtggatttag cagtaaactg |
| 1441 | agagtagagt gcttagttga acagggccct gaagcgcgta cacaccgccc gtcaccctcc |
| 1501 | tcaagtatac ttcaaaggac atttaactaa aacccctacg catttatata gaggagacaa |
| 1561 | gtcgtaacat ggtaagtgta ctggaaagtg cacttggacg aa |

APPENDIX 2
120-mer DNA rRNA Capture Probes/
Baits at 1x coverage (Note: /5Biosg/ = 5′-biotin)

| Name | | |
|---|---|---|
| (SEQ ID NO:_) | Sequence | GC content |
| KF899911.1_12SrRNA_r1_1 | /5Biosg/GCGTGCTTGATGCTTGTCCCTTTTGATCGTGGTGATTTAGAGG | 0.45 |
| (SEQ ID NO: 17) | GTGAACTCACTGGAACGGGGATGCTTGCATGTGTAATCTTACTAAGAGCTA | |
| | ATAGAAAGGCTAGGACCAAACCTATT | |
| KF899911.1_12SrRNA_r1_2 | /5Biosg/GAAATTGACCAACCCTGGGGTTAGTATAGCTTAGTTAAACTTT | 0.45 |
| (SEQ ID NO: 18) | CGTTTATTGCTAAAGGTTAATCACTGCTGTTTCCCGTGGGGGTGTGGCTAG | |
| | GCTAAGCGTTTTGAGCTGCATTGCTG | |
| KF899911.1_12SrRNA_r1_3 | /5Biosg/ACTGGAGTTTTTTACTACTCAGGTGAGTTTTAGCTTTATTGGG | 0.483333333 |
| (SEQ ID NO: 19) | GAGGGGGTGATCTAAAACACTCTTTACGCCGGCTTCTATTGACTTGGGTTA | |
| | ATCGTGTGACCGCGGTGGCTGGCACG | |
| KF899911.1_12SrRNA_r1_4 | /5Biosg/TGTTGATTTAACTGTTGAGGTTTAGGGCTAAGCATAGTGGGGT | 0.383333333 |
| (SEQ ID NO: 20) | ATCTAATCCCAGTTTGGGTCTTAGCTATTGTGTGTTCAGATATGTTAAAGC | |
| | CACTTTCGTAGTCTATTTTGTGTCAA | |
| KF899911.1_12SrRNA_r1_5 | /5Biosg/GAGGTGGTGAGGTTGATCGGGGTTTATCGATTACAGAACAGGC | 0.508333333 |
| (SEQ ID NO: 21) | TCCTCTAGAGGGATATGAAGCACCGCCAGGTCCTTTGAGTTTTAAGCTGTG | |
| | GCTCGTAGTGTTCTGGCGAGCAGTTT | |
| KF899911.1_12SrRNA_r1_6 | /5Biosg/GCCCATTTCTTGCCACCTCATGGGCTACACCTTGACCTAACGT | 0.5 |
| (SEQ ID NO: 22) | CTTTACGTGGGTACTTGCGCTTACTTTGTAGCCTTCATCAGGGTTTGCTGA | |
| | AGATGGCGGTATATAGGCTGAGCAAG | |
| KF899911.1_12SrRNA_r1_7 | /5Biosg/TGTGTACGCGCTTCAGGGCCCTGTTCAACTAAGCACTCTACTC | 0.45 |
| (SEQ ID NO: 23) | TCAGTTTACTGCTAAATCCACCTTCGACCCTTAAGTTTCATAAGGGCTATC | |
| | GTAGTTTTCTGGGGTAGAAAATGTAG | |
| KF899911.1_12SrRNA_r1_8 | /5Biosg/GTTCGTCCAAGTGCACTTTCCAGTACACTTACCATGTTACGAC | 0.441666667 |
| (SEQ ID NO: 24) | TTGTCTCCTCTATATAAATGCGTAGGGGTTTTAGTTAAATGTCCTTTGAAG | |
| | TATACTTGAGGAGGGTGACGGGCGGT | |
| KF899911.1_16SrRNA_r1_1 | /5Biosg/TTGCGGTACTATATCTATTGCGCCAGGTTTCAATTTCTATCGC | 0.425 |
| (SEQ ID NO: 25) | CTATACTTTATTTGGGTAAATGGTTTGGTTAAGGTTGTCTGGTAGTAAGGT | |
| | GGAGTGGGTTTGGGGCTAGGTTTAGC | |
| KF899911.1_16SrRNA_r1_2 | /5Biosg/GTTTCGGGGGTCTTAGCTTTGGCTCTCCTTGCAAAGTTATTTC | 0.375 |
| (SEQ ID NO: 26) | TAGTTAATTCATTATGCAGAAGGTATAGGGGTTAGTCCTTGCTATATTATG | |
| | CTTGGTTATAATTTTTCATCTTTCCC | |
| KF899911.1_16SrRNA_r1_3 | /5Biosg/ATCTTGGACAACCAGCTATCACCAGGCTCGGTAGGTTTGTCGC | 0.466666667 |
| (SEQ ID NO: 27) | CTCTACCTATATATCTTCCCACTATTTTGCTACATAGACGGGTGTGCTCTT | |
| | TTAGCTGTTCTTAGGTAGCTCGTCTG | |
| KF899911.1_16SrRNA_r1_4 | /5Biosg/CTCTCTCTACAAGGTTTTTTCCTAGTGTCCAAAGAGCTGTTCC | 0.375 |
| (SEQ ID NO: 28) | TCTTTGGACTAACAGTTAAATTTACAAGGGGATTTAGAGGGTTCTGTGGGC | |
| | AAATTTAAAGTTGAACTAAGATTCTA | |
| KF899911.1_16SrRNA_r1_5 | /5Biosg/CCAATTGGGTGTGAGGAGTTCAGTTATATGTTTGGGATTTTTT | 0.391666667 |
| (SEQ ID NO: 29) | AGGTAGTGGGTGTTGAGCTTGAACGCTTTCTTAATTGGTGGCTGCTTTTAG | |
| | GCCTACTATGGGTGTTAAATTTTTTA | |
| KF899911.1_16SrRNA_r1_6 | /5Biosg/GTAGATATTGGGCTGTTAATTGTCAGTTCAGTGTTTTAATCTG | 0.366666667 |
| (SEQ ID NO: 30) | ACGCAGGCTTATGCGGAGGAGTATGTTTTCATGTTACTTATACTAACATTA | |
| | GTTCTTCTATAGGGTGATAGATTGGT | |
| KF899911.1_16SrRNA_r1_7 | /5Biosg/TGTTTTTGGTAAACAGGCGGGGTAAGATTTGCCGAGTTCCTTT | 0.4 |
| (SEQ ID NO: 31) | TACTTTTTTTAACCTTTCCTTATGAGCATGCCTGTGTTGGGTTGACAGTGA | |
| | GGGTAATAATGACTTGTTGGTTGATT | |
| KF899911.1_16SrRNA_r1_8 | /5Biosg/TACAGGTCCCTATTTAAGGAACAAGTGATTATGCTACCTTTGC | 0.5 |
| (SEQ ID NO: 32) | ACGGTTAGGGTACCGCGGCCGTTAAACATGTGTCACTGGGCAGGCGGTGCC | |
| | TCTAATACTGGTGATGCTAGAGGTGA | |
| KF899911.1_16SrRNA_r1_9 | /5Biosg/TTAATAAATTAAAGCTCCATAGGGTCTTCTCGTCTTGCTGTGT | 0.466666667 |
| (SEQ ID NO: 33) | CATGCCCGCCTCTTCACGGGCAGGTCAATTTCACTGGTTAAAAGTAAGAGA | |
| | CAGCTGAACCCTCGTGGAGCCATTCA | |
| KF899911.1_16SrRNA_r1_10 | /5Biosg/TGGTGAAGTCTTAGCATGTACTGCTCGGAGGTTGGGTTCTGCT | 0.483333333 |
| (SEQ ID NO: 34) | CCGAGGTCGCCCCAACCGAAATTTTTAATGCAGGTTTGGTAGTTTAGGACC | |
| | TGTGGGTTTGTTAGGTACTGTTTGCA | |
| KF899911.1_16SrRNA_r1_11 | /5Biosg/GAGGTCGTAAACCCTATTG7TGATATGGACTCTAGAATAGGAT | 0.408333333 |
| (SEQ ID NO: 35) | TGCGCTGTTATCCCTAGGGTAACTTGTTCCGTTGGTCAAGTTATTGGATCA | |
| | ATTGAGTATAGTAGTTCGCTTTGACT | |
| KF899911.1_16SrRNA_r1_12 | /5Biosg/AAGTAGATAGAAACCGACCTGGATTACTCCGGTCTGAACTCAG | 0.458333333 |
| (SEQ ID NO: 36) | ATCACGTAGGACTTTAATCGTTGAACAAACGAACCTTTAATAGCGGCTGCA | |
| | CCATCGGGATGTCCTGATCCAACATC | |
| KF899911.1_16SrRNA_r1_13 | /5Biosg/AAACCCTGTTCTTGGGTGGGTGTGGGTATAATACTAAGTTGAG | 0.441666667 |
| (SEQ ID NO: 37) | ATGATATCATTTACGGGGGAAGGCGCTTTGTGAAGTAGGCCTTATTTCTCT | |
| | TGTCCTTTCGTACAGGGAGGAATTTG | |
| NR_003286.2_RNA18S5_r1_1 | /5Biosg/AAGGAACCATAACTGATTTAATGAGCCATTCGCAGTTTCACTG | 0.458333333 |
| (SEQ ID NO: 38) | TACCGGCCGTGCGTACTCAGACATGCATGGCTTAATCTTTGAGACAAGCAT | |
| | ATGCTACTGGCAGGATCAACCAGGTA | |
| NR_003286.2_RNA18S5_r1_2 | /5Biosg/GTTGGTTTTGATCTGATAAATGCACGCATCCCCCCCGCGAAGG | 0.533333333 |
| (SEQ ID NO: 38) | GGGTCAGCGCCCGTCGGCATGTATTAGCTCTAGAATTACCACAGTTATCCA | |
| | AGTAGGAGAGGAGCGAGCGACCAAAG | |
| NR_003286.2_RNA18S5_r1_3 | /5Biosg/GACGTTCGAATGGGTCGTCGCCGCCACGGGGGGCGTGCGATCG | 0.741666667 |
| (SEQ ID NO: 39) | GCCCGAGGTTATCTAGAGTCACCAAAGCCGCCGGCGCCCGCCCCCCGGCCG | |
| | GGGCCGGAGAGGGGCTGACCGGGTTG | |
| NR_003286.2_RNA18S5_r1_4 | /5Biosg/CTTCCTTGGATGTGGTAGCCGTTTCTCAGGCTCCCTCTCCGGA | 0.575 |
| (SEQ ID NO: 40) | ATCGAACCCTGATTCCCCGTCACCCGTGGTCACCATGGTAGGCACGGCGAC | |
| | TACCATCGAAAGTTGATAGGGCAGAC | |
| NR_003286.2_RNA18S5_r1_5 | /5Biosg/CGTTAAAGGATTTRAAGTGGACTCATTCCRATTACAGGGCCTC | 0.491666667 |
| (SEQ ID NO: 41) | GARAGAGTCCTGTATTGTTATTTTTCGTCACTACCTCCCCGGGTCGGGAGT | |
| | GGGTAATTTGCGCGCCTGCTGCCTTC | |
| NR_003286.2_RNA18S5_r1_6 | /5Biosg/GCCCGCCCGCTCCCAAGATCCLACTACGAGCTTTTTAACTGCA | 0.533333333 |
| (SEQ ID NO: 42) | GCAACTTTAATATACGCTATTGGAGCTGGAATTACCGCGGCTGCTGGCACC | |
| | AGACTTGCCCTCCAATGGATCCTCGT | |
| NR_003286.2_RNA18S5_r1_7 | /5Biosg/ACTCTRATTTTTTCAAAGTAAACGCTTCGGGCCCCGCGGGACA | 0.666666667 |
| (SEQ ID NO: 43) | CTCAGCTAAGAGCATCGAGGGGGCGCCGAGAGGCAAGGGGCGGGGACGGGC | |
| | GGTGGCTCGCCTCGCGGCGGACCGCC | |
| NR_003286.2_RNA18S5_r1_8 | /5Biosg/CCCCCGGCCGTCCCTCTTAATCATGGCCTCAGTTCCGAAAACC | 0.55 |
| (SEQ ID NO: 44) | AACAAAATAGAACCGCGGTCCTATTCCATTATTCCTAGCTGCGGTATCCAG | |
| | GCGGCTCGGGCCTGCTTTGAACACTC | |
| NR_003286.2_RNA18S5_r1_9 | /5Biosg/GTCTTCGAACCTCCGACTTTCGTTCTTGATTAATGAAAACATT | 0.491666667 |
| (SEQ ID NO: 45) | CTTGGCAAATGCTTTCGCTCTGGTCCGTCTTGCGCCGGTCCAAGAATTTCA | |
| | CCTCTAGCGGCGCAATACGAATGCCC | |
| NR_003286.2_RNA18S5_r1_10 | /5Biosg/CCCCCCGGAACCCAAAGACTTTGGTTTCCCGGAAGCTGCCCGG | 0.608333333 |
| (SEQ ID NO: 46) | CGGGTCATGGGAATAACGCCGCCGCATCGCCGGTCGGCATCGTTTATGGTC | |
| | GGAACTACGACGGTATCTGATCGTCT | |
| NR_003286.2_RNA18S5_r1_11 | /5Biosg/CCTGTCCGTGTCCGGGCCGGGTGAGGTTTCCCGTGTTGAGTCA | 0.558333333 |
| (SEQ ID NO: 47) | AATTAAGCCGCAGGCTCCACTCCTGGTGGTGCCCTTCCGTCAATTCCTTTA | |
| | AGTTTCAGCTTTGCAACCATACTCCC | |
| NR_003286.2_RNA18S5_r1_12 | /5Biosg/GTTAGCATGCCAGAGTCTCGTTCGTTATCGGAATTAACCAGAC | 0.483333333 |
| (SEQ ID NO: 48) | AAATCGCTCCACCAACTAAGAACGGCCATGCACCACCACCCACGGAATCGA | |
| | GAAAGAGCTATCAATCTGTCAATCCT | |
| NR_003286.2_RNA18S5_r1_13 | /5Biosg/AGCCCCGGACATCTAAGGGCATCACAGACCTGTTATTGCTCAA | 0.575 |
| (SEQ ID NO: 49) | TCTCGGGTGGCTGAACGCCACTTGTCCCTCTAAGAAGTTGGGGGACGCCGA | |
| | CCGCTCGGGGGTCGCGTAACTAGTTA | |
| NR_003286.2_RNA18S5_r1_14 | /5Biosg/CGTTCATGGGGAATAATTGCAATCCCCGATCCCCATCACGAAT | 0.616666667 |
| (SEQ ID NO: 50) | GGGGTTCAACGGGTTACCCGCGCCTGCCGGCGTAGGGTAGGCACACGCTGA | |
| | GCCAGTCAGTGTAGCGCGCGTGCAGC | |
| NR_003286.2_RNA18S5_r1_15 | /5Biosg/GGGCCGATCCGAGGGCCTCACTAAACCATCCAATCGGTAGTAG | 0.55 |
| (SEQ ID NO: 51) | CGACGGGCGGTGTGTACAAAGGGCAGGGACTTAATCAACGCAAGCTTATGA | |
| | CCCGCACTTACTGGGAATTCCTCGTT | |
| NR_003286.2_RNA18S5_r1_16 | /5Biosg/TAATGATCCTTCCGCAGGTTCACCTACGGTAACCTTGTTACGA | 0.575 |
| (SEQ ID NO: 52) | CTTTTACTTCCTCTAGATAGTCAAGTTCGACCGTCTTCTCAGCGCTCCGCC | |
| | AGGGCCGTGGGCCGACCCCGGCGGGG | |
| NR_003287.2_RNA28S5_r1.1_1 | /5Biosg/TCGGCGCTGGGCTCTTCCCTGTTCACTCGCCGTTACTGAGGGA | 0.55 |
| (SEQ ID NO: 53) | ATCCTGGTTAGTTTCTTCTCCTCCGCTGACTAATATGCTTAAATTCAGCGG | |
| | GTCGCCACGTCTGATCTGAGGTCGCG | |
| NR_003287.2_RNA28S5_r1.1_2 | /5Biosg/TCCACGGGCTGGGCCTCGATCAGAAGGACTTGGGCCCCCCACG | 0.758333333 |
| (SEQ ID NO: 54) | AGCGGCGCCGGGGAGCGGGTCTTCCGTACGCCACATGTCCCGCGCCCCGCC | |
| | GCGGGGCGGGGATTCGGCGCTGGGCT | |
| NR_003287.2_RNA28S5_r1.1_3 | /5Biosg/TAGCCTTAGATGGAGTTTACCACCCGCTTTGGGCTGCATTCCC | 0.691666667 |
| (SEQ ID NO: 55) | AAGCAACCCGACTCCGGGAAGACCCGGGCCCGGCGCGCCGGGGGCCGCTAC | |
| | CGGCCTCACACCGTCCACGGGCTGGG | |
| NR_003287.2_RNA28S5_r1.1_4 | /5Biosg/CACCCGTTTACCTCTTAACGGTTTCACGCCCTCTTGAACTCTC | 0.458333333 |
| (SEQ ID NO: 56) | TCTTCAAAGTTCTTTTCAACTTTCCCTTACGGTACTTGTTGACTATCGGTC | |
| | TCGTGCCGGTATTTAGCCTTAGATGG | |
| NR_003287.2_RNA28S5_r1.1_5 | /5Biosg/CGGGTGGAGGGGTCGGGAGGAACGGGGGGCGGGAAAGATCCGC | 0.758333333 |
| (SEQ ID NO: 57) | CGGGCCGCCGACACGGCCGGACCCGCCGCCGGGTTGAATCCTCCGGGCGGA | |
| | CTGCGCGGACCCCACCCGTTTACCTC | |
| NR_003287.2_RNA28S5_r1.1_6 | /5Biosg/GACGGTCCCCCGCCGACCCCACCCCCGGCCCCGCCCGCCCACC | 0.85 |
| (SEQ ID NO: 58) | CCCGCACCCGCCGGAGCCCGCCCCCTCCGGGGAGGAGGAGGAGGGGCGGCG | |
| | GGGGAAGGGAGGGCGGGTGGAGGGGT | |
| NR_003287.2_RNA28S5_r1.1_7 | /5Biosg/CCCCCCGAGCCACCTTCCCCGCCGGGCCTTCCCAGCCGTCCCG | 0.833333333 |
| (SEQ ID NO: 59) | GAGCCGGTCGCGGCGCACCGCCGCGGTGGAAATGCGCCCGGCGGCGGCCGG | |
| | TCGCCGGTCGGGGGACGGTCCCCCGC | |
| NR_003287.2_RNA28S5_r1.1_8 | /5Biosg/ACCGCCGCCGCCGCCGCCGCCCCGACCCGCGCGCCCTCCCGAG | 0.85 |
| (SEQ ID NO: 60) | GGAGGACGCGGGGCCGGGGGGCGGAGACGGGGGAGGAGGAGGACGGACGGA | |
| | CGGACGGACGGGGCCCCCCGAGCCAC | |
| NR_003287.2_RNA28S5_r1.2_1 | /5Biosg/GAGGGGGGAGAGCGCGGCGACGGGTCTCGCTCCCTCGGCCCCG | 0.791666667 |
| (SEQ ID NO: 61) | GGATTCGGCGAGTGCTGCTGCCGGGGGGGCTGTAACACTCGGGGGGGGTTT | |
| | CGGTCCCGCCGCCGCCGCCGCCGCCG | |
| NR_003287.2_RNA28S5_r1.2_2 | /5Biosg/GGGGTGGGAGAGCGGTCGCGCCGTGGGAGGGGTGGCCCGGCCC | 0.85 |
| (SEQ ID NO: 62) | CCCCACGAGGAGACGCCGGCGCGCCCCCGCGGGGGAGACCCCCCTCGCGGG | |
| | GGATTCCCCGCGGGGGTGGGCGCCGG | |
| NR_003287.2_RNA28S5_r1.2_3 | /5Biosg/AGAGAACCTCCCCCGGGCCCGACGGCGCGACCCGCCCGGGGCG | 0.858333333 |
| (SEQ ID NO: 63) | CACTGGGGACAGTCCGCCCCGCCCCCCGACCCGCGCGCGGCACCCCCCCCG | |
| | TCGCCGGGGCGGGGGCGCGGGGAGGA | |
| NR_003287.2_RNA28S5_r1.2_4 | /5Biosg/GCACGTGTTAGACTCCTTGGTCCGTGTTTCAAGACGGGTCGGG | 0.725 |
| (SEQ ID NO: 64) | TGGGTAGCCGACGTCGCCGCCGACCCCGTGCGCTCGCTCCGCCGTCCCCCT | |
| | CTTCGGGGGACGCGCGCGTGGCCCCG | |
| NR_003287.2_RNA28S5_r1.2_5 | /5Biosg/CGGGCCGGTGGTGCGCCCTCGGCGGACTGGAGAGGCCTCGGGA | 0.758333333 |
| (SEQ ID NO: 65) | TCCCACCTCGGCCGGCGAGCGCGCCGGCCTTCACCTTCATTGCGCCACGGC | |
| | GGCTTTCGTGCGAGCCCCCGACTCGC | |
| NR_003287.2_RNA28S5_r1.2_6 | /5Biosg/GGACCGCTACGGACCTCCACCAGAGTTTCCTCTGGCTTCGCCC | 0.658333333 |
| (SEQ ID NO: 66) | TGCCCAGGCATAGTTCACCATCTTTCGGGTCCTAACACGTGCGCTCGTGCT | |
| | CCACCTCCCCGGCGCGGCGGGCGAGA | |
| NR_003287.2_RNA28S5_r1.2_7 | /5Biosg/TGCGTCGGGTCTGCGAGAGCGCCAGCTATCCTGAGGGAAACTT | 0.566666667 |
| (SEQ ID NO: 67) | CGGAGGGAACCAGCTACTAGATGGTTCGATTAGTCTTTCGCCCCTATACCC | |
| | AGGTCGGACGACCGATTTGCACGTCA | |
| NR_003287.2_RNA28S5_r1.2_8 | /5Biosg/CCGGCTCCACGCCAGCGAGCCGGGCTTCTTACCCATTTAAAGT | 0.541666667 |
| (SEQ ID NO: 68) | TTGAGAATAGGTTGAGATCGTTTCGGCCCCAAGACCTCTAATCATTCGCTT | |
| | TACCGGATAAAACTGCGTGGCGGGGG | |
| NR_003287.2_RNA28S5_r1.2_9 | /5Biosg/AACACCTTTTCTGGGGTCTGATGAGCGTCGGCATCGGGCGCCT | 0.6 |
| (SEQ ID NO: 69) | TAACCCGGCGTTCGGTTCATCCCGCAGCGCCAGTTCTGCTTACCAAAAGTG | |
| | GCCCACTAGGCACTCGCATTCCACGC | |
| NR_003287.2_RNA28S5_r1.2_10 | /5Biosg/ATGGGCCCGACGCTCCAGCGCCATCCATTTTCAGGGCTAGTTG | 0.55 |
| (SEQ ID NO: 70) | ATTCGGCAGGTGAGTTGTTACACACTCCTTAGCGGATTCCGACTTCCATGG | |
| | CCACCGTCCTGCTGTCTATATCAACC | |
| NR_003287.2_RNA28S5_r1.2_11 | /5Biosg/GAAGGACCCCACACCCCCGCCGCCGCCGCCGCCGCCGCCCTCC | 0.833333333 |
| (SEQ ID NO: 71) | GACGCACACCACACGCGCGCGCGCGCGCGCCGCCCCCGCCGCTCCCGTCCA | |
| | CTCTCGACTGCCGGCGACGGCCGGGT | |
| NR_003287.2_RNA28S5_r1.2_12 | /5Biosg/GCTTCAAGGCTCACCGCAGCGGCCCTCCTACTCGTCGCGGCGT | 0.808333333 |
| (SEQ ID NO: 72) | AGCGTCCGCGGGGCTCCGGGGGCGGGGAGCGGGGCGTGGGCGGGAGGAGGG | |
| | GAGGAGGCGTGGGGGGGGGGGCGGGG | |
| NR_003287.2_RNA28S5_r1.2_13 | /5Biosg/GTTCAACTGCTGTTCACATGGAACCCTTCTCCACTTCGGCCTT | 0.558333333 |
| (SEQ ID NO: 73) | CAAAGTTCTCGTTTGAATATTTGCTACTACCACCAAGATCTGCACCTGCGG | |
| | CGGCTCCACCCGGGCCCGCGCCCTAG | |
| NR_003287.2_RNA28S5_r1.2_14 | /5Biosg/CCGCCACTCCGGATTCGGGGATCTGAACCCGACTCCCTTTCGA | 0.666666667 |
| (SEQ ID NO: 74) | TCGGCCGAGGGCAACGGAGGCCATCGCCCGTCCCTTCGGAACGGCGCTCGC | |
| | CCATCTCTCAGGACCGACTGACCCAT | |
| NR_003287.2_RNA28S5_r1.2_15 | /5Biosg/GGCGAACCCATTCCAGGGCGCCCTGCCCTTCACAAAGAAAAGA | 0.675 |
| (SEQ ID NO: 75) | GAACTCTCCCCGGGGCTCCCGCCGGCTTCTCCGGGATCGGTCGCGTTACCG | |
| | CACTGGACGCCTCGCGGCGCCCATCT | |
| NR_003287.2_RNA28S5_r1.2_16 | /5Biosg/ATATGGGTACGGCCCGGCGCGAGATTTACACCCTCTCCCCCGG | 0.666666667 |
| (SEQ ID NO: 76) | ATTTTCAAGGGCCAGCGAGAGCTCACCGGACGCCGCCGGAACCGCGACGCT | |
| | TTCCAAGGCACGGGCCCCTCTCTCGG | |
| NR_003287.2_RNA28S5_r1.2_17 | /5Biosg/CGACCGACCCAGCCCTTAGAGCCAATCCTTATCCCGAAGTTAC | 0.566666667 |
| (SEQ ID NO: 77) | GGATCCGGCTTGCCGACTTCCCTTACCTACATTGTTCCAACATGCCAGAGG | |
| | CTGTTCACCTTGGAGACCTGCTGCGG | |
| NR_003287.2_RNA28S5_r1.2_18 | /5Biosg/CGAGCGGCGCGCGCGGGGTGGGGCGGGGGAGGGCCGCGAGGGG | 0.891666667 |
| (SEQ ID NO: 78) | GGTGCCCCGGGCGTGGGGGGGGCGGCGGCGCCTCGTCCAGCCGCGGCGCGC | |
| | GCCCAGCCCCGCTTCGCGCCCCAGCC | |
| NR_003287.2_RNA28S5_r1.2_19 | /5Biosg/GCCCCTGCCGCCCCGACCCTTCTCCCCCCGCCGCCGCCCCCAC | 0.825 |
| (SEQ ID NO: 79) | GCGGCGCTCCCCCGGGGAGGGGGGAGGACGGGGAGCGGGGGAGAGAGAGAG | |
| | AGAGAGGGCGCGGGGCGGGGAGGGAG | |
| NR_003287.2_RNA28S5_r1.2_20 | /5Biosg/CCACGGGAAGGGCCCGGCTCGCGTCCAGAGTCGCCGCCGCCGC | 0.9 |
| (SEQ ID NO: 80) | CGGCCCCCCGGGTGCCCGGGCCCCCCTCGCGGGGGACCGTGCCCCCGCCGC | |
| | CGGGGCCCCGCGGCGGGCCGCCGCCG | |
| NR_003287.2_RNA28S5_r1.2_21 | /5Biosg/CGACCGCTCCCCGCCCCCAGCGGACGCGCGCGCGACGAGACGT | 0.875 |
| (SEQ ID NO: 81) | GGGGTGGGGGGGGGGGCGCGCCGGCGCCCGCCGGGCTCCCCGGGGGCGGCC | |
| | GCGACGCCCGCCGCAGCTGGGGCGAT | |
| NR_003287.2_RNA28S5_r1.2_22 | /5Biosg/CGCCTGCCGCCGCCGCCGCCGCGCGCCGAGGAGGAGGGGGGAA | 0.891666667 |
| (SEQ ID NO: 52) | CGGGGGGCGGACGGGGCCGGGGGGGTAGGGCGGGGGGACGAACCGCCCCGC | |
| | CCCGCCGCCCGCCGACCGCCGCCGCC | |
| NR_003287.2_RNA28S5_r1.2_23 | /5Biosg/TTCCCCTGGTCCGCACCAGTTCTAAGTCGGCTGCTAGGCGCCG | 0.816666667 |
| (SEQ ID NO: 83) | GCCGAGGCGAGGCGCCGCGCGGAACCGCGGCCCCGGGGGCGGACCCGGCGG | |
| | GGGGGACCGGCCCGCGGCCCCTCCGC | |
| NR_003287.2_RNA28S5_r1.2_24 | /5Biosg/CGTTTACCCGCGCTTCATTGAATTTCTTCACTTTGACATTCAG | 0.491666667 |
| (SEQ ID NO: 84) | AGCACTGGGCAGAAATCACATCGCGTCAACACCCGCCGCGGGCCTTCGCGA | |
| | TGCTTTGTTTTAATTAAACAGTCGGA | |
| NR_003287.2_RNA28S5_r1.2_25 | /5Biosg/GGCTGTGGTTTCGCTGGATAGTAGGTAGGGACAGTGGGAATCT | 0.491666667 |
| (SEQ ID NO: 85) | CGTTCATCCATTCATGCGCGTCACTAATTAGATGACGAGGCATTTGGCTAC | |
| | CTTAAGAGAGTCATAGTTACTCCCGC | |
| NR_003287.2_RNA28S5_r1.2_26 | /5Biosg/GGGGGGGCGCCGGGGGCCTCCCACTTATTCTACACCTCTCATG | 0.6 |
| (SEQ ID NO: 56) | TCTCTTCACCGTGCCAGACTAGAGTCAAGCTCAACAGGGTCTTCTTTCCCC | |
| | GCTGATTCCGCCAAGCCCGTTCCCTT | |
| NR_003287.2_RNA28S5_r1.2_27 | /5Biosg/GCCCCTCGGGGCTCGCCCCCCCGCCTCACCGGGTCAGTGAAAA | 0.741666667 |
| (SEQ ID NO: 87) | AACGATCAGAGTAGTGGTATTTCACCGGCGGCCCGCAGGGCCGGCGGACCC | |
| | CGCCCCGGGCCCCTCGCGGGGACACC | |
| NR_003287.2_RNA28S5_r1.2_28 | /5Biosg/GACACCTGCGTTACCGTTTGACAGGTGTACCGCCCCAGTCAAA | 0.716666667 |
| (SEQ ID NO: 88) | CTCCCCACCTGGCACTGTCCCCGGAGCGGGTCGCGCCCGGCCGGCGCGCGG | |
| | CCGGGCGCTTGGCGCCAGAAGCGAGA | |
| NR_003287.2_RNA28S5_r1.2_29 | /5Biosg/GGTCAGAAGGATCGTGAGGCCCCGCTTTCACGGTCTGTATTCG | 0.541666667 |
| (SEQ ID NO: 89) | TACTGAAAATCAAGATCAAGCGAGCTTTTGCCCTTCTGCTCCACGGGAGGT | |
| | TTCTGTCCTCCCTGAGCTCGCCTTAG | |
| NR_003287.2_RNA28S5_r1.2_30 | /5Biosg/CAATGATAGGAAGAGCCGACATCGAAGGATCAAAAAGCGACGT | 0.483333333 |
| (SEQ ID NO: 90) | CGCTATGAACGCTTGGCCGCCACAAGCCAGTTATCCCTGTGGTAACTTTTC | |
| | TGACACCTCCTGCTTAAAACCCAAAA | |
| NR_003287.2_RNA28S5_r1.2_31 | /5Biosg/TTACCATGGCAACAACACATCATCAGTAGGGTAAAACTAACCT | 0.458333333 |
| (SEQ ID NO: 91) | GTCTCACGACGGTCTAAACCCAGCTCACGTTCCCTATTAGTGGGTGAACAA | |
| | TCCAACGCTTGGTGAATTCTGCTTCA | |
| NR_003287.2_RNA28S5_r1.2_32 | /5Biosg/GATTCTGACTTAGAGGCGTTCAGTCATAATCCCACAGATGGTA | 0.516666667 |
| (SEQ ID NO: 92) | GCTTCGCCCCATTGGCTCCTCAGCCAAGCACATACACCAAATGTCTGAACC | |
| | TGCGGTTCCTCTCGTACTGAGCAGGA | |
| NR_003287.2_RNA28S5_r1.2_33 | /5Biosg/TCCCGCGCGCGCGGGGCGCGTGGAGGGGGGGGGCGGCCCGCCG | 0.825 |
| (SEQ ID NO: 93) | GCGGGGACAGGCGGGGGACCGGCTATCCGAGGCCAACCGAGGCTCCGCGGC | |
| | GCTGCCGTATCGTTCCGCCTGGGCGG | |
| NR_003287.2_RNA28S5_r1.2_34 | /5Biosg/ACGAACGTGCGGTGCGTGACGGGCGAGGGGGCGGCCGCCTCTC | 0.808333333 |
| (SEQ ID NO: 94) | CGGCCGCGCCCCGTTTCCCAGGACGAAGGGCACTCCGCACCGGACCCCGGT | |
| | CCCGGCGCGCGGCGGGGCACGCGCCC | |
| NR_003287.2_RNA28S5_r1.2_35 | /5Biosg/GACAAACCCTTGTGTCGAGGGCTGACTTTCAATAGATCGCAGC | 0.566666667 |
| (SEQ ID NO: 95) | GAGGGAGCTGCTCTGCTACGTACGAAACCCCGACCCAGAAGCAGGTCGTCT | |
| | ACGAATGGTTTAGCGCCAGGTTCCCC | |
| 5SrRNA_X71801_r1_1 | /5Biosg/CTTCCACCACATCGGGCCCGCTCGGAGCAGGGAGTGCTCCGAG | 0.716666667 |
| (SEQ ID NO: 96) | GCGTCAGGGCCCAGGGCCCACGATCCTGGGACGCCCTCCGGTCCTCCGCCC | |
| | TGTCGCGGAGGCAGCGTTTTGGATCC | |
| 5SrRNA_X71801_r1_2 | /5Biosg/CCGCCGGATTGCAGCCGACACCGCCAGCCCGGGGCCGCGGGGC | 0.8 |
| (SEQ ID NO: 97) | TCGGATCGGGGACCCCCGAGCCGCTGGCCCGCGGCCTTCCCCCGGCTCCCG | |
| | CGCTCCCGAGCTTCCACCACATCGGG | |
| 5SrRNA_X71801_r1_3 | /5Biosg/AGCTTCCGAGATCAGACGAGATCGGGCGCGTTCAGGGTGGTAT | 0.7 |
| (SEQ ID NO: 98) | GGCCGTAGACGCTGAAGGAGGCGCCTGGCTGCCCCAAGAGCCCAGCCCCGC | |
| | CCGGCCGTGCCCGCCGGATTGCAGCC | |
| 5SrRNA_X71801_r1_4 | /5Biosg/GGAAGAAAAGGAAAGAAACAGCAAAAAGCCAAAGAAAAAGCCT | 0.503333333 |
| (SEQ ID NO: 99) | ACAGCACCCGGTATTCCCAGGCGGTCTCCCATCCAAGTACTAACCAGGCCC | |
| | GACCCTGCTTAGCTTCCGAGATCAGA | |
| 5SrRNA_X71801_r1_5 | /5Biosg/GAGGCTGAGGCCGGGGAATGGCGTGGACCCGGGAGGCGGAGCT | 0.666666667 |
| (SEQ ID NO: 100) | TGCAGTGAGCCGAGATGGCGCCACCGCACTCCAGCCTGGGCGACAGGGCGA | |
| | GACTCCGTCTGGAAGAAAAGGAAAGA | |
| 5SrRNA_X71801_r1_6 | /5Biosg/GAGCTCCAGACCATCCCGGCTAACAGGGTGAAAGCCCGTCTCT | 0.633333333 |
| (SEQ ID NO: 101) | AGGAAAAATAGAACAAAGTAGCCGGGCGTGGTGGCGGGCGCCTGTAGGCCC | |
| | AGCTACTCGGGAGGCTGAGGCCGGGG | |
| RNA5-8S5_NR_003285_r1_1 | /5Biosg/CCGGGGCCGCAAGTGCGTTCGAAGTGTCGATGATCAATGTGTC | 0.541666667 |
| (SEQ ID NO: 102) | CTGCAATTCACATTAATTCTCGCAGCTAGCTCCGTTCTTCATCGACGCACG | |
| | AGCCGAGTGATCCACCGCTAAGAGTC | |
| RNA5-8S5_NR_003285_r1_2 | /5Biosg/AAGCGACGCTCAGACAGGCGTAGCCCCGGGAGGAACCCGGGGC | 0.558333333 |
| (SEQ ID NO: 103) | CGCAAGTGCGTTCGAAGTGTCGATGATCAATGTGTCCTGCAATTCACATTA | |
| | ATTCTCGCAGCTAGCTGCGTTCTTCA | |

APPENDIX 3
120-mer DNA rRNA Capture Probes/
Baits at 1x coverage, 85% GC restriction
(Note: /5Biosg/ = 5′-biotin)

| Name | Sequence | GC content |
|---|---|---|
| KF899911.1_12SrRNA_r1_1 | /5Biosg/GCGTGCTTGATGCTTGTCCCTTTTGATCGTGGTGATTTAGAGG | 0.45 |
| (SEQ ID NO: 104) | GTGAACTCACTGGAACGGGGATGCTTGCATGTGTAATCTTACTAAGAGCTA | |
| | ATAGAAAGGCTAGGACCAAACCTATT | |
| KF899911.1_12SrRNA_r1_2 | /5Biosg/GAAATTGACCTACCCTGGGGTTAGTATAGCTTAGTTAAACTTT | 0.45 |
| (SEQ ID NO: 105) | CGTTTATTGCTAAAGGTTAATCACTGCTGTTTCCCGTGGGGGTGTGGCTAG | |
| | GCTAAGCGTTTTGAGCTGCATTGCTG | |
| KF899911.1_12SrRNA_r1_3 | /5Biosg/ACTGGAGTTTTTTACAACTCAGGTGAGTTTTAGCTTTATTGGG | 0.483333333 |
| (SEQ ID NO: 106) | GAGGGGGTGATCTAAAACACTCTTTACGCCGGCTTCTATTGACTTGGGTTA | |
| | ATCGTGTGACCGCGGTGGCTGGCACG | |
| KF899911.1_12SrRNA_r1_4 | /5Biosg/TGTTGATTTAACTGTTGAGGTTTAGGGCTAAGCATAGTGGGGT | 0.383333333 |
| (SEQ ID NO: 107) | ATCTAATCCCAGTTTGGGTCTTAGCTATTGTGTGTTCAGATATGTTAAAGC | |
| | CACTTTCGTAGTCTATTTTGTGTCAA | |
| KF899911.1_12SrRNA_r1_5 | /5Biosg/GAGGTGGTGAGGTTGATCGGGGTTTATCGATTACAGAACAGGC | 0.508333333 |
| (SEQ ID NO: 108) | TCCTCTAGAGGGATATGAAGCACCGCCAGGTCCTTTGAGTTTTAAGCTGTG | |
| | GCTCGTAGTGTTCTGGCGAGCAGTTT | |
| KF699911.1_12SrRNA_r1_6 | /5Biosg/GCCCATTTCTTGCCACCTCATGGGCTACACCTTGACCTAACGT | 0.5 |
| (SEQ ID NO: 109) | CTTTACGTGGGTACTTGCGCTTACTTTGTAGCCTTCATCAGGGTTTGCTGA | |
| | AGATGGCGGTATATAGGCTGAGCAAG | |
| KF899911.1_12SrRNA_r1_7 | /5Biosg/TGTGTACGCGCTTCAGGGCCCTGTTCAACTAAGCACTCTACTC | 0.45 |
| (SEQ ID NO: 110) | TCAGTTTACTGCTAAATCCACCTTCGACCCTTAAGTTTCATAAGGGCTATC | |
| | GTAGTTTTCTGGGGTAGAAAATGTAG | |
| KF899911.1_12SrRNA_r1_8 | /5Biosg/GTTCGTCCAAGTGCACTTTCCAGTACACTTACCATGTTACGAC | 0.441666667 |
| (SEQ ID NO: 111) | TTGTCTCCTCTATATAAATGCGTAGGGGTTTTAGTTAAATGTCCTTTGAAG | |
| | TATACTTGAGGAGGGTGACGGGCGGT | |
| KF899911.1_16SrRNA_r1_1 | /5Biosg/TTGCGGTACTATATCTATTGCGCCAGGTTTCAATTTCTATCGC | 0.425 |
| (SEQ ID NO: 112) | CTATACTTTATTTGGGTAAATGGTTTGGTTAAGGTTGTCTGGTAGTAAGGT | |
| | GGAGTGGGTTTGGGGCTAGGTTTAGC | |
| KF699911.1_16SrRNA_r1_2 | /5Biosg/GTTTCGGGGGTCTTAGCTTTGGCTCTCCTTGCAAAGTTATTTC | 0.375 |
| (SEQ ID NO: 113) | TAGTTAATTCATTATGCAGAAGGTATAGGGGTTAGTCCTTGCTATATTATG | |
| | CTTGGTTATAATTTTTCATCTTTCCC | |
| KF899911.1_16SrRNA_r1_3 | /5Biosg/ATCTTGGACAACCAGCTATCACCAGGCTCGGTAGGTTTGTCGC | 0.466666667 |
| (SEQ ID NO: 114) | CTCTACCTATAAATCTTCCCACTATTTTGCTACATAGACGGGTGTGCTCTT | |
| | TTAGCTGTTCTTAGGTAGCTCGTCTG | |
| KF899911.1_16SrRNA_r1_4 | /5Biosg/CTCTCTCTACAAGGTTTTTTCCTAGTGTCCAAAGAGCTGTTCC | 0.375 |
| (SEQ ID NO: 115) | TCTTTGGACTAACAGTTAAATTTACAAGGGGATTTAGAGGGTTCTGTGGGC | |
| | AAATTTAAAGTTGAACTAAGATTCTA | |
| KF899911.1_16SrRNA_r1_5 | /5Biosg/CCAATTGGGTGTGAGGAGTTCAGTTATATGTTTGGGATTTTTT | 0.391666667 |
| (SEQ ID NO: 116) | AGGTAGTGGGTGTTGAGCTTGAACGCTTTCTTAATTGGTGGCTGCTTTTAG | |
| | GCCTACTATGGGTGTTAAATTTTTTA | |
| KF699911.1_16SrRNA_r1_6 | /5Biosg/GTAGATATTGGGCTGT1AATTGTCAGTTCAGTGTTTTAATCTG | 0.366666667 |
| (SEQ ID NO: 117) | ACGCAGGCTTATGCGGAGGAGAATGTTTTCATGTTACTTATACTAACATTA | |
| | GTTCTTCTATAGGGTGATAGATTGGT | |
| KF899911.1_16SrRNA_r1_7 | /5Biosg/TGTTTTTGGTAAACAGGCGGGGTAAGATTTGCCGAGTTCCTTT | 0.4 |
| (SEQ ID NO: 116) | TACTTTTTTTAACCTTTCCTTATGAGCATGCCTGTGTTGGGTTGACAGTGA | |
| | GGGTAATAATGACTTGTTGGTTGATT | |
| KF899911.1_16SrRNA_r1_8 | /5Biosg/TACAGGTCCCTATTTAAGGAACAAGTGATTATGCTACCTTTGC | 0.5 |
| (SEQ ID NO: 119) | ACGGTTAGGGTACCGCGGCCGTTAAACATGTGTCACTGGGCAGGCGGTGCC | |
| | TCTAATACTGGTGATGCTAGAGGTGA | |
| KF899911.1_16SrRNA_r1_9 | /5Biosg/TTAATAAATTAAAGCTCCATAGGGTCTTCTCGTCTTGCTGTGT | 0.466666667 |
| (SEQ ID NO: 120) | CATGCCCGCCTCTTCACGGGCAGGTCAATTTCACTGGTTAAAAGTAAGAGA | |
| | CAGCTGAACCCTCGTGGAGCCATTCA | |
| KF699911.1_16SrRNA_r1_10 | /5Biosg/TGGTGAAGTCTTAGCATGTACTGCTCGGAGGTTGGGTTCTGCT | 0.483333333 |
| (SEQ ID NO: 121) | CCGAGGTCGCCCCAACCGAAATTTTTAATGCAGGTTTGGTAGTTTAGGACC | |
| | TGTGGGTTTGTTAGGTACTGTTTGCA | |
| KF899911.1_16SrRNA_r1_11 | /5Biosg/GAGGTCGTAAACCCTATTGTTGATATGGACTCTAGAATAGGAT | 0.408333333 |
| (SEQ ID NO: 122) | TGCGCTGTTATCCCTAGGGTAACTTGTTCCGTTGGTCAAGTTATTGGATCA | |
| | ATTGAGTATAGTAGTTCGCTTTGACT | |
| KF899911.1_16SrRNA_r1_12 | /5Biosg/AAGTAGATAGAAACCGACCTGGATTACTCCGGTCTGAACTCAG | 0.458333333 |
| (SEQ ID NO: 123) | ATCACGTAGGACTTTAATCGTTGAACAAACGAACCTTTAATAGCGGCTGCA | |
| | CCATCGGGATGTCCTGATCCAACATC | |
| KF899911.1_16SrRNA_r1_13 | /5Biosg/AAACCCTGTTCTTGGGTGGGTGTGGGTATAATACTAAGTTGAG | 0.441666667 |
| (SEQ ID NO: 124) | ATGATATCATTTACGGGGGAAGGCGCTTTGTGAAGTAGGCCTTATTTCTCT | |
| | TGTCCTTTCGTACAGGGAGGAATTTG | |
| NR_003286.2_RNA18S5_r1_l | /5Biosg/AAGGAACCATAACTGATTTAATGAGCCATTCGCAGTTTCACTG | 0.458333333 |
| (SEQ ID NO: 125) | TACCGGCCGTGCGTACTCAGACATGCATGGCTTAATCTTTGAGACAAGCAT | |
| | ATGCTACTGGCAGGATCAACCAGGTA | |
| NR_003286.2_RNA18S5_r1_2 | /5Biosg/GTTGGTTTTGATCTGATAAATGCACGCATCCCCCCCGCGAAGG | 0.533333333 |
| (SEQ ID NO: 126) | GGGTCAGCGCCCGTCGGCATGTATTAGCTCTAGAATTACCACAGTTATCCA | |
| | AGTAGGAGAGGAGCGAGCGACCAAAG | |
| NR_003286.2_RNA18S5_r1_3 | /5Biosg/GACGTTCGAATGGGTCGTCGCCGCCACGGGGGGCGTGCGATCG | 0.741666667 |
| (SEQ ID NO: 127) | GCCCGAGGTTATCTAGAGTCACCAAAGCCGCCGGCGCCCGCCCCCCGGCCG | |
| | GGGCCGGAGAGGGGCTGACCGGGTTG | |
| NR_003286.2_RNA18S5_r1_4 | /5Biosg/CTTCCTTGGATGTGGTAGCCGTTTCTCAGGCTCCCTCTCCGGA | 0.575 |
| (SEQ ID NO: 128) | ATCGAACCCTGATTCCCCGTCACCCGTGGTCACCATGGTAGGCACGGCGAC | |
| | TACCATCGAAAGTTGATAGGGCAGAC | |
| NR_003286.2_RNA18S5_r1_5 | /5Biosg/CGTTAAAGGATTTAAAGTGGACTCATTCCAATTACAGGGCCTC | 0.491666667 |
| (SEQ ID NO: 129) | GAAAGAGTCCTGTATTGTTATTTTTCGTCACTACCTCCCCGGGTCGGGAGT | |
| | GGGTAATTTGCGCGCCTGCTGCCTTC | |
| NR_003286.2_RNA18S5_r1_6 | /5Biosg/GCCCGCCCGCTCCCAAGATCCAACTACGAGCTTTTTAACTGCA | 0.533333333 |
| (SEQ ID NO: 130) | GCAACTTTAATATACGCTATTGGAGCTGGAATTACCGCGGCTGCTGGCACC | |
| | AGACTTGCCCTCCAATGGATCCTCGT | |
| NR_003286.2_RNA18S5_r1_7 | /5Biosg/ACTCTAATTTTTTCAAAGTAAACGCTTCGGGCCCCGCGGGACA | 0.666666667 |
| (SEQ ID NO: 131) | CTCAGCTAAGAGCATCGAGGGGGCGCCGAGAGGCAAGGGGCGGGGACGGGC | |
| | GGTGGCTCGCCTCGCGGCGGACCGCC | |
| NR_003286.2_RNA18S5_r1_8 | /5Biosg/CCCCCGGCCGTCCCTCTTAATCATGGCCTCAGTTCCGAAAACC | 0.55 |
| (SEQ ID NO: 132) | AACAAAATAGAACCGCGGTCCTATTCCATTATTCCTAGCTGCGGTATCCAG | |
| | GCGGCTCGGGCCTGCTTTGAACACTC | |
| NR_003286.2_RNA18S5_r1_9 | /5Biosg/GTCTTCGAACCTCCGACTTTCGTTCTTGATTAATGAAAACATT | 0.491666667 |
| (SEQ ID NO: 133) | CTTGGCAAATGCTTTCGCTCTGGTCCGTCTTGCGCCGGTCCAAGAATTTCA | |
| | CCTCTAGCGGCGCAATACGAATGCCC | |
| NR_003286.2_RNA18S5_r1_10 | /5Biosg/CCCCCCGGAACCCAAAGACTTTGGTTTCCCGGAAGCTGCCCGG | 0.608333333 |
| (SEQ ID NO: 134) | CGGGTCATGGGAATAACGCCGCCGCATCGCCGGTCGGCATCGTTTATGGTC | |
| | GGAACTACGACGGTATCTGATCGTCT | |
| NR_003286.2_RNA18S5_r1_11 | /5Biosg/CCTGTCCGTGTCCGGGCCGGGTGAGGTTTCCCGTGTTGAGTCA | 0.558333333 |
| (SEQ ID NO: 135) | AATTAAGCCGCAGGCTCCACTCCTGGTGGTGCCCTTCCGTCAATTCCTTTA | |
| | AGTTTCAGCTTTGCAACCATACTCCC | |
| NR_003286.2_RNA18S5_r1_12 | /5Biosg/GTTAGCATGCCAGAGTCTCGTTCGTTATCGGAATTAACCAGAC | 0.483333333 |
| (SEQ ID NO: 136) | AAATCGCTCCACCAACTAAGAACGGCCATGCACCACCACCCACGGAATCGA | |
| | GAAAGAGCTATCAATCTGTCAATCCT | |
| NR_003286.2_RNA18S5_r1_13 | /5Biosg/AGCCCCGGACATCTAAGGGCATCACAGACCTGTTATTGCTCAA | 0.575 |
| (SEQ ID NO: 137) | TCTCGGGTGGCTGAACGCCACTTGTCCCTCTAAGAAGTTGGGGGACGCCGA | |
| | CCGCTCGGGGGTCGCGTAACTAGTTA | |
| NR_003286.2_RNA18S5_r1_14 | /5Biosg/CGTTCATGGGGAATAATTGCAATCCCCGATCCCCATCACGAAT | 0.616666667 |
| (SEQ ID NO: 136) | GGGGTTCAACGGGTTACCCGCGCCTGCCGGCGTAGGGTAGGCACACGCTGA | |
| | GCCAGTCAGTGTAGCGCGCGTGCAGC | |
| NR_003286.2_RNA18S5_r1_15 | /5Biosg/GGGCCGATCCGAGGGCCTCACTAAACCATCCAATCGGTAGTAG | 0.55 |
| (SEQ ID NO: 139) | CGACGGGCGGTGTGTACAAAGGGCAGGGACTTAATCAACGCAAGCTTATGA | |
| | CCCGCACTTACTGGGAATTCCTCGTT | |
| NR_003286.2_RNA18S5_r1_16 | /5Biosg/TAATGATCCTTCCGCAGGTTCACCTACGGAAACCTTGTTACGA | 0.575 |
| (SEQ ID NO: 140) | CTTTTACTTCCTCTAGATAGTCAAGTTCGACCGTCTTCTCAGCGCTCCGCC | |
| | AGGGCCGTGGGCCGACCCCGGCGGGG | |
| NR_003287.2_RNA28S5_r1.1_1 | /5Biosg/TCGGCGCTGGGCTCTTCCCTGTTCACTCGCCGTTACTGAGGGA | 0.55 |
| (SEQ ID NO: 141) | ATCCTGGTTAGTTTCTTCTCCTCCGCTGACTAATATGCTTAAATTCAGCGG | |
| | GTCGCCACGTCTGATCTGAGGTCGCG | |
| NR_003287.2_RNA28S5_r1.1_2 | /5Biosg/TCCACGGGCTGGGCCTCGATCAGAAGGACTTGGGCCCCCCACG | 0.758333333 |
| (SEQ ID NO: 142) | AGCGGCGCCGGGGAGCGGGTCTTCCGTACGCCACATGTCCCGCGCCCCGCC | |
| | GCGGGGCGGGGATTCGGCGCTGGGCT | |
| NR_003287.2_RNA28S5_r1.1_3 | /5Biosg/TAGCCTTAGATGGAGTTTACCACCCGCTTTGGGCTGCATTCCC | 0.691666667 |
| (SEQ ID NO: 143) | AAGCAACCCGACTCCGGGAAGACCCGGGCCCGGCGCGCCGGGGGCCGCTAC | |
| | CGGCCTCACACCGTCCACGGGCTGGG | |
| NR_003287.2_RNA28S5_r1.1_4 | /5Biosg/CACCCGTTTACCTCTTAACGGTTTCACGCCCTCTTGAACTCTC | 0.458333333 |
| (SEQ ID NO: 144) | TCTTCAAAGTTCTTTTCAACTTTCCCTTACGGTACTTGTTGACTATCGGTC | |
| | TCGTGCCGGTATTTAGCCTTAGATGG | |
| NR_003287.2_RNA28S5_r1.1_5 | /5Biosg/CGGGTGGAGGGGTCGGGAGGAACGGGGGGCGGGAAAGATCCGC | 0.758333333 |
| (SEQ ID NO: 145) | CGGGCCGCCGACACGGCCGGACCCGCCGCCGGGTTGAATCCTCCGGGCGGA | |
| | CTGCGCGGACCCCACCCGTTTACCTC | |
| NR_003287.2_RNA28S5_r1.1_7 | /5Biosg/CCCCCCGAGCCACCTTCCCCGCCGGGCCTTCCCAGCCGTCCCG | 0.833333333 |
| (SEQ ID NO: 146) | GAGCCGGTCGCGGCGCACCGCCGCGGTGGAAATGCGCCCGGCGGCGGCCGG | |
| | TCGCCGGTCGGGGGACGGTCCCCCGC | |
| NR_003287.2_RNA28S5_r1.2_1 | /5Biosg/GAGGGGGGAGAGCGCGGCGACGGGTCTCGCTCCCTCGGCCCCG | 0.791666667 |
| (SEQ ID NO: 147) | GGATTCGGCGAGTGCTGCTGCCGGGGGGGCTGTAACACTCGGGGGGGGTTT | |
| | CGGTCCCGCCGCCGCCGCCGCCGCCG | |
| NR_003287.2_RNA28S5_r1.2_4 | /5Biosg/GCACGTGTTAGACTCCTTGGTCCGTGTTTCAAGACGGGTCGGG | 0.725 |
| (SEQ ID NO: 148) | TGGGTAGCCGACGTCGCCGCCGACCCCGTGCGCTCGCTCCGCCGTCCCCCT | |
| | CTTCGGGGGACGCGCGCGTGGCCCCG | |
| NR_003287.2_RNA28S5_r1.2_5 | /5Biosg/CGGGCCGGTGGTGCGCCCTCGGCGGACTGGAGAGGCCTCGGGA | 0.758333333 |
| (SEQ ID NO: 149) | TCCCACCTCGGCCGGCGAGCGCGCCGGCCTTCACCTTCATTGCGCCACGGC | |
| | GGCTTTCGTGCGAGCCCCCGACTCGC | |
| NR_003287.2_RNA28S5_r1.2_6 | /5Biosg/GGACCGCTACGGACCTCCACCAGAGTTTCCTCTGGCTTCGCCC | 0.658333333 |
| (SEQ ID NO: 150) | TGCCCAGGCATAGTTCACCATCTTTCGGGTCCTAACACGTGCGCTCGTGCT | |
| | CCACCTCCCCGGCGCGGCGGGCGAGA | |
| NR_003287.2_RNA28S5_r1.2_7 | /5Biosg/TGCGTCGGGTCTGCGAGAGCGCCAGCTATCCTGAGGGAAACTT | 0.666667566 |
| (SEQ ID NO: 151) | CGGAGGGAACCAGCTACTAGATGGTTCGATTAGTCTTTCGCCCCTATACCC | |
| | AGGTCGGACGACCGATTTGCACGTCA | |
| NR_003287.2_RNA28S5_r1.2_8 | /5Biosg/CCGGCTCCACGCCAGCGAGCCGGGCTTCTTACCCATTTAAAGT | 0.541666667 |
| (SEQ ID NO: 152) | TTGAGAATAGGTTGAGATCGTTTCGGCCCCAAGACCTCTAATCATTCGCTT | |
| | TACCGGATAAAACTGCGTGGCGGGGG | |
| NR_003287.2_RNA28S5_r1.2_9 | /5Biosg/AACACCTTTTCTGGGGTCTGATGAGCGTCGGCATCGGGCGCCT | 0.6 |
| (SEQ ID NO: 153) | TAACCCGGCGTTCGGTTCATCCCGCAGCGCCAGTTCTGCTTACCAAAAGTG | |
| | GCCCACTAGGCACTCGCATTCCACGC | |
| NR_003287.2_RNA28S5_r1.2_10 | /5Biosg/ATGGGCCCGACGCTCCAGCGCCATCCATTTTCAGGGCTAGTTG | 0.55 |
| (SEQ ID NO: 154) | ATTCGGCAGGTGAGTTGTTACACACTCCTTAGCGGATTCCGACTTCCATGG | |
| | CCACCGTCCTGCTGTCTATATCAACC | |
| NR_003287.2_RNA28S5_r1.2_11 | /5Biosg/GAAGGACCCCACACCCCCGCCGCCGCCGCCGCCGCCGCCCTCC | 0.833333333 |
| (SEQ ID NO: 155) | GACGCACACCACACGCGCGCGCGCGCGCGCCGCCCCCGCCGCTCCCGTCCA | |
| | CTCTCGACTGCCGGCGACGGCCGGGT | |
| NR_003287.2_RNA28S5_r1.2_12 | /5Biosg/GCTTCAAGGCTCACCGCAGCGGCCCTCCTACTCGTCGCGGCGT | 0.808333333 |
| (SEQ ID NO: 156) | AGCGTCCGCGGGGCTCCGGGGGCGGGGAGCGGGGCGTGGGCGGGAGGAGGG | |
| | GAGGAGGCGTGGGGGGGGGGGCGGGG | |
| NR_003287.2_RNA28S5_r1.2_13 | /5Biosg/GTTCAACTGCTGTTCACATGGAACCCTTCTCCACTTCGGCCTT | 0.558333333 |
| (SEQ ID NO: 157) | CAAAGTTCTCGTTTGAATATTTGCTACTACCACCAAGATCTGCACCTGCGG | |
| | CGGCTCCACCCGGGCCCGCGCCCTAG | |
| NR_003287.2_RNA28S5_r1.2_14 | /5Biosg/CCGCCACTCCGGATTCGGGGATCTGAACCCGACTCCCTTTCGA | 0.666666667 |
| (SEQ ID NO: 158) | TCGGCCGAGGGCAACGGAGGCCATCGCCCGTCCCTTCGGAACGGCGCTCGC | |
| | CCATCTCTCAGGACCGACTGACCCAT | |
| NR_003287.2_RNA28S5_r1.2_15 | /5Biosg/GGCGAACCCATTCCAGGGCGCCCTGCCCTTCACAAAGAAAAGA | 0.675 |
| (SEQ ID NO: 159) | GAACTCTCCCCGGGGCTCCCGCCGGCTTCTCCGGGATCGGTCGCGTTACCG | |
| | CACTGGACGCCTCGCGGCGCCCATCT | |
| NR_003287.2_RNA28S5_r1.2_16 | /5Biosg/ATATGGGTACGGCCCGGCGCGAGATTTACACCCTCTCCCCCGG | 0.666666667 |
| (SEQ ID NO: 160) | ATTTTCAAGGGCCAGCGAGAGCTCACCGGACGCCGCCGGAACCGCGACGCT | |
| | TTCCAAGGCACGGGCCCCTCTCTCGG | |
| NR_003287.2_RNA28S5_r1.2_17 | /5Biosg/CGACCGACCCAGCCCTTAGAGCCAATCCTTATCCCGAAGTTAC | 0.566666667 |
| (SEQ ID NO: 161) | GGATCCGGCTTGCCGACTTCCCTTACCTACATTGTTCCAACATGCCAGAGG | |
| | CTGTTCACCTTGGAGACCTGCTGCGG | |
| NR_003287.2_RNA28S5_r1.2_19 | /5Biosg/GCCCCTGCCGCCCCGACCCTTCTCCCCCCGCCGCCGCCCCCAC | 0.825 |
| (SEQ ID NO: 162) | GCGGCGCTCCCCCGGGGAGGGGGGAGGACGGGGAGCGGGGGAGAGAGAGAG | |
| | AGAGAGGGCGCGGGGCGGGGAGGGAG | |
| NR_003287.2_RNA28S5_r1.2_23 | /5Biosg/TTCCCCTGGTCCGCACCAGTTCTAAGTCGGCTGCTAGGCGCCG | 0.816666667 |
| (SEQ ID NO: 163) | GCCGAGGCGAGGCGCCGCGCGGAACCGCGGCCCCGGGGGCGGACCCGGCGG | |
| | GGGGGACCGGCCCGCGGCCCCTCCGC | |
| NR_003287.2_RNA28S5_r1.2_24 | /5Biosg/CGTTTACCCGCGCTTCATTGAATTTCTTCACTTTGACATTCAG | 0.491666667 |
| (SEQ ID NO: 164) | AGCACTGGGCAGAAATCACATCGCGTCAACACCCGCCGCGGGCCTTCGCGA | |
| | TGCTTTGTTTTAATTAAACAGTCGGA | |
| NR_003287.2_RNA28S5_r1.2_25 | /5Biosg/GGCTGTGGTTTCGCTGGATAGTAGGTAGGGACAGTGGGAATCT | 0.491666667 |
| (SEQ ID NO: 165) | CGTTCATCCATTCATGCGCGTCACTAATTAGATGACGAGGCATTTGGCTAC | |
| | CTTAAGAGAGTCATAGTTACTCCCGC | |
| NR_003287.2_RNA28S5_r1.2_26 | /5Biosg/GGGGGGGCGCCGGGGGCCTCCCACTTATTCTACACCTCTCATG | 0.6 |
| (SEQ ID NO: 166) | TCTCTTCACCGTGCCAGACTAGAGTCAAGCTCAACAGGGTCTTCTTTCCCC | |
| | GCTGATTCCGCCAAGCCCGTTCCCTT | |
| NR_003287.2_RNA28S5_r1.2_27 | /5Biosg/GCCCCTCGGGGCTCGCCCCCCCGCCTCACCGGGTCAGTGAAAA | 0.741666667 |
| (SEQ ID NO: 167) | AACGATCAGAGTAGTGGTATTTCACCGGCGGCCCGCAGGGCCGGCGGACCC | |
| | CGCCCCGGGCCCCTCGCGGGGACACC | |
| NR_003287.2_RNA28S5_r1.2_28 | /5Biosg/GACACCTGCGTTACCGTTTGACAGGTGTACCGCCCCAGTCAAA | 0.716666667 |
| (SEQ ID NO: 168) | CTCCCCACCTGGCACTGTCCCCGGAGCGGGTCGCGCCCGGCCGGCGCGCGG | |
| | CCGGGCGCTTGGCGCCAGAAGCGAGA | |
| NR_003287.2_RNA28S5_r1.2_29 | /5Biosg/GGTCAGAAGGATCGTGAGGCCCCGCTTTCACGGTCTGTATTCG | 0.541666667 |
| (SEQ ID NO: 169) | TACTGAAAATCAAGATCAAGCGAGCTTTTGCCCTTCTGCTCCACGGGAGGT | |
| | TTCTGTCCTCCCTGAGCTCGCCTTAG | |
| NR_003287.2_RNA28S5_r1.2_30 | /5Biosg/CAATGATAGGAAGAGCCGACATCGAAGGATCAAAAAGCGACGT | 0.483333333 |
| (SEQ ID NO: 170) | CGCTATGAACGCTTGGCCGCCACAAGCCAGTTATCCCTGTGGTAACTTTTC | |
| | TGACACCTCCTGCTTAAAACCCAAAA | |
| NR_003287.2_RNA28S5_r1.2_31 | /5Biosg/TTACCATGGCAACAACACATCATCAGTAGGGTAAAACTAACCT | 0.458333333 |
| (SEQ ID NO: 171) | GTCTCACGACGGTCTAAACCCAGCTCACGTTCCCTATTAGTGGGTGAACAA | |
| | TCCAACGCTTGGTGAATTCTGCTTCA | |
| NR_003287.2_RNA28S5_r1.2_32 | /5Biosg/GATTCTGACTTAGAGGCGTTCAGTCATAATCCCACAGATGGTA | 0.516666667 |
| (SEQ ID NO: 172) | GCTTCGCCCCATTGGCTCCTCAGCCAAGCACATACACCAAATGTCTGAACC | |
| | TGCGGTTCCTCTCGTACTGAGCAGGA | |
| NR_003287.2_RNA28S5_r1.2_33 | /5Biosg/TCCCGCGCGCGCGGGGCGCGTGGAGGGGGGGGGCGGCCCGCCG | 0.825 |
| (SEQ ID NO: 173) | GCGGGGACAGGCGGGGGACCGGCTATCCGAGGCCAACCGAGGCTCCGCGGC | |
| | GCTGCCGTATCGTTCCGCCTGGGCGG | |
| NR_003287.2_RNA28S5_r1.2_34 | /5Biosg/ACGAACGTGCGGTGCGTGACGGGCGAGGGGGCGGCCGCCTCTC | 0.808333333 |
| (SEQ ID NO: 174) | CGGCCGCGCCCCGTTTCCCAGGACGAAGGGCACTCCGCACCGGACCCCGGT | |
| | CCCGGCGCGCGGCGGGGCACGCGCCC | |
| NR_003287.2_RNA28S5_r1.2_35 | /5Biosg/GACAAACCCTTGTGTCGAGGGCTGACTTTCAATAGATCGCAGC | 0.566666667 |
| (SEQ ID NO: 175) | GAGGGAGCTGCTCTGCTACGTACGAAACCCCGACCCAGAAGCAGGTCGTCT | |
| | ACGAATGGTTTAGCGCCAGGTTCCCC | |
| 5SrRNA_X71801_r1_1 | /5Biosg/CTTCCACCACATCGGGCCCGCTCGGAGCAGGGAGTGCTCCGAG | 0.716666667 |
| (SEQ ID NO: 176) | GCGTCAGGGCCCAGGGCCCACGATCCTGGGACGCCCTCCGGTCCTCCGCCC | |
| | TGTCGCGGAGGCAGCGTTTTGGATCC | |
| 5SrRNA_X71801_r1_2 | /5Biosg/CCGCCGGATTGCAGCCGACACCGCCAGCCCGGGGCCGCGGGGC | 0.8 |
| (SEQ ID NO: 177) | TCGGATCGGGGACCCCCGAGCCGCTGGCCCGCGGCCTTCCCCCGGCTCCCG | |
| | CGCTCCCGAGCTTCCACCACATCGGG | |
| 5SrRNA_X71801_r1_3 | /5Biosg/AGCTTCCGAGATCAGACGAGATCGGGCGCGTTCAGGGTGGTAT | 0.7 |
| (SEQ ID NO: 178) | GGCCGTAGACGCTGAAGGAGGCGCCTGGCTGCCCCAAGAGCCCAGCCCCGC | |
| | CCGCCCGTGCCCGCCGGATTGCAGCC | |
| 5SrRNA_X71801_r1_4 | /5Biosg/GGAAGAAAAGGAAAGAAACAGCAAAAAGCCAAAGAAAAAGCCT | 0.508333333 |
| (SEQ ID NO: 179) | ACAGCACCCGGTATTCCCAGGCGGTCTCCCATCCAAGTACTAACCAGGCCC | |
| | GACCCTGCTTAGCTTCCGAGATCAGA | |
| 5SrRNA_X71801_r1_5 | /5Biosg/GAGGCTGAGGCCGGGGAATGGCGTGGACCCGGGAGGCGGAGCT | 0.666666667 |
| (SEQ ID NO: 180) | TGCAGTGAGCCGAGATGGCGCCACCGCACTCCAGCCTGGGCGACAGGGCGA | |
| | GACTCCGTCTGGAAGAAAAGGAAAGA | |
| 5SrRNA_X71801_r1_6 | /5Biosg/GAGCTCCAGACCATCCCGGCTAACAGGGTGAAAGCCCGTCTCT | 0.633333333 |
| (SEQ ID NO: 181) | AGGAAAAATAGAACAAAGTAGCCGGGCGTGGTGGCGGGCGCCTGTAGGCCC | |
| | AGCTACTCGGGAGGCTGAGGCCGGGG | |
| RNA5-8S5_NR_003285_r1_1 | /5Biosg/CCGGGGCCGCAAGTGCGTTCGAAGTGTCGATGATCAATGTGTC | 0.541666667 |
| (SEQ ID NO: 182) | CTGCAATTCACATTAATTCTCGCAGCTAGCTGCGTTCTTCATCGACGCACG | |
| | AGCCGAGTGATCCACCGCTAAGAGTC | |
| RNA5-8S5_NR_003285_r1_2 | /5Biosg/AAGCGACGCTCAGACAGGCGTAGCCCCGGGAGGAACCCGGGGC | 0.558333333 |
| (SEQ ID NO: 183) | CGCAAGTGCGTTCGAAGTGTCGATGATCAATGTGTCCTGCAATTCACATTA | |
| | ATTCTCGCAGCTAGCTGCGTTCTTCA | |

APPENDIX 4
120-mer DNA rRNA Capture Probes/
Baits at 2x coverage
(Note: /5Biosg/ = 5′-biotin)

| Name | Sequence | GC content |
|---|---|---|
| KF899911.1_12SrRNA_r1_1 | /5Biosg/GCGTGCTTGATGCTTGTCCCTTTTGATCGTGGTGATTTAGAGG | 0.45 |
| (SEQ ID NO: 184) | GTGAACTCACTGGAACGGGGATGCTTGCATGTGTAATCTTACTAAGAGCTA | |
| | ATAGAAAGGCTAGGACCAAACCTATT | |
| KF899911.1_12SrRNA_r1_2 | /5Biosg/TTAATCACTGCTGTTTCCCGTGGGGGTGTGGCTAGGCTAAGCG | 0.508333333 |
| (SEQ ID NO: 185) | TTTTGAGCTGCATTGCTGCGTGCTTGATGCTTGTCCCTTTTGATCGTGGTG | |
| | ATTTAGAGGGTGAACTCACTGGAACG | |
| KF899911.1_12SrRNA_r1_3 | /5Biosg/GAAATTGACCAACCCTGGGGTTAGTATAGCTTAGTTAAACTTT | 0.45 |
| (SEQ ID NO: 186) | CGTTTATTGCTAAAGGTTAATCACTGCTGTTTCCCGTGGGGGTGTGGCTAG | |
| | GCTAAGCGTTTTGAGCTGCATTGCTG | |
| KF899911.1_12SrRNA_r1_4 | /5Biosg/ACACTCTTTACGCCGGCTTCTATTGACTTGGGTTTATCGTGTG | 0.458333333 |
| (SEQ ID NO: 187) | ACCGCGGTGGCTGGCACGAAATTGACCAACCCTGGGGTTAGTATAGCTTAG | |
| | TTAAACTTTCGTTTATTGCTAAAGGT | |
| KF899911.1_12SrRNA_r1_5 | /5Biosg/ACTGGAGTTTTTTACAACTCAGGTGAGTTTTAGCTTTATTGGG | 0.483333333 |
| (SEQ ID NO: 188) | GAGGGGGTGATCTAAAACACTCTTTACGCCGGCTTCTATTGACTTGGGTTA | |
| | ATCGTGTGACCGCGGTGGCTGGCACG | |
| KF899911.1_12SrRNA_r1_6 | /5Biosg/GGTCTTAGCTATTGTGTGTTCAGATATGTTAAAGCCACTTTCG | 0.383333333 |
| (SEQ ID NO: 189) | TAGTCTATTTTCTGTCAACTGGAGTTTTTTACAACTCAGGTGAGTTTTAGC | |
| | TTTATTGGGGAGGGGGTGATCTAAAA | |
| KF899911.1_12SrRNA_r1_7 | /5Biosg/TGTTGATTTAACTGTTGAGGTTTAGGGCTAAGCATAGTGGGGT | 0.383333333 |
| (SEQ ID NO: 190) | ATCTAATCCCAGTTTGGGTCTTAGCTATTGTGTGTTCAGATATGTTAAAGC | |
| | CACTTTCGTAGTCTATTTTGTGTCAA | |
| KF899911.1_12SrRNA_r1_8 | /5Biosg/AAGCACCGCCAGGTCCTTTGAGTTTTAAGCTGTGGCTCGTAGT | 0.466666667 |
| (SEQ ID NO: 191) | GTTCTGGCGAGCAGTTTTGTTGATTTAACTGTTGAGGTTTAGGGCTAAGCA | |
| | TAGTGGGGTATCTAATCCCAGTTTGG | |
| KF899911.1_12SrRNA_r1_9 | /5Biosg/GAGGTGGTGAGGTTGATCGGGGTTTATCGATTACAGAACAGGC | 0.508333333 |
| (SEQ ID NO: 192) | TCCTCTAGAGGGATATGAAGCACCGCCAGGTCCTTTGAGTTTTAAGCTGTG | |
| | GCTCGTAGTGTTCTGGCGAGCAGTTT | |
| KF899911.1_12SrRNA_r1_10 | /5Biosg/CGCTTACTTTGTAGCCTTCATCAGGGTTTGCTGAAGATGGCGG | 0.483333333 |
| (SEQ ID NO: 193) | TATATAGGCTGAGCAAGAGGTGGTGAGGTTGATCGGGGTTTATCGATTACA | |
| | GAACAGGCTCCTCTAGAGGGATATGA | |
| KF899911.1_12SrRNA_r1_11 | /5Biosg/GCCCATTTCTTGCCACCTCATGGGCTACACCTTGACCTAACGT | 0.5 |
| (SEQ ID NO: 194) | CTTTACGTGGGTACTTGCGCTTACTTTGTAGCCTTCATCAGGGTTTGCTGA | |
| | AGATGGCGGTATATAGGCTGAGCAAG | |
| KF899911.1_12SrRNA_r1_12 | /5Biosg/CCACCTTCGACCCTTAAGTTTCATAAGGGCTATCGTAGTTTTC | 0.475 |
| (SEQ ID NO: 195) | TGGGGTAGAAAATGTAGCCCATTTCTTGCCACCTCATGGGCTACACCTTGA | |
| | CCTAACGTCTTTACGTGGGTACTTGC | |
| KF899911.1_12SrRNA_r1_13 | /5Biosg/TGTGTACGCGCTTCAGGGCCCTGTTCAACTAAGCACTCTACTC | 0.45 |
| (SEQ ID NO: 196) | TCAGTTTACTGCTAAATCCACCTTCGACCCTTAAGTTTCATAAGGGCTATC | |
| | GTAGTTTTCTGGGGTAGAAAATGTAG | |
| KF899911.1_12SrRNA_r1_14 | /5Biosg/ATGCGTAGGGGTTTTAGTTAAATGTCCTTTGAAGTATACTTGA | 0.475 |
| (SEQ ID NO: 197) | GGAGGGTGACGGGCGGTGTGTACGCGCTTCAGGGCCCTGTTCAACTAAGCA | |
| | CTCTACTCTCAGTTTACTGCTAAATC | |
| KF899911.1_12SrRNA_r1_15 | /5Biosg/GTTCGTCCAAGTGCACTTTCCAGTACACTTACCATGTTACGAC | 0.441666667 |
| (SEQ ID NO: 198) | TTGTCTCCTCTATATAAATGCGTAGGGGTTTTAGTTAAATGTCCTTTGAAG | |
| | TATACTTGAGGAGGGTGACGGGCGGT | |
| KF899911.1_16SrRNA_r1_1 | /5Biosg/TTGCGGTACTATATCTATTGCGCCAGGTTTCAATTTCTATCGC | 0.425 |
| (SEQ ID NO: 199) | CTATACTTTATTTGGGTAAATGGTTTGGTTAAGGTTGTCTGGTAGTAAGGT | |
| | GGAGTGGGTTTGGGGCTAGGTTTAGC | |
| KF899911.1_16SrRNA_r1_2 | /5Biosg/AGAAGGTATAGGGGTTAGTCCTTGCTATATTATGCTTGGTTAT | 0.366666667 |
| (SEQ ID NO: 200) | AATTTTTCATCTTTCCCTTGCGGTACTATATCTATTGCGCCAGGTTTCAAT | |
| | TTCTATCGCCTATACTTTATTTGGGT | |
| KF899911.1_16SrRNA_r1_3 | /5Biosg/GTTTCGGGGGTCTTAGCTTTGGCTCTCCTTGCAAAGTTATTTC | 0.375 |
| (SEQ ID NO: 201) | TAGTTAATTCATTATGCAGAAGGTATAGGGGTTAGTCCTTGCTATATTATG | |
| | CTTGGTTATAATTTTTCATCTTTCCC | |
| KF899911.1_16SrRNA_r1_4 | /5Biosg/CCCACTATTTTGCTACATAGACGGGTGTGCTCTTTTAGCTGTT | 0.433333333 |
| (SEQ ID NO: 202) | CTTAGGTAGCTCGTCTGGTTTCGGGGGTCTTAGCTTTGGCTCTCCTTGCAA | |
| | AGTTATTTCTAGTTAATTCATTATGC | |
| KF899911.1_16SrRNA_r1_5 | /5Biosg/ATCTTGGACAACCAGCTATCACCAGGCTCGGTAGGTTTGTCGC | 0.466666667 |
| (SEQ ID NO: 203) | CTCTACCTATAAATCTTCCCACTATTTTGCTACATAGACGGGTGTGCTCTT | |
| | TTAGCTGTTCTTACGTAGCTCGTCTG | |
| KF899911.1_16SrRNA_r1_6 | /5Biosg/TAAATTTACAAGGGGATTTAGAGGGTTCTGTGGGCAAATTTAA | 0.4 |
| (SEQ ID NO: 204) | AGTTGAACTAAGATTCTATCTTGGACAACCAGCTATCACCAGGCTCGGTAG | |
| | GTTTGTCGCCTCTACCTATAAATCTT | |
| KF899911.1_16SrRNA_r1_7 | /5Biosg/CTCTCTCTACAAGGTTTTTTCCTAGTGTCCAAAGAGCTGTTCC | 0.375 |
| (SEQ ID NO: 205) | TCTTTGGACTAACAGTTAAATTTACAAGGGGATTTAGAGGGTTCTGTGGGC | |
| | AAATTTAAAGTTGAACTAAGATTCTA | |
| KF899911.1_16SrRNA_r1_8 | /5Biosg/CTTGAACGCTTTCTTAATTGGTGGCTGCTTTTAGGCCTACTAT | 0.391666667 |
| (SEQ ID NO: 206) | GGGTGTTAAATTTTTTACTCTCTCTACAAGGTTTTTTCCTAGTGTCCAAAG | |
| | AGCTGTTCCTCTTTGGACTAACAGTT | |
| KF899911.1_16SrRNA_r1_9 | /5Biosg/CCAATTGGGTGTGAGGAGTTCAGTTATATGTTTGGGATTTTTT | 0.391666667 |
| (SEQ ID NO: 207) | AGGTAGTGGGTGTTGACCTTGAACGCTTTCTTAATTGGTGGCTGCTTTTAG | |
| | GCCTACTATGGGTGTTAAATTTTTTA | |
| KF899911.1_16SrRNA_r1_10 | /5Biosg/GGAGAATGTTTTCATGTTACTTATACTAACATTAGTTCTTCTA | 0.366666667 |
| (SEQ ID NO: 208) | TAGGGTGATAGATTGGTCCAATTGGGTGTGAGGAGTTCAGTTATATGTTTG | |
| | GGATTTTTTAGGTAGTGGGTGTTGAG | |
| KF899911.1_16SrRNA_r1_11 | /5Biosg/GTAGATATTGGGCTGTTAATTGTCAGTTCAGTGTTTTAATCTG | 0.366666667 |
| (SEQ ID NO: 209) | ACGAAGGCTTATGCGGAGGAGAATGTTTTCATGTTACTTATACTAACATTA | |
| | GTTCTTCTATAGGGTGATAGATTGGT | |
| KF899911.1_16SrRNA_r1_12 | /5Biosg/CCTTATGAGCATGCCTGTGTTGGGTTGACAGTGAGGGTAATAA | 0.425 |
| (SEQ ID NO: 210) | TGACTTGTTGGTTGATTGTAGATATTGGGCTGTTAATTGTCAGTTCAGTGT | |
| | TTTAATCTGACGCAGGCTTATGCGGA | |
| KF899911.1_16SrRNA_r1_13 | /5Biosg/TGTTTTTGGTAAACAGGCGGGGTAAGATTTGCCGAGTTCCTTT | 0.4 |
| (SEQ ID NO: 211) | TACTTTTTTTAACCTTTCCTTATGAGCATGCCTGTGTTGGGTTGACAGTGA | |
| | GGGTAATAATGACTTGTTGGTTGATT | |
| KF899911.1_16SrRNA_r1_14 | /5Biosg/GCCGTTAAACATGTGTCACTGGGCAGGCGGTGCCTCTAATACT | 0.45 |
| (SEQ ID NO: 212) | GGTGATGCTAGAGGTGATGTTTTTGGTAAACAGGCGGGGTAAGATTTGCCG | |
| | AGTTCCTTTTACTTTTTTTAACCTTT | |
| KF899911.1_16SrRNA_r1_15 | /5Biosg/TACAGGTCCCTATTTAAGGAACAAGTGATTATGCTACCTTTGC | 0.5 |
| (SEQ ID NO: 213) | ACGGTTAGGGTACCGCGGCCGTTAAACATGTGTCACTGGGCAGGCGGTGCC | |
| | TCTAATACTGGTGATGCTAGACGTGA | |
| KF899911.1_16SrRNA_r1_16 | /5Biosg/GGGCAGGTCAATTTCACTGGTTATTAGTAAGAGACAGCTGAAC | 0.475 |
| (SEQ ID NO: 214) | CCTCGTGGAGCCATTCATACAGGTCCCTATTTAAGGAACAAGTGATTATGC | |
| | TACCTTTGCACGGTTAGGGTACCGCG | |
| KF899911.1_16SrRNA_r1_17 | /5Biosg/TTAATAAATTAAAGCTCCATAGGGTCTTCTCGTCTTGCTGTGT | 0.466666667 |
| (SEQ ID NO: 215) | CATGCCCGCCTCTTCACGGGCAGGTCAATTTCACTGGTTAAAAGTAAGAGA | |
| | CAGCTGAACCCTCGTGGAGCCATTCA | |
| KF899911.1_16SrRNA_r1_18 | /5Biosg/GAAATTTTTAATGCAGGTTTGGTAGTTTAGGACCTGTGGGTTT | 0.416666667 |
| (SEQ ID NO: 216) | GTTAGGTACTGTTTGCATTAATAAATTAAAGCTCCATAGGGTCTTCTCGTC | |
| | TTGCTGTGTCATGCCCGCCTCTTCAC | |
| KF899911.1_16SrRNA_r1_19 | /5Biosg/GGTGAAGTCTTAGCATGTACTGCTCGGAGGTTGGGTTCTGCTC | 0.483333333 |
| (SEQ ID NO: 217) | CGAGGTCGCCCCAACCGAAATTTTTAATGCAGGTTTGGTACTTTAGGACCT | |
| | GTGGGTTTGTTAGGTACTGTTTGCAT | |
| KF899911.1_16SrRNA_r1_20 | /5Biosg/GGTAACTTGTTCCGTTGGTCAAGTTATTGGATCAATTGAGTAT | 0.491666667 |
| (SEQ ID NO: 218) | AGTAGTTCGCTTTGACTGGTGAAGTCTTAGCATGTACTGCTCGGAGGTTGG | |
| | GTTCTGCTCTGAGGTCGCCCCAACCG | |
| KF899911.1_16SrRNA_r1_21 | /5Biosg/GAGGTCGTAAACCCTATTGTTGATATGGACTCTAGAATAGGAT | 0.408333333 |
| (SEQ ID NO: 219) | TGCGCTGTTATCCCTACGGTAACTTGTTCCGTTGGTCAAGTTATTGGATCA | |
| | ATTGAGTATAGTACTTCGCTTTGACT | |
| KF899911.1_16SrRNA_r1_22 | /5Biosg/TCGTTGAACAAACGAACCTTTAATAGCGGCTGCACCATCGGGA | 0.458333333 |
| (SEQ ID NO: 220) | TGTCCTGATCCAACATCGAGGTCGTAAACCCTATTGTTGATATGGACTCTA | |
| | GAATAGGATTGCGCTGTTATCCCTAG | |
| KF899911.1_16SrRNA_r1_23 | /5Biosg/AAGTAGATAGAAACCGACCTGGATTACTCCGGTCTGAACTCAG | 0.458333333 |
| (SEQ ID NO: 221) | ATCACGTAGGACTTTAATCGTTGAACAAACaAACCTTTAATAGCGGCTGCA | |
| | CCATCGGGATGTCCTGATCCAACATC | |
| KF899911.1_16SrRNA_r1_24 | /5Biosg/GGAAGGCGCTTTGTGAAGTAGGCCTTATTTCTCTTGTCCTTTC | 0.45 |
| (SEQ ID NO: 222) | GTACAGGGAGGAATTTGAAGTAGATAGAAACCGACCTGGATTACTCCGGTC | |
| | TGAACTCAGATCACGTAGGACTTTAA | |
| KF899911.1_16SrRNA_r1_25 | /5Biosg/AAACCCTGTTCTTGGGTGGGTGTGGGTATAATACTAAGTTGAG | 0.441666667 |
| (SEQ ID NO: 223) | ATGATATCATTTACGGGGGAAGGCGCTTTGTGAAGTAGGCCTTATTTCTCT | |
| | TGTCCTTTCGTACAGGGAGGAATTTG | |
| NR_003286.2_RNA18S5_r1_1 | /5Biosg/AAGGAACCATAACTGATTTAATGAGCCATTCGCAGTTTCACTG | 0.458333333 |
| (SEQ ID NO: 224) | TACCGGCCGTGCGTACTCAGACATGCATGGCTTAATCTTTGAGACAAGCAT | |
| | ATGCTACTGGCAGGATCAACCAGGTA | |
| NR_003286.2_RNA18S5_r1_2 | /5Biosg/GCATGTATTAGCTCTAGAATTACCACAGTTATCCAAGTAGGAG | 0.466666667 |
| (SEQ ID NO: 225) | AGGAGCGAGCGACCAAAGGAACCATAACTGATTTAATGAGCCATTCGCAGT | |
| | TTCACTGTACCGGCCGTGCGTACTCA | |
| NR_003286.2_RNA18S5_r1_3 | /5Biosg/GTTGGTTTTGATCTGATAAATGCACGCATCCCCCCCGCGAAGG | 0.533333333 |
| (SEQ ID NO: 226) | GGGTCAGCGCCCGTCGGCATGTATTAGCTCTAGAATTACCACAGTTATCCA | |
| | AGTAGGAGAGGAGCGAGCGACCAAAG | |
| NR_003286.2_RNA18S5_r1_4 | /5Biosg/GAGTCACCAAAGCCGCCGGCGCCCGCCCCCCGGCCGGGGCCGG | 0.716666667 |
| (SEQ ID NO: 227) | AGAGGGGCTGACCGGGTTGGTTTTGATCTGATAAATGCACGCATCCCCCCC | |
| | GCGAAGGGGGTCAGCGCCCGTCGGCA | |
| NR_003286.2_RNA18S5_r1_5 | /5Biosg/GTCGTTCGAATGGGTCGTCGCCGCCACGGGGGGCGTGCGATCG | 0.741666667 |
| (SEQ ID NO: 228) | GCCCGAGGTTATCTAGAGTCACCAAAGCCGCCGGCGCCCGCCCCCCGGCCG | |
| | GGGCCGGAGAGGGGCTGACCGGGTTG | |
| NR_003286.2_RNA18S5_r1_6 | /5Biosg/CCGTCACCCGTGGTaACCATGGTAGGCACGGCGACTACCATCG | 0.625 |
| (SEQ ID NO: 229) | AAAGTTGATAGGGCAGACGTTCGAATGGGTCGTCGCCGCCACGGGGGGCGT | |
| | GCGATCGGCCCGAGGTTATCTAGAGT | |
| NR_003286.2_RNA18S5_r1_7 | /5Biosg/CTTCCTTGGATGTGGTAGCCGTTTCTCAGGCTCCCTCTCCGGA | 0.575 |
| (SEQ ID NO: 230) | ATCGAACCCTGATTCCCCGTCACCCGTGGTCACCATGGTAGGCACGGCGAC | |
| | TACCATCGAAAGTTGATAGGGCAGAC | |
| NR_003286.2_RNA18S5_r1_8 | /5Biosg/TGTTATTTTTCGTCACTACCTCCCCGGGTCGGGAGTGGGTAAT | 0.575 |
| (SEQ ID NO: 231) | TTGCGCGCCTGCTGCCTTCCTTGGATGTGGTAGCCGTTTCTCAGGCTCCCT | |
| | CTCCGGAATCGAACCCTGATTCCCCG | |
| NR_003286.2_RNA18S5_r1_9 | /5Biosg/CGTTAAAGGATTTAAAGTGGACTCATTCCAATTACAGGGCCTC | 0.491666667 |
| (SEQ ID NO: 232) | GAAAGAGTCCTGTATTGTTATTTTTCGTCACTACCTCCCCGGGTCGGGAGT | |
| | GGGTAATTTGCGCGCCTGCTGCCTTC | |
| NR_003286.2_RNA18S5_r1_10 | /5Biosg/GCTATTGGAGCTGGAATTACCGCGGCTGCTGGCACCAGACTTG | 0.491666667 |
| (SEQ ID NO: 233) | CCCTCCAATGGATCCTCGTTAAAGGATTTAAAGTGGACTCATTCCAATTAC | |
| | AGGGCCTCGAAAGAGTCCTGTATTGT | |
| NR_003286.2_RNA18S5_r1_11 | /5Biosg/GCCCGCCCGCTCCCAAGATCCAACTACGAGCTTTTTAACTGCA | 0.533333333 |
| (SEQ ID NO: 234) | GCAACTTTAATATACGCTATTGGAGCTGGAATTACCGCGGCTGCTGGCACC | |
| | AGACTTGCCCTCCAATGGATCCTCGT | |
| NR_003286.2_RNA18S5_r1_12 | /5Biosg/GAGGGGGCGCCGAGAGGCAAGGGGCGGGGACGGGCGGTGGCTC | 0.65 |
| (SEQ ID NO: 235) | GCCTCGCGGCGGACCGCCCGCCCGCTCCCAAGATCCAACTACGAGCTTTTT | |
| | AACTGCAGCAACTTTAATATACGCTA | |
| NR_003286.2_RNA18S5_r1_13 | /5Biosg/ACTCTAATTTTTTCAAAGTAAACGCTTCGGGCCCCGCGGGACA | 0.666666667 |
| (SEQ ID NO: 236) | CTCAGCTAAAAGCATCGAGGGGGCGCCGAGAGGCAAGGGGCGGGGACGGGC | |
| | GGTGGCTCGCCTCGCGGCGGACCGCC | |
| NR_003286.2_RNA18S5_r1_14 | /5Biosg/CGGTCCTATTCCATTATTCCTAGCTGCGGTATCCAGGCGGCTC | 0.533333333 |
| (SEQ ID NO: 237) | GGGCCTGCTTTGAACACTCTAATTTTTTCAAAGTAAACGCTTCGGGCCCCG | |
| | CGGGACACTCAGCTAAGAGCATCGAG | |
| NR_003286.2_RNA18S5_r1_15 | /5Biosg/CCCCCGGCCGTCCCTCTTAATCATGGCCTCAGTTCCGAAAACC | 0.55 |
| (SEQ ID NO: 238) | AACAAAATAGAACCGCGGTCCTATTCCATTATTCCTAGCTGCGGTATCCAG | |
| | GCGGCTCGGGCCTGCTTTGAACACTC | |
| NR_003286.2_RNA18S5_r1_16 | /5Biosg/GCTCTGGTCCGTCTTGCGCCGGTCCAAGAATTTCACCTCTAGC | 0.558333333 |
| (SEQ ID NO: 239) | GGCGCAATACGAATGCCCCCGGCCGTCCCTCTTAATCATGGCCTCAGTTCC | |
| | GAAAACCAACAAAATAGAACCGCGGT | |
| NR_003286.2_RNA18S5_r1_17 | /5Biosg/GTCTTCGAACCTCCGACTTTCGTTCTTGATTAATGAAAACATT | 0.491666667 |
| (SEQ ID NO: 240) | CTTGGCAAATGCTTTCGCTCTGGTCCGTCTTGCGCCGGTCCAAGAATTTCA | |
| | CCTCTAGCGGCGCAATACGAATGCCC | |
| NR_003286.2_RNA18S5_r1_18 | /5Biosg/ACGCCGCCGCATCGCCGGTCGGCATCGTTTATGGTCGGAACTA | 0.5 |
| (SEQ ID NO: 241) | CGACGGTATCTGATCGTCTTCGAACCTCCGACTTTCGTTCTTGATTAATGA | |
| | AAACATTCTTGGCAAATGCTTTCGCT | |
| NR_003286.2_RNA18S5_r1_19 | /5Biosg/CCCCCCGGAACCCAAAGACTTTGGTTTCCCGGAAGCTGCCCGG | 0.608333333 |
| (SEQ ID NO: 242) | CGGGTCATGGGAATAACGCCGCCGCATCGCCGGTCGGCATCGTTTATGGTC | |
| | GGAACTACGACGGTATCTGATCGTCT | |
| NR_003286.2_RNA18S5_r1_20 | /5Biosg/TCCACTCCTGGTGGTGCCCTTCCGTCAATTCCTTTAAGTTTCA | 0.55 |
| (SEQ ID NO: 243) | GCTTTGCAACCATACTCCCCCCGGAACCCAAAGACTTTGGTTTCCCGGAAG | |
| | CTGCCCGGCGGGTCATGGGAATAACG | |
| NR_003286.2_RNA18S5_r1_21 | /5Biosg/CCTGTCCGTGTCCGGGCCGGGTGAGGTTTCCCGTGTTGAGTCA | 0.558333333 |
| (SEQ ID NO: 244) | AATTAAGCCGCAGGCTCCACTCCTGGTGGTGCCCTTCCGTCAATTCCTTTA | |
| | AGTTTCAGCTTTGCAACCATACTCCC | |
| NR_003286.2_RNA18S5_r1_22 | /5Biosg/TAAGAACGGCCATGCACCACCACCCACGGAATCGAGAAAGAGC | 0.55 |
| (SEQ ID NO: 245) | TATCAATCTGTCAATCCTGTCCGTGTCCGGGCCGGGTGAGGTTTCCCGTGT | |
| | TGAGTCAAATTAAGCCGCAGGCTCCA | |
| NR_003286.2_RNA18S5_r1_23 | /5Biosg/GTTAGCATGCCAGAGTCTCGTTCGTTATCGGAATTAACCAGAC | 0.483333333 |
| (SEQ ID NO: 246) | AAATCGCTCCACCAACTAAGAACGGCCATGCACCACCACCCACGGAATCGA | |
| | GAAAGAGCTATCAATCTGTCAATCCT | |
| NR_003286.2_RNA18S5_r1_24 | /5Biosg/CGCCACTTGTCCCTCTAAGAAGTTGGGGGACGCCGACCGCTCG | 0.541666667 |
| (SEQ ID NO: 247) | GGGGTCGCGTAACTAGTTAGCATGCCAGAGTCTCGTTCGTTATCGGAATTA | |
| | ACCAGACAAATCGCTCCACCAACTAA | |
| NR_003286.2_RNA18S5_r1_25 | /5Biosg/AGCCCCGGACATCTAAGGGCATCACAGACCTGTTATTGCTCAA | 0.575 |
| (SEQ ID NO: 248) | TCTCGGGTGGCTGAACGCCACTTGTCCCTCTAAGAAGTTGGGGGACGCCGA | |
| | CCGCTCGGGGGTCGCGTAACTAGTTA | |
| NR_003286.2_RNA18S5_r1_26 | /5Biosg/CCCGCGCCTGCCGGCGTAGGGTAGGCACACGCTGAGCCAGTCA | 0.641666667 |
| (SEQ ID NO: 249) | GTGTAGCGCGCGTGCAGCCCCGGACATCTAAGGGCATCACAGACCTGTTAT | |
| | TGCTCAATCTCGGGTGGCTGAACGCC | |
| NR_003286.2_RNA18S5_r1_27 | /5Biosg/CGTTCATGGGGAATAATTGCAATCCCCGATCCCCATCACGAAT | 0.616666667 |
| (SEQ ID NO: 250) | GGGGTTCAACGGGTTACCCGCGCCTGCCGGCGTAGGGTAGGCACACGCTGA | |
| | GCCAGTCAGTGTAGCGCGCGTGCAGC | |
| NR_003286.2_RNA18S5_r1_28 | /5Biosg/ACAAAGGGCAGGGACTTAATCAACGCAAGCTTATGACCCGCAC | 0.508333333 |
| (SEQ ID NO: 251) | TTACTGGGAATTCCTCGTTCATGGGGAATAATTGCAATCCCCGATCCCCAT | |
| | CACGAATGGGGTTCAACGGGTTACCC | |
| NR_003286.2_RNA18S5_r1_29 | /5Biosg/GGGCCGATCCGAGGGCCTCACTAAACCATCCAATCGGTAGTAG | 0.55 |
| (SEQ ID NO: 252) | CGACGGGCGGTGTGTACAAAGGGCAGGGACTTAATCAACGCAAGCTTATGA | |
| | CCCGCACTTACTGGGAATTCCTCGTT | |
| NR_003286.2_RNA18S5_r1_30 | /5Biosg/GATAGTCAAGTTCGACCGTCTTCTCAGCGCTCCGCCAGGGCCG | 0.65 |
| (SEQ ID NO: 253) | TGGGCCGACCCCGGCGGGGCCGATCCGAGGGCCTCACTAAACCATCCAATC | |
| | GGTAGTAGCGACGGGCGGTGTGTACA | |
| NR_003286.2_RNA18S5_r1_31 | /5Biosg/TAATGATCCTTCCGCAGGTTCACCTACGGAAACCTTGTTACGA | 0.575 |
| (SEQ ID NO: 254) | CTTTTACTTCCTCTAGATAGTCAAGTTCGACCGTCTTCTCAGCGCTCCGCC | |
| | AGGGCCGTGGGCCGACCCCGGCGGGG | |
| NR_003287.2_RNA28S5_r1.1_1 | /5Biosg/TCGGCGCTGGGCTCTTCCCTGTTCACTCGCCGTTACTGAGGGA | 0.55 |
| (SEQ ID NO: 255) | ATCCTGGTTAGTTTCTTCTCCTCCGCTGACTAATATGCTTAAATTCAGCGG | |
| | GTCGCCACGTCTGATCTGAGGTCGCG | |
| NR_003287.2_RNA28S5_r1.1_2 | /5Biosg/GCCGGGGAGCGGGTCTTCCGTACGCCACATGTCCCGCGCCCCG | 0.666666667 |
| (SEQ ID NO: 256) | CCGCGGGGCGGGGATTCGGCGCTGGGCTCTTCCCTGTTCACTCGCCGTTAC | |
| | TGAGGGAATCCTGGTTAGTTTCTTCT | |
| NR_003287.2_RNA28S5_r1.1_3 | /5Biosg/TCACACCGTCCACGGGCTGGGCCTCGATCAGAAGGACTTGGGC | 0.75 |
| (SEQ ID NO: 257) | CCCCCACGAGCGGCGCCGGGGAGCGGGTCTTCCGTACGCCACATGTCCCGC | |
| | GCCCCGCCGCGGGGCGGGGATTCGGC | |
| NR_003287.2_RNA28S5_r1.1_4 | /5Biosg/CCAAGCAACCCGACTCCGGGAAGACCCGGGCCCGGCGCGCCGG | 0.766666667 |
| (SEQ ID NO: 258) | GGGCCGCTACCGGCCTCACACCGTCCACGGGCTGGGCCTCGATCAGAAGGA | |
| | CTTGGGCCCCCCACGAGCGGCGCCGG | |
| NR_003287.2_RNA28S5_r1.1_5 | /5Biosg/GTCTCGTGCCGGTATTTAGCCTTAGATGGAGTTTACCACCCGC | 0.658333333 |
| (SEQ ID NO: 259) | TTTGGGCTGCATTCCCAAGCAACCCGACTCCGGGAAGACCCGGGCCCGGCG | |
| | CGCCGGGGGCCGCTACCGGCCTCACA | |
| NR_003287.2_RNA28S5_r1.1_6 | /5Biosg/TTGAACTCTCTCTTCAAAGTTCTTTTCAACTTTCCCTTACGGT | 0.458333333 |
| (SEQ ID NO: 260) | ACTTGTTGACTATCGGTCTCGTGCCGGTATTTAGCCTTAGATGGAGTTTAC | |
| | CACCCGCTTTGGGCTGCATTCCCAAG | |
| NR_003287.2_RNA28S5_r1.1_7 | /5Biosg/CCTCCGGGCGGACTGCGCGGACCCCACCCGTTTACCTCTTAAC | 0.525 |
| (SEQ ID NO: 261) | GGTTTCACGCCCTCTTGAACTCTCTCTTCAAAGTTCTTTTCAACTTTCCCT | |
| | TACGGTACTTGTTGACTATCGGTCTC | |
| NR_003287.2_RNA28S5_r1.1_8 | /5Biosg/GGGGGCGGGAAAGATCCGCCGGGCCGCCGACACGGCCGGACCC | 0.7 |
| (SEQ ID NO: 262) | GCCGCCGGGTTGAATCCTCCGGGCGGACTGCGCGGACCCCACCCGTTTACC | |
| | TCTTAACGGTTTCACGCCCTCTTGAA | |
| NR_003287.2_RNA28S5_r1.1_9 | /5Biosg/GAGGAGGAGGAGGGGCGGCGGGGGAAGGGAGGGCGGGTGGAGG | 0.775 |
| (SEQ ID NO: 263) | GGTCGGGAGGAACGGGGGGCGGGAAAGATCCGCCGGGCCGCCGACACGGCC | |
| | GGACCCGCCGCCGGGTTGAATCCTCC | |
| NR_003287.2_RNA28S5_r1.1_10 | /5Biosg/CCCCACCCCCGGCCCCGCCCGCCCACCCCCGCACCCGCCGGAG | 0.85 |
| (SEQ ID NO: 264) | CCCGCCCCCTCCGGGGAGGAGGAGGAGGGGCGGCGGGGGTAGGGAGGGCGG | |
| | GTGGAGGGGTCGGGAGGAACGGGGGG | |
| NR_003287.2_RNA28S5_r1.1_11 | /5Biosg/CGGTGGAAATGCGCCCGGCGGCGGCCGGTCGCCGGTCGGGGGA | 0.858333333 |
| (SEQ ID NO: 265) | CGGTCCCCCGCCGACCCCACCCCCGGCCCCGCCCGCCCACCCCCGCACCCG | |
| | CCGGAGCCCGCCCCCTCCGGGGAGGA | |
| NR_003287.2_RNA28S5_r1.1_12 | /5Biosg/GCCACCTTCCCCGCCGGGCCTTCCCAGCCGTCCCGGAGCCGGT | 0.825 |
| (SEQ ID NO: 266) | CGCGGCGCACCGCCGCGGTGGAAATGCGCCCGGCGGCGGCCGGTCGCCGGT | |
| | CGGGGGACGGTCCCCCGCCGACCCCA | |
| NR_003287.2_RNA28S5_r1.1_13 | /5Biosg/GGGGGGCGGAGACGGGGGAGGAGGAGGACGGACGGACGGACGG | 0.816666667 |
| (SEQ ID NO: 267) | ACGGGGCCCCCCGAGCCACCTTCCCCGCCGGGCCTTCCCAGCCGTCCCGGA | |
| | GCCGGTCGCGGCGCACCGCCGCGGTG | |
| NR_003287.2_RNA28S5_r1.1_14 | /5Biosg/ACCGCCGCCGCCGCCGCCGCCCCGACCCGCGCGCCCTCCCGAG | 0.85 |
| (SEQ ID NO: 268) | GGAGGACGCGGGGCCGGGGGGCGGAGACGGGGGAGGAGGAGGACGGACGGA | |
| | CGGACGGACGGGGCCCCCCGAGCCAC | |
| NR_003287.2_RNA28S5_r1.2_1 | /5Biosg/GAGGGGGGAGAGCGCGGCGACGGGTCTCGCTCCCTCGGCCCCG | 0.791666667 |
| (SEQ ID NO: 269) | GGATTCGGCGAGTGCTGCTGCCGGGGGGGCTGTAACACTCGGGGGGGGTTT | |
| | CGGTCCCGCCGCCGCCGCCGCCGCCG | |
| NR_003287.2_RNA28S5_r1.2_2 | /5Biosg/GGCGCGCCCCCGCGGGGGAGACCCCCCTCGCGGGGGATTCCCC | 0.825 |
| (SEQ ID NO: 270) | GCGGGGGTGGGCGCCGGGAGGGGGGAGAGCGCGGCGACGGGTCTCGCTCCC | |
| | TCGGCCCCGGGATTCGGCGAGTGCTG | |
| NR_003287.2_RNA28S5_r1.2_3 | /5Biosg/GGGGTGGGAGAGCGGTCGCGCCGTGGGAGGGGTGGCCCGGCCC | 0.85 |
| (SEQ ID NO: 271) | CCCCACGAGGAGACGCCGGCGCGCCCCCGCGGGGGAGACCCCCCTCGCGGG | |
| | GGATTCCCCGCGGGGGTGGGCGCCGG | |
| NR_003287.2_RNA28S5_r1.2_4 | /5Biosg/CCCGCCCCCCGACCCGCGCGCGGCACCCCCCCCCGTCGCCGGG | 0.866666667 |
| (SEQ ID NO: 272) | GCGGGGGCGCGGGAGGAGGGGTGGGAGAGCGGTCGCGCCGTGGGAGGGGTG | |
| | GCCCGGCCCCCCCACGAGGAGACGCC | |
| NR_003287.2_RNA28S5_r1.2_5 | /5Biosg/AGAGAACCTCCCCCGGGCCCGACGGCGCGACCCGCCCGGGGCG | 0.858333333 |
| (SEQ ID NO: 273) | CACTGGGGACAGTCCGCCCCGCCCCCCGACCCGCGCGCGGCACCCCCCCCG | |
| | TCGCCGGGGCGGGGGCGCGGGGAGGA | |
| NR_003287.2_RNA28S5_r1.2_6 | /5Biosg/CGCCGACCCCGTGCGCTCGCTCCGCCGTCCCCCTCTTCGGGGG | 0.816666667 |
| (SEQ ID NO: 274) | ACGCGCGCGTGGCCCCGAGAGAACCTCCCCCGGGCCCGACGGCGCGACCCG | |
| | CCCGGGGCGCACTGGGGACAGTCCGC | |
| NR_003287.2_RNA28S5_r1.2_7 | /5Biosg/GCACGTGTTAGACTCCTTGGTCCGTGTTTCAAGACGGGTCGGG | 0.725 |
| (SEQ ID NO: 275) | TGGGTAGCCGACGTCGCCGCCGACCCCGTGCGCTCGCTCCGCCGTCCCCCT | |
| | CTTCGGGGGACGCGCGCGTGGCCCCG | |
| NR_003287.2_RNA28S5_r1.2_8 | /5Biosg/AGCGCGCCGGCCTTCACCTTCATTGCGCCACGGCGGCTTTCGT | 0.666666667 |
| (SEQ ID NO: 276) | GCGAGCCCCCGACTCGCGCACGTGTTAGACTCCTTGGTCCGTGTTTCAAGA | |
| | CGGGTCGGGTGGGTAGCCGACGTCGC | |
| NR_003287.2_RNA28S5_r1.2_9 | /5Biosg/CGGGCCGGTGGTGCGCCCTCGGCGGACTGGAGAGGCCTCGGGA | 0.758333333 |
| (SEQ ID NO: 277) | TCCCACCTCGGCCGGCGAGCGCGCCGGCCTTCACCTTCATTGCGCCACGGC | |
| | GGCTTTCGTGCGAGCCCCCGACTCGC | |
| NR_003287.2_RNA28S5_r1.2_10 | /5Biosg/CCATCTTTCGGGTCCTAACACGTGCGCTCGTGCTCCACCTCCC | 0.75 |
| (SEQ ID NO: 278) | CGGCGCGGCGGGCGAGACGGGCCGGTGGTGCGCCCTCGGCGGACTGGAGAG | |
| | GCCTCGGGATCCCACCTCGGCCGGCG | |
| NR_003287.2_RNA28S5_r1.2_11 | /5Biosg/GGACCGCTACGGACCTCCACCAGAGTTTCCTCTGGCTTCGCCC | 0.658333333 |
| (SEQ ID NO: 279) | TGCCCAGGCATAGTTCACCATCTTTCGGGTCCTAACACGTGCGCTCGTGCT | |
| | CCACCTCCCCGGCGCGGCGGGCGAGA | |
| NR_003287.2_RNA28S5_r1.2_12 | /5Biosg/TAGATGGTTCGATTAGTCTTTCGCCCCTATACCCAGGTCGGAC | 0.566666667 |
| (SEQ ID NO: 280) | GACCGATTTGCACGTCAGGACCGCTACGGACCTCCACCAGAGTTTCCTCTG | |
| | GCTTCGCCCTGCCCAGGCATAGTTCA | |
| NR_003287.2_RNA28S5_r1.2_13 | /5Biosg/TGCGTCGGGTCTGCGAGAGCGCCAGCTATCCTGAGGGAAACTT | 0.566666667 |
| (SEQ ID NO: 281) | CGGAGGGAACCAGCTACTAGATGGTTCGATTAGTCTTTCGCCCCTATACCC | |
| | AGGTCGGACGACCGATTTGCACGTCA | |
| NR_003287.2_RNA28S5_r1.2_14 | /5Biosg/TCGTTTCGGCCCCAAGACCTCTAATCATTCGCTTTACCGGATA | 0.583333333 |
| (SEQ ID NO: 282) | AAACTGCGTGGCGGGGGTGCGTCGGGTCTGCGAGAGCGCCAGCTATCCTGA | |
| | GGGAAACTTCGGAGGGAACCAGCTAC | |
| NR_003287.2_RNA28S5_r1.2_15 | /5Biosg/CCGGCTCCACGCCAGCGAGCCGGGCTTCTTACCCATTTAAAGT | 0.541666667 |
| (SEQ ID NO: 283) | TTGAGAATAGGTTGAGATCGTTTCGGCCCCAAGACCTCTAATCATTCGCTT | |
| | TACCGGATAAAACTGCGTGGCGGGGG | |
| NR_003287.2_RNA28S5_r1.2_16 | /5Biosg/CATCCCGCAGCGCCAGTTCTGCTTACCAAAAGTGGCCCACTAG | 0.566666667 |
| (SEQ ID NO: 284) | GCACTCGCATTCCACGCCCGGCTCCACGCCAGCGAGCCGGGCTTCTTACCC | |
| | ATTTAAAGTTTGAGAATAGGTTGAGA | |
| NR_003287.2_RNA28S5_r1.2_17 | /5Biosg/AACACCTTTTCTGGGGTCTGATGAGCGTCGGCATCGGGCGCCT | 0.6 |
| (SEQ ID NO: 285) | TAACCCGGCGTTCGGTTCATCCCGCAGCGCCAGTTCTGCTTACCAAAAGTG | |
| | GCCCACTAGGCACTCGCATTCCACGC | |
| NR_003287.2_RNA28S5_r1.2_18 | /5Biosg/TTACACACTCCTTAGCGGATTCCGACTTCCATGGCCACCGTCC | 0.558333333 |
| (SEQ ID NO: 286) | TGCTGTCTATATCAACCAACACCTTTTCTGGGGTCTGATGAGCGTCGGCAT | |
| | CGGGCGCCTTAACCCGGCGTTCGGTT | |
| NR_003287.2_RNA28S5_r1.2_19 | /5Biosg/ATGGGCCCGACGCTCCAGCGCCATCCATTTTCAGGGCTAGTTG | 0.55 |
| (SEQ ID NO: 287) | ATTCGGCAGGTGAGTTGTTACACACTCCTTAGCGGATTCCGACTTCCATGG | |
| | CCACCGTCCTGCTGTCTATATCAACC | |
| NR_003287.2_RNA28S5_r1.2_20 | /5Biosg/CGCGCGCGCGCGCCGCCCCCGCCGCTCCCGTCCACTCTCGACT | 0.716666667 |
| (SEQ ID NO: 288) | GCCGGCGACGGCCGGGTATGGGCCCGACGCTCCAGCGCCATCCATTTTCAG | |
| | GGCTAGTTGATTCGGCAGGTGAGTTG | |
| NR_003287.2_RNA28S5_r1.2_21 | /5Biosg/GAAGGACCCCACACCCCCGCCGCCGCCGCCGCCGCCGCCCTCC | 0.833333333 |
| (SEQ ID NO: 289) | GACGCACACCACACGCGCGCGCGCGCGCGCCGCCCCCGCCGCTCCCGTCCA | |
| | CTCTCGACTGCCGGCGACGGCCGGGT | |
| NR_003287.2_RNA28S5_r1.2_22 | /5Biosg/GGGGGCGGGGAGCGGGGCGTGGGCGGGAGGAGGGGAGGAGGCG | 0.85 |
| (SEQ ID NO: 290) | TGGGGGGGGGGGCGGGGGAAGGACCCCACACCCCCGCCGCCGCCGCCGCCG | |
| | CCGCCCTCCGACGCACACCACACGCG | |
| NR_003287.2_RNA28S5_r1.2_23 | /5Biosg/GCTTCAAGGCTCACCGCAGCGGCCCTCCTACTCGTCGCGGCGT | 0.808333333 |
| (SEQ ID NO: 291) | AGCGTCCGCGGGGCTCCGGGGGCGGGGAGCGGGGCGTGGGCGGGAGGAGGG | |
| | GAGGAGGCGTGGGGGGGGGGGCGGGG | |
| NR_003287.2_RNA28S5_r1.2_24 | /5Biosg/TATTTGCTACTACCACCAAGATCTGCACCTGCGGCGGCTCCAC | 0.691666667 |
| (SEQ ID NO: 292) | CCGGGCCCGCGCCCTAGGCTTCAAGGCTCACCGCAGCGGCCCTCCTACTCG | |
| | TCGCGGCGTAGCGTCCGCGGGGCTCC | |
| NR_003287.2_RNA28S5_r1.2_25 | /5Biosg/GTTCAACTGCTGTTCACATGGAACCCTTCTCCACTTCGGCCTT | 0.558333333 |
| (SEQ ID NO: 293) | CAAAGTTCTCGTTTGAATATTTGCTACTACCACCAAGATCTGCACCTGCGG | |
| | CGGCTCCACCCGGGCCCGCGCCCTAG | |
| NR_003287.2_RNA28S5_r1.2_26 | /5Biosg/AGGCCATCGCCCGTCCCTTCGGAACGGCGCTCGCCCATCTCTC | 0.566666667 |
| (SEQ ID NO: 294) | AGGACCGACTGACCCATGTTCAACTGCTGTTCACATGGAACCCTTCTCCAC | |
| | TTCGGCCTTCAAAGTTCTCGTTTGAA | |
| NR_003287.2_RNA28S5_r1.2_27 | /5Biosg/CCGCCACTCCGGATTCGGGGATCTGAACCCGACTCCCTTTCGA | 0.666666667 |
| (SEQ ID NO: 295) | TCGGCCGAGGGCAACGGAGGCCATCGCCCGTCCCTTCGGAACGGCGCTCGC | |
| | CCATCTCTCAGGACCGACTGACCCAT | |
| NR_003287.2_RNA28S5_r1.2_28 | /5Biosg/CCCGCCGGCTTCTCCGGGATCGGTCGCGTTACCGCACTGGACG | 0.7 |
| (SEQ ID NO: 296) | CCTCGCGGCGCCCATCTCCGCCACTCCGGATTCGGGGATCTGAACCCGACT | |
| | CCCTTTCGATCGGCCGAGGGCAACGG | |
| NR_003287.2_RNA28S5_r1.2_29 | /5Biosg/GGCGAACCCATTCCAGGGCGCCCTGCCCTTCACAAAGAAAAGA | 0.675 |
| (SEQ ID NO: 297) | GAACTCTCCCCGGGGCTCCCGCCGGCTTCTCCGGGATCGGTCGCGTTACCG | |
| | CACTGGACGCCTCGCGGCGCCCATCT | |
| NR_003287.2_RNA28S5_r1.2_30 | /5Biosg/AGAGCTCACCGGACGCCGCCGGAACCGCCACGCTTTCCAAGGC | 0.666666667 |
| (SEQ ID NO: 298) | ACGGGCCCCTCTCTCGGGGCGAACCCATTCCAGGGCGCCCTGCCCTTCACA | |
| | AAGAAAAGAGAACTCTCCCCGGGGCT | |
| NR_003287.2_RNA28S5_r1.2_31 | /5Biosg/ATATGGGTACGGCCCGGCGCGAGATTTACACCCTCTCCCCCGG | 0.666666667 |
| (SEQ ID NO: 299) | ATTTTCAAGGGCCAGCGAGAGCTCACCGGACGCCGCCGGAACCGCGACGCT | |
| | TTCCAAGGCACGGGCCCCTCTCTCGG | |
| NR_003287.2_RNA28S5_r1.2_32 | /5Biosg/TTCCCTTACCTACATTGTTCCAACATGCCAGAGGCTGTTCACC | 0.575 |
| (SEQ ID NO: 300) | TTGGAGTCCTGCTGCGGATATGGGTACGGCCCGGCGCGAGATTTACACCCT | |
| | CTCCCCCGGATTTTCAAGGGCCAGCG | |
| NR_003287.2_RNA28S5_r1.2_33 | /5Biosg/CGACCGACCCAGCCCTTAGAGCCAATCCTTATCCCGAAGTTAC | 0.566666667 |
| (SEQ ID NO: 301) | GGATCCGGCTTGCCGACTTCCCTTACCTACATTGTTCCAACATGCCAGAGG | |
| | CTGTTCACCTTGGAGACCTGCTGCGG | |
| NR_003287.2_RNA28S5_r1.2_34 | /5Biosg/GGGGGCGGCGGCGCCTCGTCCAGCCGCGGCGCGCGCCCAGCCC | 0.741666667 |
| (SEQ ID NO: 302) | CGCTTCGCGCCCCAGCCCGACCGACCCAGCCCTTAGAGCCAATCCTTATCC | |
| | CGAAGTTACGGATCCGGCTTGCCGAC | |
| NR_003287.2_RNA28S5_r1.2_35 | /5Biosg/CGAGCGGCGCGCGCGGGGTGGGGCGGGGGAGGGCCGCGAGGGG | 0.891666667 |
| (SEQ ID NO: 303) | GGTGCCCCGGGCGTGGGGGGGGCGGCGGCGCCTCGTCCAGCCGCGGCGCGC | |
| | GCCCAGCCCCGCTTCGCGCCCCAGCC | |
| NR_003287.2_RNA28S5_r1.2_36 | /5Biosg/AGGGGGGAGGACGGGGAGCGGGGGAGAGAGAGAGAGAGAGGGC | 0.833333333 |
| (SEQ ID NO: 304) | GCGGGGCGGGGAGGGAGCGAGCGGCGCGCGCGGGGTGGGGCGGGGGAGGGC | |
| | CGCGAGGGGGGTGCCCCGGGCGTGGG | |
| NR_003287.2_RNA28S5_r1.2_37 | /5Biosg/GCCCCTGCCGCCCCGACCCTTCTCCCCCCGCCGCCGCCCCCAC | 0.825 |
| (SEQ ID NO: 305) | GCGGCGCTCCCCCGGGGAGGGGGGAGGACGGGGAGCGGGGGAGAGAGAGAG | |
| | AGAGAGGGCGCGGGGCGGGGAGGGAG | |
| NR_003287.2_RNA28S5_r1.2_38 | /5Biosg/GGGCCCCCCTCGCGGGGGACCGTGCCCCCGCCGCCGGGGCCCC | 0.916666667 |
| (SEQ ID NO: 306) | GCGGCGGGCCGCCGCCGGCCCCTGCCGCCCCGACCCTTCTCCCCCCGCCGC | |
| | CGCCCCCACGCGGCGCTCCCCCGGGG | |
| NR_003287.2_RNA28S5_r1.2_39 | /5Biosg/CCACGGGAAGGGCCCGGCTCGCGTCCAGAGTCGCCGCCGCCGC | 0.9 |
| (SEQ ID NO: 307) | CGGCCCCCCGGGTGCCCGGGCCCCCCTCGCGGGGGACCGTGCCCCCGCCGC | |
| | CGGGGCCCCGCGGCGGGCCGCCGCCG | |
| NR_003287.2_RNA28S5_r1.2_40 | /5Biosg/GCGCCGGCGCCCGCCGGGCTCCCCGGGGGCGGCCGCGACGCCC | 0.875 |
| (SEQ ID NO: 308) | GCCGCAGCTGGGGCGATCCACGGGAAGGGCCCGGCTCGCGTCCAGAGTCGC | |
| | CGCCGCCGCCGGCCCCCCGGGTGCCC | |
| NR_003287.2_RNA28S5_r1.2_41 | /5Biosg/CGACCGCTCCCCGCCCCCAGCGGACGCGCGCGCGACGAGACGT | 0.875 |
| (SEQ ID NO: 309) | GGGGTGGGGGGGGGGGCGCGCCGGCGCCCGCCGGGCTCCCCGGGGGCGGCC | |
| | GCGACGCCCGCCGCAGCTGGGGCGAT | |
| NR_003287.2_RNA28S5_r1.2_42 | /5Biosg/CGGGGGGGTAGGGCGGGGGGACGAACCGCCCCGCCCCGCCGCC | 0.875 |
| (SEQ ID NO: 310) | CGCCGACCGCCGCCGCCCGACCGCTCCCAGCCCCCAGCGGACGCGCGCGCG | |
| | ACGAGACGTGGGGTGGGGGGGGGGGC | |
| NR_003287.2_RNA28S5_r1.2_43 | /5Biosg/CGCCTGCCGCCGCCGCCGCCGCGCGCCGAGGAGGAGGGGGGAA | 0.891666667 |
| (SEQ ID NO: 311) | CGGGGGGCGGACGGGGCCGGGGGGGTAGGGCGGGGGGACGAACCGCCCCGC | |
| | CCCGCCGCCCGCCGACCGCCGCCGCC | |
| NR_003287.2_RNA28S5_r1.2_44 | /5Biosg/CGCGGAACCGCGGCCCCGGGGGCGGACCCGGCGGGGGGGACCG | 0.9 |
| (SEQ ID NO: 312) | GCCCGCGGCCCCTCCGCCGCCTGCCGCCGCCGCCGCCGCGCGCCGAGGAGG | |
| | AGGGGGGAACGGGGGGCGGACGGGGC | |
| NR_003287.2_RNA28S5_r1.2_45 | /5Biosg/TTCCCCTGGTCCGCACCAGTTCTAAGTCGGCTGCTAGGCGCCG | 0.816666667 |
| (SEQ ID NO: 313) | GCCGAGGCGAGGCGCCGCGCGGAACCGCGGCCCCGGGGGCGGACCCGGCGG | |
| | GGGGGACCGGCCCGCGGCCCCTCCGC | |
| NR_003287.2_RNA28S5_r1.2_46 | /5Biosg/ACATCGCGTCAACACCCGCCGCGGGCCTTCGCGATGCTTTGTT | 0.625 |
| (SEQ ID NO: 314) | TTAATTAAACAGTCGGATTCCCCTGGTCCGCACCAGTTCTAAGTCGGCTGC | |
| | TAGGCGCCGGCCGAGGCGAGGCGCCG | |
| NR_003287.2_RNA28S5_r1.2_47 | /5Biosg/CGTTTACCCGCGCTTCATTGAATTTCTTCACTTTGACATTCAG | 0.491666667 |
| (SEQ ID NO: 315) | AGCACTGGGCAGAAATCACATCGCGTCAACACCCGCCGCGGGCCTTCGCGA | |
| | TGCTTTGTTTTAATTAAACAGTCGGA | |
| NR_003287.2_RNA28S5_r1.2_48 | /5Biosg/GCGTCACTAATTAGATGACGAGGCATTTGGCTACCTTAAGAGA | 0.458333333 |
| (SEQ ID NO: 316) | GTCATAGTTACTCCCGCCGTTTACCCGCGCTTCATTGAATTTCTTCACTTT | |
| | GACATTCAGAGCACTGGGCAGAAATC | |
| NR_003287.2_RNA28S5_r1.2_49 | /5Biosg/GGCTGTGGTTTCGCTGGATAGTAGGTAGGGACAGTGGGAATCT | 0.491666667 |
| (SEQ ID NO: 317) | CGTTCATCCATTCATGCGCGTCACTAATTAGATGACGAGGCATTTGGCTAC | |
| | CTTAAGAGAGTCATAGTTACTCCCGC | |
| NR_003287.2_RNA28S5_r1.2_50 | /5Biosg/ACTAGAGTCAAGCTCAACAGGGTCTTCTTTCCCCGCTGATTCC | 0.533333333 |
| (SEQ ID NO: 318) | GCCAAGCCCGTTCCCTTGGCTGTGGTTTCGCTGGATAGTAGGTAGGGACAG | |
| | TGGGAATCTCGTTCATCCATTCATGC | |
| NR_003287.2_RNA28S5_r1.2_51 | /5Biosg/GGGGGGGCGCCGGGGGCCTCCCACTTATTCTACACCTCTCATG | 0.6 |
| (SEQ ID NO: 319) | TCTCTTCACCGTGCCAGACTAGAGTCAAGCTCAACAGGGTCTTCTTTCCCC | |
| | GCTGATTCCGCCAAGCCCGTTCCCTT | |
| NR_003287.2_RNA28S5_r1.2_52 | /5Biosg/TATTTCACCGGCGGCCCGCAGGGCCGGCGGACCCCGCCCCGGG | 0.733333333 |
| (SEQ ID NO: 320) | CCCCTCGCGGGGACACCGGGGGGGCGCCGGGGGCCTCCCACTTATTCTACA | |
| | CCTCTCATGTCTCTTCACCGTGCCAG | |
| NR_003287.2_RNA28S5_r1.2_53 | /5Biosg/GCCCCTCGGGGCTCGCCCCCCCGCCTCACCGGGTCAGTGAAAA | 0.741666667 |
| (SEQ ID NO: 321) | AACGATCAGAGTAGTGGTATTTCACCGGCGGCCCGCAGGGCCGGCGGACCC | |
| | CGCCCCGGGCCCCTCGCGGGGACACC | |
| NR_003287.2_RNA28S5_r1.2_54 | /5Biosg/TCCCCGGAGCGGGTCGCGCCCGGCCGGCGCGCGGCCGGGCGCT | 0.75 |
| (SEQ ID NO: 322) | TGGCGCCAGAAGCGAGAGCCCCTCGGGGCTCGCCCCCCCGCCTCACCGGGT | |
| | CAGTGAAAAAACGATCAGAGTAGTGG | |
| NR_003287.2_RNA28S5_r1.2_55 | /5Biosg/GACACCTGCGTTACCGTTTGACAGGTGTACCGCCCCAGTCAAA | 0.716666667 |
| (SEQ ID NO: 323) | CTCCCCACCTGGCACTGTCCCCGGAGCGGGTCGCGCCCGGCCGGCGCGCGG | |
| | CCGGGCGCTTGGCGCCAGAAGCGAGA | |
| NR_003287.2_RNA28S5_r1.2_56 | /5Biosg/AAGCGAGCTTTTGCCCTTCTGCTCCACGGGAGGTTTCTGTCCT | 0.591666667 |
| (SEQ ID NO: 324) | CCCTGAGCTCGCCTTAGGACACCTGCGTTACCGTGTTGACAGGTGTACCGC | |
| | CCCATCAAACTCCCCACCTGGCACTG | |
| NR_003287.2_RNA28S5_r1.2_57 | /5Biosg/GGTCAGAAGGATCGTGAGGCCCCGCTTTCACGGTCTGTATTCG | 0.541666667 |
| (SEQ ID NO: 325) | TACTGAAAATCAAGATCAAGCGAGCTTTTGCCCTTCTGCTCCACGGGAGGT | |
| | TTCTGTCCTCCCTGAGCTCGCCTTAG | |
| NR_003287.2_RNA28S5_r1.2_58 | /5Biosg/CGCCACAAGCCAGTTATCCCTGTGGTAACTTTTCTGACACCTC | 0.483333333 |
| (SEQ ID NO: 326) | CTGCTTAAAACCCAAAAGGTCAGAAGGATCGTGAGGCCCCGCTTTCACGGT | |
| | CTGTATTCGTACTGAAAATCAAGATC | |
| NR_003287.2_RNA28S5_r1.2_59 | /5Biosg/CAATGATAGGAAGAGCCGACATCGAAGGATCAAAAAGCGACGT | 0.483333333 |
| (SEQ ID NO: 327) | CGCTATGAACGCTTGGCCGCCACAAGCCAGTTATCCCTGTGGTAACTTTTC | |
| | TGACACCTCCTGCTTAAAACCCAAAA | |
| NR_003287.2_RNA28S5_r1.2_60 | /5Biosg/ACCaAGCTCACGTTCCCTATTAGTGGGTGAACAATCCAACGCT | 0.491666667 |
| (SEQ ID NO: 328) | TGGTGAATTCTGCTTCACAATGATAGGAAGAGCCGACATCGAAGGATCAAA | |
| | AAGCGACGTCGCTATGAACGCTTGGC | |
| NR_003287.2_RNA28S5_r1.2_61 | /5Biosg/TTACCATGGCAACAACACATCATCAGTAGGGTALAACTAACCT | 0.458333333 |
| (SEQ ID NO: 329) | GTCTCACGACGGTCTAAACCCAGCTCACGTTCCCTATTAGTGGGTGAACAA | |
| | TCCAACGCTTGGTGAATTCTGCTTCA | |
| NR_003287.2_RNA28S5_r1.2_62 | /5Biosg/CCTCAGCCAAGCACATACACCAAATGTCTGAACCTGCGGTTCC | 0.483333333 |
| (SEQ ID NO: 330) | TCTCGTACTGAGCAGGATTACCATGGCAACAACACATCATCAGTAGGGTAA | |
| | AACTAACCTGTCTCACGACGGTCTAA | |
| NR_003287.2_RNA28S5_r1.2_63 | /5Biosg/GATTCTGACTTAGAGGCGTTCAGTCATAATCCCACAGATGGTA | 0.516666667 |
| (SEQ ID NO: 331) | GCTTCGCCCCATTGGCTCCTCAGCCAAGCACATACACCAAATGTCTGAACC | |
| | TGCGGTTCCTCTCGTACTGAGCAGGA | |
| NR_003287.2_RNA28S5_r1.2_64 | /5Biosg/ACCGGCTATCCGAGGCCAACCGAGGCTCCGCGGCGCTGCCGTA | 0.616666667 |
| (SEQ ID NO: 332) | TCGTTCCGCCTGGGCGGGATTCTGACTTAGAGGCGTTCAGTCATAATCCCA | |
| | CAGATGGTAGCTTCGCCCCATTGGCT | |
| NR_003287.2_RNA28S5_r1.2_65 | /5Biosg/TCCCGCGCGCGCGGGGCGCGTGGAGGGGGGGGGCGGCCCGCCG | 0.825 |
| (SEQ ID NO: 333) | GCGGGGACAGGCGGGGGACCGGCTATCCGAGGCCAACCGAGGCTCCGCGGC | |
| | GCTGCCGTATCGTTCCGCCTGGGCGG | |
| NR_003287.2_RNA28S5_r1.2_66 | /5Biosg/CCAGGACGAAGGGCACTCCGCACCGGACCCCGGTCCCGGCGCG | 0.875 |
| (SEQ ID NO: 334) | CGGCGGGGCACGCGCCCTCCCGCGCGCGCGGGGCGCGTGGAGGGGGGGGGC | |
| | GGCCCGCCGGCGGGGACAGGCGGGGG | |
| NR_003287.2_RNA28S5_r1.2_67 | /5Biosg/ACGAACGTGCGGTGCGTGACGGGCGAGGGGGCGGCCGCCTCTC | 0.808333333 |
| (SEQ ID NO: 335) | CGGCCGCGCCCCGTTTCCCAGGACGAAGGGCACTCCGCACCGGACCCCGGT | |
| | CCCGGCGCGCGGCGGGGCACGCGCCC | |
| NR_003287.2_RNA28S5_r1.2_68 | /5Biosg/ACGTACGAAACCCCGACCCAGAAGCAGGTCGTCTACGAATGGT | 0.683333333 |
| (SEQ ID NO: 336) | TTAGCGCCAGGTTCCCCACGAACGTGCGGTGCGTGACGGGCGAGGGGGCGG | |
| | CCGCCTCTCCGGCCGCGCCCCGTTTC | |
| NR_003287.2_RNA28S5_r1.2_69 | /5Biosg/GACAAACCCTTGTGTCGAGGGCTGACTTTCAATAGATCGCAGC | 0.566666667 |
| (SEQ ID NO: 337) | GAGGGAGCTGCTCTGCTACGTACGAAACCCCGACCCAGAAGCAGGTCGTCT | |
| | ACGAATGGTTTAGCGCCAGGTTCCCC | |
| 5SrRNA_X71801_r1_1 | /5Biosg/CTTCCACCACATCGGGCCCGCTCGGAGCAGGGAGTGCTCCGAG | 0.716666667 |
| (SEQ ID NO: 336) | GCGTCAGGGCCCAGGGCCCACGATCCTGGGACGCCCTCCGGTCCTCCGCCC | |
| | TGTCGCGGAGGCAGCGTTTTGGATCC | |
| 5SrRNA_X71801_r1_2 | /5Biosg/GATCGGGGACCCCCGAGCCGCTGGCCCGCGGCCTTCCCCCGGC | 0.783333333 |
| (SEQ ID NO: 339) | TCCCGCGCTCCCGAGCTTCCACCACATCGGGCCCGCTCGGAGCAGGGAGTG | |
| | CTCCGAGGCGTCAGGGCCCAGGGCCC | |
| 5SrRNA_X71801_r1_3 | /5Biosg/GCCCGGCCGTGCCCGCCGGATTGCAGCCGACACCGCCAGCCCG | 0.825 |
| (SEQ ID NO: 340) | GGGCCGCGGGGCTCGGATCGGGGACCCCCGAGCCGCTGGCCCGCGGCCTTC | |
| | CCCCGGCTCCCGCGCTCCCGAGCTTC | |
| 5SrRNA_X71801_r1_4 | /5Biosg/GGTGGTATGGCCGTAGACGCTGAAGGAGGCGCCTGGCTGCCCC | 0.766666667 |
| (SEQ ID NO: 341) | AAGAGCCCAGCCCCGCCCGGCCGTGCCCGCCGGATTGCAGCCGACACCGCC | |
| | AGCCCGGGGCCGCGGGGCTCGGATCG | |
| 5SrRNA_X71801_r1_5 | /5Biosg/ACTAACCAGGCCCGACCCTGCTTAGCTTCCGAGATCAGACGAG | 0.666666667 |
| (SEQ ID NO: 342) | ATCGGGCGCGTTCAGGGTGGTATGGCCGTAGACGCTGAAGGAGGCGCCTGG | |
| | CTGCCCCAAGAGCCCAGCCCCGCCCG | |
| 5SrRNA_X71801_r1_6 | /5Biosg/AAAAGCCAAAGAAAAAGCCTACAGCACCCGGTATTCCCAGGCG | 0.566666667 |
| (SEQ ID NO: 343) | GTCTCCCATCCAAGTACTAACCAGGCCCGACCCTGCTTAGCTTCCGAGATC | |
| | AGACGAGATCGGGCGCGTTCAGGGTG | |
| 5SrRNA_X71801_r1_7 | /5Biosg/CACTCCAGCCTGGGCGACAGGGCGAGACTCCGTCTGGAAGAAA | 0.533333333 |
| (SEQ ID NO: 344) | AGGAAAGAAACAGCAAAAAGCCAAAGAAAAAGCCTACAGCACCCGGTATTC | |
| | CCAGGCGGTCTCCCATCCAAGTACTA | |
| 5SrRNA_X71801_r1_8 | /5Biosg/GGGGAATGGCGTGGACCCGGGAGGCGGAGCTTGCACTGAGCCG | 0.625 |
| (SEQ ID NO: 345) | AGATGGCGCCACCGCACTCCAGCCTGGGCGACAGGGCGAGACTCCGTCTGG | |
| | AAGATAAGGAAAGAAACAGCTAAAAG | |
| 5SrRNA_X71801_r1_9 | /5Biosg/AAGTAGCCGGGCGTGGTGGCGGGCGCCTGTAGGCCCAGCTACT | 0.716666667 |
| (SEQ ID NO: 346) | CGGGAGGCTGAGGCCGGGGAATGGCGTGGACCCGGGAGGCGGAGCTTGCAG | |
| | TGAGCCGAGATGGCGCCACCGCACTC | |
| 5SrRNA_X71801_r1_10 | /5Biosg/GAGCTCCAGACCATCCCGGCTAACAGGGTGAAAGCCCGTCTCT | 0.633333333 |
| (SEQ ID NO: 347) | AGGAAAAATAGAACATAGTAGCCGGGCGTGGTGGCGGGCGCCTGTAGGCCC | |
| | AGCTACTCGGGAGGCTGAGGCCGGGG | |
| RNA5-8S5_NR_003285_r1_1 | /5Biosg/CCGGGGCCGCTAGTGCGTTCGAAGTGTCGATGATCAATGTGTC | 0.541666667 |
| (SEQ ID NO: 348) | CTGCAATTCACATTAATTCTCGCACCTAGCTGCGTTCTTCATCGACGCACG | |
| | AGCCGAGTGATCCACCGCTAAGAGTC | |
| RNA5-8S5_NR_003285_r1_2 | /5Biosg/AAGCGACGCTCAGACAGGCGTAGCCCCGGGAGGAACCCGGGGC | 0.558333333 |
| (SEQ ID NO: 349) | CGCAAGTGCGTTCGAAGTGTCGATGATCAATGTGTCCTGCAATTCACATTA | |
| | ATTCTCGCAGCTAGCTGCGTTCTTCA | |

APPENDIX 5
120-mer DNA rRNA Capture Probes/Baits at 2x coverage, 85% GC restriction (Note: /5Biosg/ = 5′-biotin)

| Name | Sequence | GC content |
|---|---|---|
| KF899911.1_12SrRNA_r1_1 | /5Biosg/GCGTGCTTGATGCTTGTCCCTTTTGATCGTGGTGATTTAGAGGGTGAACTCACTGG | 0.45 |
| (SEQ ID NO: 349) | AACGGGGATGCTTGCATGTGTAATCTTACTAAGAGCTAATAGAAAGGCTAGGACCAAACCTATT | |
| KF899911.1_12SrRNA_r1_2 | /5Biosg/TTAATCACTGCTGTTTCCCGTGGGGGTGTGGCTAGGCTAAGCGTTTTGAGCTGCAT | 0.508333333 |
| (SEQ ID NO: 350) | TGCTGCGTGCTTGATGCTTGTCCCTTTTGATCGTGGTGATTTAGAGGGTGAACTCACTGGAACG | |
| KF899911.1_12SrRNA_r1_3 | /5Biosg/GAAATTGACCAACCCTGGGGTTAGTATAGCTTAGTTAAACTTTCGTTTATTGCTAA | 0.45 |
| (SEQ ID NO: 351) | AGGTTAATCACTGCTGTTTCCCGTGGGGGTGTGGCTAGGCTAAGCGTTTTGAGCTGCATTGCTG | |
| KF899911.1_12SrRNA_r1_4 | /5Biosg/ACACTCTTTACGCCGGCTTCTATTGACTTGGGTTAATCGTGTGACCGCGGTGGCTG | 0.458333333 |
| (SEQ ID NO: 352) | GCACGAAATTGACCAACCCTGGGGTTAGTATAGCTTAGTTAAACTTTCGTTTATTGCTAAAGGT | |
| KF899911.1_12SrRNA_r1_5 | /5Biosg/ACTGGAGTTTTTTACAACTCAGGTGAGTTTTAGCTTTATTGGGGAGGGGGTGATCT | 0.483333333 |
| (SEQ ID NO: 353) | AAAACACTCTTTACGCCGGCTTCTATTGACTTGGGTTAATCGTGTGACCGCGGTGGCTGGCACG | |
| KF899911.1_12SrRNA_r1_6 | /5Biosg/GGTCTTAGCTATTGTGTGTTCAGATATGTTAAAGCCACTTTCGTAGTCTATTTTGT | 0.383333333 |
| (SEQ ID NO: 354) | GTCAACTGGAGTTTTTTACAACTCAGGTGAGTTTTAGCTTTATTGGGGAGGGGGTGATCTAAAA | |
| KF899911.1_12SrRNA_r1_7 | /5Biosg/TGTTGATTTAACTGTTGAGGTTTAGGGCTAAGCATAGTGGGGTATCTAATCCCAGT | 0.383333333 |
| (SEQ ID NO: 355) | TTGGGTCTTAGCTATTGTGTGTTCAGATATGTTAAAGCCACTTTCGTAGTCTATTTTGTGTCAA | |
| KF899911.1_12SrRNA_r1_8 | /5Biosg/AAGCACCGCCAGGTCCTTTGAGTTTTAAGCTGTGGCTCGTAGTGTTCTGGCGAGCA | 0.466666667 |
| (SEQ ID NO: 356) | GTTTTGTTGATTTAACTGTTGAGGTTTAGGGCTAAGCATAGTGGGGTATCTAATCCCAGTTTGG | |
| KF899911.1_12SrRNA_r1_9 | /5Biosg/GAGGTGGTGAGGTTGATCGGGGTTTATCGATTACAGAACAGGCTCCTCTAGAGGGA | 0.508333333 |
| (SEQ ID NO: 356) | TATGAAGCACCGCCAGGTCCTTTGAGTTTTAAGCTGTGGCTCGTAGTGTTCTGGCGAGCAGTTT | |
| KF899911.1_12SrRNA_r1_10 | /5Biosg/CGCTTACTTTGTAGCCTTCATCAGGGTTTGCTGAAGATGGCGGTATATAGGCTGAG | 0.483333333 |
| (SEQ ID NO: 357) | CAAGAGGTGGTGAGGTTGATCGGGGTTTATCGATTACAGAACAGGCTCCTCTAGAGGGATATGA | |
| KF899911.1_12SrRNA_r1_11 | /5Biosg/GCCCATTTCTTGCCACCTCATGGGCTACACCTTGACCTAACGTCTTTACGTGGGTA | 0.5 |
| (SEQ ID NO: 358) | CTTGCGCTTACTTTGTAGCCTTCATCAGGGTTTGCTGAAGATGGCGGTATATAGGCTGAGCAAG | |
| KF899911.1_12SrRNA_r1_12 | /5Biosg/CCACCTTCGACCCTTAAGTTTCATAAGGGCTATCGTAGTTTTCTGGGGTAGAAAAT | 0.475 |
| (SEQ ID NO: 359) | GTAGCCCATTTCTTGCCACCTCATGGGCTACACCTTGACCTAACGTCTTTACGTGGGTACTTGC | |
| KF899911.1_12SrRNA_r1_13 | /5Biosg/TGTGTACGCGCTTCAGGGCCCTGTTCAACTAAGCACTCTACTCTCAGTTTACTGCT | 0.45 |
| (SEQ ID NO: 360) | AAATCCACCTTCGACCCTTAAGTTTCATAAGGGCTATCGTAGTTTTCTGGGGTAGAAAATGTAG | |
| KF899911.1_12SrRNA_r1_14 | /5Biosg/ATGCGTAGGGGTTTTAGTTAAATGTCCTTTGAAGTATACTTGAGGAGGGTGACGGG | 0.475 |
| (SEQ ID NO: 361) | CGGTGTGTACGCGCTTCAGGGCCCTGTTCAACTAAGCACTCTACTCTCAGTTTACTGCTAAATC | |
| KF899911.1_12SrRNA_r1_15 | /5Biosg/GTTCGTCCAAGTGCACTTTCCAGTACACTTACCATGTTACGACTTGTCTCCTCTAT | 0.441666667 |
| (SEQ ID NO: 362) | ATAAATGCGTAGGGGTTTTAGTTAAATGTCCTTTGAAGTATACTTGAGGAGGGTGACGGGCGGT | |
| KF899911.1_16SrRNA_r1_1 | /5Biosg/TTGCGGTACTATATCTATTGCGCCAGGTTTCAATTTCTATCGCCTATACTTTATTT | 0.425 |
| (SEQ ID NO: 363) | GGGTAAATGGTTTGGTTAAGGTTGTCTGGTAGTAAGGTGGAGTGGGTTTGGGGCTAGGTTTAGC | |
| KF899911.1_16SrRNA_r1_2 | /5Biosg/AGAAGGTATAGGGGTTAGTCCTTGCTATATTATGCTTGGTTATAATTTTTCATCTT | 0.366666667 |
| (SEQ ID NO: 364) | TCCCTTGCGGTACTATATCTATTGCGCCAGGTTTCAATTTCTATCGCCTATACTTTATTTGGGT | |
| KF899911.1_16SrRNA_r1_3 | /5Biosg/GTTTCGGGGGTCTTAGCTTTGGCTCTCCTTGCAAAGTTATTTCTAGTTAATTCATT | 0.375 |
| (SEQ ID NO: 365) | ATGCAGAAGGTATAGGGGTTAGTCCTTGCTATATTATGCTTGGTTATAATTTTTCATCTTTCCC | |
| KF899911.1_16SrRNA_r1_4 | /5Biosg/CCCACTATTTTGCTACATAGACGGGTGTGCTCTTTTAGCTGTTCTTAGGTAGCTCG | 0.433333333 |
| (SEQ ID NO: 366) | TCTGGTTTCGGGGGTCTTAGCTTTGGCTCTCCTTGCAAAGTTATTTCTAGTTAATTCATTATGC | |
| KF899911.1_16SrRNA_r1_5 | /5Biosg/ATCTTGGACAACCAGCTATCACCAGGCTCGGTAGGTTTGTCGCCTCTACCTATAAA | 0.466666667 |
| (SEQ ID NO: 367) | TCTTCCCACTATTTTGCTACATAGACGGGTGTGCTCTTTTAGCTGTTCTTAGGTAGCTCGTCTG | |
| KF899911.1_16SrRNA_r1_6 | /5Biosg/TAAATTTACAAGGGGATTTAGAGGGTTCTGTGGGCAAATTTAAAGTTGAACTAAGA | 0.4 |
| (SEQ ID NO: 368) | TTCTATCTTGGACAACCAGCTATCACCAGGCTCGGTAGGTTTGTCGCCTCTACCTATAAATCTT | |
| KF899911.1_16SrRNA_r1_7 | /5Biosg/CTCTCTCTACAAGGTTTTTTCCTAGTGTCCAAAGAGCTGTTCCTCTTTGGACTAAC | 0.375 |
| (SEQ ID NO: 369) | AGTTAAATTTACAAGGGGATTTAGAGGGTTCTGTGGGCAAATTTAAAGTTGAACTAAGATTCTA | |
| KF899911.1_16SrRNA_r1_8 | /5Biosg/CTTGAACGCTTTCTTAATTGGTGGCTGCTTTTAGGCCTACTATGGGTGTTAAATTT | 0.391666667 |
| (SEQ ID NO: 370) | TTTACTCTCTCTACAAGGTTTTTTCCTAGTGTCCAAAGAGCTGTTCCTCTTTGGACTAACAGTT | |
| KF899911.1_16SrRNA_r1_9 | /5Biosg/CCAATTGGGTGTGAGGAGTTCAGTTATATGTTTGGGATTTTTTAGGTAGTGGGTGT | 0.391666667 |
| (SEQ ID NO: 371) | TGAGCTTGAACGCTTTCTTAATTGGTGGCTGCTTTTAGGCCTACTATGGGTGTTAAATTTTTTA | |
| KF899911.1_16SrRNA_r1_10 | /5Biosg/GGAGAATGTTTTCATGTTACTTATACTAACATTAGTTCTTCTATAGGGTGATAGAT | 0.366666667 |
| (SEQ ID NO: 372) | TGGTCCAATTGGGTGTGAGGAGTTCAGTTATATGTTTGGGATTTTTTAGGTAGTGGGTGTTGAG | |
| KF899911.1_16SrRNA_r1_11 | /5Biosg/GTAGATATTGGGCTGTTAATTGTCAGTTCAGTGTTTTAATCTGACGCAGGCTTATG | 0.366666667 |
| (SEQ ID NO: 373) | CGGAGGAGAATGTTTTCATGTTACTTATACTAACATTAGTTCTTCTATAGGGTGATAGATTGGT | |
| KF899911.1_16SrRNA_r1_12 | /5Biosg/CCTTATGAGCATGCCTGTGTTGGGTTGACAGTGAGGGTAATAATGACTTGTTGGTT | 0.425 |
| (SEQ ID NO: 374) | GATTGTAGATATTGGGCTGTTAATTGTCAGTTCAGTGTTTTAATCTGACGCAGGCTTATGCGGA | |
| KF899911.1_16SrRNA_r1_13 | /5Biosg/TGTTTTTGGTAAACAGGCGGGGTAAGATTTGCCGAGTTCCTTTTACTTTTTTTAAC | 0.4 |
| (SEQ ID NO: 375) | CTTTCCTTATGAGCATGCCTGTGTTGGGTTGACAGTGAGGGTAATAATGACTTGTTGGTTGATT | |
| KF899911.1_16SrRNA_r1_14 | /5Biosg/GCCGTTAAACATGTGTCACTGGGCAGGCGGTGCCTCTAATACTGGTGATGCTAGAG | 0.45 |
| (SEQ ID NO: 376) | GTGATGTTTTTGGTAAACAGGCGGGGTAAGATTTGCCGAGTTCCTTTTACTTTTTTTAACCTTT | |
| KF899911.1_16SrRNA_r1_15 | /5Biosg/TACAGGTCCCTATTTAAGGAACAAGTGATTATGCTACCTTTGCACGGTTAGGGTAC | 0.5 |
| (SEQ ID NO: 377) | CGCGGCCGTTAAACATGTGTCACTGGGCAGGCGGTGCCTCTAATACTGGTGATGCTAGACGTGA | |
| KF899911.1_16SrRNA_r1_16 | /5Biosg/GGGCAGGTCAATTTCACTGGTTAAAAGTAAGAGACAGCTGAACCCTCGTGGAGCCA | 0.475 |
| (SEQ ID NO: 378) | TTCATACAGGTCCCTATTTAAGGAACAAGTGATTATGCTACCTTTGCACGGTTAGGGTACCGCG | |
| KF899911.1_16SrRNA_r1_17 | /5Biosg/TTAATAAATTAAAGCTCCATAGGGTCTTCTCGTCTTGCTGTGTCATGCCCGCCTCT | 0.466666667 |
| (SEQ ID NO: 379) | TCACGGGCAGGTCAATTTCACTGGTTAAAAGTAAGAGACAGCTGAACCCTCGTGGAGCCATTCA | |
| KF899911.1_16SrRNA_r1_18 | /5Biosg/GAAATTTTTAATGCAGGTTTGGTAGTTTAGGACCTGTGGGTTTGTTAGGTACTGTT | 0.416666667 |
| (SEQ ID NO: 380) | TGCATTAATAAATTAAAGCTCCATAGGGTCTTCTCGTCTTGCTGTGTCATGCCCGCCTCTTCAC | |
| KF899911.1_16SrRNA_r1_19 | /5Biosg/GGTGAAGTCTTAGCATGTACTGCTCGGAGGTTGGGTTCTGCTCCGAGGTCGCCCCA | 0.483333333 |
| (SEQ ID NO: 381) | ACCGAAATTTTTAATGCAGGTTTGGTACTTTAGGACCTGTGGGTTTGTTAGGTACTGTTTGCAT | |
| KF899911.1_16SrRNA_r1_20 | /5Biosg/GGTAACTTGTTCCGTTGGTCAAGTTATTGGATCAATTGAGTATAGTAGTTCGCTTT | 0.491666667 |
| (SEQ ID NO: 382) | GACTGGTGAAGTCTTAGCATGTACTGCTCGGAGGTTGGGTTCTGCTCPGAGGTCGCCCCAACCG | |
| KF899911.1_16SrRNA_r1_21 | /5Biosg/GAGGTCGTAAACCCTATTGTTGATATGGACTCTAGAATAGGATTGCGCTGTTATCC | 0.408333333 |
| (SEQ ID NO: 383) | CTACGGTAACTTGTTCCGTTGGTCAAGTTATTGGATCAATTGAGTATAGTACTTCGCTTTGACT | |
| KF899911.1_16SrRNA_r1_22 | /5Biosg/TCGTTGAACAAACGAACCTTTAATAGCGGCTGCACCATCGGGATGTCCTGATCCAA | 0.458333333 |
| (SEQ ID NO: 384) | CATCGAGGTCGTAAACCCTATTGTTGATATGGACTCTAGAATAGGATTGCGCTGTTATCCCTAG | |
| KF899911.1_16SrRNA_r1_23 | /5Biosg/AAGTAGATAGAAACCGACCTGGATTACTCCGGTCTGAACTCAGATCACGTAGGACT | 0.458333333 |
| (SEQ ID NO: 385) | TTAATCGTTGAACAAACGAACCTTTAATAGCGGCTGCACCATCGGGATGTCCTGATCCAACATC | |
| KF899911.1_16SrRNA_r1_24 | /5Biosg/GGAAGGCGCTTTGTGAAGTAGGCCTTATTTCTCTTGTCCTTTCGTACAGGGAGGAA | 0.45 |
| (SEQ ID NO: 386) | TTTGAAGTAGATAGAAACCGACCTGGATTACTCCGGTCTGAACTCAGATCACGTAGGACTTTAA | |
| KF899911.1_16SrRNA_r1_25 | /5Biosg/AAACCCTGTTCTTGGGTGGGTGTGGGTATAATACTAAGTTGAGATGATATCATTTA | 0.441666667 |
| (SEQ ID NO: 387) | CGGGGGAAGGCGCTTTGTGAAGTAGGCCTTATTTCTCTTGTCCTTTCGTACAGGGAGGAATTTG | |
| NR_003286.2_RNA18S5_r1_1 | /5Biosg/AAGGAACCATAACTGATTTAATGAGCCATTCGCAGTTTCACTGTACCGGCCGTGCG | 0.458333333 |
| (SEQ ID NO: 388) | TACTCAGACATGCATGGCTTAATCTTTGAGACAAGCATATGCTACTGGCAGGATCAACCAGGTA | |
| NR_003286.2_RNA18S5_r1_2 | /5Biosg/GCATGTATTAGCTCTAGAATTACCACAGTTATCCAAGTAGGAGAGGAGCGAGCGAC | 0.466666667 |
| (SEQ ID NO: 389) | CAAAGGAACCATAACTGATTTAATGAGCCATTCGCAGTTTCACTGTACCGGCCGTGCGTACTCA | |
| NR_003286.2_RNA18S5_r1_3 | /5Biosg/GTTGGTTTTGATCTGATAAATGCACGCATCCCCCCCGCGAAGGGGGTCAGCGCCCG | 0.533333333 |
| (SEQ ID NO: 390) | TCGGCATGTATTAGCTCTAGAATTACCACAGTTATCCAAGTAGGAGAGGAGCGAGCGACCAAAG | |
| NR_003286.2_RNA18S5_r1_4 | /5Biosg/GAGTCACCAAAGCCGCCGGCGCCCGCCCCCCGGCCGGGGCCGGAGAGGGGCTGACC | 0.716666667 |
| (SEQ ID NO: 391) | GGGTTGGTTTTGATCTGATAAATGCACGCATCCCCCCCGCGAAGGGGGTCAGCGCCCGTCGGCA | |
| NR_003286.2_RNA18S5_r1_5 | /5Biosg/GACGTTCGAATGGGTCGTCGCCGCCACGGGGGGCGTGCGATCGGCCCGAGGTTATC | 0.741666667 |
| (SEQ ID NO: 392) | TAGAGTCACCAAAGCCGCCGGCGCCCGCCCCCCGGCCGGGGCCGGAGAGGGGCTGACCGGGTTG | |
| NR_003286.2_RNA18S5_r1_6 | /5Biosg/CCGTCACCCGTGGTCACCATGGTAGGCACGGCGACTACCATCGAAAGTTGATAGGG | 0.625 |
| (SEQ ID NO: 393) | CAGACGTTCGAATGGGTCGTCGCCGCCACGGGGGGCGTGCGATCGGCCCGAGGTTATCTAGAGT | |
| NR_003286.2_RNA18S5_r1_7 | /5Biosg/CTTCCTTGGATGTGGTAGCCGTTTCTCAGGCTCCCTCTCCGGAATCGAACCCTGAT | 0.575 |
| (SEQ ID NO: 394) | TCCCCGTCACCCGTGGTCACCATGGTAGGCACGGCGACTACCATCGAAAGTTGATAGGGCAGAC | |
| NR_003286.2_RNA18S5_r1_8 | /5Biosg/TGTTATTTTTCGTCACTACCTCCCCGGGTCGGGAGTGGGTAATTTGCGCGCCTGCT | 0.575 |
| (SEQ ID NO: 395) | GCCTTCCTTGGATGTGGTAGCCGTTTCTCAGGCTCCCTCTCCGGAATCGAACCCTGATTCCCCG | |
| NR_003286.2_RNA18S5_r1_9 | /5Biosg/CGTTAAAGGATTTAAAGTGGACTCATTCCAATTACAGGGCCTCGAAAGAGTCCTGT | 0.491666667 |
| (SEQ ID NO: 396) | ATTGTTATTTTTCGTCACTACCTCCCCGGGTCGGGAGTGGGTAATTTGCGCGCCTGCTGCCTTC | |
| NR_003286.2_RNA18S5_r1_10 | /5Biosg/GCTATTGGAGCTGGAATTACCGCGGCTGCTGGCACCAGACTTGCCCTCCAATGGAT | 0.491666667 |
| (SEQ ID NO: 397) | CCTCGTTAAAGGATTTAAAGTGGACTCATTCCAATTACAGGGCCTCGAAAGAGTCCTGTATTGT | |
| NR_003286.2_RNA18S5_r1_11 | /5Biosg/GCCCGCCCGCTCCCAAGATCCAACTACGAGCTTTTTAACTGCAGCAACTTTAATAT | 0.533333333 |
| (SEQ ID NO: 398) | ACGCTATTGGAGCTGGAATTACCGCGGCTGCTGGCACCAGACTTGCCCTCCAATGGATCCTCGT | |
| NR_003286.2_RNA18S5_r1_12 | /5Biosg/GAGGGGGCGCCGAGAGGCAAGGGGCGGGGACGGGCGGTGGCTCGCCTCGCGGCGGA | 0.65 |
| (SEQ ID NO: 399) | CCGCCCGCCCGCTCCCAAGATCCAACTACGAGCTTTTTAACTGCAGCAACTTTAATATACGCTA | |
| NR_003286.2_RNA18S5_r1_13 | /5Biosg/ACTCTAATTTTTTCAAAGTAAACGCTTCGGGCCCCGCGGGACACTCAGCTAAGAGC | 0.666666667 |
| (SEQ ID NO: 400) | ATCGAGGGGGCGCCGAGAGGCAAGGGGCGGGGACGGGCGGTGGCTCGCCTCGCGGCGGACCGCC | |
| NR_003286.2_RNA18S5_r1_14 | /5Biosg/CGGTCCTATTCCATTATTCCTAGCTGCGGTATCCAGGCGGCTCGGGCCTGCTTTGA | 0.533333333 |
| (SEQ ID NO: 401) | ACACTCTAATTTTTTCAAAGTAAACGCTTCGGGCCCCGCGGGACACTCAGCTAAGAGCATCGAG | |
| NR_003286.2_RNA18S5_r1_15 | /5Biosg/CCCCCGGCCGTCCCTCTTAATCATGGCCTCAGTTCCGAAAACCAACAAAATAGAAC | 0.55 |
| (SEQ ID NO: 402) | CGCGGTCCTATTCCATTATTCCTAGCTGCGGTATCCAGGCGGCTCGGGCCTGCTTTGAACACTC | |
| NR_003286.2_RNA18S5_r1_16 | /5Biosg/GCTCTGGTCCGTCTTGCGCCGGTCCAAGAATTTCACCTCTAGCGGCGCAATACGAA | 0.558333333 |
| (SEQ ID NO: 403) | TGCCCCCGGCCGTCCCTCTTAATCATGGCCTCAGTTCCGAAAACCAACAAAATAGAACCGCGGT | |
| NR_003286.2_RNA18S5_r1_17 | /5Biosg/GTCTTCGAACCTCCGACTTTCGTTCTTGATTAATGAAAACATTCTTGGCAAATGCT | 0.491666667 |
| (SEQ ID NO: 404) | TTCGCTCTGGTCCGTCTTGCGCCGGTCCAAGAATTTCACCTCTAGCGGCGCAATACGAATGCCC | |
| NR_003286.2_RNA18S5_r1_18 | /5Biosg/ACGCCGCCGCATCGCCGGTCGGCATCGTTTATGGTCGGAACTACGACGGTATCTGA | 0.5 |
| (SEQ ID NO: 405) | TCGTCTTCGAACCTCCGACTTTCGTTCTTGATTAATGAAAACATTCTTGGCAAATGCTTTCGCT | |
| NR_003286.2_RNA18S5_r1_19 | /5Biosg/CCCCCCGGAACCCAAAGACTTTGGTTTCCCGGAAGCTGCCCGGCGGGTCATGGGAA | 0.608333333 |
| (SEQ ID NO: 406) | TAACGCCGCCGCATCGCCGGTCGGCATCGTTTATGGTCGGAACTACGACGGTATCTGATCGTCT | |
| NR_003286.2_RNA18S5_r1_20 | /5Biosg/TCCACTCCTGGTGGTGCCCTTCCGTCAATTCCTTTAAGTTTCAGCTTTGCAACCAT | 0.55 |
| (SEQ ID NO: 407) | ACTCCCCCCGGAACCCAAAGACTTTGGTTTCCCGGAAGCTGCCCGGCGGGTCATGGGAATAACG | |
| NR_003286.2_RNA18S5_r1_21 | /5Biosg/CCTGTCCGTGTCCGGGCCGGGTGAGGTTTCCCGTGTTGAGTCAAATTAAGCCGCAG | 0.558333333 |
| (SEQ ID NO: 408) | GCTCCACTCCTGGTGGTGCCCTTCCGTCAATTCCTTTAAGTTTCAGCTTTGCAACCATACTCCC | |
| NR_003286.2_RNA18S5_r1_22 | /5Biosg/TAAGAACGGCCATGCACCACCACCCACGGAATCGAGAAAGAGCTATCAATCTGTCA | 0.55 |
| (SEQ ID NO: 409) | ATCCTGTCCGTGTCCGGGCCGGGTGAGGTTTCCCGTGTTGAGTCAAATTAAGCCGCAGGCTCCA | |
| NR_003286.2_RNA18S5_r1_23 | /5Biosg/GTTAGCATGCCAGAGTCTCGTTCGTTATCGGAATTAACCAGACAAATCGCTCCACC | 0.483333333 |
| (SEQ ID NO: 410) | AACTAAGAACGGCCATGCACCACCACCCACGGAATCGAGAAAGAGCTATCAATCTGTCAATCCT | |
| NR_003286.2_RNA18S5_r1_24 | /5Biosg/CGCCACTTGTCCCTCTAAGAAGTTGGGGGACGCCGACCGCTCGGGGGTCGCGTAAC | 0.541666667 |
| (SEQ ID NO: 411) | TAGTTAGCATGCCAGAGTCTCGTTCGTTATCGGAATTAACCAGACAAATCGCTCCACCAACTAA | |
| NR_003286.2_RNA18S5_r1_25 | /5Biosg/AGCCCCGGACATCTAAGGGCATCACAGACCTGTTATTGCTCAATCTCGGGTGGCTG | 0.575 |
| (SEQ ID NO: 412) | AACGCCACTTGTCCCTCTAAGAAGTTGGGGGACGCCGACCGCTCGGGGGTCGCGTAACTAGTTA | |
| NR_003286.2_RNA18S5_r1_26 | /5Biosg/CCCGCGCCTGCCGGCGTAGGGTAGGCACACGCTGAGCCAGTCAGTGTAGCGCGCGT | 0.641666667 |
| (SEQ ID NO: 413) | GCAGCCCCGGACATCTAAGGGCATCACAGACCTGTTATTGCTCAATCTCGGGTGGCTGAACGCC | |
| NR_003286.2_RNA18S5_r1_27 | /5Biosg/CGTTCATGGGGAATAATTGCAATCCCCGATCCCCATCACGAATGGGGTTCAACGGG | 0.616666667 |
| (SEQ ID NO: 414) | TTACCCGCGCCTGCCGGCGTAGGGTAGGCACACGCTGAGCCAGTCAGTGTAGCGCGCGTGCAGC | |
| NR_003286.2_RNA18S5_r1_28 | /5Biosg/ACAAAGGGCAGGGACTTAATCAACGCAAGCTTATGACCCGCACTTACTGGGAATTC | 0.508333333 |
| (SEQ ID NO: 415) | CTCGTTCATGGGGAATAATTGCAATCCCCGATCCCCATCACGAATGGGGTTCAACGGGTTACCC | |
| NR_003286.2_RNA18S5_r1_29 | /5Biosg/GGGCCGATCCGAGGGCCTCACTAAACCATCCAATCGGTAGTAGCGACGGGCGGTGT | 0.55 |
| (SEQ ID NO: 416) | GTACAAAGGGCAGGGACTTAATCAACGCAAGCTTATGACCCGCACTTACTGGGAATTCCTCGTT | |
| NR_003286.2_RNA18S5_r1_30 | /5Biosg/GATAGTCAAGTTCGACCGTCTTCTCAGCGCTCCGCCAGGGCCGTGGGCCGACCCCG | 0.65 |
| (SEQ ID NO: 417) | GCGGGGCCGATCCGAGGGCCTCACTAAACCATCCAATCGGTAGTAGCGACGGGCGGTGTGTACA | |
| NR_003286.2_RNA18S5_r1_31 | /5Biosg/TAATGATCCTTCCGCAGGTTCACCTACGGAAACCTTGTTACGACTTTTACTTCCTC | 0.575 |
| (SEQ ID NO: 418) | TAGATAGTCAAGTTCGACCGTCTTCTCAGCGCTCCGCCAGGGCCGTGGGCCGACCCCGGCGGGG | |
| NR_003287.2_RNA28S5_r1.1_1 | /5Biosg/TCGGCGCTGGGCTCTTCCCTGTTCACTCGCCGTTACTGAGGGAATCCTGGTTAGTT | 0.55 |
| (SEQ ID NO: 419) | TCTTCTCCTCCGCTGACTAATATGCTTAAATTCAGCGGGTCGCCACGTCTGATCTGAGGTCGCG | |
| NR_003287.2_RNA28S5_r1.1_2 | /5Biosg/GCCGGGGAGCGGGTCTTCCGTACGCCACATGTCCCGCGCCCCGCCGCGGGGCGGGG | 0.666666667 |
| (SEQ ID NO: 420) | ATTCGGCGCTGGGCTCTTCCCTGTTCACTCGCCGTTACTGAGGGAATCCTGGTTAGTTTCTTCT | |
| NR_003287.2_RNA28S5_r1.1_3 | /5Biosg/TCACACCGTCCACGGGCTGGGCCTCGATCAGAAGGACTTGGGCCCCCCACGAGCGG | 0.75 |
| (SEQ ID NO: 421) | CGCCGGGGAGCGGGTCTTCCGTACGCCACATGTCCCGCGCCCCGCCGCGGGGCGGGGATTCGGC | |
| NR_003287.2_RNA28S5_r1.1_4 | /5Biosg/CCAAGCAACCCGACTCCGGGAAGACCCGGGCCCGGCGCGCCGGGGGCCGCTACCGG | 0.766666667 |
| (SEQ ID NO: 422) | CCTCACACCGTCCACGGGCTGGGCCTCGATCAGAAGGACTTGGGCCCCCCACGAGCGGCGCCGG | |
| NR_003287.2_RNA28S5_r1.1_5 | /5Biosg/GTCTCGTGCCGGTATTTAGCCTTAGATGGAGTTTACCACCCGCTTTGGGCTGCATT | 0.658333333 |
| (SEQ ID NO: 423) | CCCAAGCAACCCGACTCCGGGAAGACCCGGGCCCGGCGCGCCGGGGGCCGCTACCGGCCTCACA | |
| NR_003287.2_RNA28S5_r1.1_6 | /5Biosg/TTGAACTCTCTCTTCAAAGTTCTTTTCAACTTTCCCTTACGGTACTTGTTGACTAT | 0.458333333 |
| (SEQ ID NO: 424) | CGGTCTCGTGCCGGTATTTAGCCTTAGATGGAGTTTACCACCCGCTTTGGGCTGCATTCCCAAG | |
| NR_003287.2_RNA28S5_r1.1_7 | /5Biosg/CCTCCGGGCGGACTGCGCGGACCCCACCCGTTTACCTCTTAACGGTTTCACGCCCT | 0.525 |
| (SEQ ID NO: 425) | CTTGAACTCTCTCTTCAAAGTTCTTTTCAACTTTCCCTTACGGTACTTGTTGACTATCGGTCTC | |
| NR_003287.2_RNA28S5_r1.1_8 | /5Biosg/GGGGGCGGGAAAGATCCGCCGGGCCGCCGACACGGCCGGACCCGCCGCCGGGTTGA | 0.7 |
| (SEQ ID NO: 426) | ATCCTCCGGGCGGACTGCGCGGACCCCACCCGTTTACCTCTTAACGGTTTCACGCCCTCTTGAA | |
| NR_003287.2_RNA28S5_r1.1_9 | /5Biosg/GAGGAGGAGGAGGGGCGGCGGGGGAAGGGAGGGCGGGTGGAGGGGTCGGGAGGAAC | 0.775 |
| (SEQ ID NO: 427) | GGGGGGCGGGAAAGATCCGCCGGGCCGCCGACACGGCCGGACCCGCCGCCGGGTTGAATCCTCC | |
| NR_003287.2_RNA28S5_r1.- | /5Biosg/GCCACCTTCCCCGCCGGGCCTTCCCAGCCGTCCCGGAGCCGGTCGCGGCGCACCGC | 0.825 |
| 1_12 (SEQ ID NO: 428) | CGCGGTGGAAATGCGCCCGGCGGCGGCCGGTCGCCGGTCGGGGGACGGTCCCCCGCCGACCCCA | |
| NR_003287.2_RNA28S5_r1.- | /5Biosg/GGGGGGCGGAGACGGGGGAGGAGGAGGACGGACGGACGGACGGACGGGGCCCCCCG | 0.816666667 |
| 1_13 (SEQ ID NO: 429) | AGCCACCTTCCCCGCCGGGCCTTCCCAGCCGTCCCGGAGCCGGTCGCGGCGCACCGCCGCGGTG | |
| NR_003287.2_RNA28S5_r1.2_1 | /5Biosg/GAGGGGGGAGAGCGCGGCGACGGGTCTCGCTCCCTCGGCCCCGGGATTCGGCGAGT | 0.791666667 |
| (SEQ ID NO: 430) | GCTGCTGCCGGGGGGGCTGTAACACTCGGGGGGGGTTTCGGTCCCGCCGCCGCCGCCGCCGCCG | |
| NR_003287.2_RNA28S5_r1.2_2 | /5Biosg/GGCGCGCCCCCGCGGGGGAGACCCCCCTCGCGGGGGATTCCCCGCGGGGGTGGGCG | 0.825 |
| (SEQ ID NO: 431) | CCGGGAGGGGGGAGAGCGCGGCGACGGGTCTCGCTCCCTCGGCCCCGGGATTCGGCGAGTGCTG | |
| NR_003287.2_RNA28S5_r1.2_6 | /5Biosg/CGCCGACCCCGTGCGCTCGCTCCGCCGTCCCCCTCTTCGGGGGACGCGCGCGTGGC | 0.816666667 |
| (SEQ ID NO: 432) | CCCGAGAGAACCTCCCCCGGGCCCGACGGCGCGACCCGCCCGGGGCGCACTGGGGACAGTCCGC | |
| NR_003287.2_RNA28S5_r1.2_7 | /5Biosg/GCACGTGTTAGACTCCTTGGTCCGTGTTTCAAGACGGGTCGGGTGGGTAGCCGACG | 0.725 |
| (SEQ ID NO: 433) | TCGCCGCCGACCCCGTGCGCTCGCTCCGCCGTCCCCCTCTTCGGGGGACGCGCGCGTGGCCCCG | |
| NR_003287.2_RNA28S5_r1.2_8 | /5Biosg/AGCGCGCCGGCCTTCACCTTCATTGCGCCACGGCGGCTTTCGTGCGAGCCCCCGAC | 0.666666667 |
| (SEQ ID NO: 434) | TCGCGCACGTGTTAGACTCCTTGGTCCGTGTTTCAAGACGGGTCGGGTGGGTAGCCGACGTCGC | |
| NR_003287.2_RNA28S5_r1.2_9 | /5Biosg/CGGGCCGGTGGTGCGCCCTCGGCGGACTGGAGAGGCCTCGGGATCCCACCTCGGCC | 0.758333333 |
| (SEQ ID NO: 435) | GGCGAGCGCGCCGGCCTTCACCTTCATTGCGCCACGGCGGCTTTCGTGCGAGCCCCCGACTCGC | |
| NR_003287.2_RNA28S5_r1.- | /5Biosg/CCATCTTTCGGGTCCTAACACGTGCGCTCGTGCTCCACCTCCCCGGCGCGGCGGGC | 0.75 |
| 2_10 (SEQ ID NO: 436) | GAGACGGGCCGGTGGTGCGCCCTCGGCGGACTGGAGAGGCCTCGGGATCCCACCTCGGCCGGCG | |
| NR_003287.2_RNA28S5_r1.- | /5Biosg/GGACCGCTACGGACCTCCACCAGAGTTTCCTCTGGCTTCGCCCTGCCCAGGCATAG | 0.658333333 |
| 2_11 (SEQ ID NO: 437) | TTCACCATCTTTCGGGTCCTAACACGTGCGCTCGTGCTCCACCTCCCCGGCGCGGCGGGCGAGA | |
| NR_003287.2_RNA28S5_r1.- | /5Biosg/TAGATGGTTCGATTAGTCTTTCGCCCCTATACCCAGGTCGGACGACCGATTTGCAC | 0.566666667 |
| 2_12 (SEQ ID NO: 438) | GTCAGGACCGCTACGGACCTCCACCAGAGTTTCCTCTGGCTTCGCCCTGCCCAGGCATAGTTCA | |
| NR_003287.2_RNA28S5_r1.- | /5Biosg/TGCGTCGGGTCTGCGAGAGCGCCAGCTATCCTGAGGGAAACTTCGGAGGGAACCAG | 0.566666667 |
| 2_13 (SEQ ID NO: 439) | CTACTAGATGGTTCGATTAGTCTTTCGCCCCTATACCCAGGTCGGACGACCGATTTGCACGTCA | |
| NR_003287.2_RNA28S5_r1.- | /5Biosg/TCGTTTCGGCCCCAAGACCTCTAATCATTCGCTTTACCGGATAAAACTGCGTGGCG | 0.583333333 |
| 2_14 (SEQ ID NO: 440) | GGGGTGCGTCGGGTCTGCGAGAGCGCCAGCTATCCTGAGGGAAACTTCGGAGGGAACCAGCTAC | |
| NR_003287.2_RNA28S5_r1.- | /5Biosg/CCGGCTCCACGCCAGCGAGCCGGGCTTCTTACCCATTTAAAGTTTGAGAATAGGTT | 0.541666667 |
| 2_15 (SEQ ID NO: 441) | GAGATCGTTTCGGCCCCAAGACCTCTAATCATTCGCTTTACCGGATAAAACTGCGTGGCGGGGG | |
| NR_003287.2_RNA28S5_r1.- | /5Biosg/CATCCCGCAGCGCCAGTTCTGCTTACCAAAAGTGGCCCACTAGGCACTCGCATTCC | 0.566666667 |
| 2_16 (SEQ ID NO: 442) | ACGCCCGGCTCCACGCCAGCGAGCCGGGCTTCTTACCCATTTAAAGTTTGAGAATAGGTTGAGA | |
| NR_003287.2_RNA28S5_r1.- | /5Biosg/AACACCTTTTCTGGGGTCTGATGAGCGTCGGCATCGGGCGCCTTAACCCGGCGTTC | 0.6 |
| 2_17 (SEQ ID NO: 443) | GGTTCATCCCGCAGCGCCAGTTCTGCTTACCAAAAGTGGCCCACTAGGCACTCGCATTCCACGC | |
| NR_003287.2_RNA28S5_r1.- | /5Biosg/TTACACACTCCTTAGCGGATTCCGACTTCCATGGCCACCGTCCTGCTGTCTATATC | 0.558333333 |
| 2_18 (SEQ ID NO: 444) | AACCAACACCTTTTCTGGGGTCTGATGAGCGTCGGCATCGGGCGCCTTAACCCGGCGTTCGGTT | |
| NR_003287.2_RNA28S5_r1.- | /5Biosg/ATGGGCCCGACGCTCCAGCGCCATCCATTTTCAGGGCTAGTTGATTCGGCAGGTGA | 0.55 |
| 2_19 (SEQ ID NO: 445) | GTTGTTACACACTCCTTAGCGGATTCCGACTTCCATGGCCACCGTCCTGCTGTCTATATCAACC | |
| NR_003287.2_RNA28S5_r1.- | /5Biosg/CGCGCGCGCGCGCCGCCCCCGCCGCTCCCGTCCACTCTCGACTGCCGGCGACGGCC | 0.716666667 |
| 2_20 (SEQ ID NO: 446) | GGGTATGGGCCCGACGCTCCAGCGCCATCCATTTTCAGGGCTAGTTGATTCGGCAGGTGAGTTG | |
| NR_003287.2_RNA28S5_r1.- | /5Biosg/GAAGGACCCCACACCCCCGCCGCCGCCGCCGCCGCCGCCCTCCGACGCACACCACA | 0.833333333 |
| 2_21 (SEQ ID NO: 447) | CGCGCGCGCGCGCGCGCCGCCCCCGCCGCTCCCGTCCACTCTCGACTGCCGGCGACGGCCGGGT | |
| NR_003287.2_RNA28S5_r1.- | /5Biosg/GCTTCAAGGCTCACCGCAGCGGCCCTCCTACTCGTCGCGGCGTAGCGTCCGCGGGG | 0.808333333 |
| 2_23 (SEQ ID NO: 448) | CTCCGGGGGCGGGGAGCGGGGCGTGGGCGGGAGGAGGGGAGGAGGCGTGGGGGGGGGGGCGGGG | |
| NR_003287.2_RNA28S5_r1.- | /5Biosg/TATTTGCTACTACCACCAAGATCTGCACCTGCGGCGGCTCCACCCGGGCCCGCGCC | 0.691666667 |
| 2_24 (SEQ ID NO: 449) | CTAGGCTTCAAGGCTCACCGCAGCGGCCCTCCTACTCGTCGCGGCGTAGCGTCCGCGGGGCTCC | |
| NR_003287.2_RNA28S5_r1.- | /5Biosg/GTTCAACTGCTGTTCACATGGAACCCTTCTCCACTTCGGCCTTCAAAGTTCTCGTT | 0.558333333 |
| 2_25 (SEQ ID NO: 450) | TGAATATTTGCTACTACCACCAAGATCTGCACCTGCGGCGGCTCCACCCGGGCCCGCGCCCTAG | |
| NR_003287.2_RNA28S5_r1.- | /5Biosg/AGGCCATCGCCCGTCCCTTCGGAACGGCGCTCGCCCATCTCTCAGGACCGACTGAC | 0.566666667 |
| 2_26 (SEQ ID NO: 451) | CCATGTTCAACTGCTGTTCACATGGAACCCTTCTCCACTTCGGCCTTCAAAGTTCTCGTTTGAA | |
| NR_003287.2_RNA28S5_r1.- | /5Biosg/CCGCCACTCCGGATTCGGGGATCTGAACCCGACTCCCTTTCGATCGGCCGAGGGCA | 0.666666667 |
| 2_27 (SEQ ID NO: 452) | ACGGAGGCCATCGCCCGTCCCTTCGGAACGGCGCTCGCCCATCTCTCAGGACCGACTGACCCAT | |
| NR_003287.2_RNA28S5_r1.- | /5Biosg/CCCGCCGGCTTCTCCGGGATCGGTCGCGTTACCGCACTGGACGCCTCGCGGCGCCC | 0.7 |
| 2_28 (SEQ ID NO: 453) | ATCTCCGCCACTCCGGATTCGGGGATCTGAACCCGACTCCCTTTCGATCGGCCGAGGGCAACGG | |
| NR_003287.2_RNA28S5_r1.- | /5Biosg/GGCGAACCCATTCCAGGGCGCCCTGCCCTTCACAAAGAAAAGAGAACTCTCCCCGG | 0.675 |
| 2_29 (SEQ ID NO: 454) | GGCTCCCGCCGGCTTCTCCGGGATCGGTCGCGTTACCGCACTGGACGCCTCGCGGCGCCCATCT | |
| NR_003287.2_RNA28S5_r1.- | /5Biosg/AGAGCTCACCGGACGCCGCCGGAACCGCGACGCTTTCCAAGGCACGGGCCCCTCTC | 0.666666667 |
| 2_30 (SEQ ID NO: 455) | TCGGGGCGAACCCATTCCAGGGCGCCCTGCCCTTCACAAAGAAAAGAGAACTCTCCCCGGGGCT | |
| NR_003287.2_RNA28S5_r1.- | /5Biosg/ATATGGGTACGGCCCGGCGCGAGATTTACACCCTCTCCCCCGGATTTTCAAGGGCC | 0.666666667 |
| 2_31 (SEQ ID NO: 456) | AGCGAGAGCTCACCGGACGCCGCCGGAACCGCGACGCTTTCCAAGGCACGGGCCCCTCTCTCGG | |
| NR_003287.2_RNA28S5_r1.- | /5Biosg/TTCCCTTACCTACATTGTTCCAACATGCCAGAGGCTGTTCACCTTGGAGACCTGCT | 0.575 |
| 2_32 (SEQ ID NO: 457) | GCGGATATGGGTACGGCCCGGCGCGAGATTTACACCCTCTCCCCCGGATTTTCAAGGGCCAGCG | |
| NR_003287.2_RNA28S5_r1.- | /5Biosg/CGACCGACCCAGCCCTTAGAGCCAATCCTTATCCCGAAGTTACGGATCCGGCTTGC | 0.566666667 |
| 2_33 (SEQ ID NO: 458) | CGACTTCCCTTACCTACATTGTTCCAACATGCCAGAGGCTGTTCACCTTGGAGACCTGCTGCGG | |
| NR_003287.2_RNA28S5_r1.- | /5Biosg/GGGGGCGGCGGCGCCTCGTCCAGCCGCGGCGCGCGCCCAGCCCCGCTTCGCGCCCC | 0.741666667 |
| 2_34 (SEQ ID NO: 459) | AGCCCGACCGACCCAGCCCTTAGAGCCAATCCTTATCCCGAAGTTACGGATCCGGCTTGCCGAC | |
| NR_003287.2_RNA28S5_r1.- | /5Biosg/AGGGGGGAGGACGGGGAGCGGGGGAGAGAGAGAGAGAGAGGGCGCGGGGCGGGGAG | 0.833333333 |
| 2_36 (SEQ ID NO: 460) | GGAGCGAGCGGCGCGCGCGGGGTGGGGCGGGGGAGGGCCGCGAGGGGGGTGCCCCGGGCGTGGG | |
| NR_003287.2_RNA28S5_r1.- | /5Biosg/GCCCCTGCCGCCCCGACCCTTCTCCCCCCGCCGCCGCCCCCACGCGGCGCTCCCCC | 0.825 |
| 2_37 (SEQ ID NO: 461) | GGGGAGGGGGGAGGACGGGGAGCGGGGGAGAGAGAGAGAGAGAGGGCGCGGGGCGGGGAGGGAG | |
| NR_003287.2_RNA28S5_r1.- | /5Biosg/TTCCCCTGGTCCGCACCAGTTCTAAGTCGGCTGCTAGGCGCCGGCCGAGGCGAGGC | 0.816666667 |
| 2_45 (SEQ ID NO: 462) | GCCGCGCGGAACCGCGGCCCCGGGGGCGGACCCGGCGGGGGGGACCGGCCCGCGGCCCCTCCGC | |
| NR_003287.2_RNA28S5_r1.- | /5Biosg/ACATCGCGTCAACACCCGCCGCGGGCCTTCGCGATGCTTTGTTTTAATTAAACAGT | 0.625 |
| 2_46 (SEQ ID NO: 463) | CGGATTCCCCTGGTCCGCACCAGTTCTAAGTCGGCTGCTAGGCGCCGGCCGAGGCGAGGCGCCG | |
| NR_003287.2_RNA28S5_r1.- | /5Biosg/CGTTTACCCGCGCTTCATTGAATTTCTTCACTTTGACATTCAGAGCACTGGGCAGA | 0.491666667 |
| 2_47 (SEQ ID NO: 464) | AATCACATCGCGTCAACACCCGCCGCGGGCCTTCGCGATGCTTTGTTTTAATTAAACAGTCGGA | |
| NR_003287.2_RNA28S5_r1.- | /5Biosg/GCGTCACTAATTAGATGACGAGGCATTTGGCTACCTTAAGAGAGTCATAGTTACTC | 0.458333333 |
| 2_48 (SEQ ID NO: 465) | CCGCCGTTTACCCGCGCTTCATTGAATTTCTTCACTTTGACATTCAGAGCACTGGGCAGAAATC | |
| NR_003287.2_RNA28S5_r1.- | /5Biosg/GGCTGTGGTTTCGCTGGATAGTAGGTAGGGACAGTGGGAATCTCGTTCATCCATTC | 0.491666667 |
| 2_49 (SEQ ID NO: 466) | ATGCGCGTCACTAATTAGATGACGAGGCATTTGGCTACCTTAAGAGAGTCATAGTTACTCCCGC | |
| NR_003287.2_RNA28S5_r1.- | /5Biosg/ACTAGAGTCAAGCTCAACAGGGTCTTCTTTCCCCGCTGATTCCGCCAAGCCCGTTC | 0.533333333 |
| 2_50 (SEQ ID NO: 467) | CCTTGGCTGTGGTTTCGCTGGATAGTAGGTAGGGACAGTGGGAATCTCGTTCATCCATTCATGC | |
| NR_003287.2_RNA28S5_r1.- | /5Biosg/GGGGGGGCGCCGGGGGCCTCCCACTTATTCTACACCTCTCATGTCTCTTCACCGTG | 0.6 |
| 2_51 (SEQ ID NO: 468) | CCAGACTAGAGTCAAGCTCAACAGGGTCTTCTTTCCCCGCTGATTCCGCCAAGCCCGTTCCCTT | |
| NR_003287.2_RNA28S5_r1.- | /5Biosg/TATTTCACCGGCGGCCCGCAGGGCCGGCGGACCCCGCCCCGGGCCCCTCGCGGGGA | 0.733333333 |
| 2_52 (SEQ ID NO: 469) | CACCGGGGGGGCGCCGGGGGCCTCCCACTTATTCTACACCTCTCATGTCTCTTCACCGTGCCAG | |
| NR_003287.2_RNA28S5_r1.- | /5Biosg/GCCCCTCGGGGCTCGCCCCCCCGCCTCACCGGGTCAGTGAAAAAACGATCAGAGTA | 0.741666667 |
| 2_53 (SEQ ID NO: 470) | GTGGTATTTCACCGGCGGCCCGCAGGGCCGGCGGACCCCGCCCCGGGCCCCTCGCGGGGACACC | |
| NR_003287.2_RNA28S5_r1.- | /5Biosg/TCCCCGGAGCGGGTCGCGCCCGGCCGGCGCGCGGCCGGGCGCTTGGCGCCAGAAGC | 0.75 |
| 2_54 (SEQ ID NO: 471) | GAGAGCCCCTCGGGGCTCGCCCCCCCGCCTCACCGGGTCAGTGAAAAAACGATCAGAGTAGTGG | |
| NR_003287.2_RNA28S5_r1.- | /5Biosg/GACACCTGCGTTACCGTTTGACAGGTGTACCGCCCCAGTCAAACTCCCCACCTGGC | 0.716666667 |
| 2_55 (SEQ ID NO: 472) | ACTGTCCCCGGAGCGGGTCGCGCCCGGCCGGCGCGCGGCCGGGCGCTTGGCGCCAGAAGCGAGA | |
| NR_003287.2_RNA28S5_r1.- | /5Biosg/AAGCGAGCTTTTGCCCTTCTGCTCCACGGGAGGTTTCTGTCCTCCCTGAGCTCGCC | 0.591666667 |
| 2_56 (SEQ ID NO: 473) | TTAGGACACCTGCGTTACCGTTTGACAGGTGTACCGCCCCAGTCAAACTCCCCACCTGGCACTG | |
| NR_003287.2_RNA28S5_r1.- | /5Biosg/GGTCAGAAGGATCGTGAGGCCCCGCTTTCACGGTCTGTATTCGTACTGAAAATCAA | 0.541666667 |
| 2_57 (SEQ ID NO: 474) | GATCAAGCGAGCTTTTGCCCTTCTGCTCCACGGGAGGTTTCTGTCCTCCCTGAGCTCGCCTTAG | |
| NR_003287.2_RNA28S5_r1.- | /5Biosg/CGCCACAAGCCAGTTATCCCTGTGGTAACTTTTCTGACACCTCCTGCTTAAAACCC | 0.483333333 |
| 2_58 (SEQ ID NO: 475) | AAAAGGTCAGAAGGATCGTGAGGCCCCGCTTTCACGGTCTGTATTCGTACTGAAAATCAAGATC | |
| NR_003287.2_RNA28S5_r1.- | /5Biosg/CAATGATAGGAAGAGCCGACATCGAAGGATCAAAAAGCGACGTCGCTATGAACGCT | 0.483333333 |
| 2_59 (SEQ ID NO: 476) | TGGCCGCCACAAGCCAGTTATCCCTGTGGTAACTTTTCTGACACCTCCTGCTTAAAACCCAAAA | |
| NR_003287.2_RNA28S5_r1.- | /5Biosg/ACCCAGCTCACGTTCCCTATTAGTGGGTGAACAATCCAACGCTTGGTGAATTCTGC | 0.491666667 |
| 2_60 (SEQ ID NO: 477) | TTCACAATGATAGGAAGAGCCGACATCGAAGGATCAAAAAGCGACGTCGCTATGAACGCTTGGC | |
| NR_003287.2_RNA28S5_r1.- | /5Biosg/TTACCATGGCAACAACACATCATCAGTAGGGTAAAACTAACCTGTCTCACGACGGT | 0.458333333 |
| 2_61 (SEQ ID NO: 478) | CTAAACCCAGCTCACGTTCCCTATTAGTGGGTGAACAATCCAACGCTTGGTGAATTCTGCTTCA | |
| NR_003287.2_RNA28S5_r1.- | /5Biosg/CCTCAGCCAAGCACATACACCAAATGTCTGAACCTGCGGTTCCTCTCGTACTGAGC | 0.483333333 |
| 2_62 (SEQ ID NO: 479) | AGGATTACCATGGCAACAACACATCATCAGTAGGGTAAAACTAACCTGTCTCACGACGGTCTAA | |
| NR_003287.2_RNA28S5_r1.- | /5Biosg/GATTCTGACTTAGAGGCGTTCAGTCATAATCCCACAGATGGTAGCTTCGCCCCATT | 0.516666667 |
| 2_63 (SEQ ID NO: 480) | GGCTCCTCAGCCAAGCACATACACCAAATGTCTGAACCTGCGGTTCCTCTCGTACTGAGCAGGA | |
| NR_003287.2_RNA28S5_r1.- | /5Biosg/ACCGGCTATCCGAGGCCAACCGAGGCTCCGCGGCGCTGCCGTATCGTTCCGCCTGG | 0.616666667 |
| 2_64 (SEQ ID NO: 481) | GCGGGATTCTGACTTAGAGGCGTTCAGTCATAATCCCACAGATGGTAGCTTCGCCCCATTGGCT | |
| NR_003287.2_RNA28S5_r1.- | /5Biosg/TCCCGCGCGCGCGGGGCGCGTGGAGGGGGGGGGCGGCCCGCCGGCGGGGACAGGCG | 0.825 |
| 2_65 (SEQ ID NO: 482) | GGGGACCGGCTATCCGAGGCCAACCGAGGCTCCGCGGCGCTGCCGTATCGTTCCGCCTGGGCGG | |
| NR_003287.2_RNA28S5_r1.- | /5Biosg/ACGAACGTGCGGTGCGTGACGGGCGAGGGGGCGGCCGCCTCTCCGGCCGCGCCCCG | 0.808333333 |
| 2_67 (SEQ ID NO: 483) | TTTCCCAGGACGAAGGGCACTCCGCACCGGACCCCGGTCCCGGCGCGCGGCGGGGCACGCGCCC | |
| NR_003287.2_RNA28S5_r1.- | /5Biosg/ACGTACGAAACCCCGACCCAGAAGCAGGTCGTCTACGAATGGTTTAGCGCCAGGTT | 0.683333333 |
| 2_68 (SEQ ID NO: 484) | CCCCACGAACGTGCGGTGCGTGACGGGCGAGGGGGCGGCCGCCTCTCCGGCCGCGCCCCGTTTC | |
| NR_003287.2_RNA28S5_r1.- | /5Biosg/GACAAACCCTTGTGTCGAGGGCTGACTTTCAATAGATCGCAGCGAGGGAGCTGCTC | 0.566666667 |
| 2_69 (SEQ ID NO: 485) | TGCTACGTACGAAACCCCGACCCAGAAGCAGGTCGTCTACGAATGGTTTAGCGCCAGGTTCCCC | |
| 5SrRNA_X71801_r1_1 | /5Biosg/CTTCCACCACATCGGGCCCGCTCGGAGCAGGGAGTGCTCCGAGGCGTCAGGGCCCA | 0.716666667 |
| (SEQ ID NO: 486) | GGGCCCACGATCCTGGGACGCCCTCCGGTCCTCCGCCCTGTCGCGGAGGCAGCGTTTTGGATCC | |
| 5SrRNA_X71801_r1_2 | /5Biosg/GATCGGGGACCCCCGAGCCGCTGGCCCGCGGCCTTCCCCCGGCTCCCGCGCTCCCG | 0.783333333 |
| (SEQ ID NO: 487) | AGCTTCCACCACATCGGGCCCGCTCGGAGCAGGGAGTGCTCCGAGGCGTCAGGGCCCAGGGCCC | |
| 5SrRNA_X71801_r1_3 | /5Biosg/GCCCGGCCGTGCCCGCCGGATTGCAGCCGACACCGCCAGCCCGGGGCCGCGGGGCT | 0.825 |
| (SEQ ID NO: 488) | CGGATCGGGGACCCCCGAGCCGCTGGCCCGCGGCCTTCCCCCGGCTCCCGCGCTCCCGAGCTTC | |
| 5SrRNA_X71801_r1_4 | /5Biosg/GGTGGTATGGCCGTAGACGCTGAAGGAGGCGCCTGGCTGCCCCAAGAGCCCAGCCC | 0.766666667 |
| (SEQ ID NO: 489) | CGCCCGGCCGTGCCCGCCGGATTGCAGCCGACACCGCCAGCCCGGGGCCGCGGGGCTCGGATCG | |
| 5SrRNA_X71801_r1_5 | /5Biosg/ACTAACCAGGCCCGACCCTGCTTAGCTTCCGAGATCAGACGAGATCGGGCGCGTTC | 0.666666667 |
| (SEQ ID NO: 490) | AGGGTGGTATGGCCGTAGACGCTGAAGGAGGCGCCTGGCTGCCCCAAGAGCCCAGCCCCGCCCG | |
| 5SrRNA_X71801_r1_6 | /5Biosg/AAAAGCCAAAGAAAAAGCCTACAGCACCCGGTATTCCCAGGCGGTCTCCCATCCAA | 0.566666667 |
| (SEQ ID NO: 491) | GTACTAACCAGGCCCGACCCTGCTTAGCTTCCGAGATCAGACGAGATCGGGCGCGTTCAGGGTG | |
| 5SrRNA_X71801_r1_7 | /5Biosg/CACTCCAGCCTGGGCGACAGGGCGAGACTCCGTCTGGAAGAAAAGGAAAGAAACAG | 0.533333333 |
| (SEQ ID NO: 492) | CAAAAAGCCAAAGAAAAAGCCTACAGCACCCGGTATTCCCAGGCGGTCTCCCATCCAAGTACTA | |
| 5SrRNA_X71801_r1_8 | /5Biosg/GGGGAATGGCGTGGACCCGGGAGGCGGAGCTTGCAGTGAGCCGAGATGGCGCCACC | 0.625 |
| (SEQ ID NO: 493) | GCACTCCAGCCTGGGCGACAGGGCGAGACTCCGTCTGGAAGAAAAGGAAAGAAACAGCAAAAAG | |
| 5SrRNA_X71801_r1_9 | /5Biosg/AAGTAGCCGGGCGTGGTGGCGGGCGCCTGTAGGCCCAGCTACTCGGGAGGCTGAGG | 0.716666667 |
| (SEQ ID NO: 494) | CCGGGGAATGGCGTGGACCCGGGAGGCGGAGCTTGCAGTGAGCCGAGATGGCGCCACCGCACTC | |
| 5SrRNA_X71801_r1_10 | /5Biosg/GAGCTCCAGACCATCCCGGCTAACAGGGTGAAAGCCCGTCTCTAGGAAAAATAGAA | 0.633333333 |
| (SEQ ID NO: 495) | CAAAGTAGCCGGGCGTGGTGGCGGGCGCCTGTAGGCCCAGCTACTCGGGAGGCTGAGGCCGGGG | |
| RNA5-8S5_NR_003285_r1_1 | /5Biosg/CCGGGGCCGCAAGTGCGTTCGAAGTGTCGATGATCAATGTGTCCTGCAATTCACAT | 0.541666667 |
| (SEQ ID NO: 496) | TAATTCTCGCAGCTAGCTGCGTTCTTCATCGACGCACGAGCCGAGTGATCCACCGCTAAGAGTC | |
| RNA5-8S5_NR_003285_r1_2 | /5Biosg/AAGCGACGCTCAGACAGGCGTAGCCCCGGGAGGAACCCGGGGCCGCAAGTGCGTTC | 0.558333333 |
| (SEQ ID NO: 497) | GAAGTGTCGATGATCAATGTGTCCTGCAATTCACATTAATTCTCGCAGCTAGCTGCGTTCTTCA | |

APPENDIX 6
60-mer DNA rRNA Capture Probes/Baits at 1x coverage, 85% GC restriction with dual 5′ + 3′-biotin
(Note: /5Biosg/ = 5′-biotin and /3Bio/ = 3′-biotin)

| Name | Sequence |
|---|---|
| KF899911.1_12SrRNA_r1_1.1 | /5Biosg/GCGTGCTTGATGCTTGTCCCTTTTGATCGTGGTGATTTAGAGGGTGAACTCACTGGAACG/ |
| (SEQ ID NO: 498) | 3Bio/ |
| KF899911.1_12SrRNA_r1_1.2 | /5Biosg/GGGATGCTTGCATGTGTAATCTTACTAAGAGCTAATAGAAAGGCTAGGACCAAACCTATT/ |
| (SEQ ID NO: 499) | 3Bio/ |
| KF899911.1_12SrRNA_r1_2.1 | /5Biosg/GAAATTGACCAACCCTGGGGTTAGTATAGCTTAGTTAAACTTTCGTTTATTGCTAAAGGT/ |
| (SEQ ID NO: 500) | 3Bio/ |
| KF899911.1_12SrRNA_r1_2.2 | /5Biosg/TAATCACTGCTGTTTCCCGTGGGGGTGTGGCTAGGCTAAGCGTTTTGAGCTGCATTGCTG/ |
| (SEQ ID NO: 501) | 3Bio/ |
| KF899911.1_12SrRNA_r1_3.1 | /5Biosg/ACTGGAGTTTTTTACAACTCAGGTGAGTTTTAGCTTTATTGGGGAGGGGGTGATCTAAAA/ |
| (SEQ ID NO: 502) | 3Bio/ |
| KF899911.1_12SrRNA_r1_3.2 | /5Biosg/CACTCTTTACGCCGGCTTCTATTGACTTGGGTTAATCGTGTGACCGCGGTGGCTGGCACG/ |
| (SEQ ID NO: 503) | 3Bio/ |
| KF899911.1_12SrRNA_r1_4.1 | /5Biosg/TGTTGATTTAACTGTTGAGGTTTAGGGCTAAGCATAGTGGGGTATCTAATCCCAGTTTGG/ |
| (SEQ ID NO: 504) | 3Bio/ |
| KF899911.1_12SrRNA_r1_4.2 | /5Biosg/GTCTTAGCTATTGTGTGTTCAGATATGTTAAAGCCACTTTCGTAGTCTATTTTGTGTCAA/ |
| (SEQ ID NO: 505) | 3Bio/ |
| KF899911.1_12SrRNA_r1_5.1 | /5Biosg/GAGGTGGTGAGGTTGATCGGGGTTTATCGATTACAGAACAGGCTCCTCTAGAGGGATATG/ |
| (SEQ ID NO: 506) | 3Bio/ |
| KF899911.1_12SrRNA_r1_5.2 | /5Biosg/AAGCACCGCCAGGTCCTTTGAGTTTTAAGCTGTGGCTCGTAGTGTTCTGGCGAGCAGTTT/ |
| (SEQ ID NO: 507) | 3Bio/ |
| KF899911.1_12SrRNA_r1_6.1 | /5Biosg/GCCCATTTCTTGCCACCTCATGGGCTACACCTTGACCTAACGTCTTTACGTGGGTACTTG/ |
| (SEQ ID NO: 508) | 3Bio/ |
| KF899911.1_12SrRNA_r1_6.2 | /5Biosg/CGCTTACTTTGTAGCCTTCATCAGGGTTTGCTGAAGATGGCGGTATATAGGCTGAGCAAG/ |
| (SEQ ID NO: 509) | 3Bio/ |
| KF899911.1_12SrRNA_r1_7.1 | /5Biosg/TGTGTACGCGCTTCAGGGCCCTGTTCAACTAAGCACTCTACTCTCAGTTTACTGCTAAAT/ |
| (SEQ ID NO: 510) | 3Bio/ |
| KF899911.1_12SrRNA_r1_7.2 | /5Biosg/CCACCTTCGACCCTTAAGTTTCATAAGGGCTATCGTAGTTTTCTGGGGTAGAAAATGTAG/ |
| (SEQ ID NO: 511) | 3Bio/ |
| KF899911.1_12SrRNA_r1_8.1 | /5Biosg/GTTCGTCCAAGTGCACTTTCCAGTACACTTACCATGTTACGACTTGTCTCCTCTATATAA/ |
| (SEQ ID NO: 512) | 3Bio/ |
| KF899911.1_12SrRNA_r1_8.2 | /5Biosg/ATGCGTAGGGGTTTTAGTTAAATGTCCTTTGAAGTATACTTGAGGAGGGTGACGGGCGGT/ |
| (SEQ ID NO: 513) | 3Bio/ |
| KF899911.1_16SrRNA_r1_1.1 | /5Biosg/TTGCGGTACTATATCTATTGCGCCAGGTTTCAATTTCTATCGCCTATACTTTATTTGGGT/ |
| (SEQ ID NO: 514) | 3Bio/ |
| KF899911.1_16SrRNA_r1_1.2 | /5Biosg/AAATGGTTTGGTTAAGGTTGTCTGGTAGTAAGGTGGAGTGGGTTTGGGGCTAGGTTTAGC/ |
| (SEQ ID NO: 515) | 3Bio/ |
| KF899911.1_16SrRNA_r1_2.1 | /5Biosg/GTTTCGGGGGTCTTAGCTTTGGCTCTCCTTGCAAAGTTATTTCTAGTTAATTCATTATGC/ |
| (SEQ ID NO: 516) | 3Bio/ |
| KF899911.1_16SrRNA_r1_2.2 | /5Biosg/AGAAGGTATAGGGGTTAGTCCTTGCTATATTATGCTTGGTTATAATTTTTCATCTTTCCC/ |
| (SEQ ID NO: 517) | 3Bio/ |
| KF899911.1_16SrRNA_r1_3.1 | /5Biosg/ATCTTGGACAACCAGCTATCACCAGGCTCGGTAGGTTTGTCGCCTCTACCTATAAATCTT/ |
| (SEQ ID NO: 518) | 3Bio/ |
| KF899911.1_16SrRNA_r1_3.2 | /5Biosg/CCCACTATTTTGCTACATAGACGGGTGTGCTCTTTTAGCTGTTCTTAGGTAGCTCGTCTG/ |
| (SEQ ID NO: 519) | 3Bio/ |
| KF899911.1_16SrRNA_r1_4.1 | /5Biosg/CTCTCTCTACAAGGTTTTTTCCTAGTGTCCAAAGAGCTGTTCCTCTTTGGACTAACAGTT/ |
| (SEQ ID NO: 520) | 3Bio/ |
| KF899911.1_16SrRNA_r1_4.2 | /5Biosg/AAATTTACAAGGGGATTTAGAGGGTTCTGTGGGCAAATTTAAAGTTGAACTAAGATTCTA/ |
| (SEQ ID NO: 521) | 3Bio/ |
| KF899911.1_16SrRNA_r1_5.1 | /5Biosg/CCAATTGGGTGTGAGGAGTTCAGTTATATGTTTGGGATTTTTTAGGTAGTGGGTGTTGAG/ |
| (SEQ ID NO: 522) | 3Bio/ |
| KF899911.1_16SrRNA_r1_5.2 | /5Biosg/CTTGAACGCTTTCTTAATTGGTGGCTGCTTTTAGGCCTACTATGGGTGTTAAATTTTTTA/ |
| (SEQ ID NO: 523) | 3Bio/ |
| KF899911.1_16SrRNA_r1_6.1 | /5Biosg/GTAGATATTGGGCTGTTAATTGTCAGTTCAGTGTTTTAATCTGACGCAGGCTTATGCGGA/ |
| (SEQ ID NO: 524) | 3Bio/ |
| KF899911.1_16SrRNA_r1_6.2 | /5Biosg/GGAGAATGTTTTCATGTTACTTATACTAACATTAGTTCTTCTATAGGGTGATAGATTGGT/ |
| (SEQ ID NO: 525) | 3Bio/ |
| KF899911.1_16SrRNA_r1_7.1 | /5Biosg/TGTTTTTGGTAAACAGGCGGGGTAAGATTTGCCGAGTTCCTTTTACTTTTTTTAACCTTT/ |
| (SEQ ID NO: 526) | 3Bio/ |
| KF899911.1_16SrRNA_r1_7.2 | /5Biosg/CCTTATGAGCATGCCTGTGTTGGGTTGACAGTGAGGGTAATAATGACTTGTTGGTTGATT/ |
| (SEQ ID NO: 527) | 3Bio/ |
| KF899911.1_16SrRNA_r1_8.1 | /5Biosg/TACAGGTCCCTATTTAAGGAACAAGTGATTATGCTACCTTTGCACGGTTAGGGTACCGCG/ |
| (SEQ ID NO: 528) | 3Bio/ |
| KF899911.1_16SrRNA_r1_8.2 | /5Biosg/GCCGTTAAACATGTGTCACTGGGCAGGCGGTGCCTCTAATACTGGTGATGCTAGAGGTGA/ |
| (SEQ ID NO: 529) | 3Bio/ |
| KF899911.1_16SrRNA_r1_9.1 | /5Biosg/TTAATAAATTAAAGCTCCATAGGGTCTTCTCGTCTTGCTGTGTCATGCCCGCCTCTTCAC/ |
| (SEQ ID NO: 530) | 3Bio/ |
| KF899911.1_16SrRNA_r1_9.2 | /5Biosg/GGGCAGGTCAATTTCACTGGTTAAAAGTAAGAGACAGCTGAACCCTCGTGGAGCCATTCA/ |
| (SEQ ID NO: 531) | 3Bio/ |
| KF899911.1_16SrRNA_r1_10.1 | /5Biosg/TGGTGAAGTCTTAGCATGTACTGCTCGGAGGTTGGGTTCTGCTCCGAGGTCGCCCCAACC/ |
| (SEQ ID NO: 532) | 3Bio/ |
| KF899911.1_16SrRNA_r1_10.2 | /5Biosg/GAAATTTTTAATGCAGGTTTGGTAGTTTAGGACCTGTGGGTTTGTTAGGTACTGTTTGCA/ |
| (SEQ ID NO: 533) | 3Bio/ |
| KF899911.1_16SrRNA_r1_11.1 | /5Biosg/GAGGTCGTAAACCCTATTGTTGATATGGACTCTAGAATAGGATTGCGCTGTTATCCCTAG/ |
| (SEQ ID NO: 534) | 3Bio/ |
| KF899911.1_16SrRNA_r1_11.2 | /5Biosg/GGTAACTTGTTCCGTTGGTCAAGTTATTGGATCAATTGAGTATAGTAGTTCGCTTTGACT/ |
| (SEQ ID NO: 535) | 3Bio/ |
| KF899911.1_16SrRNA_r1_12.1 | /5Biosg/AAGTAGATAGAAACCGACCTGGATTACTCCGGTCTGAACTCAGATCACGTAGGACTTTAA/ |
| (SEQ ID NO: 536) | 3Bio/ |
| KF899911.1_16SrRNA_r1_12.2 | /5Biosg/TCGTTGAACAAACGAACCTTTAATAGCGGCTGCACCATCGGGATGTCCTGATCCAACATC/ |
| (SEQ ID NO: 537) | 3Bio/ |
| KF899911.1_16SrRNA_r1_13.1 | /5Biosg/AAACCCTGTTCTTGGGTGGGTGTGGGTATAATACTAAGTTGAGATGATATCATTTACGGG/ |
| (SEQ ID NO: 538) | 3Bio/ |
| KF899911.1_16SrRNA_r1_13.2 | /5Biosg/GGAAGGCGCTTTGTGAAGTAGGCCTTATTTCTCTTGTCCTTTCGTACAGGGAGGAATTTG/ |
| (SEQ ID NO: 539) | 3Bio/ |
| NR_003286.2_RNA18S5_r1_1.1 | /5Biosg/AAGGAACCATAACTGATTTAATGAGCCATTCGCAGTTTCACTGTACCGGCCGTGCGTACT/ |
| (SEQ ID NO: 540) | 3Bio/ |
| NR_003286.2_RNA18S5_r1_1.2 | /5Biosg/CAGACATGCATGGCTTAATCTTTGAGACAAGCATATGCTACTGGCAGGATCAACCAGGTA/ |
| (SEQ ID NO: 541) | 3Bio/ |
| NR_003286.2_RNA18S5_r1_2.1 | /5Biosg/GTTGGTTTTGATCTGATAAATGCACGCATCCCCCCCGCGAAGGGGGTCAGCGCCCGTCGG/ |
| (SEQ ID NO: 542) | 3Bio/ |
| NR_003286.2_RNA18S5_r1_2.2 | /5Biosg/CATGTATTAGCTCTAGAATTACCACAGTTATCCAAGTAGGAGAGGAGCGAGCGACCAAAG/ |
| (SEQ ID NO: 543) | 3Bio/ |
| NR_003286.2_RNA18S5_r1_3.1 | /5Biosg/GACGTTCGAATGGGTCGTCGCCGCCACGGGGGGCGTGCGATCGGCCCGAGGTTATCTAGA/ |
| (SEQ ID NO: 544) | 3Bio/ |
| NR_003286.2_RNA18S5_r1_3.2 | /5Biosg/GTCACCAAAGCCGCCGGCGCCCGCCCCCCGGCCGGGGCCGGAGAGGGGCTGACCGGGTTG/ |
| (SEQ ID NO: 545) | 3Bio/ |
| NR_003286.2_RNA18S5_r1_4.1 | /5Biosg/CTTCCTTGGATGTGGTAGCCGTTTCTCAGGCTCCCTCTCCGGAATCGAACCCTGATTCCC/ |
| (SEQ ID NO: 546) | 3Bio/ |
| NR_003286.2_RNA18S5_r1_4.2 | /5Biosg/CGTCACCCGTGGTCACCATGGTAGGCACGGCGACTACCATCGAAAGTTGATAGGGCAGAC/ |
| (SEQ ID NO: 547) | 3Bio/ |
| NR_003286.2_RNA18S5_r1_5.1 | /5Biosg/CGTTAAAGGATTTAAAGTGGACTCATTCCAATTACAGGGCCTCGAAAGAGTCCTGTATTG/ |
| (SEQ ID NO: 548) | 3Bio/ |
| NR_003286.2_RNA18S5_r1_5.2 | /5Biosg/TTATTTTTCGTCACTACCTCCCCGGGTCGGGAGTGGGTAATTTGCGCGCCTGCTGCCTTC/ |
| (SEQ ID NO: 549) | 3Bio/ |
| NR_003286.2_RNA18S5_r1_6.1 | /5Biosg/GCCCGCCCGCTCCCAAGATCCAACTACGAGCTTTTTAACTGCAGCAACTTTAATATACGC/ |
| (SEQ ID NO: 550) | 3Bio/ |
| NR_003286.2_RNA18S5_r1_6.2 | /5Biosg/TATTGGAGCTGGAATTACCGCGGCTGCTGGCACCAGACTTGCCCTCCAATGGATCCTCGT/ |
| (SEQ ID NO: 551) | 3Bio/ |
| NR_003286.2_RNA18S5_r1_7.1 | /5Biosg/ACTCTAATTTTTTCAAAGTAAACGCTTCGGGCCCCGCGGGACACTCAGCTAAGAGCATCG/ |
| (SEQ ID NO: 552) | 3Bio/ |
| NR_003286.2_RNA18S5_r1_7.2 | /5Biosg/AGGGGGCGCCGAGAGGCAAGGGGCGGGGACGGGCGGTGGCTCGCCTCGCGGCGGACCGCC/ |
| (SEQ ID NO: 553) | 3Bio/ |
| NR_003286.2_RNA18S5_r1_8.1 | /5Biosg/CCCCCGGCCGTCCCTCTTAATCATGGCCTCAGTTCCGAAAACCAACAAAATAGAACCGCG/ |
| (SEQ ID NO: 554) | 3Bio/ |
| NR_003286.2_RNA18S5_r1_8.2 | /5Biosg/GTCCTATTCCATTATTCCTAGCTGCGGTATCCAGGCGGCTCGGGCCTGCTTTGAACACTC/ |
| (SEQ ID NO: 555) | 3Bio/ |
| NR_003286.2_RNA18S5_r1_9.1 | /5Biosg/GTCTTCGAACCTCCGACTTTCGTTCTTGATTAATGAAAACATTCTTGGCAAATGCTTTCG/ |
| (SEQ ID NO: 556) | 3Bio/ |
| NR_003286.2_RNA18S5_r1_9.2 | /5Biosg/CTCTGGTCCGTCTTGCGCCGGTCCAAGAATTTCACCTCTAGCGGCGCAATACGAATGCCC/ |
| (SEQ ID NO: 557) | 3Bio/ |
| NR_003286.2_RNA18S5_r1_10.1 | /5Biosg/CCCCCCGGAACCCAAAGACTTTGGTTTCCCGGAAGCTGCCCGGCGGGTCATGGGAATAAC/ |
| (SEQ ID NO: 558) | 3Bio/ |
| NR_003286.2_RNA18S5_r1_10.2 | /5Biosg/GCCGCCGCATCGCCGGTCGGCATCGTTTATGGTCGGAACTACGACGGTATCTGATCGTCT/ |
| (SEQ ID NO: 559) | 3Bio/ |
| NR_003286.2_RNA18S5_r1_11.1 | /5Biosg/CCTGTCCGTGTCCGGGCCGGGTGAGGTTTCCCGTGTTGAGTCAAATTAAGCCGCAGGCTC/ |
| (SEQ ID NO: 560) | 3Bio/ |
| NR_003286.2_RNA18S5_r1_11.2 | /5Biosg/CACTCCTGGTGGTGCCCTTCCGTCAATTCCTTTAAGTTTCAGCTTTGCAACCATACTCCC/ |
| (SEQ ID NO: 561) | 3Bio/ |
| NR_003286.2_RNA18S5_r1_12.1 | /5Biosg/GTTAGCATGCCAGAGTCTCGTTCGTTATCGGAATTAACCAGACAAATCGCTCCACCAACT/ |
| (SEQ ID NO: 562) | 3Bio/ |
| NR_003286.2_RNA18S5_r1_12.2 | /5Biosg/AAGAACGGCCATGCACCACCACCCACGGAATCGAGAAAGAGCTATCAATCTGTCAATCCT/ |
| (SEQ ID NO: 563) | 3Bio/ |
| NR_003286.2_RNA18S5_r1_13.1 | /5Biosg/AGCCCCGGACATCTAAGGGCATCACAGACCTGTTATTGCTCAATCTCGGGTGGCTGAACG/ |
| (SEQ ID NO: 564) | 3Bio/ |
| NR_003286.2_RNA18S5_r1_13.2 | /5Biosg/CCACTTGTCCCTCTAAGAAGTTGGGGGACGCCGACCGCTCGGGGGTCGCGTAACTAGTTA/ |
| (SEQ ID NO: 565) | 3Bio/ |
| NR_003286.2_RNA18S5_r1_14.1 | /5Biosg/CGTTCATGGGGAATAATTGCAATCCCCGATCCCCATCACGAATGGGGTTCAACGGGTTAC/ |
| (SEQ ID NO: 566) | 3Bio/ |
| NR_003286.2_RNA18S5_r1_14.2 | /5Biosg/CCGCGCCTGCCGGCGTAGGGTAGGCACACGCTGAGCCAGTCAGTGTAGCGCGCGTGCAGC/ |
| (SEQ ID NO: 567) | 3Bio/ |
| NR_003286.2_RNA18S5_r1_15.1 | /5Biosg/GGGCCGATCCGAGGGCCTCACTAAACCATCCAATCGGTAGTAGCGACGGGCGGTGTGTAC/ |
| (SEQ ID NO: 568) | 3Bio/ |
| NR_003286.2_RNA18S5_r1_15.2 | /5Biosg/AAAGGGCAGGGACTTAATCAACGCAAGCTTATGACCCGCACTTACTGGGAATTCCTCGTT/ |
| (SEQ ID NO: 569) | 3Bio/ |
| NR_003286.2_RNA18S5_r1_16.1 | /5Biosg/TAATGATCCTTCCGCAGGTTCACCTACGGAAACCTTGTTACGACTTTTACTTCCTCTAGA/ |
| (SEQ ID NO: 570) | 3Bio/ |
| NR_003286.2_RNA18S5_r1_16.2 | /5Biosg/TAGTCAAGTTCGACCGTCTTCTCAGCGCTCCGCCAGGGCCGTGGGCCGACCCCGGCGGGG/ |
| (SEQ ID NO: 571) | 3Bio/ |
| NR_003287.2_RNA28S5_r1.1_1.1 | /5Biosg/TCGGCGCTGGGCTCTTCCCTGTTCACTCGCCGTTACTGAGGGAATCCTGGTTAGTTTCTT/ |
| (SEQ ID NO: 572) | 3Bio/ |
| NR_003287.2_RNA28S5_r1.1_1.2 | /5Biosg/CTCCTCCGCTGACTAATATGCTTAAATTCAGCGGGTCGCCACGTCTGATCTGAGGTCGCG/ |
| (SEQ ID NO: 573) | 3Bio/ |
| NR_003287.2_RNA28S5_r1.1_2.1 | /5Biosg/TCCACGGGCTGGGCCTCGATCAGAAGGACTTGGGCCCCCCACGAGCGGCGCCGGGGAGCG/ |
| (SEQ ID NO: 574) | 3Bio/ |
| NR_003287.2_RNA28S5_r1.1_2.2 | /5Biosg/GGTCTTCCGTACGCCACATGTCCCGCGCCCCGCCGCGGGGCGGGGATTCGGCGCTGGGCT/ |
| (SEQ ID NO: 575) | 3Bio/ |
| NR_003287.2_RNA28S5_r1.1_3.1 | /5Biosg/TAGCCTTAGATGGAGTTTACCACCCGCTTTGGGCTGCATTCCCAAGCAACCCGACTCCGG/ |
| (SEQ ID NO: 576) | 3Bio/ |
| NR_003287.2_RNA28S5_r1.1_3.2 | /5Biosg/GAAGACCCGGGCCCGGCGCGCCGGGGGCCGCTACCGGCCTCACACCGTCCACGGGCTGGG/ |
| (SEQ ID NO: 577) | 3Bio/ |
| NR_003287.2_RNA28S5_r1.1_4.1 | /5Biosg/CACCCGTTTACCTCTTAACGGTTTCACGCCCTCTTGAACTCTCTCTTCAAAGTTCTTTTC/ |
| (SEQ ID NO: 578) | 3Bio/ |
| NR_003287.2_RNA28S5_r1.1_4.2 | /5Biosg/AACTTTCCCTTACGGTACTTGTTGACTATCGGTCTCGTGCCGGTATTTAGCCTTAGATGG/ |
| (SEQ ID NO: 579) | 3Bio/ |
| NR_003287.2_RNA28S5_r1.1_5.1 | /5Biosg/CGGGTGGAGGGGTCGGGAGGAACGGGGGGCGGGAAAGATCCGCCGGGCCGCCGACACGGC/ |
| (SEQ ID NO: 580) | 3Bio/ |
| NR_003287.2_RNA28S5_r1.1_5.2 | /5Biosg/CGGACCCGCCGCCGGGTTGAATCCTCCGGGCGGACTGCGCGGACCCCACCCGTTTACCTC/ |
| (SEQ ID NO: 581) | 3Bio/ |
| NR_003287.2_RNA28S5_r1.1_7.1 | /5Biosg/CCCCCCGAGCCACCTTCCCCGCCGGGCCTTCCCAGCCGTCCCGGAGCCGGTCGCGGCGCA/ |
| (SEQ ID NO: 582) | 3Bio/ |
| NR_003287.2_RNA28S5_r1.1_7.2 | /5Biosg/CCGCCGCGGTGGAAATGCGCCCGGCGGCGGCCGGTCGCCGGTCGGGGGACGGTCCCCCGC/ |
| (SEQ ID NO: 583) | 3Bio/ |
| NR_003287.2_RNA28S5_r1.2_1.1 | /5Biosg/GAGGGGGGAGAGCGCGGCGACGGGTCTCGCTCCCTCGGCCCCGGGATTCGGCGAGTGCTG/ |
| (SEQ ID NO: 584) | 3Bio/ |
| NR_003287.2_RNA28S5_r1.2_1.2 | /5Biosg/CTGCCGGGGGGGCTGTAACACTCGGGGGGGGTTTCGGTCCCGCCGCCGCCGCCGCCGCCG/ |
| (SEQ ID NO: 585) | 3Bio/ |
| NR_003287.2_RNA28S5_r1.2_4.1 | /5Biosg/GCACGTGTTAGACTCCTTGGTCCGTGTTTCAAGACGGGTCGGGTGGGTAGCCGACGTCGC/ |
| (SEQ ID NO: 586) | 3Bio/ |
| NR_003287.2_RNA28S5_r1.2_4.2 | /5Biosg/CGCCGACCCCGTGCGCTCGCTCCGCCGTCCCCCTCTTCGGGGGACGCGCGCGTGGCCCCG/ |
| (SEQ ID NO: 587) | 3Bio/ |
| NR_003287.2_RNA28S5_r1.2_5.1 | /5Biosg/CGGGCCGGTGGTGCGCCCTCGGCGGACTGGAGAGGCCTCGGGATCCCACCTCGGCCGGCG/ |
| (SEQ ID NO: 588) | 3Bio/ |
| NR_003287.2_RNA28S5_r1.2_5.2 | /5Biosg/AGCGCGCCGGCCTTCACCTTCATTGCGCCACGGCGGCTTTCGTGCGAGCCCCCGACTCGC/ |
| (SEQ ID NO: 589) | 3Bio/ |
| NR_003287.2_RNA28S5_r1.2_6.1 | /5Biosg/GGACCGCTACGGACCTCCACCAGAGTTTCCTCTGGCTTCGCCCTGCCCAGGCATAGTTCA/ |
| (SEQ ID NO: 590) | 3Bio/ |
| NR_003287.2_RNA28S5_r1.2_6.2 | /5Biosg/CCATCTTTCGGGTCCTAACACGTGCGCTCGTGCTCCACCTCCCCGGCGCGGCGGGCGAGA/ |
| (SEQ ID NO: 591) | 3Bio/ |
| NR_003287.2_RNA28S5_r1.2_7.1 | /5Biosg/TGCGTCGGGTCTGCGAGAGCGCCAGCTATCCTGAGGGAAACTTCGGAGGGAACCAGCTAC/ |
| (SEQ ID NO: 592) | 3Bio/ |
| NR_003287.2_RNA28S5_r1.2_7.2 | /5Biosg/TAGATGGTTCGATTAGTCTTTCGCCCCTATACCCAGGTCGGACGACCGATTTGCACGTCA/ |
| (SEQ ID NO: 593) | 3Bio/ |
| NR_003287.2_RNA28S5_r1.2_8.1 | /5Biosg/CCGGCTCCACGCCAGCGAGCCGGGCTTCTTACCCATTTAAAGTTTGAGAATAGGTTGAGA/ |
| (SEQ ID NO: 594) | 3Bio/ |
| NR_003287.2_RNA28S5_r1.2_8.2 | /5Biosg/TCGTTTCGGCCCCAAGACCTCTAATCATTCGCTTTACCGGATAAAACTGCGTGGCGGGGG/ |
| (SEQ ID NO: 595) | 3Bio/ |
| NR_003287.2_RNA28S5_r1.2_9.1 | /5Biosg/AACACCTTTTCTGGGGTCTGATGAGCGTCGGCATCGGGCGCCTTAACCCGGCGTTCGGTT/ |
| (SEQ ID NO: 596) | 3Bio/ |
| NR_003287.2_RNA28S5_r1.2_9.2 | /5Biosg/CATCCCGCAGCGCCAGTTCTGCTTACCAAAAGTGGCCCACTAGGCACTCGCATTCCACGC/ |
| (SEQ ID NO: 597) | 3Bio/ |
| NR_003287.2_RNA28S5_r1.2_10.1 | /5Biosg/ATGGGCCCGACGCTCCAGCGCCATCCATTTTCAGGGCTAGTTGATTCGGCAGGTGAGTTG/ |
| (SEQ ID NO: 598) | 3Bio/ |
| NR_003287.2_RNA28S5_r1.2_10.2 | /5Biosg/TTACACACTCCTTAGCGGATTCCGACTTCCATGGCCACCGTCCTGCTGTCTATATCAACC/ |
| (SEQ ID NO: 599) | 3Bio/ |
| NR_003287.2_RNA28S5_r1.2_11.1 | /5Biosg/GAAGGACCCCACACCCCCGCCGCCGCCGCCGCCGCCGCCCTCCGACGCACACCACACGCG/ |
| (SEQ ID NO: 600) | 3Bio/ |
| NR_003287.2_RNA28S5_r1.2_11.2 | /5Biosg/CGCGCGCGCGCGCCGCCCCCGCCGCTCCCGTCCACTCTCGACTGCCGGCGACGGCCGGGT/ |
| (SEQ ID NO: 601) | 3Bio/ |
| NR_003287.2_RNA28S5_r1.2_12.1 | /5Biosg/GCTTCAAGGCTCACCGCAGCGGCCCTCCTACTCGTCGCGGCGTAGCGTCCGCGGGGCTCC/ |
| (SEQ ID NO: 602) | 3Bio/ |
| NR_003287.2_RNA28S5_r1.2_12.2 | /5Biosg/GGGGGCGGGGAGCGGGGCGTGGGCGGGAGGAGGGGAGGAGGCGTGGGGGGGGGGGCGGGG/ |
| (SEQ ID NO: 603) | 3Bio/ |
| NR_003287.2_RNA28S5_r1.2_13.1 | /5Biosg/GTTCAACTGCTGTTCACATGGAACCCTTCTCCACTTCGGCCTTCAAAGTTCTCGTTTGAA/ |
| (SEQ ID NO: 604) | 3Bio/ |
| NR_003287.2_RNA28S5_r1.2_13.2 | /5Biosg/TATTTGCTACTACCACCAAGATCTGCACCTGCGGCGGCTCCACCCGGGCCCGCGCCCTAG/ |
| (SEQ ID NO: 605) | 3Bio/ |
| NR_003287.2_RNA28S5_r1.2_14.1 | /5Biosg/CCGCCACTCCGGATTCGGGGATCTGAACCCGACTCCCTTTCGATCGGCCGAGGGCAACGG/ |
| (SEQ ID NO: 606) | 3Bio/ |
| NR_003287.2_RNA28S5_r1.2_14.2 | /5Biosg/AGGCCATCGCCCGTCCCTTCGGAACGGCGCTCGCCCATCTCTCAGGACCGACTGACCCAT/ |
| (SEQ ID NO: 607) | 3Bio/ |
| NR_003287.2_RNA28S5_r1.2_15.1 | /5Biosg/GGCGAACCCATTCCAGGGCGCCCTGCCCTTCACAAAGAAAAGAGAACTCTCCCCGGGGCT/ |
| (SEQ ID NO: 608) | 3Bio/ |
| NR_003287.2_RNA28S5_r1.2_15.2 | /5Biosg/CCCGCCGGCTTCTCCGGGATCGGTCGCGTTACCGCACTGGACGCCTCGCGGCGCCCATCT/ |
| (SEQ ID NO: 609) | 3Bio/ |
| NR_003287.2_RNA28S5_r1.2_16.1 | /5Biosg/ATATGGGTACGGCCCGGCGCGAGATTTACACCCTCTCCCCCGGATTTTCAAGGGCCAGCG/ |
| (SEQ ID NO: 610) | 3Bio/ |
| NR_003287.2_RNA28S5_r1.2_16.2 | /5Biosg/AGAGCTCACCGGACGCCGCCGGAACCGCGACGCTTTCCAAGGCACGGGCCCCTCTCTCGG/ |
| (SEQ ID NO: 611) | 3Bio/ |
| NR_003287.2_RNA28S5_r1.2_17.1 | /5Biosg/CGACCGACCCAGCCCTTAGAGCCAATCCTTATCCCGAAGTTACGGATCCGGCTTGCCGAC/ |
| (SEQ ID NO: 612) | 3Bio/ |
| NR_003287.2_RNA28S5_r1.2_17.2 | /5Biosg/TTCCCTTACCTACATTGTTCCAACATGCCAGAGGCTGTTCACCTTGGAGACCTGCTGCGG/ |
| (SEQ ID NO: 613) | 3Bio/ |
| NR_003287.2_RNA28S5_r1.2_19.1 | /5Biosg/GCCCCTGCCGCCCCGACCCTTCTCCCCCCGCCGCCGCCCCCACGCGGCGCTCCCCCGGGG/ |
| (SEQ ID NO: 614) | 3Bio/ |
| NR_003287.2_RNA28S5_r1.2_19.2 | /5Biosg/AGGGGGGAGGACGGGGAGCGGGGGAGAGAGAGAGAGAGAGGGCGCGGGGCGGGGAGGGAG/ |
| (SEQ ID NO: 615) | 3Bio/ |
| NR_003287.2_RNA28S5_r1.2_23.1 | /5Biosg/TTCCCCTGGTCCGCACCAGTTCTAAGTCGGCTGCTAGGCGCCGGCCGAGGCGAGGCGCCG/ |
| (SEQ ID NO: 616) | 3Bio/ |
| NR_003287.2_RNA28S5_r1.2_23.2 | /5Biosg/CGCGGAACCGCGGCCCCGGGGGCGGACCCGGCGGGGGGGACCGGCCCGCGGCCCCTCCGC/ |
| (SEQ ID NO: 617) | 3Bio/ |
| NR_003287.2_RNA28S5_r1.2_24.1 | /5Biosg/CGTTTACCCGCGCTTCATTGAATTTCTTCACTTTGACATTCAGAGCACTGGGCAGAAATC/ |
| (SEQ ID NO: 618) | 3Bio/ |
| NR_003287.2_RNA28S5_r1.2_24.2 | /5Biosg/ACATCGCGTCAACACCCGCCGCGGGCCTTCGCGATGCTTTGTTTTAATTAAACAGTCGGA/ |
| (SEQ ID NO: 619) | 3Bio/ |
| NR_003287.2_RNA28S5_r1.2_25.1 | /5Biosg/GGCTGTGGTTTCGCTGGATAGTAGGTAGGGACAGTGGGAATCTCGTTCATCCATTCATGC/ |
| (SEQ ID NO: 620) | 3Bio/ |
| NR_003287.2_RNA28S5_r1.2_25.2 | /5Biosg/GCGTCACTAATTAGATGACGAGGCATTTGGCTACCTTAAGAGAGTCATAGTTACTCCCGC/ |
| (SEQ ID NO: 621) | 3Bio/ |
| NR_003287.2_RNA28S5_r1.2_26.1 | /5Biosg/GGGGGGGCGCCGGGGGCCTCCCACTTATTCTACACCTCTCATGTCTCTTCACCGTGCCAG/ |
| (SEQ ID NO: 622) | 3Bio/ |
| NR_003287.2_RNA28S5_r1.2_26.2 | /5Biosg/ACTAGAGTCAAGCTCAACAGGGTCTTCTTTCCCCGCTGATTCCGCCAAGCCCGTTCCCTT/ |
| (SEQ ID NO: 623) | 3Bio/ |
| NR_003287.2_RNA28S5_r1.2_27.1 | /5Biosg/GCCCCTCGGGGCTCGCCCCCCCGCCTCACCGGGTCAGTGAAAAAACGATCAGAGTAGTGG/ |
| (SEQ ID NO: 624) | 3Bio/ |
| NR_003287.2_RNA28S5_r1.2_27.2 | /5Biosg/TATTTCACCGGCGGCCCGCAGGGCCGGCGGACCCCGCCCCGGGCCCCTCGCGGGGACACC/ |
| (SEQ ID NO: 625) | 3Bio/ |
| NR_003287.2_RNA28S5_r1.2_28.1 | /5Biosg/GACACCTGCGTTACCGTTTGACAGGTGTACCGCCCCAGTCAAACTCCCCACCTGGCACTG/ |
| (SEQ ID NO: 626) | 3Bio/ |
| NR_003287.2_RNA28S5_r1.2_28.2 | /5Biosg/TCCCCGGAGCGGGTCGCGCCCGGCCGGCGCGCGGCCGGGCGCTTGGCGCCAGAAGCGAGA/ |
| (SEQ ID NO: 627) | 3Bio/ |
| NR_003287.2_RNA28S5_r1.2_29.1 | /5Biosg/GGTCAGAAGGATCGTGAGGCCCCGCTTTCACGGTCTGTATTCGTACTGAAAATCAAGATC/ |
| (SEQ ID NO: 628) | 3Bio/ |
| NR_003287.2_RNA28S5_r1.2_29.2 | /5Biosg/AAGCGAGCTTTTGCCCTTCTGCTCCACGGGAGGTTTCTGTCCTCCCTGAGCTCGCCTTAG/ |
| (SEQ ID NO: 629) | 3Bio/ |
| NR_003287.2_RNA28S5_r1.2_30.1 | /5Biosg/CAATGATAGGAAGAGCCGACATCGAAGGATCAAAAAGCGACGTCGCTATGAACGCTTGGC/ |
| (SEQ ID NO: 630) | 3Bio/ |
| NR_003287.2_RNA28S5_r1.2_30.2 | /5Biosg/CGCCACAAGCCAGTTATCCCTGTGGTAACTTTTCTGACACCTCCTGCTTAAAACCCAAAA/ |
| (SEQ ID NO: 631) | 3Bio/ |
| NR_003287.2_RNA28S5_r1.2_31.1 | /5Biosg/TTACCATGGCAACAACACATCATCAGTAGGGTAAAACTAACCTGTCTCACGACGGTCTAA/ |
| (SEQ ID NO: 632) | 3Bio/ |
| NR_003287.2_RNA28S5_r1.2_31.2 | /5Biosg/ACCCAGCTCACGTTCCCTATTAGTGGGTGAACAATCCAACGCTTGGTGAATTCTGCTTCA/ |
| (SEQ ID NO: 633) | 3Bio/ |
| NR_003287.2_RNA28S5_r1.2_32.1 | /5Biosg/GATTCTGACTTAGAGGCGTTCAGTCATAATCCCACAGATGGTAGCTTCGCCCCATTGGCT/ |
| (SEQ ID NO: 634) | 3Bio/ |
| NR_003287.2_RNA28S5_r1.2_32.2 | /5Biosg/CCTCAGCCAAGCACATACACCAAATGTCTGAACCTGCGGTTCCTCTCGTACTGAGCAGGA/ |
| (SEQ ID NO: 635) | 3Bio/ |
| NR_003287.2_RNA28S5_r1.2_33.1 | /5Biosg/TCCCGCGCGCGCGGGGCGCGTGGAGGGGGGGGGCGGCCCGCCGGCGGGGACAGGCGGGGG/ |
| (SEQ ID NO: 636) | 3Bio/ |
| NR_003287.2_RNA28S5_r1.2_33.2 | /5Biosg/ACCGGCTATCCGAGGCCAACCGAGGCTCCGCGGCGCTGCCGTATCGTTCCGCCTGGGCGG/ |
| (SEQ ID NO: 637) | 3Bio/ |
| NR_003287.2_RNA28S5_r1.2_34.1 | /5Biosg/ACGAACGTGCGGTGCGTGACGGGCGAGGGGGCGGCCGCCTCTCCGGCCGCGCCCCGTTTC/ |
| (SEQ ID NO: 638) | 3Bio/ |
| NR_003287.2_RNA28S5_r1.2_34.2 | /5Biosg/CCAGGACGAAGGGCACTCCGCACCGGACCCCGGTCCCGGCGCGCGGCGGGGCACGCGCCC/ |
| (SEQ ID NO: 639) | 3Bio/ |
| NR_003287.2_RNA28S5_r1.2_35.1 | /5Biosg/GACAAACCCTTGTGTCGAGGGCTGACTTTCAATAGATCGCAGCGAGGGAGCTGCTCTGCT/ |
| (SEQ ID NO: 640) | 3Bio/ |
| NR_003287.2_RNA28S5_r1.2_35.2 | /5Biosg/ACGTACGAAACCCCGACCCAGAAGCAGGTCGTCTACGAATGGTTTAGCGCCAGGTTCCCC/ |
| (SEQ ID NO: 641) | 3Bio/ |
| NR_003285.2_RNA5-8S_MB1.1 | /5Biosg/CGGGAGGAACCCGGGGCCGCAAGTGCGTTCGAAGTGTCGATGATCAATGTGTCCTGCAAT/ |
| (SEQ ID NO: 642) | 3Bio/ |
| NR_003285.2_RNA5-8S_MB1.2 | /5Biosg/TCACATTAATTCTCGCAGCTAGCTGCGTTCTTCATCGACGCACGAGCCGAGTGATCCACC/ |
| (SEQ ID NO: 643) | 3Bio/ |
| NR_023363.1_RNA5S_MB1.1 | /5Biosg/AAGCCTACAGCACCCGGTATTCCCAGGCGGTCTCCCATCCAAGTACTAACCAGGCCCGAC/ |
| (SEQ ID NO: 644) | 3Bio/ |
| NR_023363.1_RNA5S_MB1.2 | /5Biosg/CCTGCTTAGCTTCCGAGATCAGACGAGATCGGGCGCGTTCAGGGTGGTATGGCCGTAGAC/ |
| (SEQ ID NO: 645) | 3Bio/ |

APPENDIX 7
Human ribosoinai protein genes
Gene Listing (79):

| | |
|---|---|
| RPLP0 | NM_001002 |
| RPLP1 | NM_001003 |
| RPLP2 | NM_001004 |
| RPL3 | NM_000967 |
| RPL4 | NM_000968 |
| RPL5 | NM_000969 |
| RPL6 | NM_000970 |
| RPL7 | NM_000971 |
| RPL7A | NM_000972 |
| RPL8 | NM_000973 |
| RPL9 | NM_000661 |
| RPL10 | NM_006013 |
| RPL10A | NM_007104 |
| RPL11 | NM_000975 |
| RPL12 | NM_000976 |
| RPL13 | NM_033251 |
| RPL13A | NM_012423 |
| RPL14 | NM_003973 |
| RPL15 | NM_002948 |
| RPL17 | NM_000985 |
| RPL18 | NM_000979 |
| RPL19 | NM_000981 |
| MRPL20 | NM_017971 |
| RPL21 | NM_000982 |
| RPL22 | NM_000983 |
| RPL23 | NM_000978 |
| RPL23A | NM_000984 |
| RPL24 | NM_000986 |
| RPL26 | NM_000987 |
| RPL27 | NM_000988 |
| RPL27A | NM_000990 |
| RPL28 | NM_000991 |
| RPL29 | NM_000992 |
| RPL30 | NM_000989 |
| RPL31 | NM_000993 |
| RPL32 | NM_000994 |
| RPL34 | NM_000995 |
| RPL35 | NM_007209 |
| RPL36 | NM_033643 |
| RPL36A | NM_021029 |
| RPL37 | NM_000997 |
| RPL37A | NM_000998 |
| RPL38 | NM_000999 |
| RPL39 | NM_001000 |
| RPL41 | NM_021104 |
| MRPL41 | NM_032477 |
| RPSA | NM_002295 |
| RPS2 | NM_002952 |
| RPS3 | NM_001005 |
| RPS3A | NM_001006 |
| RPS4X | NM_001007 |
| RPS4Y | NM_001008 |
| RPS5 | NM_001009 |
| RPS6 | NM_001010 |
| RPS7 | NM_001011 |
| RPS8 | NM_001012 |
| RPS9 | NM_001013 |
| RPS10 | NM_001014 |
| RPS11 | NM_001015 |
| RPS12 | NM_001016 |
| RPS13 | NM_001017 |
| RPS14 | NM_005617 |
| RPS15 | NM_001018 |
| RPS15A | NM_001019 |
| RPS16 | NM_001020 |
| RPS17 | NM_001021 |
| RPS18 | NM_022551 |
| MRPS18A | NM_018135 |
| RPS19 | NM_001022 |
| RPS20 | NM_001023 |
| RPS21 | NM_001024 |
| RPS23 | NM_001025 |
| RPS24 | NM_001026 |
| RPS25 | NM_001028 |
| RPS26 | NM_001029 |
| RPS27 | NM_001030 |
| RPS27A | NM_002954 |
| RPS28 | NM_001031 |
| RPS29 | NM_001032 |

LOCUS NM_001002 1229 bp mRNA linear PRI 10-JAN-2014
DEFINITION Homo sapiens ribosomal protein, large, P0 (RPLP0), transcript
variant 1, mRNA. (SEQ ID NO: 646)

| | |
|---|---|
| 1 | gtctgacggg cgatggcgca gccaatagac aggagcgcta tccgcggttt ctgattggct |
| 61 | actttgttcg cattataaaa ggcacgcgcg ggcgcgaggc ccttctctcg ccaggcgtcc |
| 121 | tcgtggaagt gacatcgtct ttaaaccctg cgtggcaatc cctgacgcac cgccgtgatg |
| 181 | cccagggaag acagggcgac ctggaagtcc aactacttcc ttaagatcat ccaactattg |
| 241 | gatgattatc cgaaatgttt cattgtggga gcagacaatg tgggctccaa gcagatgcag |
| 301 | cagatccgca tgtcccttcg cgggaaggct gtggtgctga tgggcaagaa caccatgatg |
| 361 | cgcaaggcca tccgagggca cctggaaaac aacccagctc tggagaaact gctgcctcat |
| 421 | atccggggga atgtgggctt tgtgttcacc aaggaggacc tcactgagat cagggacatg |
| 481 | ttgctggcca ataaggtgcc agctgctgcc cgtgctggtg ccattgcccc atgtgaagtc |
| 541 | actgtgccag cccagaacac tggtctcggg cccgagaaga cctccttttt ccaggcttta |
| 601 | ggtatcacca ctaaaatctc caggggcacc attgaaatcc tgagtgatgt gcagctgatc |
| 661 | aagactggag acaaagtggg agccagcgaa gccacgctgc tgaacatgct caacatctcc |
| 721 | cccttctcct ttgggctggt catccagcag gtgttcgaca atggcagcat ctacaaccct |
| 781 | gaagtgcttg atatcacaga ggaaactctg cattctcgct tcctggaggg tgtccgcaat |
| 841 | gttgccagtg tctgtctgca gattggctac ccaactgttg catcagtacc ccattctatc |
| 901 | atcaacgggt acaaacgagt cctggccttg tctgtggaga cggattacac cttcccactt |
| 961 | gctgaaaagg tcaaggcctt cttggctgat ccatctgcct ttgtggctgc tgcccctgtg |
| 1021 | gctgctgcca ccacagctgc tcctgctgct gctgcagccc cagctaaggt tgaagccaag |
| 1081 | gaagagtcgg aggagtcgga cgaggatatg ggatttggtc tctttgacta atcaccaaaa |
| 1141 | agcaaccaac ttagccagtt ttatttgcaa aacaaggaaa taaaggctta cttctttaaa |
| 1201 | aagtaaaaaa |

LOCUS NM_001003 512 bp mRNA linear PRI 11-JAN-2014
DEFINITION Homo sapiens ribosomal protein, large, P1 (RPLP1), transcript
variant 1, mRNA. (SEQ ID NO: 647)

| | |
|---|---|
| 1 | cctttcctca gctgccgcca aggtgctcgg tccttccgag gaagctaagg ctgcgttggg |
| 61 | gtgaggccct cacttcatcc ggcgactagc accgcgtccg gcagcgccag ccctacactc |
| 121 | gcccgcgcca tggcctctgt ctccgagctc gcctgcatct actcggccct cattctgcac |
| 181 | gacgatgagg tgacagtcac ggaggataag atcaatgccc tcattaaagc agccggtgta |
| 241 | aatgttgagc ctttttggcc tggcttgttt gcaaaggccc tggccaacgt caacattggg |
| 301 | agcctcatct gcaatgtagg ggccggtgga cctgctccag cagctggtgc tgcaccagca |
| 361 | ggaggtcctg ccccctccac tgctgctgct ccagctgagg agaagaaagt ggaagcaaag |
| 421 | aaagaagaat ccgaggagtc tgatgatgac atgggctttg gtctttttga ctaaacctct |
| 481 | tttataacat gttcaataaa aagctgaact tt |

LOCUS NM_001004 511 bp mRNA linear PRI 24-NOV-2013
DEFINITION Homo sapiens ribosomal protein, large, P2 (RPLP2), mRNA.
(SEQ ID NO: 648)

| | |
|---|---|
| 1 | ggtttaaccc cgcctcttgc gtcggcgcct tccttttcct ccctgtcgcc accgaggtcg |
| 61 | cacgcgtgag acttctccgc cgcctccgcc gcagacgccg ccgcgatgcg ctacgtcgcc |
| 121 | tcctacctgc tggctgccct agggggcaac tcctccccca gcgccaagga catcaagaag |
| 181 | atcttggaca gcgtgggtat cgaggcggac gacgaccggc tcaacaaggt tatcagtgag |
| 241 | ctgaatggaa aaaacattga agacgtcatt gcccagggta ttggcaagct tgccagtgta |
| 301 | cctgctggtg gggctgtagc cgtctctgct gccccaggct ctgcagcccc tgctgctggt |
| 361 | tctgcccctg ctgcagcaga ggagaagaaa gatgagaaga aggaggagtc tgaagagtca |
| 421 | gatgatgaca tgggatttgg cctttttgat taaattcctg ctcccctgca aataaagcct |
| 481 | ttttacacat ctcaa |

LOCUS NM_000967 1348 bp mRNA linear PRI 10-JAN-2014
DEFINITION Homo sapiens ribosomal protein L3 (RPL3), transcript variant 1,
mRNA. (SEQ ID NO: 649)

| | |
|---|---|
| 1 | ccagatttgg ctttatatag cggacccgta aggccgaccg gcctctaccg gcgggatttg |
| 61 | atggcgtgat gtctcacaga aagttctccg ctcccagaca tgggtccctc ggcttcctgc |
| 121 | ctcggaagcg cagcagcagg catcgtggga aggtgaagag cttccctaag gatgacccgt |
| 181 | ccaagccggt ccacctcaca gccttcctgg gatacaaggc tggcatgact cacatcgtgc |
| 241 | gggaagtcga caggccggga tccaaggtga acaagaagga ggtggtggag gctgtgacca |
| 301 | ttgtagagac accacccatg gtggttgtgg gcattgtggg ctacgtggaa acccctcgag |
| 361 | gcctccggac cttcaagact gtctttgctg agcacatcag tgatgaatgc aagaggcgtt |
| 421 | tctataagaa ttggcataaa tctaagaaga aggcctttac caagtactgc aagaaatggc |
| 481 | aggatgagga tggcaagaag cagctggaga aggacttcag cagcatgaag aagtactgcc |
| 541 | aagtcatccg tgtcattgcc cacacccaga tgcgcctgct tcctctgcgc cagaagaagg |
| 601 | cccacctgat ggagatccag gtgaacggag gcactgtggc cgagaagctg gactgggccc |
| 661 | gcgagaggct tgagcagcag gtacctgtga accaagtgtt tgggcaggat gagatgatcg |
| 721 | acgtcatcgg ggtgaccaag ggcaaaggct acaaaggggt caccagtcgt tggcacacca |
| 781 | agaagctgcc ccgcaagacc caccgaggcc tgcgcaaggt ggcctgtatt ggggcatggc |
| 841 | atcctgctcg tgtagccttc tctgtggcac gcgctgggca gaaaggctac catcaccgca |
| 901 | ctgagatcaa caagaagatt tataagattg gccagggcta ccttatcaag gacggcaagc |
| 961 | tgatcaagaa caatgcctcc actgactatg acctatctga caagagcatc aaccctctgg |
| 1021 | gtggctttgt ccactatggt gaagtgacca atgactttgt catgctgaaa ggctgtgtgg |
| 1081 | tgggaaccaa gaagcgggtg ctcaccctcc gcaagtcctt gctggtgcag acgaagcggc |
| 1141 | gggctctgga gaagattgac cttaagttca ttgacaccac ctccaagttt ggccatggcc |
| 1201 | gcttccagac catggaggag aagaaagcat tcatgggacc actgaagaaa gaccgaattg |
| 1261 | caaaggaaga aggagcttaa tgccaggaac agattttgca gttggtgggg tctcaataaa |
| 1321 | agttattttc cactgacaaa |

LOCUS NM_000968 1458 bp mRNA linear PRI 10-JAN-2014
DEFINITION Homo sapiens ribosomal protein L4 (RPL4), mRNA. (SEQ ID NO: 650)

| | |
|---|---|
| 1 | aagcacttcc ttttcctgtg gcagcagccg ggctgagagg agcgtggctg tctcctctct |
| 61 | ccgccatggc gtgtgctcgc ccactgatat cggtgtactc cgaaaagggg gagtcatctg |
| 121 | gcaaaaatgt cactttgcct gctgtattca aggctcctat tcgaccagat attgtgaact |
| 181 | ttgttcacac caacttgcgc aaaaacaaca gacagcccta tgctgtcagt gaattagcag |
| 241 | gtcatcagac tagtgctgag tcttggggta ctggcagagc tgtggctcga attcccagag |
| 301 | ttcgaggtgg tgggactcac cgctctggcc agggtgcttt tggaaacatg tgtcgtggag |
| 361 | gccgaatgtt tgcaccaacc aaaacctggc gccgttggca tcgtagagtg aacacaaccc |
| 421 | aaaaacgata cgccatctgt tctgccctgg ctgcctcagc cctaccagca ctggtcatgt |
| 481 | ctaaaggtca tcgtattgag gaagttcctg aacttccttt ggtagttgaa gataaagttg |
| 541 | aaggctacaa gaagaccaag gaagctgttt tgctccttaa gaaacttaaa gcctggaatg |
| 601 | atatcaaaaa ggtctatgcc tctcagcgaa tgagagctgg caaaggcaaa atgagaaacc |
| 661 | gtcgccgtat ccagcgcagg ggcccgtgca tcatctataa tgaggataat ggtatcatca |
| 721 | aggccttcag aaacatccct ggaattactc tgcttaatgt aagcaagctg aacattttga |
| 781 | agcttgctcc tggtgggcat gtgggacgtt tctgcatttg gactgaaagt gctttccgga |
| 841 | agttagatga attgtacggc acttggcgta aagccgcttc cctcaagagt aactacaatc |
| 901 | ttcccatgca caagatgatt aatacagatc ttagcagaat cttgaaaagc ccagagatcc |
| 961 | aaagagccct tcgagcacca cgcaagaaga tccatcgcag agtcctaaag aagaacccac |
| 1021 | tgaaaaactt gagaatcatg ttgaagctaa acccatatgc aaagaccatg cgccggaaca |
| 1081 | ccattcttcg ccaggccagg aatcacaagc tccgggtgga taaggcagct gctgcagcag |
| 1141 | cggcactaca agccaaatca gatgagaagg cggcggttgc aggcaagaag cctgtggtag |
| 1201 | gtaagaaagg aaagaaggct gctgttggtg ttaagaagca gaagaagcct ctggtgggaa |
| 1261 | aaaaggcagc agctaccaag aaaccagccc ctgaaaagaa gcctgcagag aagaaaccta |
| 1321 | ctacagagga gaagaagcct gctgcataaa ctcttaaatt tgattattcc ataaaggtca |
| 1381 | aatcattttg gacagcttct tttgaataaa gacctgatta tacaggcagt gagaaacatg |

LOCUS NM_000969 1035 bp mRNA linear PRI 18-JAN-2014
DEFINITION Homo sapiens ribosomal protein L5 (RPL5), mRNA. (SEQ ID NO: 651)

| | |
|---|---|
| 1 | ggcccttttc ccacccccta gcgccgctgg gcctgcaggt ctctgtcgag cagcggacgc |
| 61 | cggtctctgt tccgcaggat ggggtttgtt aaagttgtta agaataaggc ctactttaag |
| 121 | agataccaag tgaaatttag aagacgacga gagggtaaaa ctgattatta tgctcggaaa |
| 181 | cgcttggtga tacaagataa aaataaatac aacacaccca aatacaggat gatagttcgt |
| 241 | gtgacaaaca gagacatcat ttgtcagatt gcttatgccc gtatagaggg ggatatgata |
| 301 | gtctgcgcag cgtatgcaca cgaaccgcca aaatatggtg tgaaggttgg cctgacaaat |
| 361 | tatgctgcag catattgtac tggcctgctg ctggcccgca ggcctctcaa taggtttggc |
| 421 | atggacaaga tctatgaagg ccaagtggag gtgactggtg atgaatacaa tgtggaaagc |
| 481 | attgatggtc agccaggtgc cttcacctgc tatttggatg caggccttgc cagaactacc |
| 541 | actggcaata aagtttttgg tgccctgaag ggagctgtgg atggaggctt gtctatccct |
| 601 | cacagtacca aacgattccc tggttatgat tctgaaagca aggaatttaa tgcagaagta |
| 661 | catcggaagc acatcatggg ccagaatgtt gcagattaca tgcgctactt aatggaagaa |
| 721 | gatgaagatg cttacaagaa acagttctct caatacataa agaacagcgt aactccagac |
| 781 | atgatggagg agatgtataa gaaagctcat gctgctatac gagagaatcc agtctatgaa |
| 841 | aagaagccca agaaagaagt taaaaagaag aggtggaacc gtcccaaaat gtcccttgct |
| 901 | cagaagaagg atcgggtagc tcaaaagaag gcaagcttcc tcagagctca ggagcgggct |
| 961 | gctgagagct aaacccagca attttctatg attttttcag atatagataa taaacttatg |
| 1021 | aacagcaact aaaaa |

LOCUS NM_000970 972 bp mRNA linear PRI 26-JAN-2014
DEFINITION Homo sapiens ribosomal protein L6 (RPL6), transcript variant 2,
mRNA. (SEQ ID NO: 652)

| | |
|---|---|
| 1 | gattgcttat agaccggaag ccgggacctt aattctcttt cccatcttgc aagatggcgg |
| 61 | gtgaaaaagt tgagaagcca gatactaaag agaagaaacc cgaagccaag aaggttgatg |
| 121 | ctggtggcaa ggtgaaaaag ggtaacctca aagctaaaaa gcccaagaag gggaagcccc |
| 181 | attgcagccg caaccctgtc cttgtcagag gaattggcag gtattcccga tctgccatgt |
| 241 | attccagaaa ggccatgtac aagaggaagt actcagccgc taaatccaag gttgaaaaga |
| 301 | aaaagaagga gaaggttctc gcaactgtta caaaaccagt tggtggtgac aagaacggcg |
| 361 | gtacccgggt ggttaaactt cgcaaaatgc ctagatatta tcctactgaa gatgtgcctc |
| 421 | gaaagctgtt gagccacggc aaaaaaccct tcagtcagca cgtgagaaaa ctgcgagcca |
| 481 | gcattacccc cgggaccatt ctgatcatcc tcactggacg ccacaggggc aagagggtgg |
| 541 | ttttcctgaa gcagctggct agtggcttat tacttgtgac tggacctctg gtcctcaatc |
| 601 | gagttcctct acgaagaaca caccagaaat ttgtcattgc cacttcaacc aaaatcgata |
| 661 | tcagcaatgt aaaaatccca aaacatctta ctgatgctta cttcaagaag aagaagctgc |
| 721 | ggaagcccag acaccaggaa ggtgagatct tcgacacaga aaaagagaaa tatgagatta |
| 781 | cggagcagcg caagattgat cagaaagctg tggactcaca aattttacca aaaatcaaag |
| 841 | ctattcctca gctccagggc tacctgcgat ctgtgtttgc tctgacgaat ggaatttatc |
| 901 | ctcacaaatt ggtgttctaa atgtcttaag aacctaatta aatagctgac tacaaaaaaa |

LOCUS NM_000971 866 bp mRNA linear PRI 10-JAN-2014
DEFINITION Homo sapiens ribosomal protein L7 (RPL7), mRNA. (SEQ ID NO: 653)

| | |
|---|---|
| 1 | tcctcttttt ccggctggaa ccatggaggg tgtagaagag aagaagaagg aggttcctgc |
| 61 | tgtgccagaa acccttaaga aaaagcgaag gaatttcgca gagctgaaga tcaagcgcct |
| 121 | gagaaagaag tttgcccaaa agatgcttcg aaaggcaagg aggaagctta tctatgaaaa |
| 181 | agcaaagcac tatcacaagg aatataggca gatgtacaga actgaaattc gaatggcgag |
| 241 | gatggcaaga aaagctggca acttctatgt acctgcagaa cccaaattgg cgtttgtcat |
| 301 | cagaatcaga ggtatcaatg gagtgagccc aaaggttcga aaggtgttgc agcttcttcg |
| 361 | ccttcgtcaa atcttcaatg gaacctttgt gaagctcaac aaggcttcga ttaacatgct |
| 421 | gaggattgta gagccatata ttgcatgggg gtaccccaat ctgaagtcag taaatgaact |
| 481 | aatctacaag cgtggttatg gcaaaatcaa taagaagcga attgctttga cagataacgc |
| 541 | tttgattgct cgatctcttg gtaaatacgg catcatctgc atggaggatt tgattcatga |
| 601 | gatccatact gttggaaaac gcttcaaaga ggcaaataac ttcctgtggc ccttcaaatt |
| 661 | gtcttctcca cgaggtggaa tgaagaaaaa gaccacccat tttgtagaag gtggagatgc |
| 721 | tggcaacagg gaggaccaga tcaacaggct tattagaaga atgaactaag gtgtctacca |
| 781 | tgattatttt tctaagctgg ttggttaata aacagtacct gctctcaaat tgaaataaaa |

LOCUS NM_000972 890 bp mRNA linear PRI 07-JUL-2013
DEFINITION Homo sapiens ribosomal protein L7a (RPL7A), mRNA. (SEQ ID NO: 654)

| | |
|---|---|
| 1 | tttcctttct ctctcctccc gccgcccaag atgccgaaag gaaagaaggc caagggaaag |
| 61 | aaggtggctc cggccccagc tgtcgtgaag aagcaggagg ctaagaaagt ggtgaatccc |
| 121 | ctgtttgaga aaaggcctaa gaattttggc attggacagg acatccagcc caaaagagac |
| 181 | ctcacccgct ttgtgaaatg gccccgctat atcaggttgc agcggcagag agccatcctc |
| 241 | tataagcggc tgaaagtgcc tcctgcgatt aaccagttca cccaggccct ggaccgccaa |
| 301 | acagctactc agctgcttaa gctggcccac aagtacagac cagagacaaa gcaagagaag |
| 361 | aagcagagac tgttggcccg ggccgagaag aaggctgctg gcaaagggga cgtcccaacg |
| 421 | aagagaccac ctgtccttcg agcaggagtt aacaccgtca ccaccttggt ggagaacaag |
| 481 | aaagctcagc tggtggtgat tgcacacgac gtggatccca tcgagctggt tgtcttcttg |
| 541 | cctgccctgt gtcgtaaaat gggggtccct tactgcatta tcaagggaaa ggcaagactg |
| 601 | ggacgtctag tccacaggaa gacctgcacc actgtcgcct tcacacaggt gaactcggaa |
| 661 | gacaaaggcg ctttggctaa gctggtggaa gctatcagga ccaattacaa tgacagatac |
| 721 | gatgagatcc gccgtcactg gggtggcaat gtcctgggtc ctaagtctgt ggctcgtatc |
| 781 | gccaagctcg aaaaggcaaa ggctaaagaa cttgccacta aactgggtta aatgtacact |
| 841 | gttgagtttt ctgtacataa aaataattga aataatacaa attttccttc |

LOCUS NM_000973 903 bp mRNA linear PRI 10-JAN-2014
DEFINITION Homo sapiens ribosomal protein L8 (RPL8), transcript variant 1,
mRNA. (SEQ ID NO: 655)

| | |
|---|---|
| 1 | agataaggcc gctcgctgac gccgtgtttc ctctttcggc cgcgctggtg aacaggaccc |
| 61 | gtcgccatgg gccgtgtgat ccgtggacag aggaagggcg ccgggtctgt gttccgcgcg |
| 121 | cacgtgaagc accgtaaagg cgctgcgcgc ctgcgcgccg tggatttcgc tgagcggcac |
| 181 | ggctacatca agggcatcgt caaggacatc atccacgacc cgggccgcgg cgcgcccctc |
| 241 | gccaaggtgg tcttccggga tccgtatcgg tttaagaagc ggacggagct gttcattgcc |
| 301 | gccgagggca ttcacacggg ccagtttgtg tattgcggca agaaggccca gctcaacatt |
| 361 | ggcaatgtgc tccctgtggg caccatgcct gagggtacaa tcgtgtgctg cctggaggag |
| 421 | aagcctggag accgtggcaa gctggcccgg gcatcaggga actatgccac cgttatctcc |
| 481 | cacaaccctg agaccaagaa gacccgtgtg aagctgccct ccggctccaa gaaggttatc |
| 541 | tcctcagcca acagagctgt ggttggtgtg gtggctggag gtggccgaat tgacaaaccc |
| 601 | atcttgaagg ctggccgggc gtaccacaaa tataaggcaa agaggaactg ctggccacga |
| 661 | gtacggggtg tggccatgaa tcctgtggag catccttttg gaggtggcaa ccaccagcac |
| 721 | atcggcaagc cctccaccat ccgcagagat gcccctgctg gccgcaaagt gggtctcatt |
| 781 | gctgcccgcc ggactggacg tctccgggga accaagactg tgcaggagaa agagaactag |
| 841 | tgctgagggc ctcaataaag tttgtgttta tgccaa |

LOCUS NM_000661 766 bp mRNA linear PRI 26-JAN-2014
DEFINITION Homo sapiens ribosomal protein L9 (RPL9), transcript variant 1,
mRNA. (SEQ ID NO: 656)

| | |
|---|---|
| 1 | acgcgataca agtacgtaat gacgacagac gttctttctt tgctgcgtct actgcgagaa |
| 61 | tgaagactat tctcagcaat cagactgtcg acattccaga aaatgtcgac attactctga |
| 121 | agggacgcac agttatcgtg aagggcccca gaggaaccct gcggagggac ttcaatcaca |
| 181 | tcaatgtaga actcagcctt cttggaaaga aaaaaaagag gctccgggtt gacaaatggt |
| 241 | ggggtaacag aaaggaactg gctaccgttc ggactatttg tagtcatgta cagaacatga |
| 301 | tcaagggtgt tacactgggc ttccgttaca agatgaggtc tgtgtatgct cacttcccca |
| 361 | tcaacgttgt tatccaggag aatgggtctc ttgttgaaat ccgaaatttc ttgggtgaaa |
| 421 | aatatatccg cagggttcgg atgagaccag gtgttgcttg ttcagtatct caagcccaga |
| 481 | aagatgaatt aatccttgaa ggaaatgaca ttgagcttgt ttcaaattca gcggctttga |
| 541 | ttcagcaagc cacaacagtt aaaaacaagg atatcaggaa atttttggat ggtatctatg |
| 601 | tctctgaaaa aggaactgtt cagcaggctg atgaataaga tctaagagtt acctggctac |
| 661 | agaaagaaga tgccagatga cacttaagac ctacttgtga tatttaaatg atgcaataaa |
| 721 | agacctattg atttggacct tcttcttaaa |

LOCUS NM_006013 2335 bp mRNA linear PRI 18-JAN-2014
DEFINITION Homo sapiens ribosomal protein L10 (RPL10), transcript variant 1,
mRNA. (SEQ ID NO: 657)

| | |
|---|---|
| 1 | gggctacgcc cgggcgcaag cgccaagagc ggctgcgtct atggtcatga cgtctgacag |
| 61 | agcgtccacc cgtcttcgac aggactctat ggttcttacg cgcgcagaca gaccgcctat |
| 121 | ataagccatg cgcaggcgga ggagcgcctc tttcccttcg gtgtgccact gaagatcctg |
| 181 | gtgtcgccat gggccgccgc cccgcccgtt gttaccggta ttgtaagaac aagccgtacc |
| 241 | caaagtctcg cttctgccga ggtgtccctg atgccaagat tcgcattttt gacctggggc |
| 301 | ggaaaaaggc aaaagtggat gagtttccgc tttgtggcca catggtgtca gatgaatatg |
| 361 | agcagctgtc ctctgaagcc ctggaggctg cccgaatttg tgccaataag tacatggtaa |
| 421 | aaagttgtgg caaagatggc ttccatatcc gggtgcggct ccaccccttc cacgtcatcc |
| 481 | gcatcaacaa gatgttgtcc tgtgctgggg ctgacaggct ccaaacaggc atgcgaggtg |
| 541 | cctttggaaa gccccagggc actgtggcca gggttcacat tggccaagtt atcatgtcca |
| 601 | tccgcaccaa gctgcagaac aaggagcatg tgattgaggc cctgcgcagg gccaagttca |
| 661 | agtttcctgg ccgccagaag atccacatct caaagaagtg gggcttcacc aagttcaatg |
| 721 | ctgatgaatt tgaagacatg gtggctgaaa agcggctcat cccagatggc tgtggggtca |
| 781 | agtacatccc caatcgtggc cctctggaca agtggcgggc cctgcactca tgagggcttc |
| 841 | caatgtgctg cccccctctt aatactcacc aataaattct acttcctgtc cacctatgtc |
| 901 | tttgtatcta cattcttgac ggggaaggaa cttcctctgg gaacctttgg gtcattgccc |
| 961 | tttcacttca gaaacaggtt gacaactcag ccctgctcat gaggcagcaa accctgcaaa |
| 1021 | gggctgggac tggtggcctt atgtcagttg tctactctgg agcttgactt ggacctcccc |
| 1081 | aggtcctagg cagtaggttg aaaaacactg aagtgctttt catgaagcac agctgcagca |
| 1141 | aagccttgca atcccaggct ggggtcagcc tacagttgtg ttgcttatta caacacatgc |
| 1201 | ggaccaagag gggcttgtgg gctagaggct gaccagcagc gtttatttag caagggtagg |
| 1261 | tgtgcatcac attgggcttg ttctcaccca tctggtttgg ccattcctcc ttggtgggaa |
| 1321 | tcatccaggt actgctgagg tcacctgcga tttgccccat ttcctatctc tagcaacctc |
| 1381 | ctgggcccca tgcccccacc ccttctagaa cctgcattcc cagggccttc accacctgac |
| 1441 | caaaggtcta ggctaacctt tggtcatttg taacaagacc tcggaacaga cacgtgtgtg |
| 1501 | gcatggtttg gcctggggat cttagatgtc tgacctgaac tattgtagaa cagcgctggc |
| 1561 | ttttggggga gcagcaaaaa tgagaggagt gctaggtggg tggcctgagc atctgtatcc |
| 1621 | agggacagga ctccaaaggc ttttggtccc agagctgggg tatgttggcc ccagccccca |
| 1681 | gcctgtggct cccaaaaggc ctctggtttt ttgtaatctc agtttacagc catttcttag |
| 1741 | gtttttaatt acctttattt tattttgcca aacatacctg ggaatacctt ttattttttt |
| 1801 | tttaccttgg ggtgatggtt ccaaaccata aatgtgatta tagttaacac atgacccttc |
| 1861 | tagcgtccca gccagtgttt ttcctgacct ctgttctttg gagaggagga tggaagggag |
| 1921 | gggtccggca cgctgctggc attttgctgt gtcctgcagc ccctttccgg gacacctggg |
| 1981 | ttcacacagc tttttagctt acataactgg tgcagatttt ctgtgtggag atgttgcctt |
| 2041 | gaccagcctt ggctggactt taccaggcat gcagaagcct gtaccaacac agactacagc |
| 2101 | acccaggagg tgcgagtgtg gctgctcagc ggttataaca ggcctgactg cattgttcac |
| 2161 | cggattataa tgagccaaaa tgtttcccgg tgtttgctgg tttcagggaa ggagtttgat |
| 2221 | atagcagatt aaccaccctc cttgtagcta ttggggctta atggtttcct ggtgattctt |
| 2281 | accaatccac aataaacatg gcccattggc atatctgcaa |

LOCUS NM_007104 719 bp mRNA linear PRI 26-JAN-2014
DEFINITION Homo sapiens ribosomal protein L10a (RPL10A), mRNA.
(SEQ ID NO: 658)

| | |
|---|---|
| 1 | agtctctttt ccggttagcg cggcgtgaga agccatgagc agcaaagtct ctcgcgacac |
| 61 | cctgtacgag gcggtgcggg aagtcctgca cgggaaccag cgcaagcgcc gcaagttcct |
| 121 | ggagacggtg gagttgcaga tcagcttgaa gaactatgat ccccagaagg acaagcgctt |
| 181 | ctcgggcacc gtcaggctta agtccactcc ccgccctaag ttctctgtgt gtgtcctggg |
| 241 | ggaccagcag cactgtgacg aggctaaggc cgtggatatc ccccacatgg acatcgaggc |
| 301 | gctgaaaaaa ctcaacaaga ataaaaaact ggtcaagaag ctggccaaga agtatgatgc |
| 361 | gtttttggcc tcagagtctc tgatcaagca gattccacga atcctcggcc caggtttaaa |
| 421 | taaggcagga aagttccctt ccctgctcac acacaacgaa aacatggtgg ccaaagtgga |
| 481 | tgaggtgaag tccacaatca agttccaaat gaagaaggtg ttatgtctgg ctgtagctgt |
| 541 | tggtcacgtg aagatgacag acgatgagct tgtgtataac attcacctgg ctgtcaactt |
| 601 | cttggtgtca ttgctcaaga aaaactggca gaatgtccgg gccttatata tcaagagcac |
| 661 | catgggcaag ccccagcgcc tatattaagg cacatttgaa taaattctat taccagttc |

LOCUS NM_000975 644 bp mRNA linear PRI 19-JAN-2014
DEFINITION Homo sapiens ribosomal protein L11 (RPL11), transcript variant 1,
mRNA. (SEQ ID NO: 659)

| | |
|---|---|
| 1 | aaggccctcg gccggaagct ccgctttctc ttcctgctct ccatcatggc gcaggatcaa |
| 61 | ggtgaaaagg agaaccccat gcgggaactt cgcatccgca aactctgtct caacatctgt |
| 121 | gttggggaga gtggagacag actgacgcga gcagccaagg tgttggagca gctcacaggg |
| 181 | cagacccctg tgttttccaa agctagatac actgtcagat cctttggcat ccggagaaat |
| 241 | gaaaagattg ctgtccactg cacagttcga ggggccaagg cagaagaaat cttggagaag |
| 301 | ggtctaaagg tgcgggagta tgagttaaga aaaaacaact tctcagatac tggaaacttt |
| 361 | ggttttggga tccaggaaca catcgatctg ggtatcaaat atgacccaag cattggtatc |
| 421 | tacggcctgg acttctatgt ggtgctgggt aggccaggtt tcagcatcgc agacaagaag |
| 481 | cgcaggacag gctgcattgg ggccaaacac agaatcagca aagaggaggc catgcgctgg |
| 541 | ttccagcaga agtatgatgg gatcatcctt cctggcaaat aaattcccgt ttctatccaa |
| 601 | aagagcaata aaaagttttc agtgaaatgt gcaaaaa |

LOCUS NM_000976 674 bp mRNA linear PRI 10-JAN-2014
DEFINITION Homo sapiens ribosomal protein L12 (RPL12), mRNA. (SEQ ID NO: 660)

| | |
|---|---|
| 1 | atctggcttg tccgcgcgat ttccggcctc tcggctttcg gctcggagga ggccaaggtg |
| 61 | caacttcctt cggtcgtccc gaatccgggt tcatccgaca ccagccgcct ccaccatgcc |
| 121 | gccgaagttc gaccccaacg agatcaaagt cgtatacctg aggtgcaccg gaggtgaagt |
| 181 | cggtgccact tctgccctgg cccccaagat cggccccctg ggtctgtctc caaaaaaagt |
| 241 | tggtgatgac attgccaagg caacgggtga ctggaagggc ctgaggatta cagtgaaact |
| 301 | gaccattcag aacagacagg cccagattga ggtggtgcct tctgcctctg ccctgatcat |
| 361 | caaagccctc aaggaaccac caagagacag aaagaaacag aaaaacatta aacacagtgg |
| 421 | gaatatcact tttgatgaga ttgtcaacat tgctcgacag atgcggcacc gatccttagc |
| 481 | cagagaactc tctggaacca ttaaagagat cctggggact gcccagtcag tgggctgtaa |
| 541 | tgttgatggc cgccatcctc atgacatcat cgatgacatc aacagtggtg ctgtggaatg |
| 601 | cccagccagt taagcacaaa ggaaaacatt tcaataaagg atcatttgac aactggtgga |

LOCUS NM_033251 4672 bp mRNA linear PRI 17-APR-2013
DEFINITION Homo sapiens ribosomal protein L13 (RPL13), transcript variant 2,
mRNA. (SEQ ID NO: 661)

| | |
|---|---|
| 1 | aggtgaggga gactgggtcc tggcctttgg gcatcatcca gcgccatcgg cctggcgctt |
| 61 | cagccaacgc gggagtggat gggccccttc ttcttcgcag acagcgttcg gccgctgccc |
| 121 | gggctctagg cgcggccgga cggcccagtc tggagggttc ggggcggagg cccggggggg |
| 181 | tgcgcgcgcc cggggtccgg cctctcactc gctcccctct cgtccgcagc cgcagggccg |
| 241 | taggcagcca tggcgcccag ccggaatggc atggtcttga agccccactt ccacaaggac |
| 301 | tggcagcggc gcgtggccac gtggttcaac cagccggccc gtaagatccg cagacgtaag |
| 361 | gcccggcaag ccaaggcgcg ccgcatcgcc ccgcgccccg cgtcgggtcc catccggccc |
| 421 | atcgtgcgct gccccacggt tcggtaccac acgaaggtgc gcgccggccg cggcttcagc |
| 481 | ctggaggagc tcagggtggc cggcattcac aagaaggtgg cccggaccat cggcatttct |
| 541 | gtggatccga ggaggcggaa caagtccacg gagtccctgc aggccaacgt gcagcggctg |
| 601 | aaggagtacc gctccaaact catcctcttc cccaggaagc cctcggcccc caagaaggga |
| 661 | gacagttctg ctgaagaact gaaactggcc acccagctga ccggaccggt catgcccgtc |
| 721 | cggaacgtct ataagaagga gaaagctcga gtcatcactg aggaagagaa gaatttcaaa |
| 781 | gccttcgcta gtctccgtat ggcccgtgcc aacgcccggc tcttcggcat acgggcaaaa |
| 841 | agagccaagg aagccgcaga acaggatgtt gaaaagaaaa aataaagccc tcctggggac |
| 901 | ttggaatcag tcggcagtca tgctgggtct ccacgtggtg tgtttcgtgg gaacaactgg |
| 961 | gcctgggatg gggcttcact gctgtgactt cctcctgcca ggggatttgg ggctttcttg |
| 1021 | aaagacagtc caagccctgg ataatgcttt actttctgtg ttgaagcact gttggttgtt |
| 1081 | tggttagtga ctgatgtaaa acggttttct tgtggggagg ttacagaggc tgacttcaga |
| 1141 | gtggacttgt gttttttctt tttaaagagg caaggttggg ctggtgctca cagctgtaat |
| 1201 | cccagcactt tgaggttggc tgggagttca agaccagcct ggccaacatg tcagaactac |
| 1261 | taaaaataaa gaaatcagcc atgcttggtg ctgcacactt gtagttgcag ctcctgggag |
| 1321 | gcagaggtga gggatcactt aacccaggag gcagaggctg cactgagcca ggatcacgcc |
| 1381 | actgcactct agcctgggca acagtgagac tgtctcaaaa aaaaaaaaag agacagggtc |
| 1441 | ttcggcaccc aggctggagt acagtgccac aatcatggct cactgcagtc ttgaactcat |
| 1501 | ggcctcaagc agtcctccct cagcctccca agtagagggg tttataggca cgagaccctg |
| 1561 | cacccaacct agagttgcct tttttaagca aagcagtttc tagttaatgt agcatcttgg |
| 1621 | actttggggc gtcattctta agcttgttgt gcccggtaac catggtcctc ttgctctgat |
| 1681 | taacccttcc ttcaatgggc ttcttcaccc agacaccaag gtatgagatg gccctgccaa |
| 1741 | gtgtcggcct ctcctgttaa acaaaaacat tctaaagcca ttgttcttgc ttcatggaca |
| 1801 | agaggcagcc agagagagtg ccagggtgcc ctggtctgag ctggcatccc catgtcttct |
| 1861 | gtgtccgagg gcagcatggt ttctcgtgca gtgctcagac acagcctgcc ctagtcctac |
| 1921 | cagctcacag cagcacctgc tctccttggc agctatggcc atgacaaccc cagagaagca |
| 1981 | gcttcaggga ccgagtcaga ttctgttttg tctacatgcc tctgccgggt gccggtattg |
| 2041 | aggcacccag ggagctgtta ctggcgtgga aataggtgat gctgctacct ctgctgctgc |
| 2101 | actcacagcc acacttgata cacgatgaca ccttgcttgt ttggaaacat ctaaacatct |
| 2161 | agtagatgac ttgcaggctg ttggctacca gtttcctgtc tgaggtgtat atgttaactt |
| 2221 | cgtgatcagt ttgtatgttt gggactcttg tcctatgtaa agttaaggtg ggccgggtgc |
| 2281 | agtggctcac gcctgtaatc ctaacactgg gaggccgagg cgggtggatc acctgatggt |
| 2341 | gaaacctcat ctctactgaa aatacaaaaa ttagctgagt ggtgacacac gcctgtaatc |
| 2401 | ccagctactt ggtaggcttg aacccaggag gcagagattg cagtgagccg agctgcacca |
| 2461 | ctgtgctcca gcctgggtga cagcgagact cagtctcaaa aaaagttgta caaggtggat |
| 2521 | ggttggaagc ttgagcctag gctcgaatcc ctctcacgtg agagggcctg aagatttctg |
| 2581 | gtggattcca acctggctga agactggccg tggggggtgc aggggtctcc agcgctctgc |
| 2641 | cctccagcct gcttcctccc tgcccacacc gcactagggg aagggccttt cctgctgcct |
| 2701 | gcggggccgc acctggagta ggtaatgcca tgtggtgacg tgaatggagc agaggtctgt |
| 2761 | gccccatcac accgccttgc tgtttttact gtgggacaaa agcactctga tctgcgtgtt |
| 2821 | ccgggggccc tcctaccagc cgacttgacg ggaagtcagg gttcaggtat catctgtgca |
| 2881 | cctggggcgg ggtagtctgc actgaacctg ccagagtccc ctcctcattt cactgaaagt |
| 2941 | cacagtctcc agggctgtgt tgctaacctt acgttctctc cgtttgctta atctattaag |
| 3001 | agccctaaca ggagaggatg ggctttctct gttgtctggg gccctgctgt tggccggtgc |
| 3061 | tcttagcaag aggtcatttt tctaggttgc gctgggacat tgtgagtttg gtgagggtca |
| 3121 | tggatgtggg ctgggctggg ctgggctggg ccgggctgcc tgctgcctgc tgctccccta |
| 3181 | cctgaaatgc agctagtgcg gctctgccct tcctggggct gaggaaggct tctgcaggat |
| 3241 | agctgggggg ctgggcaggt gggtgaggca gcctccctgc tgacactcag tccttgtagc |
| 3301 | tggagcaaga tctcctgatc caggtacggg cctgtctgct ccaagaaaga ctctgccacc |
| 3361 | agatgcaaag gggccctttg ttttaactta gtccctgggg accgcctgat tcagcacctg |
| 3421 | tcggcccagg ataccccgct ggtggggaca agtgcctgag tgtgggccgt gcccgagtgt |
| 3481 | ggccatccct gagtggggcc gtcctgacta ggaagtggct tttcagttgt gatgtgtggg |
| 3541 | cctgacctag ggggcgctgt ggaacccggg ctggaaccag ccctctgtgc caggccgcag |
| 3601 | acaggttccg ccggccctga ggggcagctg ccatggcgtg ggtcactggg agctgagagg |
| 3661 | aagggccccc accgcacctc aggcaaagcg gctctgggaa caccttgatt tcgtccatgt |
| 3721 | gagccgtccc agggagggca gccaagctgt gaagcctgag aaactgacct gtgtgccacg |
| 3781 | agcttgtggt ctgctgcccg gtggaggaag tgcaggtgcg cccaggctcc tcattccgtt |
| 3841 | ttgcaggatt ccttcggggt gtgagcattt cctattcagc ctgtcgcccc cggggagcac |
| 3901 | gggctggctc tgtggtgccc gtggcctttt gtagaagcgt tggttttacg gcaggttcat |
| 3961 | ctctggggca gcctcccaca gtgggtgggg ctttgccagc agtgcccacg ggggtcatgg |
| 4021 | ggccaggcgc gctccggcgc ctgcagaact gatcggggat agtctcagga ggcgctagtc |
| 4081 | acgtgccccg gtgatcgggg atagtctcag aaggcgctag tctcctgccc cggtgatcgg |
| 4141 | ggatagtctc aggaggcacg agtcgcctgc ctcggtgatg caccgtttct cacaccggct |
| 4201 | gctctggccc gagctaaagg ggaagacgtg tgcggatagg agctgcacac aattttcctc |
| 4261 | catgtattgt ttattttgct ttttcttttg gctagacatt aggaatttca gttttcccaa |
| 4321 | gttgtatttt tccttttcta ttttaaaatt atcatgcagg gctgggtgag gtcgctcacg |
| 4381 | cctatagtct caaaactttg ggaggctgag gggggaggat ggcatgagcc caggagttta |
| 4441 | aggctgcagt gagccgagat cgctccactg tcctccagcc tgcatgacag agcgagaccc |
| 4501 | tatctcagga aaaaaaaaaa caaaactatt atgcagtagt ttcgaccctg gaagacgagt |
| 4561 | gtgcatcttt gagttgtaac acgtgtacct cgcccatcca ggcgtagttt catttggaat |
| 4621 | ctggttatcc tgtagttgct ttgttaaaaa tatatgtaat tgcaaatcat tt |

LOCUS NM_012423 1196 bp mRNA linear PRI 03-NOV-2013
DEFINITION Homo sapiens ribosomal protein L13a (RPL13A), transcript variant
1, mRNA. (SEQ ID NO: 662)

| | |
|---|---|
| 1 | cacttctgcc gcccctgttt caagggataa gaaaccctgc gacaaaacct cctccttttc |
| 61 | caagcggctg ccgaagatgg cggaggtgca ggtcctggtg cttgatggtc gaggccatct |
| 121 | cctgggccgc ctggcggcca tcgtggctaa acaggtactg ctgggccgga aggtggtggt |
| 181 | cgtacgctgt gaaggcatca acatttctgg caatttctac agaaacaagt tgaagtacct |
| 241 | ggctttcctc cgcaagcgga tgaacaccaa cccttcccga ggcccctacc acttccgggc |
| 301 | ccccagccgc atcttctggc ggaccgtgcg aggtatgctg ccccacaaaa ccaagcgagg |
| 361 | ccaggccgct ctggaccgtc tcaaggtgtt tgacggcatc ccaccgccct acgacaagaa |
| 421 | aaagcggatg gtggttcctg ctgccctcaa ggtcgtgcgt ctgaagccta caagaaagtt |
| 481 | tgcctatctg gggcgcctgg ctcacgaggt tggctggaag taccaggcag tgacagccac |
| 541 | cctggaggag aagaggaaag agaaagccaa gatccactac cggaagaaga aacagctcat |
| 601 | gaggctacgg aaacaggccg agaagaacgt ggagaagaaa attgacaaat acacagaggt |
| 661 | cctcaagacc cacggactcc tggtctgagc ccaataaaga ctgttaattc ctcatgcgtt |
| 721 | gcctgccctt cctccattgt tgccctggaa tgtacgggac ccaggggcag cagcagtcca |
| 781 | ggtgccacag gcagccctgg gacataggaa gctgggagca aggaaagggt cttagtcact |
| 841 | gcctcccgaa gttgcttgaa agcactcgga gaattgtgca ggtgtcattt atctatgacc |
| 901 | aataggaaga gcaaccagtt actatgagtg aaagggagcc agaagactga ttggagggcc |
| 961 | ctatcttgtg agtggggcat ctgttggact ttccacctgg tcatatactc tgcagctgtt |
| 1021 | agaatgtgca agcacttggg gacagcatga gcttgctgtt gtacacaggg tatttctaga |
| 1081 | agcagaaata gactgggaag atgcacaacc aaggggttac aggcatcgcc catgctcctc |
| 1141 | acctgtattt tgtaatcaga aataaattgc ttttaaagaa |

LOCUS NM_003973 875 bp mRNA linear PRI 17-APR-2013
DEFINITION Homo sapiens ribosomal protein L14 (RPL14), transcript variant 2,
mRNA. (SEQ ID NO: 663)

| | |
|---|---|
| 1 | ggcggtgcgt tcttctacac atgcgcaggg ttgggcgggt cttcttcctt ctcgcctaac |
| 61 | gccgccaaca tggtgttcag gcgcttcgtg gaggttggcc gggtggccta tgtctccttt |
| 121 | ggacctcatg ccggaaaatt ggtcgcgatt gtagatgtta ttgatcagaa cagggctttg |
| 181 | gtcgatggac cttgcactca agtgaggaga caggccatgc ctttcaagtg catgcagctc |
| 241 | actgatttca tcctcaagtt tccgcacagt gcccaccaga agtatgtccg acaagcctgg |
| 301 | cagaaggcag acatcaatac aaaatgggca gccacacgat gggccaagaa gattgaagcc |
| 361 | agagaaagga aagccaagat gacagatttt gatcgtttta aagttatgaa ggcaaagaaa |
| 421 | atgaggaaca gaataatcaa gaatgaagtt aagaagcttc aaaaggcagc tctcctgaaa |
| 481 | gcttctccca aaaaagcacc tggtactaag ggtactgctg ctgctgctgc tgctgctgct |
| 541 | gctgctaaag ttccagcaaa aaagatcacc gccgcgagta aaaaggctcc agcccagaag |
| 601 | gttcctgccc agaaagccac aggccagaaa gcagcgcctg ctccaaaagc tcagaagggt |
| 661 | caaaaagctc cagcccagaa agcacctgct ccaaaggcat ctggcaagaa agcataagtg |
| 721 | gcaatcataa aaagtaataa aggttctttt tgacctgttg acaaatgtat ttaagccttt |
| 781 | ggatttaaag cctgttgagg ctggagttag gaggcagatt gatagtagga ttataataaa |
| 841 | cattaaataa tcagttcaaa |

LOCUS NM_002948 2350 bp mRNA linear PRI 18-JAN-2014
DEFINITION Homo sapiens ribosomal protein L15 (RPL15), transcript variant 1,
mRNA. (SEQ ID NO: 664)

| | |
|---|---|
| 1 | aaagacagcg gctccaccgc ggtacgcggc caccggcttt ggagcctgga ccccaacttg |
| 61 | cctcctctcg cggagagaca gtcgccgacg ctcgcttagc cgccgagacc tcgccgccaa |
| 121 | ctctctcacc tctcgagacg cccaggccgc tcaggctcga atcttgcgga gcagggggcg |
| 181 | ggacaatagc ggccgcggcg ccccactcgg cagaactccg ccaccaggcg cgatgccgga |
| 241 | actacatgtc ccatgacgct ctgggaggcc gcagctttcc accggaaaga gggtggctga |
| 301 | ggtgggggag gagcccaaaa ggcattgtgg gagtacagct ctttcctttc cgtctggcgg |
| 361 | cagccatcag gtaagccaag atgggtgcat acaagtacat ccaggagcta tggagaaaga |
| 421 | agcagtctga tgtcatgcgc tttcttctga gggtccgctg ctggcagtac cgccagctct |
| 481 | ctgctctcca cagggctccc cgccccaccc ggcctgataa agcgcgccga ctgggctaca |
| 541 | aggccaagca aggttacgtt atatatagga ttcgtgttcg ccgtggtggc cgaaaacgcc |
| 601 | cagttcctaa gggtgcaact tacggcaagc ctgtccatca tggtgttaac cagctaaagt |
| 661 | ttgctcgaag ccttcagtcc gttgcagagg agcgagctgg acgccactgt ggggctctga |
| 721 | gagtcctgaa ttcttactgg gttggtgaag attccacata caaatttttt gaggttatcc |
| 781 | tcattgatcc attccataaa gctatcagaa gaaatcctga cacccagtgg atcaccaaac |
| 841 | cagtccacaa gcacagggag atgcgtgggc tgacatctgc aggccgaaag agccgtggcc |
| 901 | ttggaaaggg ccacaagttc caccacacta ttggtggctc tcgccgggca gcttggagaa |
| 961 | ggcgcaatac tctccagctc caccgttacc gctaatataa gtaaagtttg taaaattcat |
| 1021 | acttaataaa caatttagga cagtcatgtc tgcttacagg tgttatttgt ctgttaaaac |
| 1081 | tagtctgcag atgtttcttg aatgctttgt caaattaaga aagttaaagt gcaataatgt |
| 1141 | ttgaagacaa taagtggtgg tgtatcttgt ttctaataag ataaactttt ttgtctttgc |
| 1201 | tttatcttat tagggagttg tatgtcagtg tataaaacat actgtgtggt ataacaggct |
| 1261 | taataaattc tttaaaagga gagaactgaa actagccctg tagatttgtc tggtgcatgt |
| 1321 | gatgaaacct gcagctttat cggagtgatg gcaatgctct gctggtttat tttcaagtgg |
| 1381 | ctgcgttttt tttagtttgg caggtgtaga ctttttaagt tgggctttag aaaatctggg |
| 1441 | ttagcctgaa gaaaattgcc tcagcctcca cagtaccatt ttaaattcac ataaaaggtg |
| 1501 | aaagctcctg gttcagtgcc atggcttcat ggcattcagt gattagtggt aatggtaaac |
| 1561 | actggtgtgt tttgaagttg aatgtgcgat aaaattatta gccttaagat tggtaagcta |
| 1621 | gcaatgaatg ctagggtggg aagctggtga gccagtggcc attagataaa tacctttcaa |
| 1681 | gtgtgagctt agacgtcaac cctaaaatac ttaaccgtaa tgccaattgt gatcattatg |
| 1741 | aatcccttca gtcacattag ggggaaagta gttggctata agtacgtcat tcttagtcca |
| 1801 | gtcagtctta aaaacatctt gggttaccca ctctgtccac tcccataggc tacagaaaaa |
| 1861 | gtcacaagcg catggtttcc aaccatatgt gttttctgca gttatttctc ttgttctggc |
| 1921 | caaacaaccc taaaaatcct taccattcca caaagttgga ccatcacttg tgcacccact |
| 1981 | ttgactatga gtataccacc acattgcatt tctgtttgca ccatgtcttc caggagacta |
| 2041 | gactactgtt gtccagggtc aatttgagtg taaagaaaat gtagacaagg aattgcccaa |
| 2101 | ttttaaattc tgactttgct gacttaattt aaatgctcgt tctgaaccaa ttttctccta |
| 2161 | tcttctctag gggtttcaaa agactcagtt aattgatttc caggaagtac tcatagcaag |
| 2221 | ttcataaaag ttcttgagac ctaaatttct tcacaaaaaa agaaaagatc ttaagtcata |
| 2281 | cattttaatt gtgtagaggt tgttcaactg aaggaataaa tgtctattaa actaaaacaa |
| 2341 | atggaccttc |

LOCUS NM_000985 985 bp mRNA linear PRI 17-APR-2013
DEFINITION Homo sapiens ribosomal protein L17 (RPL17), transcript variant 1,
mRNA. (SEQ ID NO: 665)

| | |
|---|---|
| 1 | cctgcctcct cagatctcgt ttcttcggct acgaatctcg cgagaagtca agttctcatg |
| 61 | agttctccca aaatccaccg ctcttcctct ttccctaagc agcctgaggg ttgactggat |
| 121 | tggtgaggcc cgtgtggcta cttctgtgga agcagtgctg tagttactgg aagataaaag |
| 181 | ggaaagcaag cccttggtgg gggaaagtat ggctgcgatg atggcatttc ttaggacacc |
| 241 | tttggattaa taatgaaaac aactactctc tgagcagctg ttcgaatcat ctgatattta |
| 301 | tactgaatga gttactgtaa gtacgtattg acagaattac actgtacttt cctctaggtg |
| 361 | atctgtgaaa atggttcgct attcacttga cccggagaac cccacgaaat catgcaaatc |
| 421 | aagaggttcc aatcttcgtg ttcactttaa gaacactcgt gaaactgctc aggccatcaa |
| 481 | gggtatgcat atacgaaaag ccacgaagta tctgaaagat gtcactttac agaaacagtg |
| 541 | tgtaccattc cgacgttaca atggtggagt tggcaggtgt gcgcaggcca agcaatgggg |
| 601 | ctggacacaa ggtcggtggc ccaaaaagag tgctgaattt ttgctgcaca tgcttaaaaa |
| 661 | cgcagagagt aatgctgaac ttaagggttt agatgtagat tctctggtca ttgagcatat |
| 721 | ccaagtgaac aaagcaccta agatgcgccg ccggacctac agagctcatg gtcggattaa |
| 781 | cccatacatg agctctccct gccacattga gatgatcctt acggaaaagg aacagattgt |
| 841 | tcctaaacca gaagaggagg ttgcccagaa gaaaaagata tcccagaaga aactgaagaa |
| 901 | acaaaaactt atggcacggg agtaaattca gcattaaaat aaatgtaatt aaaaggaaaa |

LOCUS NM_000979 893 bp mRNA linear PRI 11-JAN-2014
DEFINITION Homo sapiens ribosomal protein L18 (RPL18), transcript variant 1,
mRNA. (SEQ ID NO: 666)

| | |
|---|---|
| 1 | gaaagctatc gagaacacgg cctgggtagg gccagagagg cccccgacgt gctggccctt |
| 61 | ccctcttgga cgttgcgctt gttcctgcgc tctatgctct ctgccgttat cgcccggcta |
| 121 | gtcagtcgtc caactcacca cagagaagtc cggatcgtgg tagagcgccg cgtcgcaccc |
| 181 | atgtgacgtc acggcggcgc cactcgcttg aggctttccc cgcccacccc agcccgttct |
| 241 | ctctttccgg acctggccga gcaggaggcg ccatcatggg agtggacatc cgccataaca |
| 301 | aggaccgaaa ggttcggcgc aaggagccca agagccagga tatctacctg aggctgttgg |
| 361 | tcaagttata caggtttctg gccagaagaa ccaactccac attcaaccag gttgtgttga |
| 421 | agaggttgtt tatgagtcgc accaaccggc cgcctctgtc cctttcccgg atgatccgga |
| 481 | agatgaagct tcctggccgg gaaaacaaga cggccgtggt tgtggggacc ataactgatg |
| 541 | atgtgcgggt tcaggaggta cccaaactga aggtatgtgc actgcgcgtg accagccggg |
| 601 | cccgcagccg catcctcagg gcagggggca agatcctcac tttcgaccag ctggccctgg |
| 661 | actcccctaa gggctgtggc actgtcctgc tctccggtcc tcgcaagggc cgagaggtgt |
| 721 | accggcattt cggcaaggcc ccaggaaccc cgcacagcca caccaaaccc tacgtccgct |
| 781 | ccaagggccg gaagttcgag cgtgccagag gccgacgggc cagccgaggc tacaaaaact |
| 841 | aaccctggat cctactctct tattaaaaag atttttgctg acagtgcaaa |

LOCUS NM_000981 748 bp mRNA linear PRI 17-APR-2013
DEFINITION Homo sapiens ribosomal protein L19 (RPL19), mRNA. (SEQ ID NO: 667)

| | |
|---|---|
| 1 | gcagataatg ggaggagccg ggcccgagcg agctctttcc tttcgctgct gcggccgcag |
| 61 | ccatgagtat gctcaggctt cagaagaggc tcgcctctag tgtcctccgc tgtggcaaga |
| 121 | agaaggtctg gttagacccc aatgagacca atgaaatcgc caatgccaac tcccgtcagc |
| 181 | agatccggaa gctcatcaaa gatgggctga tcatccgcaa gcctgtgacg gtccattccc |
| 241 | gggctcgatg ccggaaaaac accttggccc gccggaaggg caggcacatg ggcataggta |
| 301 | agcggaaggg tacagccaat gcccgaatgc cagagaaggt cacatggatg aggagaatga |
| 361 | ggattttgcg ccggctgctc agaagatacc gtgaatctaa gaagatcgat cgccacatgt |
| 421 | atcacagcct gtacctgaag gtgaagggga atgtgttcaa aaacaagcgg attctcatgg |
| 481 | aacacatcca caagctgaag gcagacaagg cccgcaagaa gctcctggct gaccaggctg |
| 541 | aggcccgcag gtctaagacc aaggaagcac gcaagcgccg tgaagagcgc ctccaggcca |
| 601 | agaaggagga gatcatcaag actttatcca aggaggaaga gaccaagaaa taaaacctcc |
| 661 | cactttgtct gtacatactg gcctctgtga ttacatagat cagccattaa aataaaacaa |
| 721 | gccttaatct gcaa |

LOCUS NM_017971 741 bp mRNA linear PRI 10-JAN-2 014
DEFINITION Homo sapiens mitochondrial ribosomal protein L20 (MRPL20), mRNA.
(SEQ ID NO: 668)

| | |
|---|---|
| 1 | cggaagcgct cgtgaccacc atttccgacc cgggcaagat ggcagcggcg ctgcgcgtgc |
| 61 | gttgttgagt gttcgggacg ccggcctgca ggcgccatgg tcttcctcac cgcgcagctc |
| 121 | tggctgcgga atcgcgtcac cgaccgctac tttcggatcc aggaggtgct gaagcacgcc |
| 181 | aggcacttcc ggggaaggaa aaatcgctgc tacaggttgg cggtcagaac cgtgattcga |
| 241 | gcctttgtga aatgcaccaa agcccgatac ctgaagaaaa agaacatgag gaccctctgg |
| 301 | attaatcgaa ttacagctgc tagccaggaa catggactga agtatccagc gctcattggg |
| 361 | aatttagtta agtgccaggt ggagctcaac aggaaagtcc tagcggatct ggccatctac |
| 421 | gagccaaaga ctttcaaatc tttggctgcc ttggccagta ggaggcgaca cgaaggattt |
| 481 | gctgctgcct tgggggatgg gaaggaacct gaaggcattt tttccagagt ggtgcagtac |
| 541 | cactgaggac tgttgctgta ttgattagga aaagagacag agtaatttgc agtttgtttg |
| 601 | atttatactt ttgtttatct acaacccaat aacagacatg agggatggcc ctgtctctct |
| 661 | gggacagagc ctcacagatg atgtccatgt tttgtgtgaa tgaaactcaa acactcttca |
| 721 | gtttttagag tcaaaaaaaa |

LOCUS NM_000982 582 bp mRNA linear PRI 26-JAN-2014
DEFINITION Homo sapiens ribosomal protein L21 (RPL21), mRNA. (SEQ ID NO: 669)

| | |
|---|---|
| 1 | tttcctttcg gccggaaccg ccatcttcca gtaattcgcc aaaatgacga acacaaaggg |
| 61 | aaagaggaga ggcacccgat atatgttctc taggcctttt agaaaacatg gagttgttcc |
| 121 | tttggccaca tatatgcgaa tctataagaa aggtgatatt gtagacatca agggaatggg |
| 181 | tactgttcaa aaaggaatgc cccacaagtg ttaccatggc aaaactggaa gagtctacaa |
| 241 | tgttacccag catgctgttg gcattgttgt aaacaaacaa gttaagggca agattcttgc |
| 301 | caagagaatt aatgtgcgta ttgagcacat taagcactct aagagccgag atagcttcct |
| 361 | gaaacgtgtg aaggaaaatg atcagaaaaa gaaagaagcc aaagagaaag gtacctgggt |
| 421 | tcaactaaag cgccagcctg ctccacccag agaagcacac tttgtgagaa ccaatgggaa |
| 481 | ggagcctgag ctgctggaac ctattcccta tgaattcatg gcataatagg tgttaaaaaa |
| 541 | aaaaataaag gacctctggg ctacaaaaaa |

LOCUS NM_000983 2099 bp mRNA linear PRI 10-JAN-2014
DEFINITION Homo sapiens ribosomal protein L22 (RPL22), mRNA. (SEQ ID NO: 670)

| | |
|---|---|
| 1 | gcgtctgcgt agttcgctca cctccctttc taactccgct gccgccatgg ctcctgtgaa |
| 61 | aaagcttgtg gtgaaggggg gcaaaaaaaa gaagcaagtt ctgaagttca ctcttgattg |
| 121 | cacccaccct gtagaagatg gaatcatgga tgctgccaat tttgagcagt ttttgcaaga |
| 181 | aaggatcaaa gtgaacggaa aagctgggaa ccttggtgga ggggtggtga ccatcgaaag |
| 241 | gagcaagagc aagatcaccg tgacatccga ggtgcctttc tccaaaaggt atttgaaata |
| 301 | tctcaccaaa aaatatttga agaagaataa tctacgtgac tggttgcgcg tagttgctaa |
| 361 | cagcaaagag agttacgaat tacgttactt ccagattaac caggacgaag aagaggagga |
| 421 | agacgaggat taaatttcat ttatctggaa aattttgtat gagttcttga ataaaacttg |
| 481 | ggaaccaaaa tggtggttta tccttgtatc tctgcagtgt ggattgaaca gaaaattgga |
| 541 | aatcatagtc aaagggcttc ccttggttcg ccactcattt atttgtaact tgacttcttt |
| 601 | ttttttctgc ttaaaaattt caattctcgt ggtaatacca gagtagaagg agagggtgac |
| 661 | tttaccgaac tgacagccat tggggaggca gatgcgggtg tggaggtgtg ggctgaaggt |
| 721 | agtgactgtt tgattttaaa aagtgtgact gtcagttgta tctgttgctt ttctcaatga |
| 781 | ttcagggata caaatgggct tctctcattc attaaaagaa aacgcgacat ctttctaaga |
| 841 | ttctctgtgg gaaaatgact gtcaataaaa tgcgggtttc tgggccattc gtcttacttt |
| 901 | cattttttga ttacaaattt ctcttgacgc acacaattat gtctgctaat cctcttcttc |
| 961 | ctagagagag aaactgtgct ccttcagtgt tgctgccata aaggggtttg gggaatcgat |
| 1021 | tgtaaaagtc ccaggttcta aattaactaa atgtgtacag aaatgaacgt gtaagtaatg |
| 1081 | tttctacagg tctttgcaac aaactgtcac tttcgtctcc agcagaggga gctgtaggaa |
| 1141 | tagtgcttcc agatgtggtc tcccgtgtgg ggcccagcaa tgggggcccc tgatgccaag |
| 1201 | agctctggag gttcttgaaa gaggggacac gaaggaggag tgactgggaa gcctcccatg |
| 1261 | ccaaggaggt gggaggtgcc ctggaaatag ctgcctcatg ccacttaggc catgactgga |
| 1321 | tttaatgtca gtggtgtgcc acagtgcaga ggctagacaa ctgaaagggg ctaccaaggc |
| 1381 | tgggaaaaaa atgcaattgt tgctgtgagt gactttgaaa gactctggtg ccttgtggtg |
| 1441 | cccttctgaa attcaaacag taatgcaaaa gtgtctgcat tagaatttac ggtgtctaaa |
| 1501 | attcatgttt ttaaaagagc ttgcctacag atggtttcca cacttgaaat tgtgccctgc |
| 1561 | gagttgcata gctggaagtt caatgctcag tcctaccttg gctcccatta aacatttggt |
| 1621 | gctctgtgga ttgagttgaa cgtgttgagg ctttgcaatt tcacttgtgt taaaggctct |
| 1681 | ggcatttttc catttctatg caaatttctt tgaagcagaa ttgcttgcat atttcttctc |
| 1741 | tgccgtcaca gaaagcagag tttctttcaa acttcactga ggcatcagtt gctctttggc |
| 1801 | aatgtccctt aaccatgatt attaactaag tttgtggctt gagtttacaa attctacttg |
| 1861 | ttgcattgat gttcccatgt agtaagtcat ttttagtttg gttgtgaaaa aaccctgggc |
| 1921 | tgaagttggc atttcagtta aaagaaaaaa agaaactagt cccagatttg aaaacttgta |
| 1981 | ataaaattga aactcactgg ttttctatgt ctttttgaac tcttgtaatc gagttttgat |
| 2041 | catattttct attaaagtgg ctaacacctg gctactctta ctgtaaaaaa |

LOCUS NM_000978 594 bp mRNA linear PRI 26-JAN-2014
DEFINITION Homo sapiens ribosomal protein L23 (RPL23), mRNA. (SEQ ID NO: 671)

| | |
|---|---|
| 1 | ggccacgtga ggagggtggg cggggcgtta aagttcatat cccagtgtcc tttgaatcga |
| 61 | cttccttttt tcttttttcc ggcgttcaag atgtcgaagc gaggacgtgg tgggtcctct |
| 121 | ggtgcgaaat tccggatttc cttgggtctt ccggtaggag ctgtaatcaa ttgtgctgac |
| 181 | aacacaggag ccaaaaacct gtatatcatc tccgtgaagg ggatcaaggg acggctgaac |
| 241 | agacttcccg ctgctggtgt gggtgacatg gtgatggcca cagtcaagaa aggcaaacca |
| 301 | gagctcagaa aaaaggtaca tccagcagtg gtcattcgac aacgaaagtc ataccgtaga |
| 361 | aaagatggcg tgtttcttta ttttgaagat aatgcaggag tcatagtgaa caataaaggc |
| 421 | gagatgaaag gttctgccat tacaggacca gtagcaaagg agtgtgcaga cttgtggccc |
| 481 | cggattgcat ccaatgctgg cagcattgca tgattctcca gtatatttgt aaaaaataaa |
| 541 | aaaaaaaact aaacccatta aaaagtattt gtttgcaaaa |

LOCUS NM_000984 979 bp mRNA linear PRI 17-APR-2013
DEFINITION Homo sapiens ribosomal protein L23a (RPL23A), mRNA.
(SEQ ID NO: 672)

| | |
|---|---|
| 1 | agcattggag acccttttca caagatggcg ccgaaagcga agaaggaagc tcctgcccct |
| 61 | cctaaagctg aagccaaagc gaaggcttta aaggccaaga aggcagtgtt gaaaggtgtc |
| 121 | cacagccaca aaaagaagaa gatccgcacg tcacccacct tccggcggcc gaagacactg |
| 181 | cgactccgga gacagcccaa atatcctcgg aagagcgctc ccaggagaaa caagcttgac |
| 241 | cactatgcta tcatcaagtt tccgctgacc actgagtctg ccatgaagaa gatagaagac |
| 301 | aacaacacac ttgtgttcat tgtggatgtt aaagccaaca agcaccagat taaacaggct |
| 361 | gtgaagaagc tgtatgacat tgatgtggcc aaggtcaaca ccctgattcg gcctgatgga |
| 421 | gagaagaagg catatgttcg actggctcct gattacgatg ctttggatgt tgccaacaaa |
| 481 | attgggatca tctaaactga gtccagctgc ctaattctga atatatatat atatatatct |
| 541 | tttcaccata tacatgcctg tctgtcaatt tctggttggg ctgggaggcc acacacacac |
| 601 | actgacatga cagggcttgg gcaagactcc tgttctactt atccttttga aatacctcac |
| 661 | cctgccactc caccatgtat gatcattcca gagatctttg tgactagagt tagtgtccta |
| 721 | ggaaaaccag aactcagaac ttgcctccat ggttgagtaa caagctgtac aagaacccct |
| 781 | tttatccctg gaagaggctg tgtatgaaac caatgcccag ggtttgaagg gtgttagcat |
| 841 | ccatttcagg ggagtgtgga ttggctggct ctctggtagc attttgtcct cacacaccca |
| 901 | tctactatgt ccaaccggtc tgtctgcttc cctcacccct tgcccaataa aggacaagga |
| 961 | cttcagagga |

LOCUS NM_000986 579 bp mRNA linear PRI 26-JAN-2014
DEFINITION Homo sapiens ribosomal protein L24 (RPL24), mRNA. (SEQ ID NO: 673)

| | |
|---|---|
| 1 | tctttctttt cgccatcttt tgtctttccg tggagctgtc gccatgaagg tcgagctgtg |
| 61 | cagttttagc gggtacaaga tctaccccgg acacgggagg cgctacgcca ggaccgacgg |
| 121 | gaaggttttc cagtttctta atgcgaaatg cgagtcggct ttcctttcca agaggaatcc |
| 181 | tcggcagata aactggactg tcctctacag aaggaagcac aaaaagggac agtcggaaga |
| 241 | aattcaaaag aaaagaaccc gccgagcagt caaattccag agggccatta ctggtgcatc |
| 301 | tcttgctgat ataatggcca agaggaatca gaaacctgaa gttagaaagg ctcaacgaga |
| 361 | acaagctatc agggctgcta aggaagcaaa aaaggctaag caagcatcta aaaagactgc |
| 421 | aatggctgct gctaaggcac ctacaaaggc agcacctaag caaaagattg tgaagcctgt |
| 481 | gaaagtttca gctccccgag ttggtggaaa acgctaaact ggcagattag atttttaaat |
| 541 | aaagattgga ttataactct agaaaaaaaa |

LOCUS NM_000987 602 bp mRNA linear PRI 18-JAN-2014
DEFINITION Homo sapiens ribosomal protein L26 (RPL26), mRNA. (SEQ ID NO: 674)

| | |
|---|---|
| 1 | ataggtctcg cgagatcttt ggtaaactta cagaaccgga agcagcgtgt agttctcttc |
| 61 | ccttttgcgg ccatcaccga agcgggagcg gccaaaatga agtttaatcc ctttgtgact |
| 121 | tccgaccgaa gcaagaatcg caaaaggcat ttcaatgcac cttcccacat tcgaaggaag |
| 181 | attatgtctt cccctctttc caaagagctg agacagaagt acaacgtgcg atccatgccc |
| 241 | atccgaaagg atgatgaagt tcaggttgta cgtggacact ataaaggtca gcaaattggc |
| 301 | aaagtagtcc aggtttacag gaagaaatat gttatctaca ttgaacgggt gcagcgggaa |
| 361 | aaggctaatg gcacaactgt ccacgtaggc attcacccca gcaaggtggt tatcactagg |
| 421 | ctaaaactgg acaaagaccg caaaaagatc ctcgaacgga aagccaaatc tcgccaagta |
| 481 | ggaaaggaaa agggcaaata caaggaagaa accattgaga agatgcagga ataaagtaat |
| 541 | cttatataca agctttgatt aaaacttgaa acaaagagcc tgaaaaaaaa |

LOCUS NM_000988 514 bp mRNA linear PRI 26-JAN-2014
DEFINITION Homo sapiens ribosomal protein L27 (RPL27), mRNA. (SEQ ID NO: 675)

| | |
|---|---|
| 1 | tccttctttc ctttttgctg gtagggccgg gtggttgctg ccgaaatggg caagttcatg |
| 61 | aaacctggga aggtggtgct tgtcctggct ggacgctact ccggacgcaa agctgtcatc |
| 121 | gtgaagaaca ttgatgatgg cacctcagat cgcccctaca gccatgctct ggtggctgga |
| 181 | attgaccgct acccccgcaa agtgacagct gccatgggca agaagaagat cgccaagaga |
| 241 | tcaaagataa aatcttttgt gaaagtgtat aactacaatc acctaatgcc cacaaggtac |
| 301 | tctgtggata tccccttgga caaaactgtc gtcaataagg atgtcttcag agatcctgct |
| 361 | cttaaacgca aggcccgacg ggaggccaag gtcaagtttg aagagagata caagacaggc |
| 421 | aagaacaagt ggttcttcca gaaactgcgg ttttagatgc tttgttttga tcattaaaaa |

LOCUS NM_000990 4906 bp mRNA linear PRI 11-JAN-2014
DEFINITION Homo sapiens ribosomal protein L27a (RPL27A), mRNA.
(SEQ ID NO: 676)

| | |
|---|---|
| 1 | aacgaggccc ttagggtcgg cttaggcggt tccctgacca aggcgccaga aaagggcctg |
| 61 | gctcaagcaa gcacgggcgg cgtgcagtac agcacaccta gccccgattc ttcaacagtt |
| 121 | ctcgccctcc gagcctagca caacgagcct caccgaaacc gtacaccgcc accaggacac |
| 181 | tccgtgatgg gggatcacca ccctcagaaa gaggaagcga ctagcaggcg cgcaatcccg |
| 241 | cgagaccagg aggccccgcc cgaagcccgg cctctgtgac cggaagtgag gcgttttgcc |
| 301 | ccgcccccgt ggccgatacc tcgcgagact tggcgaaggc cttccttttt cgtctgggct |
| 361 | gccaacatgc catccagact gaggaagacc cggaaactta ggggccacgt gagccacggc |
| 421 | cacggccgca taggcaagca ccggaagcac cccggcggcc gcggtaatgc tggtggtctg |
| 481 | catcaccacc ggatcaactt cgacaaatac cacccaggct actttgggaa agttggtatg |
| 541 | aagcattacc acttaaagag gaaccagagc ttctgcccaa ctgtcaacct tgacaaattg |
| 601 | tggactttgg tcagtgaaca gacacgggtg aatgctgcta aaaacaagac tggggctgct |
| 661 | cccatcattg atgtggtgcg atcgggctac tacaaagttc tgggaaaggg aaagctccca |
| 721 | aagcagcctg tcatcgtgaa ggccaaattc ttcagcagaa gagctgagga gaagattaag |
| 781 | agtgttgggg gggcctgtgt cctggtggct tgaagccaca tggagggagt ttcattaaat |
| 841 | gctaactact ttttccttgt ggtgtgagtg taggttcttc agtggcacct ctacatcctg |
| 901 | tgtgcattgg gagcccaggt tctagtactt agggtatgaa gacatggggt cctctcctga |
| 961 | cttccctcaa atatatggta aacgtaagac caacacagac gttggccagt taaacatttc |
| 1021 | tgtttataaa gtcagaataa tacctgttga tcactgaaag gcctgcatgt attgtactct |
| 1081 | gaattttaca gtgaatgaga gaatgtaccc taattgttca acagggctca aaaggaaaga |
| 1141 | ttccattttg atgggtcaca ttctaaagag gggcagtgtg ataggaatga gatggtcctt |
| 1201 | taggacttaa gttctcagcc caaggttttt ccacgtggcc ccctcatctt tttttttttt |
| 1261 | ttaaacggag tctctcttgc caggctggag tgcagtggca cgatctcggc tcactgcagc |
| 1321 | ctccgcctcc caggttaagc gattctcctg cctcagcttc ctgactaact gggattacag |
| 1381 | gcgcccacca ccatgcccag ctaatttttg tattttcagt agagatgggg tttcaccatg |
| 1441 | ttggccatgc tggtctctaa ctcctaacct caagtgatct gcccacatcg gcctccaaaa |
| 1501 | gttctgggat tatagtgtga gccactgcgc ccggccatgg ctccttaatc ttgatccaaa |
| 1561 | ttattgttac atccagaatg tgatgaatca aaatctcgag atgggggtcc agcaatctga |
| 1621 | aatttcagta tgccagggct tttctgtatg tcaaagtggg tttgaaatag ttaatttttc |
| 1681 | ttctagtctg aaatgtatcg ggaaaatttg gaaatcctga aggctggaaa ttgaaataag |
| 1741 | tttttctagg atttgtgtct cttgctattg gaaaactgat ggtgaccaat tcatgtttac |
| 1801 | aaataagatc ctcatagatc tcggtaaatt ataatttgct acagttttat ggttcttcct |
| 1861 | gtgattttga gctttttttg acccaaaata atacagtcta aaactataga caaataagat |
| 1921 | ggcacttaga ctcctgggtt ttagttagtg gaggtttcct tagtgcactg tggggtcata |
| 1981 | ataagccgag aaccatggct gtctatggga cacatctgtc aggacaacct ttagaggatg |
| 2041 | ttggggatca aatagaaggc acagagaagc actgaattgg cttacataag aataggctag |
| 2101 | aattacaagt agtgaaacct cgattcagct ggacaatttt aaacaaatgt atcatttggc |
| 2161 | ttgtatcttc tgttgtgctg gagaagttag aaataagggc tctccagacc agcctgacca |
| 2221 | acctggagaa accttgtctc tactaaatac acaaaattag ccaggcgtgg tggcacatgc |
| 2281 | ctgtaatccc agctactttg gaggctgagc caggagaatc tccaggaggc ggaggttgct |
| 2341 | gtgagccgag atcgtgccat tgcactccag cttgggcaac aagagtgaaa ctctgtccac |
| 2401 | cccccccaaa aaaagtaagg gctctccatt agggcccata gaggacttgt aatatggaac |
| 2461 | ctgaatccaa ggatcccaca ataagtggtc agtagttcat gatgaattaa aagactcaat |
| 2521 | atttggtctt cacccaatac ctgtgtgact tttagtccta atttcctcat ctttaaaatt |
| 2581 | tcagtgaaag tgcctacctg aggattgtgt agattaaaat ggaaaccgtg cacttaattt |
| 2641 | tttgttttgt tttgagacgg agtctcgctc tgtcgcccag gctggagtgc agtggtgcga |
| 2701 | tctcagatca ctgcaagctc cgcctcctag gttcagacca ttctcctgcc tcagcttccc |
| 2761 | aagtagctgg gactacaggc gcccgccact gcgcccggct aattttttgc atttttagta |
| 2821 | gagacagggt ttcaccgtgt tagccaggat ggtctcgatc tcctgatctg cccgcctcag |
| 2881 | cctcccaaag tgctgggatt acaggcatga gccaccgcgc ccggcccagg cacttaattt |
| 2941 | ttgtgtttga cttagtaact taagtgcaaa ctattacggg agcagatgga gtcaattggc |
| 3001 | cttcatgtga ttgtcagtgg gaaattggtc caagcagagg gaatactggt tcaggaaact |
| 3061 | ggtttgggaa ggttaggcaa acgggaagtg ctatggtgga gagaaagatt actctggccg |
| 3121 | ggctgtaaag gacggctaca atgggaggct gaaggcagaa ccaagaaaat gggagtgagt |
| 3181 | atggaaaagg tacgattcag acggcataat ggacgggact tggagactga attgtagtgg |
| 3241 | gccgaccaca aaatgataag gcatggaagg aagtagagtt tggggggaag gatccctagt |
| 3301 | cccttaatgg ctaccttctt ccccaggagt tgttaggcca tccgatcccc tggcctggga |
| 3361 | aagaaacact gatttcgttg ctggcttgtt cactcaccag aagctacagc tactaacagt |
| 3421 | tctaaaaact gtttcatgtg atgaggaaca gacgaaaata gttttgagcc ctaagtccgc |
| 3481 | cgattccagt gctttcttga acccgcattt actaaaatat tttcatgact gccaagcttt |
| 3541 | gaatagcctg ctgtgttcat ggaggctcat actggcgatc tctagtggct ggctaaagct |
| 3601 | tgaattgcaa aagatctaat ttctggtcta atgtatatat gccttaaata tagttgcgtt |
| 3661 | caaacgtggg agctgcaggt gcaacttgat tttatgacaa atggctgcca cataatttgc |
| 3721 | acaagcagtg ctcgtcaagg gcagctaaat caggcgagct ttcaatcaaa ataaatgtac |
| 3781 | tactaaaccc tacttagcgg ctaactagcc caagagcaga cagcccacgg acggactgca |
| 3841 | agtcggaagc gcgggcggaa gctgtgcagc gcccacctgg tggctccatc ggccgcgttc |
| 3901 | atcagtcagc acgacccgac ctcagtggcg tcctcacaac acagaccgga ccttgggtct |
| 3961 | taccccggca cctgagaacc acttccggtg agtagcttct acttccggag acgatgactc |
| 4021 | ccccgcgtcc cagaccggaa gaagcccggc ggagaccggc ctcgctcggc cacttccggc |
| 4081 | aagggcggag ccggccagtg gtgcgcgagc gcagataact cccctggaga ggcgggatgt |
| 4141 | tcaactccac ccctggtcct tgggcggccg tgggtcccct tcgaagcgga ggaatggcca |
| 4201 | acctcgccgc acttcgagcc cctttagggt gcgtttaaga acagtgggcg tggcctttac |
| 4261 | gtaaatcttc gagatgggaa cctccagaat ttgtctcaat tgtctaaaag gtaatgagcg |
| 4321 | tcagcgacat tcaagggcac tttgggctaa aaaagaaagt gcttgtacac ggatggaaat |
| 4381 | attctagaag aacataaaag gaatttcctc ttaggaggtt agggaaatga gcacgaagta |
| 4441 | tgttttggtg cagttttttg ttcaacccaa tgcgtatttt catattgaga ggcaatataa |
| 4501 | atggagcgaa agtatcttga gaaaaaaaaa aaaactacca gaacttgccg ttgctgaaaa |
| 4561 | gtaatatttt ctctttcgag agttttcatg gccttttaaa ttacaccccc acctccacag |
| 4621 | gcaaataaat ttgttttgga atgcatacca catcatctgg ctctagaaac gtattttgtg |
| 4681 | tagctcccct agcaagaata taggttaaag cgtaaattta attcctggct ctattttaca |
| 4741 | tcccaatttt tcattttcctc tcattcccac tttacgttgt ttcaaataac ctagtttgtg |
| 4801 | tatccctgta agtcattttg gtataaagta ggttataagt gtacatgcga aaagatgttt |
| 4861 | ttaacaaaaa tgtaactgaa |

LOCUS NM_000991 4245 bp mRNA linear PRI 17-APR-2013
DEFINITION Homo sapiens ribosomal protein L28 (RPL28), transcript variant 2,
mRNA. (SEQ ID NO: 677)

| | |
|---|---|
| 1 | ctctttccgt ctcaggtcgc cgctgcgaag ggagccgccg ccatgtctgc gcatctgcaa |
| 61 | tggatggtcg tgcggaactg ctccagtttc ctgatcaaga ggaataagca gacctacagc |
| 121 | actgagccca ataacttgaa ggcccgcaat tccttccgct acaacggact gattcaccgc |
| 181 | aagactgtgg gcgtggagcc ggcagccgac ggcaaaggtg tcgtggtggt cattaagcgg |
| 241 | agatccggcc agcggaagcc tgccacctcc tatgtgcgga ccaccatcaa caagaatgct |
| 301 | cgcgccacgc tcagcagcat cagacacatg atccgcaaga acaagtaccg ccccgacctg |
| 361 | cgcatggcag ccatccgcag ggccagcgcc atcctgcgca gccagaagcc tgtgatggtg |
| 421 | aagaggaagc ggacccgccc caccaagagc tcctgagccc cctgccccca gagcaataaa |
| 481 | gtcagctggc tttctcacct gcctcgactg ggcctccctt tttgaaacgc tctggggagc |
| 541 | tctggccctg tgtgttgtca ttcaggccat gtcatcaaaa ctctgcatgt caccttgtcc |
| 601 | atctggaggt gatgtcaatg gctggccatg caggaggggt ggggtagctg ccttgtccct |
| 661 | ggtgagggca agggtcactg tcttcacaga aaaagtttgc tgacttgtga ttgagaccta |
| 721 | ctgtcccatt gtgaggtggc ctgaagaatc ccagctgggg cagtggcttc cattcagaag |
| 781 | aagaaaggcc ttttctagcc cagaagggtg caggctgagg gctgggccct gggccctggt |
| 841 | gctgtagcac ggtttgggga cttggggtgt tcccaagacc tgggggacga cagacatcac |
| 901 | gggaggaaga tgagatgact tttgcatcca gggagtgggt gcagccacat ttggagggga |
| 961 | tgggctttac ttgatgcaac ctcatctctg agatgggcaa cttggtgggt ggtggcttat |
| 1021 | aactgtaagg gagatggcag ccccagggta cagccagcag gcattgagca gccttagcat |
| 1081 | tgtcccccta ctcccgtcct ccaggtgtcc ccatccctcc cctgtctctt tgagctggct |
| 1141 | cttgtcactt aggtctcatc tcagtggccg ctcctgggcc accctgtcac ccaagctttc |
| 1201 | ctgattgccc agccctcttg tttcctttgg cctgtttgct ccctagtgtt tattacagct |
| 1261 | tgtgaggcca ggagtttgag accatcctag gcaacataat gagacaccgt ctctaaaata |
| 1321 | aaattagctg ggtgtggtgg tgcaccgcct gtggtcccag ctcctcagag gttgagtaga |
| 1381 | ggctgaggtg agcggagcac ttgagccaag agtatgaggc tgcagtgagc ccatgagccc |
| 1441 | caccactaca ctccagcctg gaagacacca tgacacacag tgaggcctgg atggggaaag |
| 1501 | agtcctgctg ttgatcctca catgtttcct gggcacctaa ctctgtcagc cactgccagg |
| 1561 | gaccaaggat ccagcatcca tggcacccct ggttcctgcc atcctggggt acccgattca |
| 1621 | aagaaggact ctgctccctg tctgagacca cccccggctc tgactgagag taaggggact |
| 1681 | gtcagggcct cgacttgcca ttggttgggg tcgtacgggg ctgggagccc tgcgttttga |
| 1741 | ggcagaccac tgcccttccg acctcagtcc tgtctgctcc agtcttgccc agctcgaagg |
| 1801 | agagcagatc tgaccacttg ccagcccctg tctgctgtga attaccattt cctttgtcct |
| 1861 | tcccttagtt gggtctatta gctcagattg agaggtgttg ccttaaaact gagttgggtg |
| 1921 | acttggtacc tgctcaggac cccccgcact gtcccaatcc cactcaggcc cacctccagc |
| 1981 | tggcctcact ccgctggtga cttcgtacct gctcaggagc ccccactgtc ccagtcccac |
| 2041 | tcaggcccat ctctggctgg cctcactgcg ctgggactcc gccttcataa ggagagctca |
| 2101 | ctgctcacgt tagtagatgg ccccttctcg tgaggcctct cccctggcac ctgcttcagt |
| 2161 | tgtcctccac agcactgatt tgcagcccac aagctggcag gtttatctgt ctcatgtttg |
| 2221 | tcttgtgctg gtgggcaagg ggtttgtcta gcacaccagc atataatgag atgcttgatg |
| 2281 | aatggtgcat attgaatgta taaagcccac cggtcctgag agtttgctca ctggagactt |
| 2341 | tctggagatg gagtctcgct ctgttgccca ggctggcgag tgcaatggcg cgatcttggc |
| 2401 | tcactgcagc ctccacctcc tgggttcaag cgattctcct gcctcagcct cccgagtagc |
| 2461 | tgggattaca ggtgggtgtc accacaccca gctcagtatt gtatttttag cagagatggg |
| 2521 | gtttcaccat tttgcccagg ctggtttgga actcctgact tcaaattacc cacctgcctc |
| 2581 | agcctcccaa agtgctggca ttacaggcgc tcgaggcttt ctgatgtggc tgctgctgct |
| 2641 | cagaaggcct tgtccttaac cacctccttg cctgccctgg aggcttgtgc ctctaggccc |
| 2701 | caccccctgt ggagtcctgc tggctttctc catccctatc tgaatcctcc ctgctgtgtg |
| 2761 | gcctcccctg gtctcatccg taacacagcc cagcttagtg ggcctctgtt cctgcgggtg |
| 2821 | gccagcctgt ctgtgtggct gggctgggga ggccacgtct ggtatctgaa tgctatcggt |
| 2881 | gggttggggt ggaggaacca ggagagggct ggagggaggg agatggtctc agccccacag |
| 2941 | agtttggagt cctcagtgtg ctgagcaaac gtggagacac catttccctc ctctagacct |
| 3001 | catcttggag agagagatgt tggatggggc catctattcc agctttattc acacaaatca |
| 3061 | tgtctgttgg cctggaaatt ggaaaaccag ttaaaccaaa aacatgatat taagaaaaca |
| 3121 | ggcaggctca ccatagtaaa aatgctgaaa gccaaagaca aaattgggag aacaaaagaa |
| 3181 | aagcgtcttg tcacatacag aaggtccctg ataaagttag tagctgccct catcagaaac |
| 3241 | caggcccagg cagtggggac acatccagag tgctgaaaga acctccccca ggtcatccta |
| 3301 | tccccaagag tgatgcccgg cagcattccc agctcagggc taatggttca cggaagccag |
| 3361 | gaatcaaact gcctgggttc cagtcccagc tctgccagtt atgcccagct gtggggactt |
| 3421 | gggcagctcg tttagtagca ccgtgcctca gtttcccata tgtaaaaggc cattttgagt |
| 3481 | gcctttcaca gccctgcata aggcaggtgt ctcagtgttc actgctgtct ctccagctct |
| 3541 | tagtccagta gctgcatggt gagtgagcgt agggcgcacc ctggaaggct gccaagccca |
| 3601 | aagttgtgca gagcgctggg gactccagac tccccacagc agcagagact cgggactgag |
| 3661 | gcatcctctg ttcacaggac atgctggcat ctactgggtc agggctctgc tgctcggtgg |
| 3721 | ctgtgcaacc ttgggcaagt tcctcaacct ctctgtgtct tcgtaccctc atctgtaaca |
| 3781 | tgcgtgtcga tagaccctac tactcagggt tgatgagaag attaaatgtg caaaacctgc |
| 3841 | ttgactgtgc ccacaaatcc tgattgtagg aataaattaa tgacttttta taaatatttt |
| 3901 | gatcagatgg actcatgatc acagatgtct tcacatgcct atgactaatt tgtacacaaa |
| 3961 | ctaatgctcg tgtttcccaa gcacctggaa gacatgccag atccatgtgc agtaatgcct |
| 4021 | ggtggctcca ggtctgcccc gccgtcctgt ggggctgtga gctttcccag cctcctgccc |
| 4081 | gtgtttgtga atatcattct gtcctcagct gcatttccag cccaggctgt ttggcgctgc |
| 4141 | ccaggaatgg tatcaattcc cctgtttctc ttgtagccag ttactagaat aaaatcatct |
| 4201 | actttaaaaa |

LOCUS NM_000992 737 bp mRNA linear PRI 10-JAN-2014
DEFINITION Homo sapiens ribosomal protein L29 (RPL29), mRNA. (SEQ ID NO: 678)

| | |
|---|---|
| 1 | ttccggcgtt gttgacccta tttcccgtgc tgcaccgcag cccctttctc ttccggttct |
| 61 | aggcgcttcg ggagccgcgg cttatggtgc agacatggcc aagtccaaga accacaccac |
| 121 | acacaaccag tcccgaaaat ggcacagaaa tggtatcaag aaaccccgat cacaaagata |
| 181 | cgaatctctt aagggggtgg accccaagtt cctgaggaac atgcgctttg ccaagaagca |
| 241 | caacaaaaag ggcctaaaga agatgcaggc caacaatgcc aaggccatga gtgcacgtgc |
| 301 | cgaggctatc aaggccctcg taaagcccaa ggaggttaag cccaagatcc caaagggtgt |
| 361 | cagccgcaag ctcgatcgac ttgcctacat tgcccacccc aagcttggga agcgtgctcg |
| 421 | tgcccgtatt gccaaggggc tcaggctgtg cggccaaag gccaaggcca aggccaaggc |
| 481 | caaggatcaa accaaggccc aggctgcagc cccagcttca gttccagctc aggctcccaa |
| 541 | acgtacccag gcccctacaa aggcttcaga gtagatatct ctgccaacat gaggacagaa |
| 601 | ggactggtgc gaccccccac ccccgcccct gggctaccat ctgcatgggg ctggggtcct |
| 661 | cctgtgctat ttgtacaaat aaacctgagg caggaaa |

LOCUS NM_000989 571 bp mRNA linear PRI 10-JAN-2014
DEFINITION Homo sapiens ribosomal protein L30 (RPL30), mRNA. (SEQ ID NO: 679)

| | |
|---|---|
| 1 | agttccggct ctgccgtgaa gagctttgca ttgtgggaag tctttccttt ctcgttcccc |
| 61 | ggccatctta gcggctgctg ttggttgggg gccgtcccgc tcctaaggca ggaagatggt |
| 121 | ggccgcaaag aagacgaaaa agtcgctgga gtcgatcaac tctaggctcc aactcgttat |
| 181 | gaaaagtggg aagtacgtcc tggggtacaa gcagactctg aagatgatca gacaaggcaa |
| 241 | agcgaaattg gtcattctcg ctaacaactg cccagctttg aggaaatctg aaatagagta |
| 301 | ctatgctatg ttggctaaaa ctggtgtcca tcactacagt ggcaataata ttgaactggg |
| 361 | cacagcatgc ggaaaatact acagagtgtg cacactggct atcattgatc caggtgactc |
| 421 | tgacatcatt agaagcatgc cagaacagac tggtgaaaag taaacctttt cacctacaaa |
| 481 | atttcacctg caaaccttaa acctgcaaaa ttttccttta ataaaatttg cttgttttaa |
| 541 | aaacattgta tcta |

LOCUS NM_000993 524 bp mRNA linear PRI 17-NOV-2013
DEFINITION Homo sapiens ribosomal protein L31 (RPL31), transcript variant 1,
mRNA. (SEQ ID NO: 680)

| | |
|---|---|
| 1 | tggcgacccg gaagttgtac ttgcaactgc ggctttcctt ctcccacaat ccttcgcgct |
| 61 | cttcctttcc aacttggacg ctgcagaatg gctcccgcaa agaagggtgg cgagaagaaa |
| 121 | aagggccgtt ctgccatcaa cgaagtggta acccgagaat acaccatcaa cattcacaag |
| 181 | cgcatccatg gagtgggctt caagaagcgt gcacctcggg cactcaaaga gattcggaaa |
| 241 | tttgccatga aggagatggg aactccagat gtgcgcattg acaccaggct caacaaagct |
| 301 | gtctgggcca aaggaataag gaatgtgcca taccgaatcc gtgtgcggct gtccagaaaa |
| 361 | cgtaatgagg atgaagattc accaaataag ctatatactt tggttaccta tgtacctgtt |
| 421 | accactttca aaaatctaca gacagtcaat gtggatgaga actaatcgct gatcgtcaga |
| 481 | tcaaataaag ttataaaatt gccttcaaaa |

LOCUS NM_000994 1668 bp mRNA linear PRI 11-JAN-2014
DEFINITION Homo sapiens ribosomal protein L32 (RPL32), transcript variant 1,
mRNA. (SEQ ID NO: 681)

| | |
|---|---|
| 1 | aggggttacg acccatcagc ccttgcgcgc caccgtccct tctctcttcc tcggcgctgc |
| 61 | ctacggaggt ggcagccatc tccttctcgg catcatggcc gccctcagac cccttgtgaa |
| 121 | gcccaagatc gtcaaaaaga gaaccaagaa gttcatccgg caccagtcag accgatatgt |
| 181 | caaaattaag cgtaactggc ggaaacccag aggcattgac aacagggttc gtagaagatt |
| 241 | caagggccag atcttgatgc ccaacattgg ttatggaagc aacaaaaaaa caaagcacat |
| 301 | gctgcccagt ggcttccgga agttcctggt ccacaacgtc aaggagctgg aagtgctgct |
| 361 | gatgtgcaac aaatcttact gtgccgagat cgctcacaat gtttcctcca agaaccgcaa |
| 421 | agccatcgtg gaaagagctg cccaactggc catcagagtc accaacccca atgccaggct |
| 481 | gcgcagtgaa gaaaatgagt aggcagctca tgtgcacgtt ttctgtttaa ataaatgtaa |
| 541 | aaactgccat ctggcatctt ccttccttga ttttaagtct tcagcttctt ggccaactta |
| 601 | gtttgccaca gagattgttc ttttgcttaa gcccctttgg aatctcccat ttggagggga |
| 661 | tttgtaaagg acactcagtc cttgaacagg ggaatgtggc ctcaagtgca cagactagcc |
| 721 | ttagtcatct ccagttgagg ctgggtatga ggggtacaga cttggccctc acaccaggta |
| 781 | ggttctgaga cacttgaaga agcttgtggc tcccaagcca caagtagtca ttcttagcct |
| 841 | tgcttttgta aagttaggtg acaagttatt ccatgtgatg cttgtgagaa ttgagaaaat |
| 901 | atgcatggaa atatccagat gaatttctta cacagattct tacgggatgc ctaaattgca |
| 961 | tcctgtaact tctgtccaaa aagaacagga tgatgtacaa attgctcttc caggtaatcc |
| 1021 | accacggtta actggaaaag cactttcagt ctcctataac cctcccacca gctgctgctt |
| 1081 | caggtataat gttacagcag tttgccaagg cggggaccta actggtgaca attgagcctc |
| 1141 | ttgactggta ctcagaattt agtgacacgt ggtcctgatt ttttttggag acggggtctt |
| 1201 | gctctcaccc aggctgggag tgcagtggca cactgactac agccttgacc tccccaggct |
| 1261 | caggtgatct tcccacctca gccttccaag tagctgggac tacagatgca cacctccaaa |
| 1321 | cctgggtagt ttttgaagtt tttttgtaga ggtggtctag ccatgttgcc taggctcccg |
| 1381 | aactcctgag ctcaagcaat cctgcttcag cctcccaaag tactgggatt acaggcatct |
| 1441 | tctgtagtat ataggtcatg agggatatgg gatgtggtac ttatgagaca gaaatgctta |
| 1501 | caggatgttt ttctgtaacc atcctggtca acttagcaga aatgctgcgc tgggtataat |
| 1561 | aaagcttttc tacttctagt ctagacagga atcttacaga ttgtctcctg ttcaaaacct |
| 1621 | agtcataaat atttataatg caaactggtc aaaaaaaaaa |

LOCUS NM_000995 918 bp mRNA linear PRI 26-JAN-2014
DEFINITION Homo sapiens ribosomal protein L34 (RPL34), transcript variant 1,
mRNA. (SEQ ID NO: 682)

| | |
|---|---|
| 1 | cttttttctt cctcttccgg ggacgttgtc tgcaggcact cagaatggtc cagcgtttga |
| 61 | cataccgacg taggctttcc tacaatacag cctctaacaa aactaggctg tcccgaaccc |
| 121 | ctggtaatag aattgtttac ctttatacca agaaggttgg gaaagcacca aaatctgcat |
| 181 | gtggtgtgtg cccaggcaga cttcgagggg ttcgtgctgt aagacctaaa gttcttatga |
| 241 | gattgtccaa aacaaagaaa catgtcagca gggcctatgg tggttccatg tgtgctaaat |
| 301 | gtgttcgtga caggatcaag cgtgctttcc ttatcgagga gcagaaaatc gttgtgaaag |
| 361 | tgttgaaggc acaagcacag agtcagaaag ctaaataaaa aaatgaaact tttttgagta |
| 421 | ataaaaatga aaagacgctg tccaatagaa aaagttggtg tgctggagct acctcacctc |
| 481 | agcttgagag agccagttgt gtgcatctct ttccagtttt gcatccagtg acgtctgctt |
| 541 | ggcatcttga gattgttatg gtgagagtat ttacacctca gcaaatgctg caaaatcctg |
| 601 | ttttccccca gagagctgga ggttaaatac taccagcaca tccctagata ctactcaagt |
| 661 | tacagtatat gatcactaat atagtatgct cttggtacca ggagctctga tatatatctg |
| 721 | gtacatgttt gataatgact tgattgttat tataagtact tattaatact tcgattctgt |
| 781 | aaagagttta gggtttgatt ttataaaatc caaaatgagc cttttattga atccagttct |
| 841 | ctatgtgacc agttctctgt atgaatggaa gggaaaagaa ttaaaaatct tgcaaagggg |

LOCUS NM_007209 475 bp mRNA linear PRI 26-JAN-2014
DEFINITION Homo sapiens ribosomal protein L35 (RPL35), mRNA. (SEQ ID NO: 683)

| | |
|---|---|
| 1 | cttcctcttt ccctcggagc gggcggcggc gttggcggct tgtgcagcaa tggccaagat |
| 61 | caaggctcga gatcttcgcg ggaagaagaa ggaggagctg ctgaaacagc tggacgacct |
| 121 | gaaggtggag ctgtcccagc tgcgcgtcgc caaagtgaca ggcggtgcgg cctccaagct |
| 181 | ctctaagatc cgagtcgtcc ggaaatccat tgcccgtgtt ctcacagtta ttaaccagac |
| 241 | tcagaaagaa aacctcagga aattctacaa gggcaagaag tacaagcccc tggacctgcg |
| 301 | gcctaagaag acacgtgcca tgcgccgccg gctcaacaag cacgaggaga acctgaagac |
| 361 | caagaagcag cagcggaagg agcggctgta cccgctgcgg aagtacgcgg tcaaggcctg |
| 421 | aggggcgcat tgtcaataaa gcacagctgg ctgagactgc |

LOCUS NM_033643 453 bp mRNA linear PRI 26-JAN-2014
DEFINITION Homo sapiens ribosomal protein L36 (RPL36), transcript variant 1,
mRNA. (SEQ ID NO: 684)

| | |
|---|---|
| 1 | gagtagatat cccggagttc cgcgcggcgc cagcccttcc gccacggccg tctctggaga |
| 61 | gcagcagcca tggccctacg ctaccctatg gccgtgggcc tcaacaaggg ccacaaagtg |
| 121 | accaagaacg tgagcaagcc caggcacagc cgacgccgcg ggcgtctgac caaacacacc |
| 181 | aagttcgtgc gggacatgat tcgggaggtg tgtggctttg ccccgtacga gcggcgcgcc |
| 241 | atggagttac tgaaggtctc caaggacaaa cgggccctca aatttatcaa gaaaagggtg |
| 301 | gggacgcaca tccgcgccaa gaggaagcgg gaggagctga gcaacgtact ggccgccatg |
| 361 | aggaaagctg ctgccaagaa agactgagcc cctcccctgc cctctccctg aaataaagaa |
| 421 | cagcttgaca gaaaa |

LOCUS NM_021029 881 bp mRNA linear PRI 27-JUN-2013
DEFINITION Homo sapiens ribosomal protein L36a (RPL36A), transcript variant
1, mRNA. (SEQ ID NO: 685)

| | |
|---|---|
| 1 | gagtcctctc agccgcccga gggcgctgcg ctgagcctta cactctatga ttgctcctac |
| 61 | cgactcccat gaggaagtgc gatcgggaac ctcctatata cttccgtttg cctcgcggtt |
| 121 | tctttctttc cgcgccgata gcgctcacgc aagcatggtt aacgtcccta aaacccgccg |
| 181 | gactttctgt aagaagtgtg gcaagcacca accccataaa gtgacacagt acaagaaggg |
| 241 | caaggattct ctgtacgccc agggaaagcg gcgttatgac aggaagcaga gtggctatgg |
| 301 | tgggcaaact aagccgattt tccggaaaaa ggctaaaact acaaagaaga ttgtgctaag |
| 361 | gcttgagtgc gttgagccca actgcagatc taagagaatg ctggctatta aaagatgcaa |
| 421 | gcattttgaa ctgggaggag ataagaagag aaagggccaa gtgatccagt tctaagtgtc |
| 481 | atcttttatt atgaagacaa taaaatcttg agtttatgtt cacttcattt gtttgctgtt |
| 541 | catcttttgg gagggaataa gctagagcca tcaatacaat tccgcttgtg gggaaattta |
| 601 | tgcctcttac tggtactact tgttttgcat tgaagctgac tggttgagtt cacatcatat |
| 661 | gttgcaattt tctaatttgg cacttcaatc actaggggcc ttatgaggca gtttgtcatt |
| 721 | atgcaatggt tattggttat catgtgagta gacacatttc aggctaatag ggagaagtca |
| 781 | gtaacacatt catagtgaat atgagatgtc tttgctaaga gttaagtgtc agatctttgt |
| 841 | tataacagtt aatttaataa agaattttgg cattgttctt c |

LOCUS NM_000997 1586 bp mRNA linear PRI 26-JAN-2014
DEFINITION Homo sapiens ribosomal protein L37 (RPL37), mRNA. (SEQ ID NO: 686)

| | |
|---|---|
| 1 | gcgccccgca ggaagtgctt ccctgggcgg aagcttctga gcgtgatata gcggaagtgc |
| 61 | cttctcttcc ggtctttctg gtctcggccg cagaagcgag atgacgaagg gaacgtcatc |
| 121 | gtttggaaag cgtcgcaata agacgcacac gttgtgccgc cgctgtggct ctaaggccta |
| 181 | ccaccttcag aagtcgacct gtggcaaatg tggctaccct gccaagcgca agagaaagta |
| 241 | taactggagt gccaaggcta aaagacgaaa taccaccgga actggtcgaa tgaggcacct |
| 301 | aaaaattgta taccgcagat tcaggcatgg attccgtgaa ggaacaacac ctaaacccaa |
| 361 | gagggcagct gttgcagcat ccagttcatc ttaagaatgt caacgattag tcatgcaata |
| 421 | aatgttctgg ttttaaaaaa tacatatctg gttttggtaa ggtattttta atcaattagg |
| 481 | cttgtagtat cagtgaaata ctgtaggttt agggactggg ctagcttcat atcagattta |
| 541 | cttgttaagt gactgttttg gaatgtttac ttttggactg ggtttgtaac acggttaaag |
| 601 | gcaatgagaa acaagcagaa ttccaggagt ccttgaagca gagggcactg gaagacaata |
| 661 | tagcagatta aaatagcaca gctcatgtgg cataggtggg tattttagat gtttgagtaa |
| 721 | atttgaaaga gtatgatgtt taaattacct ttagcaacat gttcatctgc tatgctgtca |
| 781 | tgactagggg gatgattatt agtcacatag agcttgggag taccactgga aacgtatggg |
| 841 | taggagttta ggtggcttct gtttttcaaa agatgatctt atcctagtat ctgtaatgct |
| 901 | cacttggcac acctgacttg tgggctgtgt gtaaggtggc tagctaagtg aaaaaagcct |
| 961 | gctaggtgtg agtcaactta agaatatgta aataggtttg agaaaaagta gggcttgggt |
| 1021 | gcaagtaaag attgagcagg aaataaagga aaatcaagta taatccctga gatttgtaga |
| 1081 | ctaaaggcaa tgatgtggga ctacttggtc gaattttttt agccctcaac ttggtaattg |
| 1141 | ggtgtttctg tgttaaagca ctgaaacttg ctgtcgtgcc ttcctagttt tcgtggttta |
| 1201 | ttgacagggt tgggggtttt ttttgttttt ttaaaatgaa gggacaaagt caactggact |
| 1261 | gctgagtgag agggcagggg cagttgaagg gaacatgaat tgctggaaca gctacataaa |
| 1321 | atagtgatgt agccaagtca tgctatttaa attataattc tccactgtgt ttagaataac |
| 1381 | atctgaggtt cttaacctgg ccttggaagg gtatcacttt tacttgtaac ctggaatggc |
| 1441 | tttataatgt gctagctaat tgctactctc atcttgtatt ttaactccta atttaccctt |
| 1501 | caggtctcag cttcagaaca ttcacttata aagaaaccct gctgattaaa tctctcttgg |
| 1561 | gcttcctccc aaaa |

LOCUS NM_000998 434 bp mRNA linear PRI 17-APR-2013
DEFINITION Homo sapiens ribosomal protein L37a (RPL37A), mRNA. (SEQ ID NO: 687)

| | |
|---|---|
| 1 | acttccgctc gtccgcctaa taccgcgcct gcgcaccgcg tctcttcctt tctgggctcg |
| 61 | gacctaggtc gcggcgacat ggccaaacgt accaagaaag tcgggatcgt cggtaaatac |
| 121 | gggacccgct atggggcctc cctccggaaa atggtgaaga aaattgaaat cagccagcac |
| 181 | gccaagtaca cttgctcttt ctgtggcaaa accaagatga agagacgagc tgtggggatc |
| 241 | tggcactgtg gttcctgcat gaagacagtg gctggcggtg cctggacgta caataccact |
| 301 | tccgctgtca cggtaaagtc cgccatcaga agactgaagg agttgaaaga ccagtagacg |
| 361 | ctcctctact ctttgagaca tcactggcct ataataaatg ggttaattta tgtaacaaaa |

LOCUS NM_000999 411 bp mRNA linear PRI 17-APR-2013
DEFINITION Homo sapiens ribosomal protein L38 (RPL38), transcript variant 1,
mRNA. (SEQ ID NO: 688)

| | |
|---|---|
| 1 | gcccggaaac ggaagtctcg ttctttttcg tccttttccc cggttgctgc ttgctgtgag |
| 61 | tgtctctagg gtgatacgtg ggtgagaaag gtcctggtcc gcgccagagc ccagcgcgcc |
| 121 | tcgtcgccat gcctcggaaa attgaggaaa tcaaggactt cctgctcaca gcccgacgaa |
| 181 | aggatgccaa atctgtcaag atcaagaaaa ataaggacaa cgtgaagttt aaagttcgat |
| 241 | gcagcagata cctttacacc ctggtcatca ctgacaaaga gaaggcagag aaactgaagc |
| 301 | agtccctgcc ccccggtttg gcagtgaagg aactgaaatg aaccagacac actgattgga |
| 361 | actgtattat attaaaatac taaaaatcct aaa |

LOCUS NM_001000 439 bp mRNA linear PRI 11-JAN-2014
DEFINITION Homo sapiens ribosomal protein L39 (RPL39), mRNA. (SEQ ID NO: 689)

| | |
|---|---|
| 1 | gccttctaag ctcgttcttc cgccagcttc cctcctcttc ctttctccgc catcgtggtg |
| 61 | tgttcttgac tccgctgctc gccatgtctt ctcacaagac tttcaggatt aagcgattcc |
| 121 | tggccaagaa acaaaagcaa aatcgtccca ttccccagtg gattcggatg aaaactggaa |
| 181 | ataaaatcag gtacaactcc aaaaggagac attggagaag aaccaagctg ggtctataag |
| 241 | gaattgcaca tgagatggca cacatattta tgctgtctga aggtcacgat catgttacca |
| 301 | tatcaagctg aaaatgtcac cactatctgg agatttcgac gtgttttcct ctctgaatct |
| 361 | gttatgaaca cgttggttgg ctggattcag taataaatat gtaaggcctt tctttttaaa |

LOCUS NM_021104 478 bp mRNA linear PRI 26-JAN-2014
DEFINITION Homo sapiens ribosomal protein L41 (RPL41), transcript variant 1,
mRNA. (SEQ ID NO: 690)

| | |
|---|---|
| 1 | acccggcgct ccattaaata gccgtagacg gaacttcgcc tttctctcgg ccttagcgcc |
| 61 | atttttttgg aaacctctgc gccatgagag ccaagtggag gaagaagcga atgcgcaggc |
| 121 | tgaagcgcaa aagaagaaag atgaggcaga ggtccaagta aaccgctagc ttgttgcacc |
| 181 | gtggaggcca caggagcaga aacatggaat gccagacgct ggggatgctg gtacaagttg |
| 241 | tgggactgca tgctactgtc tagagcttgt ctcaatggat ctagaacttc atcgccctct |
| 301 | gatcgccgat cacctctgag acccaccttg ctcataaaca aaatgcccat gttggtcctc |
| 361 | tgccctggac ctgtgacatt ctggactatt tctgtgttta tttgtggccg agtgtaacaa |
| 421 | ccatataata aatcacctct tccgctgttt tagctgaaga attaaatcaa |

LOCUS NM_032477 632 bp mRNA linear PRI 22-SEP-2013
DEFINITION Homo sapiens mitochondrial ribosomal protein L41 (MRPL41), mRNA.
(SEQ ID NO: 691)

| | |
|---|---|
| 1 | gcggccgcgg ccaatcggag ccgctcttgc tgcgacgcag cggtcggaag cggagcaagg |
| 61 | tcgaggccgg gttggcgccg gagccggggc cgcttggagc tcgtgtgggg tctccggtcc |
| 121 | agggcgcggc atgggcgtcc tggccgcagc ggcgcgctgc ctggtccggg gtgcggaccg |
| 181 | aatgagcaag tggacgagca agcggggccc gcgcagcttc aggggccgca agggccgggg |
| 241 | cgccaagggc atcggcttcc tcacctcggg ctggaggttc gtgcagatca aggagatggt |
| 301 | cccggagttc gtcgtcccgg atctgaccgg cttcaagctc aagccctacg tgagctacct |
| 361 | cgcccctgag agcgaggaga cgcccctgac ggccgcgcag ctcttcagcg aagccgtggc |
| 421 | gcctgccatc gaaaaggact tcaaggacgg taccttcgac cctgacaacc tggaaaagta |
| 481 | cggcttcgag cccacacagg agggaaagct cttccagctc taccccagga acttcctgcg |
| 541 | ctagctgggc gggggagggg cggcctgccc tcatctcatt tctattaaac gcctttgcca |
| 601 | gctaaa |

LOCUS NM_002295 1155 bp mRNA linear PRI 26-JAN-2014
DEFINITION Homo sapiens ribosomal protein SA (RPSA), transcript variant 1,
mRNA. (SEQ ID NO: 692)

| | |
|---|---|
| 1 | cgcctgtctt ttccgtgcta cctgcagagg ggtccatacg gcgttgttct ggattcccgt |
| 61 | cgtaacttaa agggaaattt tcacaatgtc cggagccctt gatgtcctgc aaatgaagga |
| 121 | ggaggatgtc cttaagttcc ttgcagcagg aacccactta ggtggcacca atcttgactt |
| 181 | ccagatggaa cagtacatct ataaaaggaa aagtgatggc atctatatca taaatctcaa |
| 241 | gaggacctgg gagaagcttc tgctggcagc tcgtgcaatt gttgccattg aaaaccctgc |
| 301 | tgatgtcagt gttatatcct ccaggaatac tggccagagg gctgtgctga agtttgctgc |
| 361 | tgccactgga gccactccaa ttgctggccg cttcactcct ggaaccttca ctaaccagat |
| 421 | ccaggcagcc ttccgggagc cacggcttct tgtggttact gaccccaggg ctgaccacca |
| 481 | gcctctcacg gaggcatctt atgttaacct acctaccatt gcgctgtgta acacagattc |
| 541 | tcctctgcgc tatgtggaca ttgccatccc atgcaacaac aagggagctc actcagtggg |
| 601 | tttgatgtgg tggatgctgg ctcgggaagt tctgcgcatg cgtggcacca tttcccgtga |
| 661 | acacccatgg gaggtcatgc ctgatctgta cttctacaga gatcctgaag agattgaaaa |
| 721 | agaagagcag gctgctgctg agaaggcagt gaccaaggag gaatttcagg gtgaatggac |
| 781 | tgctcccgct cctgagttca ctgctactca gcctgaggtt gcagactggt ctgaaggtgt |
| 841 | acaggtgccc tctgtgccta ttcagcaatt ccctactgaa gactggagcg ctcagcctgc |
| 901 | cacggaagac tggtctgcag ctcccactgc tcaggccact gaatgggtag gagcaaccac |
| 961 | tgactggtct taagctgttc ttgcataggc tcttaagcag catggaaaaa tggttgatgg |
| 1021 | aaaataaaca tcagtttcta aaagttgtct tcatttagtt tgctttttac tccagatcag |
| 1081 | aatacctggg attgcatatc aaagcataat aataaataca tgtctcgaca tgagttgtac |
| 1141 | ttctaaaaaa |

LOCUS NM_002952 962 bp mRNA linear PRI 19-JAN-2014
DEFINITION Homo sapiens ribosomal protein S2 (RPS2), mRNA. (SEQ ID NO: 693)

| | |
|---|---|
| 1 | cttcttttcc gacaaaacac caaatggcgg atgacgccgg tgcagcgggg gggcccgggg |
| 61 | gccctggtgg ccctgggatg gggaaccgcg gtggcttccg cggaggtttc ggcagtggca |
| 121 | tccggggccg gggtcgcggc cgtggacggg gccggggccg aggccgcgga gctcgcggag |
| 181 | gcaaggccga ggataaggag tggatgcccg tcaccaagtt gggccgcttg gtcaaggaca |
| 241 | tgaagatcaa gtccctggag gagatctatc tcttctccct gcctattaag gaatcagaga |
| 301 | tcattgattt cttcctgggg gcctctctca aggatgaggt tttgaagatt atgccagtgc |
| 361 | agaagcagac ccgtgccggc cagcgcacca ggttcaaggc atttgttgct atcggggact |
| 421 | acaatggcca cgtcggtctg ggtgttaagt gctccaagga ggtggccacc gccatccgtg |
| 481 | gggccatcat cctggccaag ctctccatcg tccccgtgcg cagaggctac tgggggaaca |
| 541 | agatcggcaa gccccacact gtcccttgca aggtgacagg ccgctgcggc tctgtgctgg |
| 601 | tacgcctcat ccctgcaccc aggggcactg gcatcgtctc cgcacctgtg cctaagaagc |
| 661 | tgctcatgat ggctggtatc gatgactgct acacctcagc ccggggctgc actgccaccc |
| 721 | tgggcaactt cgccaaggcc acctttgatg ccatttctaa gacctacagc tacctgaccc |
| 781 | ccgacctctg gaaggagact gtattcacca agtctcccta tcaggagttc actgaccacc |
| 841 | tcgtcaagac ccacaccaga gtctccgtgc agcggactca ggctccagct gtggctacaa |
| 901 | catagggttt ttatacaaga aaaataaagt gaattaagcg tg |

LOCUS NM_001005 2108 bp mRNA linear PRI 19-JAN-2014
DEFINITION Homo sapiens ribosomal protein S3 (RPS3), transcript variant 1,
mRNA. (SEQ ID NO: 694)

| | |
|---|---|
| 1 | atactcactt ccgcccgcga gccacttcct ttcctttcag cggagcgcgg cggcaagatg |
| 61 | gcagtgcaaa tatccaagaa gaggaagttt gtcgctgatg gcatcttcaa agctgaactg |
| 121 | aatgagtttc ttactcggga gctggctgaa gatggctact ctggagttga ggtgcgagtt |
| 181 | acaccaacca ggacagaaat cattatctta gccaccagaa cacagaatgt tcttggtgag |
| 241 | aagggccggc ggattcggga actgactgct gtagttcaga agaggtttgg ctttccagag |
| 301 | ggcagtgtag agctttatgc tgaaaaggtg gccactagag gtctgtgtgc cattgcccag |
| 361 | gcagagtctc tgcgttacaa actcctagga gggcttgctg tgcggagggc ctgctatggt |
| 421 | gtgctgcggt tcatcatgga gagtggggcc aaaggctgcg aggttgtggt gtctgggaaa |
| 481 | ctccgaggac agagggctaa atccatgaag tttgtggatg gcctgatgat ccacagcgga |
| 541 | gaccctgtta actactacgt tgacactgct gtgcgccacg tgttgctcag acagggtgtg |
| 601 | ctgggcatca aggtgaagat catgctgccc tgggacccaa ctggtaagat tggccctaag |
| 661 | aagcccctgc ctgaccacgt gagcattgtg gaacccaaag atgagatact gcccaccacc |
| 721 | cccatctcag aacagaaggg tgggaagcca gagccgcctg ccatgcccca gccagtcccc |
| 781 | acagcataac agggtctcct tggcagctgt attctggagt ctggatgttg ctctctaaag |
| 841 | acctttaata aaattttgta caaagacaca aggtctgact agactgttca gtattcagac |
| 901 | tgaggggcat gttggcctct ggagcattac atatcttctt ggttttaacc atacttgtgg |
| 961 | tatttgcaag ggccagaaca gtaagaccca agcagagcca accagagaaa taatatttgt |
| 1021 | gtgatagaga aggctgatag caagcaaggc agcaccttga ttcgttgtcc tgtagttcag |
| 1081 | gattgtaggt ttagaagagg gatatgtttg agtttttcct atgcataagg cgatccacgt |
| 1141 | tgcacataga aagtgaatat aaatggccat tatattttgt gtcatgctgt gctctaagtg |
| 1201 | ttctttacat atgtactcgt taatcaacct ctctaaagtg taaaggaaat ttgcttgcac |
| 1261 | cactgaaggc acataaggct cagaagtaaa tttgcctaag cagtataaag ctatcattag |
| 1321 | aatccacatt cctaagttgt gttctcttag gggatcatgg aaccagtcat tggtactaca |
| 1381 | ggctattatg ttctggagaa ctgtgaagaa catttaaatt gtctctgatt ttatctatca |
| 1441 | atgttttgaa gtattttcta ccagtgtctg tacttcacaa gaaattcggc actatttttt |
| 1501 | caggcaaaac tagtgaggga caggttggct tgaaaatcat gagactgttg ttaaatcaga |
| 1561 | tgctggttga tcacagaggg gacttccagg gaaagctgtt atcaggtggc tgcttcctgg |
| 1621 | tgatgcagcc tggctgatga gataaccctg gctccacaga tggcttagca ggtgctgtga |
| 1681 | tgatttggtt ttcttctcaa ttagactgag ctgcacatgg tgtttatatt gcttggcaca |
| 1741 | tggtaagggc ttaatatttg aggtaattat gtagggcgta cactgacaag tatctgaccc |
| 1801 | ccccttcctt tttgactcat aaattggtca tcttaaccat ttaagtgtac acttctatag |
| 1861 | tgacagagtt agccctctgt ccaagggatt tgcatctgtg gattcaacca actttgggtc |
| 1921 | aaaaataatc aaaaaggatg gttgtgtgtg tattgaacat gtagacttat ttttcttatt |
| 1981 | ttcaaaatac tatattttct tgtcacttat tttcttgtac actgcagttg taacagctat |
| 2041 | gtagcatgta cattaggtat taaaagtaat ccagtgaaga ttgaaagtct aaaa |

LOCUS NM_001006 950 bp mRNA linear PRI 17-APR-2013
DEFINITION Homo sapiens ribosomal protein S3A (RPS3A), transcript variant 1,
mRNA. (SEQ ID NO: 695)

| | |
|---|---|
| 1 | tagacggcgc gccccgcccc cgtacgccta agttctcgcg cgactcccac ttccgccctt |
| 61 | ttggctctct gaccagcacc atggcggttg gcaagaacaa gcgccttacg aaaggcggca |
| 121 | aaaagggagc caagaagaaa gtggttgatc cattttctaa gaaagattgg tatgatgtga |
| 181 | aagcacctgc tatgttcaat ataagaaata ttggaaagac gctcgtcacc aggacccaag |
| 241 | gaaccaaaat tgcatctgat ggtctcaagg gtcgtgtgtt tgaagtgagt cttgctgatt |
| 301 | tgcagaatga tgaagttgca tttagaaaat tcaagctgat tactgaagat gttcagggta |
| 361 | aaaactgcct gactaacttc catggcatgg atcttacccg tgacaaaatg tgttccatgg |
| 421 | tcaaaaaatg gcagacaatg attgaagctc acgttgatgt caagactacc gatggttact |
| 481 | tgcttcgtct gttctgtgtt ggttttacta aaaaacgcaa caatcagata cggaagacct |
| 541 | cttatgctca gcaccaacag gtccgccaaa tccggaagaa gatgatggaa atcatgaccc |
| 601 | gagaggtgca gacaaatgac ttgaaagaag tggtcaataa attgattcca gacagcattg |
| 661 | gaaaagacat agaaaaggct tgccaatcta tttatcctct ccatgatgtc ttcgttagaa |
| 721 | aagtaaaaat gctgaagaag cccaagtttg aattgggaaa gctcatggag cttcatggtg |
| 781 | aaggcagtag ttctggaaaa gccactggag acgagacagg tgctaaagtt gaacgagctg |
| 841 | atagatatga accaccaatc caagaatctg tttaaagttc agacttcaaa tagtggcaaa |
| 901 | taaaaaatgc tatttgtgat ggtttgcttc tga |

LOCUS NM_001007 956 bp mRNA linear PRI 11-JAN-2014
DEFINITION Homo sapiens ribosomal protein S4, X-linked (RPS4X), mRNA.
(SEQ ID NO: 696)

| | |
|---|---|
| 1 | gggcggagca gctgaaaatc cggcgcgcgc agtctccagc cccaatttct acgcgcaccg |
| 61 | gaagacggag gtcctctttc cttgcctaac gcagccatgg ctcgtggtcc caagaagcat |
| 121 | ctgaagcggg tggcagctcc aaagcattgg atgctggata aattgaccgg tgtgtttgct |
| 181 | cctcgtccat ccaccggtcc ccacaagttg agagagtgtc tccccctcat cattttcctg |
| 241 | aggaacagac ttaagtatgc cctgacagga gatgaagtaa agaagatttg catgcagcgg |
| 301 | ttcattaaaa tcgatggcaa ggtccgaact gatataacct accctgctgg attcatggat |
| 361 | gtcatcagca ttgacaagac gggagagaat ttccgtctga tctatgacac caagggtcgc |
| 421 | tttgctgtac atcgtattac acctgaggag gccaagtaca agttgtgcaa agtgagaaag |
| 481 | atctttgtgg gcacaaaagg aatccctcat ctggtgactc atgatgcccg caccatccgc |
| 541 | taccccgatc ccctcatcaa ggtgaatgat accattcaga ttgatttgga gactggcaag |
| 601 | attactgatt tcatcaagtt cgacactggt aacctgtgta tggtgactgg aggtgctaac |
| 661 | ctaggaagaa ttggtgtgat caccaacaga gagaggcacc ctggatcttt tgacgtggtt |
| 721 | cacgtgaaag atgccaatgg caacagcttt gccactcgac tttccaacat ttttgttatt |
| 781 | ggcaagggca acaaaccatg gatttctctt ccccgaggaa agggtatccg cctcaccatt |
| 841 | gctgaagaga gagacaaaag actggcggcc aaacagagca gtgggtgaaa tgggtccctg |
| 901 | ggtgacatgt cagatctttg tacgtaatta aaaatattgt ggcaggatta atagca |

LOCUS NM_001008 910 bp mRNA linear PRI 26-JAN-2014
DEFINITION Homo sapiens ribosomal protein S4, Y-linked 1 (RPS4Y1), mRNA.
(SEQ ID NO: 697)

| | |
|---|---|
| 1 | gcaccggaaa agaacagatt ctcttccgtc gcagagtttc gccatggccc ggggccccaa |
| 61 | gaagcactta aagcgtgttg cagcgccgaa gcattggatg cttgacaaac taacgggtgt |
| 121 | atttgcacct cgtccatcga caggtcccca caagctgagg gaatgtcttc ctctgatcgt |
| 181 | cttcctcagg aatagactca agtatgcgtt gactggagat gaggtaaaga agatatgtat |
| 241 | gcaacgtttc atcaaaattg atggcaaggt tcgagtggat gtcacatacc ctgctggatt |
| 301 | catggatgtc atcagcatcg agaagacagg tgaacatttc cgcctggtct atgacaccaa |
| 361 | gggccgtttt gctgttcacc gcatcacagt ggaagaggca aagtacaagt tgtgcaaagt |
| 421 | gaggaagatt actgtgggag tgaagggaat ccctcacctg gtgactcatg atgctcgaac |
| 481 | catccgctac ccagatcctg tcatcaaggt gaacgatact gtgcagattg atttagggac |
| 541 | tggcaagata atcaacttta tcaaatttga tacaggcaat ttgtgtatgg tgattggtgg |
| 601 | agccaacctc ggtcgtgttg gtgtgatcac caacagggaa agacatcctg gttcttttga |
| 661 | tgtggtgcat gtgaaggatg ccaatggcaa cagctttgcc acgaggcttt ccaacatttt |
| 721 | tgtcattggc aatggcaata aaccttggat ttccctgccc aggggaaagg gcattcgact |
| 781 | tactgttgct gaagagagag ataagaggct ggccaccaaa cagagcagtg gctaaattgc |
| 841 | agtagcagca tatctttttt tctttgcaca aataaacagt gaattctcgt ttcttaaaaa |

LOCUS NM_001009 755 bp mRNA linear PRI 17-APR-2013
DEFINITION Homo sapiens ribosomal protein S5 (RPS5), mRNA. (SEQ ID NO: 698)

| | |
|---|---|
| 1 | ctcttcctgt ctgtaccagg gcggcgcgtg gtctacgccg agtgacagag acgctcaggc |
| 61 | tgtgttctca ggatgaccga gtgggagaca gcagcaccag cggtggcaga gaccccagac |
| 121 | atcaagctct ttgggaagtg gagcaccgat gatgtgcaga tcaatgacat ttccctgcag |
| 181 | gattacattg cagtgaagga gaagtatgcc aagtacctgc ctcacagtgc agggcggtat |
| 241 | gccgccaaac gcttccgcaa agctcagtgt cccattgtgg agcgcctcac taactccatg |
| 301 | atgatgcacg gccgcaacaa cggcaagaag ctcatgactg tgcgcatcgt caagcatgcc |
| 361 | ttcgagatca tacacctgct cacaggcgag aaccctctgc aggtcctggt gaacgccatc |
| 421 | atcaacagtg gtccccggga ggactccaca cgcattgggc gcgccgggac tgtgagacga |
| 481 | caggctgtgg atgtgtcccc cctgcgccgt gtgaaccagg ccatctggct gctgtgcaca |
| 541 | ggcgctcgtg aggctgcctt ccggaacatt aagaccattg ctgagtgcct ggcagatgag |
| 601 | ctcatcaatg ctgccaaggg ctcctcgaac tcctatgcca ttaagaagaa ggacgagctg |
| 661 | gagcgtgtgg ccaagtccaa ccgctgattt tcccagctgc tgcccaataa acctgtctgc |
| 721 | cctttggggc agtcccagcc aaaa |

LOCUS NM_001010 829 bp mRNA linear PRI 10-JAN-2014
DEFINITION Homo sapiens ribosomal protein 36 (RPS6), mRNA. (SEQ ID NO: 699)

| | |
|---|---|
| 1 | cctcttttcc gtggcgcctc ggaggcgttc agctgcttca agatgaagct gaacatctcc |
| 61 | ttcccagcca ctggctgcca gaaactcatt gaagtggacg atgaacgcaa acttcgtact |
| 121 | ttctatgaga agcgtatggc cacagaagtt gctgctgacg ctctgggtga agaatggaag |
| 181 | ggttatgtgg tccgaatcag tggtgggaac gacaaacaag gtttccccat gaagcagggt |
| 241 | gtcttgaccc atggccgtgt ccgcctgcta ctgagtaagg ggcattcctg ttacagacca |
| 301 | aggagaactg gagaaagaaa gagaaaatca gttcgtggtt gcattgtgga tgcaaatctg |
| 361 | agcgttctca acttggttat tgtaaaaaaa ggagagaagg atattcctgg actgactgat |
| 421 | actacagtgc ctcgccgcct gggccccaaa agagctagca gaatccgcaa acttttcaat |
| 481 | ctctctaaag aagatgatgt ccgccagtat gttgtaagaa agcccttaaa taaagaaggt |
| 541 | aagaaaccta ggaccaaagc acccaagatt cagcgtcttg ttactccacg tgtcctgcag |
| 601 | cacaaacggc ggcgtattgc tctgaagaag cagcgtacca agaaaaataa agaagaggct |
| 661 | gcagaatatg ctaaactttt ggccaagaga atgaaggaga ctaaggagaa gcgccaggaa |
| 721 | caaattgcga agagacgcag actttcctct ctgcgagctt ctacttctaa gtctgaatcc |
| 781 | agtcagaaat aagatttttt gagtaacaaa taaataagat cagactctg |

LOCUS NM_001011 745 bp mRNA linear PRI 18-JAN-2014
DEFINITION Homo sapiens ribosomal protein S7 (RPS7), mRNA. (SEQ ID NO: 700)

| | |
|---|---|
| 1 | gcgctgtttc cgcctcttgc cttcggacgc cggattttga cgtgctctcg cgagatttgg |
| 61 | gtctcttcct aagccggcgc tcggcaagtt ctcccaggag aaagccatgt tcagttcgag |
| 121 | cgccaagatc gtgaagccca atggcgagaa gccggacgag ttcgagtccg gcatctccca |
| 181 | ggctcttctg gagctggaga tgaactcgga cctcaaggct cagctcaggg agctgaatat |
| 241 | tacggcagct aaggaaattg aagttggtgg tggtcggaaa gctatcataa tctttgttcc |
| 301 | cgttcctcaa ctgaaatctt tccagaaaat ccaagtccgg ctagtacgcg aattggagaa |
| 361 | aaagttcagt gggaagcatg tcgtctttat cgctcagagg agaattctgc ctaagccaac |
| 421 | tcgaaaaagc cgtacaaaaa ataagcaaaa gcgtcccagg agccgtactc tgacagctgt |
| 481 | gcacgatgcc atccttgagg acttggtctt cccaagcgaa attgtgggca agagaatccg |
| 541 | cgtcaaacta gatggcagcc ggctcataaa ggttcatttg gacaaagcac agcagaacaa |
| 601 | tgtggaacac aaggttgaaa ctttttctgg tgtctataag aagctcacgg gcaaggatgt |
| 661 | taattttgaa ttcccagagt ttcaattgta aacaaaaatg actaaataaa aagtatatat |
| 721 | tcacagtaaa |

LOCUS NM_001012 705 bp mRNA linear PRI 17-APR-2013
DEFINITION Homo sapiens ribosomal protein S8 (RPS8), mRNA. (SEQ ID NO: 701)

| | |
|---|---|
| 1 | ctctttccag ccagcgccga gcgatgggca tctctcggga caactggcac aagcgccgca |
| 61 | aaaccggggg caagagaaag ccctaccaca agaagcggaa gtatgagttg gggcgcccag |
| 121 | ctgccaacac caagattagc ccccaccgca tccacacagt ccgtgtgcgg ggaagtaaca |
| 181 | agaaataccg tgccctgagg ttggacgtgg ggaatttctc ctggggctca gagtgttgta |
| 241 | ctcgtaaaac aaggatcatc gatgttgtct acaatgcatc taataacgag ctggttcgta |
| 301 | ccaagaccct ggtgaagaat tgcatcgtgc tcatcgacag cacaccgtac cgacagtggt |
| 361 | acgagtccca ctatgcgctg cccctgggcc gcaagaaggg agccaagctg actcctgagg |
| 421 | aagaagagat tttaaacaaa aaacgatcta aaaaaattca gaagaaatat gatgaaagaa |
| 481 | aaaagaatgc caaaatcagc agtctcctgg aggagcagtt ccagcagggc aagcttcttg |
| 541 | cgtgcatcgc ttcaaggccg ggacagtgtg gccgagcaga tggctatgtg ctagagggca |
| 601 | aagagttgga gttctatctt aggaaaatca aggcccgcaa aggcaaataa atccttgttt |
| 661 | tgtcttcacc catgtaataa aggtgtttat tgttttgttc ccaca |

LOCUS NM_001013 753 bp mRNA linear PRI 16-JUN-2013
DEFINITION Homo sapiens ribosomal protein S9 (RPS9), mRNA. (SEQ ID NO: 702)

| | |
|---|---|
| 1 | ctctttctca gtgaccgggt ggtttgctta ggcgcagacg gggaagcgga gccaacatgc |
| 61 | cagtggcccg gagctgggtt tgtcgcaaaa cttatgtgac cccgcggaga cccttcgaga |
| 121 | aatctcgtct cgaccaagag ctgaagctga tcggcgagta tgggctccgg aacaaacgtg |
| 181 | aggtctggag ggtcaaattt accctggcca agatccgcaa ggccgcccgg gaactgctga |
| 241 | cgcttgatga gaaggaccca cggcgtctgt tcgaaggcaa cgccctgctg cggcggctgg |
| 301 | tccgcattgg ggtgctggat gagggcaaga tgaagctgga ttacatcctg ggcctgaaga |
| 361 | tagaggattt cttagagaga cgcctgcaga cccaggtctt caagctgggc ttggccaagt |
| 421 | ccatccacca cgctcgcgtg ctgatccgcc agcgccatat cagggtccgc aagcaggtgg |
| 481 | tgaacatccc gtccttcatt gtccgcctgg attcccagaa gcacatcgac ttctctctgc |
| 541 | gctctcccta cgggggtggc cgcccgggcc gcgtgaagag gaagaatgcc aagaagggcc |
| 601 | agggtggggc tggggctgga gacgacgagg aggaggatta agtccacctg tccctcctgg |
| 661 | gctgctggat tgtctcgttt tcctgccaaa taaacaggat cagcgcttta caa |

LOCUS NM_001014 660 bp mRNA linear PRI 18-JAN-2014
DEFINITION Homo sapiens ribosomal protein S10 (RPS10), transcript variant 2,
mRNA. (SEQ ID NO: 703)

| | |
|---|---|
| 1 | ggcggggggc gggtccacgc cagcccggaa gagacgcagc accgcgcatg ctccttcctt |
| 61 | tccagccccg gtaccggacc ctgcagccgc agagatgttg atgcctaaga agaaccggat |
| 121 | tgccatttat gaactccttt ttaaggaggg agtcatggtg gccaagaagg atgtccacat |
| 181 | gcctaagcac ccggagctgg cagacaagaa tgtgcccaac cttcatgtca tgaaggccat |
| 241 | gcagtctctc aagtcccgag gctacgtgaa ggaacagttt gcctggagac atttctactg |
| 301 | gtaccttacc aatgagggta tccagtatct ccgtgattac cttcatctgc ccccggagat |
| 361 | tgtgcctgcc accctacgcc gtagccgtcc agagactggc aggcctcggc ctaaaggtct |
| 421 | ggagggtgag cgacctgcga gactcacaag aggggaagct gacagagata cctacagacg |
| 481 | gagtgctgtg ccacctggtg ccgacaagaa agccgaggct ggggctgggt cagcaaccga |
| 541 | attccagttt agaggcggat ttggtcgtgg acgtggtcag ccacctcagt aaaattggag |
| 601 | aggattcttt tgcattgaat aaacttacag ccaaaaaacc tta |

LOCUS NM_001015 646 bp mRNA linear PRI 07-JUL-2013
DEFINITION Homo sapiens ribosomal protein S11 (RPS11), mRNA. (SEQ ID NO: 704)

| | |
|---|---|
| 1 | tctccttacg tcacttcctc tccagcccct gcgtaatcga taaggaaacc cggacgctgc |
| 61 | tgcccctttc tttttttcag gcggccggga agatggcgga cattcagact gagcgtgcct |
| 121 | accaaaagca gccgaccatc tttcaaaaca agaagagggt cctgctggga gaaactggca |
| 181 | aggagaagct cccgcggtac tacaagaaca tcggtctggg cttcaagaca cccaaggagg |
| 241 | ctattgaggg cacctacatt gacaagaaat gccccttcac tggtaatgtg tccattcgag |
| 301 | ggcggatcct ctctggcgtg gtgaccaaga tgaagatgca gaggaccatt gtcatccgcc |
| 361 | gagactatct gcactacatc cgcaagtaca accgcttcga gaagcgccac aagaacatgt |
| 421 | ctgtacacct gtccccctgc ttcagggacg tccagatcgg tgacatcgtc acagtgggcg |
| 481 | agtgccggcc tctgagcaag acagtgcgct tcaacgtgct caaggtcacc aaggctgccg |
| 541 | gcaccaagaa gcagttccag aagttctgag gctggacatc ggcccgctcc ccacaatgaa |
| 601 | ataaagttat tttctcattc ccaggccaga cttgggatct tccgcg |

LOCUS NM_001016 532 bp mRNA linear PRI 03-NOV-2013
DEFINITION Homo sapiens ribosomal protein S12 (RPS12), mRNA. (SEQ ID NO: 705)

| | |
|---|---|
| 1 | ctctttccct gccgccgccg agtcgcgcgg aggcgaaggc ttgggtgcgt tcaagattca |
| 61 | acttcacccg taacccaccg ccatggccga ggaaggcatt gctgctggag gtgtaatgga |
| 121 | cgttaatact gctttacaag aggttctgaa gactgccctc atccacgatg gcctagcacg |
| 181 | tggaattcgc gaagctgcca aagccttaga caagcgccaa gcccatcttt gtgtgcttgc |
| 241 | atccaactgt gatgagccta tgtatgtcaa gttggtggag gccctttgtg ctgaacacca |
| 301 | aatcaaccta attaaggttg atgacaacaa gaaactagga gaatgggtag gcctttgtaa |
| 361 | aattgacaga gaggggaaac cccgtaaagt ggttggttgc agttgtgtag tagttaagga |
| 421 | ctatggcaag gagtctcagg ccaaggatgt cattgaagag tatttcaaat gcaagaaatg |
| 481 | aagaaataaa tctttggctc acaaa |

LOCUS NM_001017 529 bp mRNA linear PRI 10-JAN-2014
DEFINITION Homo sapiens ribosomal protein S13 (RPS13), mRNA. (SEQ ID NO: 706)

| | |
|---|---|
| 1 | cgctctcctt tcgttgcctg atcgccgcca tcatgggtcg catgcatgct cccgggaagg |
| 61 | gcctgtccca gtcggcttta ccctatcgac gcagcgtccc cacttggttg aagttgacat |
| 121 | ctgacgacgt gaaggagcag atttacaaac tggccaagaa gggccttact ccttcacaga |
| 181 | tcggtgtaat cctgagagat tcacatggtg ttgcacaagt acgttttgtg acaggcaata |
| 241 | aaattttaag aattcttaag tctaagggac ttgctcctga tcttcctgaa gatctctacc |
| 301 | atttaattaa gaaagcagtt gctgttcgaa agcatcttga gaggaacaga aaggataagg |
| 361 | atgctaaatt ccgtctgatt ctaatagaga gccggattca ccgtttggct cgatattata |
| 421 | agaccaagcg agtcctccct cccaattgga aatatgaatc atctacagcc tctgccctgg |
| 481 | tcgcataaat ttgtctgtgt actcaagcaa taaaatgatt gtttaacta |

LOCUS NM_005617 576 bp mRNA linear PRI 10-JAN-2014
DEFINITION Homo sapiens ribosomal protein S14 (RPS14), transcript variant 3,
mRNA. (SEQ ID NO: 707)

| | |
|---|---|
| 1 | ctccgccccc tcccactctc tctttccggt gtggagtctg gagacgacgt gcagaaatgg |
| 61 | cacctcgaaa ggggaaggaa aagaaggaag aacaggtcat cagcctcgga cctcaggtgg |
| 121 | ctgaaggaga gaatgtattt ggtgtctgcc atatctttgc atccttcaat gacacttttg |
| 181 | tccatgtcac tgatctttct ggcaaggaaa ccatctgccg tgtgactggt gggatgaagg |
| 241 | taaaggcaga ccgagatgaa tcctcaccat atgctgctat gttggctgcc caggatgtgg |
| 301 | cccagaggtg caaggagctg ggtatcaccg ccctacacat caaactccgg gccacaggag |
| 361 | gaaataggac caagacccct ggacctgggg cccagtcggc cctcagagcc cttgcccgct |
| 421 | cgggtatgaa gatcgggcgg attgaggatg tcacccccat cccctctgac agcactcgca |
| 481 | ggaagggggg tcgccgtggt cgccgtctgt gaacaagatt cctcaaaata ttttctgtta |
| 541 | ataaattgcc ttcatgtaaa ctgtttca |

LOCUS NM_001018 531 bp mRNA linear PRI 05-AUG-2013
DEFINITION Homo sapiens ribosomal protein S15 (RPS15), mRNA. (SEQ ID NO: 708)

| | |
|---|---|
| 1 | ggcagtctcg cgataactgc gcagacgcgg accaaagcga tctcttctga ggatccggca |
| 61 | agatggcaga agtagagcag aagaagaagc ggaccttccg caagttcacc taccgcggcg |
| 121 | tgaacctcga ccagctgctg gacatgtcct acgagcagct gatgcagctg tacagtgcgc |
| 181 | gccagcggcg gcggctgaac cggggcctgc ggcggaagca gcactccctg ctgaagcgcc |
| 241 | tgcgcaaggc caagaaggag gcgccgccca tggagaagcc ggaagtggtg aagacgcacc |
| 301 | tgcgggacat gatcatccta cccgagatag tgggcagcat ggtgggcgtc tacaacggca |
| 361 | agaccttcaa ccaggtgaag atcaagcccg agatgatcgg ccactacctg ggcgagttct |
| 421 | ccatcaccta caagcccgta aagcatggcc ggcccggcat cggggccacc cactcctccc |
| 481 | gcttcatccc tctcaagtaa tggctcagct aataaaggcg cacatgactc c |

LOCUS NM_001019 554 bp mRNA linear PRI 10-JAN-2014
DEFINITION Homo sapiens ribosomal protein S15a (RPS15A), transcript variant
2, mRNA. (SEQ ID NO: 709)

| | |
|---|---|
| 1 | ctctttccgc catctttccg cgccggtgag tagcactctc tgagagctcc aatttcatcc |
| 61 | gtctgccatc ggcgccatcc tgcaatctaa gccacaatgg tgcgcatgaa tgtcctggca |
| 121 | gatgctctca agagtatcaa caatgccgaa aagagaggca aacgccaggt gcttattagg |
| 181 | ccgtgctcca aagtcatcgt ccggtttctc actgtgatga tgaagcatgg ttacattggc |
| 241 | gaatttgaaa tcattgatga ccacagagct gggaaaattg ttgtgaacct cacaggcagg |
| 301 | ctaaacaagt gtggggtgat cagccccaga tttgacgtgc aactcaaaga cctggaaaaa |
| 361 | tggcagaata atctgcttcc atcccgccag tttggtttca ttgtactgac aacctcagct |
| 421 | ggcatcatgg accatgaaga agcaagacga aaacacacag gagggaaaat cctgggattc |
| 481 | tttttctagg gatgtaatac atatatttac aaataaaatg cctcatggac tctggtgctt |
| 541 | ccaa |

LOCUS NM_001020 603 bp mRNA linear PRI 26-JAN-2014
DEFINITION Homo sapiens ribosomal protein S16 (RPS16), mRNA. (SEQ ID NO: 710)

| | |
|---|---|
| 1 | gaaaagcggc cagggtggcc cctagctttc cttttccggt tgcggcgccg cgcggtgagg |
| 61 | ttgtctagtc cacgctcgga gccatgccgt ccaagggccc gctgcagtct gtgcaggtct |
| 121 | tcggacgcaa gaagacagcg acagctgtgg cgcactgcaa acgcggcaat ggtctcatca |
| 181 | aggtgaacgg gcggcccctg gagatgattg agccgcgcac gctacagtac aagctgctgg |
| 241 | agccagttct gcttctcggc aaggagcgat ttgctggtgt agacatccgt gtccgtgtaa |
| 301 | agggtggtgg tcacgtggcc cagatttatg ctatccgtca gtccatctcc aaagccctgg |
| 361 | tggcctatta ccagaaatat gtggatgagg cttccaagaa ggagatcaaa gacatcctca |
| 421 | tccagtatga ccggaccctg ctggtagctg accctcgtcg ctgcgagtcc aaaaagtttg |
| 481 | gaggccctgg tgcccgcgct cgctaccaga aatcctaccg ataagcccat cgtgactcaa |
| 541 | aactcacttg tataataaac agtttttgag ggattttaaa gtttcaagaa |

LOCUS NM_001021 511 bp mRNA linear PRI 18-JAN-2014
DEFINITION Homo sapiens ribosomal protein S17 (RPS17), mRNA. (SEQ ID NO: 711)

| | |
|---|---|
| 1 | gtttcctctt ttaccaagga cccgccaaca tgggccgcgt tcgcaccaaa accgtgaaga |
| 61 | aggcggcccg ggtcatcata gaaaagtact acacgcgcct gggcaacgac ttccacacga |
| 121 | acaagcgcgt gtgcgaggag atcgccatta tccccagcaa aaagctccgc aacaagatag |
| 181 | caggttatgt cacgcatctg atgaagcgaa ttcagagagg cccagtaaga ggtatctcca |
| 241 | tcaagctgca ggaggaggag agagaaagga gagacaatta tgttcctgag gtctcagcct |
| 301 | tggatcagga gattattgaa gtagatcctg acactaagga aatgctgaag cttttggact |
| 361 | tcggcagtct gtccaacctt caggtcactc agcctacagt tgggatgaat ttcaaaacgc |
| 421 | ctcggggacc tgtttgaatt ttttctgtag tgctgtatta ttttcaataa atctgggaca |
| 481 | acagcaaaaa |

LOCUS NM_022551 549 bp mRNA linear PRI 26-JAN-2014
DEFINITION Homo sapiens ribosomal protein S18 (RPS18), mRNA. (SEQ ID NO: 712)

| | |
|---|---|
| 1 | ctctcttcca caggaggcct acacgccgcc gcttgtgctg cagccatgtc tctagtgatc |
| 61 | cctgaaaagt tccagcatat tttgcgagta ctcaacacca acatcgatgg gcggcggaaa |
| 121 | atagcctttg ccatcactgc cattaagggt gtgggccgaa gatatgctca tgtggtgttg |
| 181 | aggaaagcag acattgacct caccaagagg gcgggagaac tcactgagga tgaggtggaa |
| 241 | cgtgtgatca ccattatgca gaatccacgc cagtacaaga tcccagactg gttcttgaac |
| 301 | agacagaagg atgtaaagga tggaaaatac agccaggtcc tagccaatgg tctggacaac |
| 361 | aagctccgtg aagacctgga gcgactgaag aagattcggg cccatagagg gctgcgtcac |
| 421 | ttctggggcc ttcgtgtccg aggccagcac accaagacca ctggccgccg tggccgcacc |
| 481 | gtgggtgtgt ccaagaagaa ataagtctgt aggccttgtc tgttaataaa tagtttatat |
| 541 | acaa |

LOCUS NM_018135 1207 bp mRNA linear PRI 10-JAN-2014
DEFINITION Homo sapiens mitochondrial ribosomal protein S18A (MRPS18A),
transcript variant 1, mRNA. (SEQ ID NO: 713)

| | |
|---|---|
| 1 | aaatggacga gaggtcaggg taggtttttg aagatggcgc ccctcaaggc tctggtgtcc |
| 61 | ggctgtgggc ggcttctCCg tgggctacta gcgggcccgg cagcgaccag ctggtctcgg |
| 121 | cttccagctc gcgggttcag ggaagtggtg gagacccaag aagggaagac aactataatt |
| 181 | gaaggccgta tcacagcgac tcccaaggag agtccaaatc ctcctaaccc ctctggccag |
| 241 | tgccccatct gccgttggaa cctgaagcac aagtataact atgacgatgt tctgctgctt |
| 301 | agccagttca tccggcctca tggaggcatg ctgccccgaa agatcacagg cctatgccag |
| 361 | gaagaacacc gcaagatcga ggagtgtgtg aagatggccc accgagcagg tctattacca |
| 421 | aatcacaggc ctcggcttcc tgaaggagtt gttccgaaga gcaaacccca actcaaccgg |
| 481 | tacctgacgc gctgggctcc tggctccgtc aagcccatct acaaaaaagg cccccgctgg |
| 541 | aacagggtgc gcatgcccgt ggggtcaccc cttctgaggg acaatgtctg ctactcaaga |
| 601 | acaccttgga agctgtatca ctgacagaga gcagtgcttc cagagttcct cctgcacctg |
| 661 | tgctggggag taggaggccc actcacaagc ccttggccac aactatactc ctgtcccacc |
| 721 | ccaccacgat ggcctggtcc ctccaacatg catggacagg ggacagtggg actaacttca |
| 781 | gtacccttgg cctgcacagt agcaatgctg ggagctagag gcaggcaggg cagttgggtc |
| 841 | ccttgccagc tgctatgggg cttaggccat gctcagtgct ggggacagga gttttgccca |
| 901 | acgcagtgtc ataaactggg ttcatgggct tacccattgg gtgtgcgctc actgcttggg |
| 961 | aagtgcaggg ggtcctgggc acattgccag ctgggtgctg agcattgagt cactgatctc |
| 1021 | ttgtgatggg gccaatgagt caattgaatt catgggccaa acaggtccca tcctcttcat |
| 1081 | gacagctgtg agctccttac tgtgggagag ctgcagggag ccaaggtggg ctgcctgaca |
| 1141 | cacttgccgc tctcgtgtga atccaagaaa ctgcgttcct caaaggggca |

LOCUS NM_001022 872 bp mRNA linear PRI 18-JAN-2014
DEFINITION Homo sapiens ribosomal protein S19 (RPS19), mRNA. (SEQ ID NO: 714)

| | |
|---|---|
| 1 | gtactttcgc catcatagta ttctccacca ctgttccttc cagccacgaa cgacgcaaac |
| 61 | gaagccaagt tcccccagct ccgaacagga gctctctatc ctctctctat tacactccgg |
| 121 | gagaaggaaa cgcgggagga aacccaggcc tccacgcgcg accccttggc cctccccttt |
| 181 | acctctccac ccctcactag acaccctccc ctctaggcgg ggacgaactt tcgccctgag |
| 241 | agaggcggag cctcagcgtc taccctcgct ctcgcgagct ttcggaactc tcgcgagacc |
| 301 | ctacgcccga cttgtgcgcc cgggaaaccc cgtcgttccc tttcccctgg ctggcagcgc |
| 361 | ggaggccgca cgatgcctgg agttactgta aaagacgtga accagcagga gttcgtcaga |
| 421 | gctctggcag ccttcctcaa aaagtccggg aagctgaaag tccccgaatg ggtggatacc |
| 481 | gtcaagctgg ccaagcacaa agagcttgct ccctacgatg agaactggtt ctacacgcga |
| 541 | gctgcttcca cagcgcggca cctgtacctc cggggtggcg ctggggttgg ctccatgacc |
| 601 | aagatctatg ggggacgtca gagaaacggc gtcatgccca gccacttcag ccgaggctcc |
| 661 | aagagtgtgg cccgccgggt cctccaagcc ctggaggggc tgaaaatggt ggaaaaggac |
| 721 | caagatggcg gccgcaaact gacacctcag ggacaaagag atctggacag aatcgccgga |
| 781 | caggtggcag ctgccaacaa gaagcattag aacaaaccat gctgggttaa taaattgcct |
| 841 | cattcgtaaa |

LOCUS NM_001023 857 bp mRNA linear PRI 11-JAN-2014
DEFINITION Homo sapiens ribosomal protein S20 (RPS20), transcript variant 2,
mRNA. (SEQ ID NO: 715)

| | |
|---|---|
| 1 | atatttcctg ttccggggcg tgtgggaccc ggatgcaagc gtgctatata agcgttgctc |
| 61 | aagtcccacc cctttctttt tgaggaagac gcggtcgtaa gggctgagga tttttggtcc |
| 121 | gcacgctcct gctcctgact caccgctgtt cgctctcgcc gaggaacaag tcggtcagga |
| 181 | agcccgcgcg caacagccat ggcttttaag gataccggaa aaacacccgt ggagccggag |
| 241 | gtggcaattc accgaattcg aatcacccta acaagccgca acgtaaaatc cttggaaaag |
| 301 | gtgtgtgctg acttgataag aggcgcaaaa gaaaagaatc tcaaagtgaa aggaccagtt |
| 361 | cgaatgccta ccaagacttt gagaatcact acaagaaaaa ctccttgtgg tgaaggttct |
| 421 | aagacgtggg atcgtttcca gatgagaatt cacaagcgac tcattgactt gcacagtcct |
| 481 | tctgagattg ttaagcagat tacttccatc agtattgagc caggagttga ggtggaagtc |
| 541 | accattgcag atgcttaagt caactatttt aataaattga tgaccagttg ttaacttctg |
| 601 | ttggttttta ttcagaatac tggcagattt taggaatata aaggtgtact atgagacttc |
| 661 | cacttttcag gtggaatata tgggtatctt agagtggtct atcctgtttt cgttgtcgtt |
| 721 | tgagtcattt gaaaactgga ttccgttaac tacataatat gtgagacctg actggtttta |
| 781 | ttggacactg gcagtttata actttggcat actctagata aattctgatt ggtatgggga |

LOCUS NM_001024 418 bp mRNA linear PRI 26-JAN-2014
DEFINITION Homo sapiens ribosomal protein S21 (RPS21), mRNA. (SEQ ID NO: 716)

| | |
|---|---|
| 1 | cttgcccgcc gatatctctg ccgggtgact agctgcttcc tttctctctc gcgcgcggtg |
| 61 | tggtggcagc aggcgcagcc cagcctcgaa atgcagaacg acgccggcga gttcgtggac |
| 121 | ctgtacgtgc cgcggaaatg ctccgctagc aatcgcatca tcggtgccaa ggaccacgca |
| 181 | tccatccaga tgaacgtggc cgaggttgac aaggtcacag gcaggtttaa tggccagttt |
| 241 | aaaacttatg ctatctgcgg ggccattcgt aggatgggtg agtcagatga ttccattctc |
| 301 | cgattggcca aggccgatgg catcgtctca aagaactttt gactggagag aatcacagat |
| 361 | gtggaatatt tgtcataaat aaataatgaa aacctaaaaa |

LOCUS NM_001025 3325 bp mRNA linear PRI 10-JAN-2014
DEFINITION Homo sapiens ribosomal protein S23 (RPS23), mRNA. (SEQ ID NO: 717)

| | |
|---|---|
| 1 | ggggtccttg gctgggcggg gcttgctcgc ggtggcttgt ggctccttcc tgcggtgctt |
| 61 | ctctctttcg ctcaggcccg tggcgccgac aggatgggca agtgtcgtgg acttcgtact |
| 121 | gctaggaagc tccgtagtca ccgacgagac cagaagtggc atgataaaca gtataagaaa |
| 181 | gctcatttgg gcacagccct aaaggccaac ccttttggag gtgcttctca tgcaaaagga |
| 241 | atcgtgctgg aaaaagtagg agttgaagcc aaacagccaa attctgccat taggaagtgt |
| 301 | gtaagggtcc agctgatcaa gaatggcaag aaaatcacag cctttgtacc caatgacggt |
| 361 | tgcttgaact ttattgagga aaatgatgaa gttctggttg ctggatttgg tcgcaaaggt |
| 421 | catgctgttg gtgatattcc tggagtccgc tttaaggttg tcaaagtagc caatgtttct |
| 481 | cttttggccc tatacaaagg caagaaggaa agaccaagat cataaatatt aatggtgaaa |
| 541 | acactgtagt aataaatttt catatgccaa aaaatgtttg tatcttactg tcccctgttc |
| 601 | tcaccacgaa gatcatgttc attaccacca ccaccccccc ttattttttt tatcctaaac |
| 661 | cagcaaacgc aggacctgta ccaattttag gagacaataa gacagggttg tttcaggatt |
| 721 | ctctagagtt aataacattt gtaacctggc acagtttccc tcatcctgtg gaataagaaa |
| 781 | atgggataga tctggaataa atgtgcagta ttgtagtatt actttaagaa ctttaaggga |
| 841 | acttcaaaaa ctcactgaaa ttctagtgag atactttctt ttttattctt ggtattttcc |
| 901 | atatcgggtg caacacttca gttaccaaat ttcattgcac atagattatc ttaggtaccc |
| 961 | ttggaaatgc acattcttgt atccatctta caggggccca agatgataaa tagtaaactc |
| 1021 | aaaattgctc cccactctgt ttattattta aaggtgtcag gatctgtgtt gtaatgtgtc |
| 1081 | tacattaatg tgtttaggag aatacaggca ttggatcatt tagttgatgg aagtatatgc |
| 1141 | caggcaaggg agataaggta tacgacaaga ctgatgtttt cagtatcttc tcatgaggtt |
| 1201 | gtcagagacc ttcatgtctt caaagactag tcagcaaatg aagtggttta gtgtagagac |
| 1261 | aagattggtt gtgttttgat aatttaagct aggtattgag tacatgtgga ttttgctgtc |
| 1321 | cacaaatact tgtttcagag ttttcatgga tacagtggca tggttgaaat gaagctgtga |
| 1381 | gccttctgct ttaaatctga tgtaagaaac tcctgttaac aaatagtaag tatgggttaa |
| 1441 | ttagcccttt gatcaaagcc tagctttaca ttgtttagga tctttggaaa acaattggtt |
| 1501 | tggttgccca ctttccgtag gatcaagagc agaacctttc acatggcaca gaagaaccca |
| 1561 | ggttgcgctt catacctgca tattccagcc ttagcctgcc atttctctcc ttggcacttt |
| 1621 | gtgctccagc aacactggtc tcagttggtc atcctcaaac ttgggttcca tatccagcct |
| 1681 | caggacctct gttcctgtta ctatggttcc ttgcatgtcg cctgctctta ctaaagagct |
| 1741 | cgtgtgtttt ccagcacact tcggtttatc tcttgatgat gatgctagtc tctccctccg |
| 1801 | caagggcgga aaggctgcct gttggtttgt accagtgttt cctaacgtgt agctgcagtc |
| 1861 | agtatttggc taagctgttc ccaggggctc aacagatgct ttcggatgag ccttaactga |
| 1921 | cccaatcctt tgtgatgcgg gagagattgc taggcctcgc tcacctggcc agaaccaggg |
| 1981 | aaagaggccg cggttgcagc gcgattccag gccctgggcg tcaggcgcgg ggtgggcagc |
| 2041 | tctccccggg cggtggggcc cttgtgaccg cgaggcgggg cgcaccagga agggagtggg |
| 2101 | acagcgcggg cgcccaggga tgtggcctgg ttacctgcct tctctgatac gtcaagacac |
| 2161 | cttcaacaat ggcttgcagc tgtaccctgt tggctgcacc caggacgccc ttttcactgc |
| 2221 | taagcagtcc tacctgaggc ccaggggctg ccagattgac ccataaataa tctccggcgc |
| 2281 | ctcagatcca gaagctgctg agcctgatct tagtgccttc tcctttctct gtgtggcccc |
| 2341 | ccagcccctt tccccactgc cttgtgtcca aggccctttc ctteatgtat ccatggagga |
| 2401 | gagacaaaaa tacacatcaa taaaataaga tagggaatcc ataaatagac attcagaagt |
| 2461 | atggccaacg gatttatctt aaaaccaatg gaggaagaag agtttcaata aatgttgtgg |
| 2521 | acttccattt gtcaaagacc aaaacaaagg aaccccaacc ttacatgtaa tacaaactta |
| 2581 | actcaaaatg gatcatatat ctaaatgtaa aatggaaagc tataaaactg aaaacagact |
| 2641 | atctttacaa cctaggcgta ggtatagttt ttagacatta caccaaaagc acatgccgta |
| 2701 | aaagaaaaaa tagataaatt ggtggatttc attaaaatta aaaaactttt tctctctgaa |
| 2761 | aaatcctgtt aagctgggcg ctgtggttca tgcctgtaat cccagcactt tgggaggctg |
| 2821 | agttgggaag aaattaatag cttgaggcca ggagttcaag atcatcctgg gcagcaaagt |
| 2881 | catacactct tgagggaaga gagagacctt ctcatattgt tttatattgt tttatactca |
| 2941 | gtacctgttt taagaaaaaa acaaggaagt gaaatcaaag acaggcagcc cggcaccagg |
| 3001 | cctgaaacca gccctgggcc tgcctggcct aaacctagta gttaaaaatc aacttacgac |
| 3061 | ttagaacctg atgttatccg tagattccaa gcattgtata aaaaaattgt gaaactccct |
| 3121 | gttgtgttct gtaccagtgc atgaaacccc tgtcacatat cccctagatt gctcaatcaa |
| 3181 | tcacgaccct ttcatgtgaa atctttagtg ttgtgagccc ttaaaaggga cagaaattgt |
| 3241 | gcacttgagg agctcagatt ttaaggctgt agcttgccga tgctcccagc tgaataaagc |
| 3301 | ccttccttct aca |

LOCUS NM_001026 649 bp mRNA linear PRI 18-JAN-2014
DEFINITION Homo sapiens ribosomal protein S24 (RPS24), transcript variant c,
mRNA. (SEQ ID NO: 718)

| | |
|---|---|
| 1 | aggcatcggc gcggtcagcc tcgtggcgcg cccacgcccc cacgccggct cttcccgggg |
| 61 | tccttccgtg cgcgttgata tgattggccg gcgaatcgtg gttctctttt cctccttggc |
| 121 | tgtctgaaga tagatcgcca tcatgaacga caccgtaact atccgcacta gaaagttcat |
| 181 | gaccaaccga ctacttcaga ggaaacaaat ggtcattgat gtccttcacc ccgggaaggc |
| 241 | gacagtgcct aagacagaaa ttcgggaaaa actagccaaa atgtacaaga ccacaccgga |
| 301 | tgtcatcttt gtatttggat tcagaactca ttttggtggt ggcaagacaa ctggctttgg |
| 361 | catgatttat gattccctgg attatgeaaa gaaaaatgaa cccaaacata gacttgcaag |
| 421 | acatggcctg tatgagaaga aaaagacctc aagaaagcaa cgaaaggaac gcaagaacag |
| 481 | aatgaagaaa gtcaggggga ctgcaaaggc caatgttggt gctggcaaaa agccgaagga |
| 541 | gtaaaggtgc tgcaatgatg ttagctgtgg ccactgtgga tttttcgcaa gaacattaat |
| 601 | aaactaaaaa cttcatgtgt ctggttgttt gaaa |

LOCUS NM_001028 514 bp mRNA linear PRI 10-JAN-2014
DEFINITION Homo sapiens ribosomal protein S25 (RFS25), mRNA. (SEQ ID NO: 719)

| | |
|---|---|
| 1 | cttccttttt gtccgacatc ttgacgaggc tgcggtgtct gctgctattc tccgagcttc |
| 61 | gcaatgccgc ctaaggacga caagaagaag aaggacgctg gaaagteggc caagaaagac |
| 121 | aaagacccag tgaacaaatc cgggggcaag gccaaaaaga agaagtggtc caaaggcaaa |
| 181 | gttcgggaca agctcaataa cttagtcttg tttgacaaag ctacctatga taaactctgt |
| 241 | aaggaagttc ccaactataa acttataacc ccagctgtgg tctctgagag actgaagatt |
| 301 | cgaggct.ccc tggccagggc agcccttcag gagctcctta gtaaaggact tatcaaactg |
| 361 | gtttcaaagc acagagctca agtaatttac accagaaata ccaagggtgg agatgctcca |
| 421 | gctgctggtg aagatgcatg aataggtcca accagctgta catttggaaa aataaaactt |
| 481 | tattaaatca |

LOCUS NM_001029 699 bp mRNA linear PRI 18-JAN-2014
DEFINITION Homo sapiens ribosomal protein S26 (RPS26), mRNA. (SEQ ID NO: 720)

| | |
|---|---|
| 1 | ggagacacat aacctcgatt ttcttccgcc atccggctaa atagtcccat gtgcactttg |
| 61 | ttccatggat aaataaacac taggaacgca tttccaccct agatttcagc agaaatgctg |
| 121 | aatgtaaagg aatatttgag taaagtgagt tgccgttctt gaagcccgtc tcctaaggat |
| 181 | tctcccggtg tccgcgtagg gatctcatgc tatataggag ggccctgcca ggcaccgtct |
| 241 | cctctctccg gtccgtgcct ccaagatgac aaagaaaaga aggaacaatg gtcgtgccaa |
| 301 | aaagggccgc ggccacgtgc agcctattcg ctgcactaac tgtgcccgat gcgtgcccaa |
| 361 | ggacaaggcc attaagaaat tcgtcattcg aaacatagtg gaggccgcag cagtcaggga |
| 421 | catttctgaa gcgagcgtct tcgatgccta tgtgcttccc aagctgtatg tgaagctaca |
| 481 | ttactgtgtg agttgtgcaa ttcacagcaa agtagtcagg aatcgatctc gtgaagcccg |
| 541 | caaggaccga acacccccac cccgatttag acctgcgggt gctgccccac gtcccccacc |
| 601 | aaagcccatg taaggagctg agttcttaaa gactgaagac aggctattct ctggagaaaa |
| 661 | ataaaatgga aattgtactt |

LOCUS NM_001030 361 bp mRNA linear PRI 17-APR-2013
DEFINITION Homo sapiens ribosomal protein S27 (RPS27), mRNA. (SEQ ID NO: 721)

| | |
|---|---|
| 1 | ctttccggcg gtgacgacct acgcacacga gaacatgect ctcgcaaagg atctccttca |
| 61 | tccctctcca gaagaggaga agaggaaaca caagaagaaa cgcctggtgc agagccccaa |
| 121 | ttcctacttc atggatgtga aatgcccagg atgctataaa atcaccacgg tctttagcca |
| 181 | tgcacaaacg gtagttttgt gtgttggctg ctccactgtc ctctgccagc ctacaggagg |
| 241 | aaaagcaagg cttacagaag gatgttcctt caggaggaag cagcactaaa agcactctga |
| 301 | gtcaagatga gtgggaaacc atctcaataa acacattttg gataaatcct ga |

LOCUS NM_002954 1068 bp mRNA linear PRI 09-JAN-2014
DEFINITION Homo sapiens ribosomal protein S27a (RPS27A), transcript variant
1, mRNA. (SEQ ID NO: 722)

| | |
|---|---|
| 1 | ctcgacctcc ttttaaaaat tctcttagcc acgttgattg tacgggaaaa gcctttttaa |
| 61 | aacatctttt acgttgctta aacctacagt ttcgaaagca ttccgaaggc taaagtgaga |
| 121 | aataagccca ggctagggag aggagaaacg aagttcacgt cctagtctgg caccgggttg |
| 181 | gattgtcgct gggacggcag tcaggcattt ggtgtggtcg cctaaggggt gggtccttcg |
| 241 | gcgggagctc cgggaaaccc cgtgggcctg cgcggcgttc ttccttttcg atccgccatc |
| 301 | tgcggtggag ccgccaccaa aatgcagatt ttcgtgaaaa cccttacggg gaagaccatc |
| 361 | accctcgagg ttgaaccctc ggatacgata gaaaatgtaa aggccaagat ccaggataag |
| 421 | gaaggaattc ctcctgatca gcagagactg atctttgctg gcaagcagct ggaagatgga |
| 481 | cgtactttgt ctgactacaa tattcaaaag gagtctactc ttcatcttgt gttgagactt |
| 541 | cgtggtggtg ctaagaaaag gaagaagaag tcttacacca ctcccaagaa gaataagcac |
| 601 | aagagaaaga aggttaagct ggctgtcctg aaatattata aggtggatga gaatggcaaa |
| 661 | attagtcgcc ttcgtegaga gtgcccttct gatgaatgtg gtgctggggt gtttatggca |
| 721 | agtcactttg acagacatta ttgtggcaaa tgttgtctga cttactgttt caacaaacca |
| 781 | gaagacaagt aactgtatga gttaataaaa gacatgaact aacatttatt gttgggtttt |
| 841 | attgcagtaa aaagaatggt ttttaagcac caaattgatg gtcacaccat ttccttttag |
| 901 | tagtgctact gctatcgctg tgtgaatgtt gcctctgggg attatgtgac ccagtggttc |
| 961 | tgtatacctg ccaggtgcca accacttgta aaggtcttga tattttcaat tcttagacta |
| 1021 | cctatacttt ggcagaagtt atatttaatg taagttgtct aaatataa |

LOCUS NM_001031 401 bp mRNA linear PRI 17-APR-2013
DEFINITION Homo sapiens ribosomal protein S28 (RFS28), mRNA. (SEQ ID NO: 723)

| | |
|---|---|
| 1 | ctctccgcca gaccgccgcc gcgccgccat catggacacc agccgtgtgc agcctatcaa |
| 61 | gctggccagg gtcaccaagg tcctgggcag gaccggttct cagggacagt gcacgcaggt |
| 121 | gcgcgtggaa ttcatggacg acacgagccg atccatcatc cgcaatgtaa aaggccccgt |
| 181 | gcgcgagggc gacgtgctca cccttttgga gtcagagcga gaagcccgga ggttgcgctg |
| 241 | agcttggctg ctcgctgggt cttggatgtc gggttcgacc acttggccga tgggaatggt |
| 301 | ctgtcacagt ctgctccttt tttttgtccg ccacacgtaa ctgagatgct cctttaaata |
| 361 | aagcgtttgt gtttcaagtt aa |

LOCUS NM_001032 386 bp mRNA linear PRI 11-JAN-2014
DEFINITION Homo sapiens ribosomal protein S29 (RPS29), transcript variant 1,
mRNA. (SEQ ID NO: 724)

| | |
|---|---|
| 1 | ctcaaaaatt tgaagagcgc atgcgtgggc cagcttcttc cttttacctc gttgcactgc |
| 61 | tgagagcaag atgggtcacc agcagctgta ctggagccac ccgcgaaaat tcggccaggg |
| 121 | ttctcgctct tgtcgtgtct gttcaaaccg gcacggtctg atccggaaat atggcctcaa |
| 181 | tatgtgccgc cagtgtttcc gtcagtacgc gaaggatatc ggtttcatta agttggacta |
| 241 | aatgctcttc cttcagagga ttatccgggg catctactca atgaaaaacc atgataattc |
| 301 | tttgtatata aaataaacat ttgaaaaaac ccttcaaaaa |

APPENDIX 8
DNA ribosomal protein mRNA Capture Probes

| Name | Sequence |
|---|---|
| MRPL20_NM_017971_r1_1 | /5Biosg/GAGCTGCGCGGTGAGGAAGACCATGGCGCCTGCAGGCCGGCGTCCCGAACACTCAA |
| (SEQ ID NO: 725) | CAACGCACGCGCAGCGCCGCTGCCATCTTGCCCGGGTCGGAAATGGTGGTCACGAGCGCTTCCG |
| MRPL20_NM_017971_r1_2 | /5Biosg/CCGCCAACCTGTAGCAGCGATTTTTCCTTCCCCGGAAGTGCCTGGCGTGCTTCAGC |
| (SEQ ID NO: 726) | ACCTCCTGGATCCGAAAGTAGCGGTCGGTGACGCGATTCCGCAGCCAGAGCTGCGCGGTGAGGA |
| MRPL20_NM_017971_r1_3 | /5Biosg/CTGGCTAGCAGCTGTAATTCGATTAATCCAGAGGGTCCTCATGTTCTTTTTCTTCA |
| (SEQ ID NO: 727) | GGTATCGGGCTTTGGTGCATTTCACAAAGGCTCGAATCACGGTTCTGACCGCCAACCTGTAGCA |
| MRPL20_NM_017971_r1_4 | /5Biosg/TCTTTGGCTCGTAGATGGCCAGATCCGCTAGGACTTTCCTGTTGAGCTCCACCTGG |
| (SEQ ID NO: 728) | CACTTAACTAAATTCCCAATGAGCGCTGGATACTTCAGTCCATGTTCCTGGCTAGCAGCTGTAA |
| MRPL20_NM_017971_r1_5 | /5Biosg/ACCACTCTGGAAAAAATGCCTTCAGGTTCCTTCCCATCCCCCAAGGCAGCAGCAAA |
| (SEQ ID NO: 729) | TCCTTCGTGTCGCCTCCTACTGGCCAAGGCAGCCAAAGATTTGAAAGTCTTTGGCTCGTAGATG |
| MRPL20_NM_017971_r1_6 | /5Biosg/GTCTGTTATTGGGTTGTAGATAAACAAAAGTATAAATCAAACAAACTGCAAATTAC |
| (SEQ ID NO: 730) | TCTGTCTCTTTTCCTAATCAATACAGCAACAGTCCTCAGTGGTACTGCACCACTCTGGAAAAAA |
| MRPL20_NM_017971_r1_7 | /5Biosg/TTTTTTTTGACTCTAAAAACTGAAGAGTGTTTGAGTTTCATTCACACAAAACATGG |
| (SEQ ID NO: 731) | ACATCATCTGTGAGGCTCTGTCCCAGAGAGACAGGGCCATCCCTCATGTCTGTTATTGGGTTGT |
| MRPL41_NM_032477_r1_1 | /5Biosg/GGACCGGAGACCCCACACGAGCTCCAAGCGGCCCCGGCTCCGGCGCCAACCCGGCC |
| (SEQ ID NO: 732) | TCGACCTTGCTCCGCTTCCGACCGCTGCGTCGCAGCAAGAGCGGCTCCGATTGGCCGCGGCCGC |
| MRPL41_NM_032477_r1_2 | /5Biosg/GCTGCGCGGGCCCCGCTTGCTCGTCCACTTGCTCATTCGGTCCGCACCCCGGACCA |
| (SEQ ID NO: 733) | GGCAGCGCGCCGCTGCGGCCAGGACGCCCATGCCGCGCCCTGGACCGGAGACCCCACACGAGCT |
| MRPL41_NM_032477_r1_3 | /5Biosg/CGACGAACTCCGGGACCATCTCCTTGATCTGCACGAACCTCCAGCCCGAGGTGAGG |
| (SEQ ID NO: 734) | AAGCCGATGCCCTTGGCGCCCCGGCCCTTGCGGCCCCTGAAGCTGCGCGGGCCCCGCTTGCTCG |
| MRPL41_NM_032477_r1_4 | /5Biosg/TTCGCTGAAGAGCTGCGCGGCCGTCAGGGGCGTCTCCTCGCTCTCAGGGGCGAGGT |
| (SEQ ID NO: 735) | AGCTCACGTAGGGCTTGAGCTTGAAGCCGGTCAGATCCGGGACGACGAACTCCGGGACCATCTC |
| MRPL41_NM_032477_r1_5 | /5Biosg/GCTTTCCCTCCTGTGTGGGCTCGAAGCCGTACTTTTCCAGGTTGTCAGGGTCGAAG |
| (SEQ ID NO: 736) | GTACCGTCCTTGAAGTCCTTTTCGATGGCAGGCGCCACGGCTTCGCTGAAGAGCTGCGCGGCCG |
| MRPL41_NM_032477_r1_6 | /5Biosg/TTTAGCTGGCAAAGGCGTTTAATAGAAATGAGATGAGGGCAGGCCGCCCCTCCCCC |
| (SEQ ID NO: 737) | GCCCAGCTAGCGCAGGAAGTTCCTGGGGTAGAGCTGGAACAGCTTTCCCTCCTGTGTGGGCTCG |
| MRPS18A_NM_018135_r1_1 | /5Biosg/CCGAGACCAGCTGGTCGCTGCCGGGCCCGCTAGTAGCCCACGGAGAAGCCGCCCAC |
| (SEQ ID NO: 738) | AGCCGGACACCAGAGCCTTGAGGGCCGCCATCTTCAAAAACCTACCCTGACCTCTCGTCCATTT |
| MRPS18A_NM_018135_r1_2 | /5Biosg/TGGCCAGAGGGGTTAGGAGGATTTGGACTCTCCTTGGGAGTCGCTGTGATACGGCC |
| (SEQ ID NO: 739) | TTCAATTATAGTTGTCTTCCCTTCTTGGGTCTCCACCACTTCCCTGAACCCGCGAGCTGGAAGC |
| MRPS18A_NM_018135_r1_3 | /5Biosg/GGCATAGGCCTGTGATCTTTCGGGGCAGCATGCCTCCATGAGGCCGGATGAACTGG |
| (SEQ ID NO: 740) | CTAAGCAGCAGAACATCGTCATAGTTATACTTGTGCTTCAGGTTCCAACGGCAGATGGGGCACT |
| MRPS18A_NM_018135_r1_4 | /5Biosg/GTTGAGTTGGGGTTTGCTCTTCGGAACAACTCCTTCAGGAAGCCGAGGCCTGTGAT |
| (SEQ ID NO: 741) | TTGGTAATAGACCTGCTCGGTGGGCCATCTTCACACACTCCTCGATCTTGCGGTGTTCTTCCTG |
| MRPS18A_NM_018135_r1_5 | /5Biosg/GAGTAGCAGACATTGTCCCTCAGAAGGGGTGACCCCACGGGCATGCGCACCCTGTT |
| (SEQ ID NO: 742) | CCAGCGGGGGCCTTTTTTGTAGATGGGCTTGACGGAGCCAGGAGCCCAGCGCGTCAGGTACCGG |
| MRPS18A_NM_018135_r1_6 | /5Biosg/ACAGGAGTATAGTTGTGGCCAAGGGCTTGTGAGTGGGCCTCCTACTCCCCAGCACA |
| (SEQ ID NO: 743) | GGTGCAGGAGGAACTCTGGAAGCACTGCTCTCTGTCAGTGATACAGCTTCCAAGGTGTTCTTGA |
| MRPS18A_NM_018135_r1_7 | /5Biosg/CTGCCCTGCCTGCCTCTAGCTCCCAGCATTGCTACTGTGCAGGCCAAGGGTACTGA |
| (SEQ ID NO: 744) | AGTTAGTCCCACTGTCCCCTGTCCATGCATGTTGGAGGGACCAGGCCATCGTGGTGGGGTGGGA |
| MRPS18A_NM_018135_r1_8 | /5Biosg/GTGAGCGCACACCCAATGGGTAAGCCCATGAACCCAGTTTATGACACTGCGTTGGG |
| (SEQ ID NO: 745) | CAAAACTCCTGTCCCCAGCACTGAGCATGGCCTAAGCCCCATAGCAGCTGGCAAGGGACCCAAC |
| MRPS18A_NM_018135_r1_9 | /5Biosg/ATGGGACCTGTTTGGCCCATGAATTCAATTGACTCATTGGCCCCATCACAAGAGAT |
| (SEQ ID NO: 746) | CAGTGACTCAATGCTCAGCACCCAGCTGGCAATGTGCCCAGGACCCCCTGCACTTCCCAAGCAG |
| MRPS18A_NM_018135_r1_10 | /5Biosg/TGCCCCTTTGAGGAACGCAGTTTCTTGGATTCACACGAGAGCGGCAAGTGTGTCAG |
| (SEQ ID NO: 747) | GCAGCCCACCTTGGCTCCCTGCAGCTCTCCCACAGTAAGGAGCTCACAGCTGTCATGAAGAGGA |
| RPS27A_NM_002954_r1_1 | /5Biosg/TCTCACTTTAGCCTTCGGAATGCTTTCGAAACTGTAGGTTTAAGCAACGTAAAAGA |
| (SEQ ID NO: 748) | TGTTTTAAAAAGGCTTTTCCCGTACAATCAACGTGGCTAAGAGAATTTTTAAAAGGAGGTCGAG |
| RPS27A_NM_002954_r1_2 | /5Biosg/GAAGGACCCACCCCTTAGGCGACCACACCAAATGCCTGACTGCCGTCCCAGCGACA |
| (SEQ ID NO: 749) | ATCCAACCCGGTGCCAGACTAGGACGTGAACTTCGTTTCTCCTCTCCCTAGCCTGGGCTTATTT |
| RPS27A_NM_002954_r1_3 | /5Biosg/GGTCTTCCCCGTAAGGGTTTTCACGAAAATCTGCATTTTGGTGGCGGCTCCACCGC |
| (SEQ ID NO: 750) | AGATGGCGGATCGAAAAGGAAGAACGCCGCGCAGGCCCACGGGGTTTCCCGGAGCTCCCGCCGA |
| RPS27A_NM_002954_r1_4 | /5Biosg/CTTCCAGCTGCTTGCCAGCAAAGATCAGTCTCTGCTGATCAGGAGGAATTCCTTCC |
| (SEQ ID NO: 751) | TTATCCTGGATCTTGGCCTTTACATTTTCTATCGTATCCGAGGGTTCAACCTCGAGGGTGATGG |
| RPS27A_NM_002954_r1_5 | /5Biosg/ATTCTTCTTGGGAGTGGTGTAAGACTTCTTCTTCCTTTTCTTAGCACCACCACGAA |
| (SEQ ID NO: 752) | GTCTCAACACAAGATGAAGAGTAGACTCCTTTTGAATATTGTAGTCAGACAAAGTACGTCCATC |
| RPS27A_NM_002954_r1_6 | /5Biosg/AACACCCCAGCACCACATTCATCAGAAGGGCACTCTCGACGAAGGCGACTAATTTT |
| (SEQ ID NO: 753) | GCCATTCTCATCCACCTTATAATATTTCAGGACAGCCAGCTTAACCTTCTTTCTCTTGTGCTTA |
| RPS27A_NM_002954_r1_7 | /5Biosg/CAATAAATGTTAGTTCATGTCTTTTATTAACTCATACAGTTACTTGTCTTCTGGTT |
| (SEQ ID NO: 754) | TGTTGAAACAGTAAGTCAGACAACATTTGCCACAATAATGTCTGTCAAAGTGACTTGCCATAAA |
| RPS27A_NM_002954_r1_8 | /5Biosg/TCACATAATCCCCAGAGGCAACATTCACACAGCGATAGCAGTAGCACTACTAAAAG |
| (SEQ ID NO: 755) | GAAATGGTGTGACCATCAATTTGGTGCTTAAAAACCATTCTTTTTACTGCAATAAAACCCAACA |
| RPS27A_NM_002954_r1_9 | /5Biosg/TTATATTTAGACAACTTACATTAAATATAACTTCTGCCAAAGTATAGGTAGTCTAA |
| (SEQ ID NO: 756) | GAATTGAAAATATCAAGACCTTTACAAGTGGTTGGCACCTGGCAGGTATACAGAACCACTGGGT |
| RPL10A_NM_007104_r1_1 | /5Biosg/AGGAACTTGCGGCGCTTGCGCTGGTTCCCGTGCAGGACTTCCCGCACCGCCTCGTA |
| (SEQ ID NO: 757) | CAGGGTGTCGCGAGAGACTTTGCTGCTCATGGCTTCTCACGCCGCGCTAACCGGAAAAGAGACT |
| RPL10A_NM_007104_r1_2 | /5Biosg/CCCAGGACACACACAGAGAACTTAGGGCGGGGAGTGGACTTAAGCCTGACGGTGCC |
| (SEQ ID NO: 758) | CGAGAAGCGCTTGTCCTTCTGGGGATCATAGTTCTTCAAGCTGATCTGCAACTCCACCGTCTCC |
| RPL10A_NM_007104_r1_3 | /5Biosg/GCATCATACTTCTTGGCCAGCTTCTTGACCAGTTTTTTATTCTTGTTGAGTTTTTT |
| (SEQ ID NO: 759) | CAGCGCCTCGATGTCCATGTGGGGGATATCCACGGCCTTAGCCTCGTCACAGTGCTGCTGGTCC |
| RPL10A_NM_007104_r1_4 | /5Biosg/CCACTTTGGCCACCATGTTTTCGTTGTGTGTGAGCAGGGAAGGGAACTTTCCTGCC |
| (SEQ ID NO: 760) | TTATTTAAACCTGGGCCGAGGATTCGTGGAATCTGCTTGATCAGAGACTCTGAGGCCAAAAACG |
| RPL10A_NM_007104_r1_5 | /5Biosg/AGTTGACAGCCAGGTGAATGTTATACACAAGCTCATCGTCTGTCATCTTCACGTGA |
| (SEQ ID NO: 761) | CCAACAGCTACAGCCAGACATAACACCTTCTTCATTTGGAACTTGATTGTGGACTTCACCTCAT |
| RPL10A_NM_007104_r1_6 | /5Biosg/GAACTGGTAATAGAATTTATTCAAATGTGCCTTAATATAGGCGCTGGGGCTTGCCC |
| (SEQ ID NO: 762) | ATGGTGCTCTTGATATATAAGGCCCGGACATTCTGCCAGTTTTTCTTGAGCAATGACACCAAGA |
| RPL10_NM_006013_r1_1 | /5Biosg/ATAGGCGGTCTGTCTGCGCGCGTAAGAACCATAGAGTCCTGTCGAAGACGGGTGGA |
| (SEQ ID NO: 763) | CGCTCTGTCAGACGTCATGACCATAGACGCAGCCGCTCTTGGCGCTTGCGCCCGGGCGTAGCCC |
| RPL10_NM_006013_r1_2 | /5Biosg/CGGCTTGTTCTTACAATACCGGTAACAACGGGCGGGGCGGCGGCCCATGGCGACAC |
| (SEQ ID NO: 764) | CAGGATCTTCAGTGGCACACCGAAGGGAAAGAGGCGCTCCTCCGCCTGCGCATGGCTTATATAG |
| RPL10_NM_006013_r1_3 | /5Biosg/TCTGACACCATGTGGCCACAAAGCGGAAACTCATCCACTTTTGCCTTTTTCCGCCC |
| (SEQ ID NO: 765) | CAGGTCAAAAATGCGAATCTTGGCATCAGGGACACCTCGGCAGAAGCGAGACTTTGGGTACGGC |
| RPL10_NM_006013_r1_4 | /5Biosg/GGGGTGGAGCCGCACCCGGATATGGAAGCCATCTTTGCCACAACTTTTTACCATGT |
| (SEQ ID NO: 766) | ACTTATTGGCACAAATTCGGGCAGCCTCCAGGGCTTCAGAGGACAGCTGCTCATATTCATCTGA |
| RPL10_NM_006013_r1_5 | /5Biosg/CCAATGTGAACCCTGGCCACAGTGCCCTGGGGCTTTCCAAAGGCACCTCGCATGCC |
| (SEQ ID NO: 767) | TGTTTGGAGCCTGTCAGCCCCAGCACAGGACAACATCTTGTTGATGCGGATGACGTGGAAGGGG |
| RPL10_NM_006013_r1_6 | /5Biosg/ACTTCTTTGAGATGTGGATCTTCTGGCGGCCAGGAAACTTGAACTTGGCCCTGCGC |
| (SEQ ID NO: 768) | AGGGCCTCAATCACATGCTCCTTGTTCTGCAGCTTGGTGCGGATGGACATGATAACTTGGCCAA |
| RPL10_NM_006013_r1_7 | /5Biosg/CCACTTGTCCAGAGGGCCACGATTGGGGATGTACTTGACCCCACAGCCATCTGGGA |
| (SEQ ID NO: 769) | TGAGCCGCTTTTCAGCCACCATGTCTTCAAATTCATCAGCATTGAACTTGGTGAAGCCCCACTT |
| RPL10_NM_006013_r1_8 | /5Biosg/GTTCCTTCCCCGTCAAGAATGTAGATACAAAGACATAGGTGGACAGGAAGTAGAAT |
| (SEQ ID NO: 770) | TTATTGGTGAGTATTAAGAGGGGGGCAGCACATTGGAAGCCCTCATGAGTGCAGGGCCCGCCAC |
| RPL10_NM_006013_r1_9 | /5Biosg/TGACATAAGGCCACCAGTCCCAGCCCTTTGCAGGGTTTGCTGCCTCATGAGCAGGG |
| (SEQ ID NO: 771) | CTGAGTTGTCAACCTGTTTCTGAAGTGAAAGGGCAATGACCCAAAGGTTCCCAGAGGAAGTTCC |
| RPL10_NM_006013_r1_10 | /5Biosg/CCAGCCTGGGATTGCAAGGCTTTGCTGCAGCTGTGCTTCATGAAAAGCACTTCAGT |
| (SEQ ID NO: 772) | GTTTTTCAACCTACTGCCTAGGACCTGGGGAGGTCCAAGTCAAGCTCCAGAGTAGACAACTGAC |
| RPL10_NM_006013_r1_11 | /5Biosg/AGCCCAATGTGATGCACACCTACCCTTGCTAAATAAACGCTGCTGGTCAGCCTCTA |
| (SEQ ID NO: 773) | GCCCACAAGCCCCTCTTGGTCCGCATGTGTTGTAATAAGCAACACAACTGTAGGCTGACCCCAG |
| RPL10_NM_006013_r1_12 | /5Biosg/GGCATGGGGCCCAGGAGGTTGCTAGAGATAGGAAATGGGGCAAATCGCAGGTGACC |
| (SEQ ID NO: 774) | TCAGCAGTACCTGGATGATTCCCACCAAGGAGGAATGGCCAAACCAGATGGGTGAGAACAAGCC |
| RPL10_NM_006013_r1_13 | /5Biosg/AAACCATGCCACACACGTGTCTGTTCCGAGGTCTTGTTACAAATGACCAAAGGTTA |
| (SEQ ID NO: 775) | GCCTAGACCTTTGGTCAGGTGGTGAAGGCCCTGGGAATGCAGGTTCTAGAAGGGGTGGGGGCAT |
| RPL10_NM_006013_r1_14 | /5Biosg/TCCCTGGATACAGATGCTCAGGCCACCCACCTAGCACTCCTCTCATTTTTGCTGCT |
| (SEQ ID NO: 776) | CCCCCAAAAGCCAGCGCTGTTCTACAATAGTTCAGGTCAGACATCTAAGATCCCCAGGCCAAAC |
| RPL10_NM_006013_r1_15 | /5Biosg/CCTAAGAAATGGCTGTAAACTGAGATTACAAAAAACCAGAGGCCTTTTGGGAGCCA |
| (SEQ ID NO: 777) | CAGGCTGGGGGCTGGGGCCAACATACCCCAGCTCTGGGACCAAAAGCCTTTGGAGTCCTGTCCC |
| RPL10_NM_006013_r1_16 | /5Biosg/GGGTCATGTGTTAACTATAATCACATTTATGGTTTGGAACCATCACCCCAAGGTAA |
| (SEQ ID NO: 778) | AAAAAAAATAAAAGGTATTCCCAGGTATGTTTGGCAAAATAAAATAAAGGTAATTAAAAACCTA |
| RPL10_NM_006013_r1_17 | /5Biosg/GTCCCGGAAAGGGGCTGCAGGACACAGCAAAATGCCAGCAGCGTGCCGGACCCCTC |
| (SEQ ID NO: 779) | CCTTCCATCCTCCTCTCCAAAGAACAGAGGTCAGGAAAAACACTGGCTGGGACGCTAGAAGGGT |
| RPL10_NM_006013_r1_18 | /5Biosg/GTTGGTACAGGCTTCTGCATGCCTGGTAAAGTCCAGCCAAGGCTGGTCAAGGCAAC |
| (SEQ ID NO: 780) | ATCTCCACACAGAAAATCTGCACCAGTTATGTAAGCTAAAAAGCTGTGTGAACCCAGGTGTCCC |
| RPL10_NM_006013_r1_19 | /5Biosg/GAAACCAGCAAACACCGGGAAACATTTTGGCTCATTATAATCCGGTGAACAATGCA |
| (SEQ ID NO: 781) | GTCAGGCCTGTTATAACCGCTGAGCAGCCACACTCGCACCTCCTGGGTGCTGTAGTCTGTGTTG |
| RPL10_NM_006013_r1_20 | /5Biosg/TTGCAGATATGCCAATGGGCCATGTTTATTGTGGATTGGTAAGAATCACCAGGAAA |
| (SEQ ID NO: 782) | CCATTAAGCCCCAATAGCTACAAGGAGGGTGGTTAATCTGCTATATCAAACTCCTTCCCTGAAA |
| RPL11_NM_000575_r1_1 | /5Biosg/ACAGATGTTGAGACAGAGTTTGCGGATGCGAAGTTCCCGCATGGGGTTCTCCTTTT |
| (SEQ ID NO: 783) | CACCTTGATCCTGCGCCATGATGGAGAGCAGGAAGAGAAAGCGGAGCTTCCGGCCGAGGGCCTT |
| RPL11_NM_000975_r1_2 | /5Biosg/AGGATCTGACAGTGTATCTAGCTTTGGAAAACACAGGGGTCTGCCCTGTGAGCTGC |
| (SEQ ID NO: 784) | TCCAACACCTTGGCTGCTCGCGTCAGTCTGTCTCCACTCTCCCCAACACAGATGTTGAGACAGA |
| RPL11_NM_000975_r1_3 | /5Biosg/TAACTCATACTCCCGCACCTTTAGACCCTTCTCCAAGATTTCTTCTGCCTTGGCCC |
| (SEQ ID NO: 785) | CTCGAACTGTGCAGTGGACAGCAATCTTTTCATTTCTCCGGATGCCAAAGGATCTGACAGTGTA |
| RPL11_NM_000975_r1_4 | /5Biosg/CCAGGCCGTAGATACCAATGCTTGGGTCATATTTGATACCCAGATCGATGTGTTCC |
| (SEQ ID NO: 786) | TGGATCCCAAAACCAAAGTTTCCAGTATCTGAGAAGTTGTTTTTTCTTAACTCATACTCCCGCA |
| RPL11_NM_000975_r1_5 | /5Biosg/CATGGCCTCCTCTTTGCTGATTCTGTGTTTGGCCCCAATGCAGCCTGTCCTGCGCT |
| (SEQ ID NO: 787) | TCTTGTCTGCGATGCTGAAACCTGGCCTACCCAGCACCACATAGAAGTCCAGGCCGTAGATACC |
| RPL11_NM_000975_r1_6 | /5Biosg/TTTTTGCACATTTCACTGAAAACTTTTTATTGCTCTTTTGGATAGAAACGGGAATT |
| (SEQ ID NO: 788) | TATTTGCCAGGAAGGATGATCCCATCATACTTCTGCTGGAACCAGCGCATGGCCTCCTCTTTGC |
| RPL12_NM_000976_r1_1 | /5Biosg/GGCATGGTGGAGGCGGCTGGTGTCGGATGAACCCGGATTCGGGACGACCGAAGGAA |
| (SEQ ID NO: 789) | GTTGCACCTTGGCCTCCTCCGAGCCGAAAGCCGAGAGGCCGGAAATCGCGCGGACAAGCCAGAT |
| RPL12_NM_000976_r1_2 | /5Biosg/GACAGACCCAGGGGGCCGATCTTGGGGGCCAGGGCAGAAGTGGCACCGACTTCACC |
| (SEQ ID NO: 790) | TCCGGTGCACCTCAGGTATACGACTTTGATCTCGTTGGGGTCGAACTTCGGCGGCATGGTGGAG |
| RPL12_NM_000976_r1_3 | /5Biosg/ACCACCTCAATCTGGGCCTGTCTGTTCTGAATGGTCAGTTTCACTGTAATCCTCAG |
| (SEQ ID NO: 791) | GCCCTTCCAGTCACCCGTTGCCTTGGCAATGTCATCACCAACTTTTTTTGGAGACAGACCCAGG |
| RPL12_NM_000976_r1_4 | /5Biosg/ACAATCTCATCAAAAGTGATATTCCCACTGTGTTTAATGTTTTTCTGTTTCTTTCT |
| (SEQ ID NO: 792) | GTCTCTTGGTGGTTCCTTGAGGGCTTTGATGATCAGGGCAGAGGCAGAAGGCACCACCTCAATC |
| RPL12_NM_000976_r1_5 | /5Biosg/CGGCCATCAACATTACAGCCCACTGACTGGGCAGTCCCCAGGATCTCTTTAATGGT |
| (SEQ ID NO: 793) | TCCAGAGAGTTCTCTGGCTAAGGATCGGTGCCGCATCTGTCGAGCAATGTTGACAATCTCATCA |
| RPL12_NM_000976_r1_6 | /5Biosg/TCCACCAGTTGTCAAATGATCCTTTATTGAAATGTTTTCCTTTGTGCTTAACTGGC |
| (SEQ ID NO: 794) | TGGGCATTCCACAGCACCACTGTTGATGTCATCGATGATGTCATGAGGATGGCGGCCATCAACA |
| RPL13A_NM_012423_r1_1 | /5Biosg/AGATGGCCTCGACCATCAAGCACCAGGACCTGCACCTCCGCCATCTTCGGCAGCCG |
| (SEQ ID NO: 795) | CTTGGAAAAGGAGGAGGTTTTGTCGCAGGGTTTCTTATCCCTTGAAACAGGGGCGGCAGAAGTG |
| RPL13A_NM_012423_r1_2 | /5Biosg/GTACTTCAACTTGTTTCTGTAGAAATTGCCAGAAATGTTGATGCCTTCACAGCGTA |
| (SEQ ID NO: 796) | CGACCACCACCTTCCGGCCCAGCAGTACCTGTTTAGCCACGATGGCCGCCAGGCGGCCCAGGAG |
| RPL13A_NM_012423_r1_3 | /5Biosg/GCTTGGTTTTGTGGGGCAGCATACCTCGCACGGTCCGCCAGAAGATGCGGCTGGGG |
| (SEQ ID NO: 797) | GCCCGGAAGTGGTAGGGGCCTCGGGAAGGGTTGGTGTTCATCCGCTTGCGGAGGAAAGCCAGGT |
| RPL13A_NM_012423_r1_4 | /5Biosg/TTGTAGGCTTCAGACGCACGACCTTGAGGGCAGCAGGAACCACCATCCGCTTTTTC |
| (SEQ ID NO: 798) | TTGTCGTAGGGCGGTGGGATGCCGTCAAACACCTTGAGACGGTCCAGAGCGGCCTGGCCTCGCT |
| RPL13A_NM_012423_r1_5 | /5Biosg/TTCTTCTTCCGGTAGTGGATCTTGGCTTTCTCTTTCCTCTTCTCCTCCAGGGTGGC |
| (SEQ ID NO: 799) | TGTCACTGCCTGGTACTTCCAGCCAACCTCGTGAGCCAGGCGCCCCAGATAGGCAAACTTTCTT |
| RPL13A_NM_012423_r1_6 | /5Biosg/AATTAACAGTCTTTATTGGGCTCAGACCAGGAGTCCGTGGGTCTTGAGGACCTCTG |
| (SEQ ID NO: 800) | TGTATTTGTCAATTTTCTTCTCCACGTTCTTCTCGGCCTGTTTCCGTAGCCTCATGAGCTGTTT |
| RPL13A_NM_012423_r1_7 | /5Biosg/CTTTCCTTGCTCCCAGCTTCCTATGTCCCAGGGCTGCCTGTGGCACCTGGACTGCT |
| (SEQ ID NO: 801) | GCTGCCCCTGGGTCCCGTACATTCCAGGGCAACAATGGAGGAAGGGCAGGCAACGCATGAGGAA |
| RPL13A_NM_012423_r1_8 | /5Biosg/TTCTGGCTCCCTTTCACTCATAGTAACTGGTTGCTCTTCCTATTGGTCATAGATAA |
| (SEQ ID NO: 802) | ATGACACCTGCACAATTCTCCGAGTGCTTTCAAGCAACTTCGGGAGGCAGTGACTAAGACCCTT |
| RPL13A_NM_012423_r1_9 | /5Biosg/ACAACAGCAAGCTCATGCTGTCCCCAAGTGCTTGCACATTCTAACAGCTGCAGAGT |
| (SEQ ID NO: 803) | ATATGACCAGGTGGAAAGTCCAACAGATGCCCCACTCACAAGATAGGGCCCTCCAATCAGTCTT |
| RPL13A_NM_012423_r1_10 | /5Biosg/TTCTTTAAAAGCAATTTATTTCTGATTACAAAATACAGGTGAGGAGCATGGGCGAT |
| (SEQ ID NO: 804) | GCCTGTAACCCCTTGGTTGTGCATCTTCCCAGTCTATTTCTGCTTCTAGAAATACCCTGTGTAC |
| RPL13_NM_033251_r1_1 | /5Biosg/GGGCAGCGGCCGAACGCTGTCTGCGAAGAAGAAGGGGCCCATCCACTCCCGCGTTG |
| (SEQ ID NO: 805) | GCTGAAGCGCCAGGCCGATGGCGCTGGATGATGCCCAAAGGCCAGGACCCAGTCTCCCTCACCT |
| RPL13_NM_033251_r1_2 | /5Biosg/CGGCCCTGCGGCTGCGGACGAGAGGGGAGCGAGTGAGAGGCCGGACCCCGGGCGCG |
| (SEQ ID NO: 806) | CGCACCCCCCCGGGCCTCCGCCCCGAACCCTCCAGACTGGGCCGTCCGGCCGCGCCTAGAGCCC |
| RPL13_NM_033251_r1_3 | /5Biosg/CTTACGTCTGCGGATCTTACGGGCCGGCTGGTTGAACCACGTGGCCACGCGCCGCT |
| (SEQ ID NO: 807) | GCCAGTCCTTGTGGAAGTGGGGCTTCAAGACCATGCCATTCCGGCTGGGCGCCATGGCTGCCTA |
| RPL13_NM_033251_r1_4 | /5Biosg/CTGAAGCCGCGGCCGGCGCGCACCTTCGTGTGGTACCGAACCGTGGGGCAGCGCAC |
| (SEQ ID NO: 808) | GATGGGCCGGATGGGACCCGACGCGGGGCGCGGGGCGATGCGGCGCGCCTTGGCTTGCCGGGCC |
| RPL13_NM_033251_r1_5 | /5Biosg/AGCCGCTGCACGTTGGCCTGCAGGGACTCCGTGGACTTGTTCCGCCTCCTCGGATC |
| (SEQ ID NO: 809) | CACAGAAATGCCGATGGTCCGGGCCACCTTCTTGTGAATGCCGGCCACCCTGAGCTCCTCCAGG |
| RPL13_NM_033251_r1_6 | /5Biosg/ACGGGCATGACCGGTCCGGTCAGCTGGGTGGCCAGTTTCAGTTCTTCAGCAGAACT |
| (SEQ ID NO: 810) | GTCTCCCTTCTTGGGGGCCGAGGGCTTCCTGGGGAAGAGGATGAGTTTGGAGCGGTACTCCTTC |
| RPL13_NM_033251_r1_7 | /5Biosg/TTTGCCCGTATGCCGAAGAGCCGGGCGTTGGCACGGGCCATACGGAGACTAGCGAA |
| (SEQ ID NO: 811) | GGCTTTGAAATTCTTCTCTTCCTCAGTGATGACTCGAGCTTTCTCCTTCTTATAGACGTTCCGG |
| RPL13_NM_033251_r1_8 | /5Biosg/CAGTTGTTCCCACGAAACACACCACGTGGAGACCCAGCATGACTGCCGACTGATTC |
| (SEQ ID NO: 812) | CAAGTCCCCAGGAGGGCTTTATTTTTTCTTTTCAACATCCTGTTCTGCGGCTTCCTTGGCTCTT |
| RPL13_NM_033251_r1_9 | /5Biosg/CAACCAACAGTGCTTCAACACAGAAAGTAAAGCATTATCCAGGGCTTGGACTGTCT |
| (SEQ ID NO: 813) | TTCAAGAAAGCCCCAAATCCCCTGGCAGGAGGAAGTCACAGCAGTGAAGCCCCATCCCAGGCCC |
| RPL13_NM_033251_r1_10 | /5Biosg/TACAGCTGTGAGCACCAGCCCAACCTTGCCTCTTTAAAAAGAAAAAACACAAGTCC |
| (SEQ ID NO: 814) | ACTCTGAAGTCAGCCTCTGTAACCTCCCCACAAGAAAACCGTTTTACATCAGTCACTAACCAAA |
| RPL13_NM_033251_r1_11 | /5Biosg/CCCAGGAGCTGCAACTACAAGTGTGCAGCACCAAGCATGGCTGATTTCTTTATTTT |
| (SEQ ID NO: 815) | TAGTAGTTCTGACATGTTGGCCAGGCTGGTCTTGAACTCCCAGCCAACCTCAAAGTGCTGGGAT |
| RPL13_NM_033251_r1_12 | /5Biosg/CCCTGTCTCTTTTTTTTTTTTTGAGACAGTCTCACTGTTGCCCAGGCTAGAGTGCA |
| (SEQ ID NO: 816) | GTGGCGTGATCCTGGCTCAGTGCAGCCTCTGCCTCCTGGGTTAAGTGATCCCTCACCTCTGCCT |
| RPL13_NM_033251_r1_13 | /5Biosg/GGTCTCGTGCCTATAAACCCCTCTACTTGGGAGGCTGAGGGAGGACTGCTTGAGGC |
| (SEQ ID NO: 817) | CATGAGTTCAAGACTGCAGTGAGCCATGATTGTGGCACTGTACTCCAGCCTGGGTGCCGAAGAC |
| RPL13_NM_033251_r1_14 | /5Biosg/AGAGCAAGAGGACCATGGTTACCGGGCACAACAAGCTTAAGAATGACGCCCCAAAG |
| (SEQ ID NO: 818) | TCCAAGATGCTACATTAACTAGAAACTGCTTTGCTTAAAAAAGGCAACTCTAGGTTGGGTGCAG |
| RPL13_NM_033251_r1_15 | /5Biosg/CCATGAAGCAAGAACAATGGCTTTAGAATGTTTTTGTTTAACAGGAGAGGCCGACA |
| (SEQ ID NO: 819) | CTTGGCAGGGCCATCTCATACCTTGGTGTCTGGGTGAAGAAGCCCATTGAAGGAAGGGTTAATC |
| RPL13_NM_033251_r1_16 | /5Biosg/GGACTAGGGCAGGCTGTGTCTGAGCACTGCACGAGAAACCATGCTGCCCTCGGACA |
| (SEQ ID NO: 820) | CAGAAGACATGGGGATGCCAGCTCAGACCAGGGCACCCTGGCACTCTCTCTGGCTGCCTCTTGT |
| RPL13_NM_033251_r1_17 | /5Biosg/TACCGGCACCCGGCAGAGGCATGTAGACAAAACAGAATCTGACTCGGTCCCTGAAG |
| (SEQ ID NO: 821) | CTGCTTCTCTGGGGTTGTCATGGCCATAGCTGCCAAGGAGAGCAGGTGCTGCTGTGAGCTGGTA |
| RPL13_NM_033251_r1_18 | /5Biosg/GTTTAGATGTTTCCAAACAAGCAAGGTGTCATCGTGTATCAAGTGTGGCTGTGAGT |
| (SEQ ID NO: 822) | GCAGCAGCAGAGGTAGCAGCATCACCTATTTCCACGCCAGTAACAGCTCCCTGGGTGCCTCAAT |
| RPL13_NM_033251_r1_19 | /5Biosg/CCGGCCCACCTTAACTTTACATAGGACAAGAGTCCCAAACATACAAACTGATCACG |
| (SEQ ID NO: 823) | AAGTTAACATATACACCTCAGACAGGAAACTGGTAGCCAACAGCCTGCAAGTCATCTACTAGAT |
| RPL13_NM_033251_r1_20 | /5Biosg/ACAGGCGTGTGTCACCACTCAGCTAATTTTTGTATTTTCAGTAGAGATGAGGTTTC |
| (SEQ ID NO: 824) | ACCATCAGGTGATCCACCCGCCTCGGCCTCCCAGTGTTAGGATTACAGGCGTGAGCCACTGCAC |
| RPL13_NM_033251_r1_21 | /5Biosg/ACCTTGTACAACTTTTTTTGAGACTGAGTCTCGCTGTCACCCAGGCTGGAGCACAG |
| (SEQ ID NO: 825) | TGGTGCAGCTCGGCTCACTGCAATCTCTGCCTCCTGGGTTCAAGCCTACCAAGTAGCTGGGATT |
| RPL13_NM_033251_r1_22 | /5Biosg/AGCGCTGGAGACCCCTGCACCCCCCACGGCCAGTCTTCAGCCAGGTTGGAATCCAC |
| (SEQ ID NO: 826) | CAGAAATCTTCAGGCCCTCTCACGTGAGAGGGATTCGAGCCTAGGCTCAAGCTTCCAACCATCC |
| RPL13_NM_033251_r1_23 | /5Biosg/CCTCTGCTCCATTCACGTCACCACATGGCATTACCTACTCCAGGTGCGGCCCCGCA |
| (SEQ ID NO: 827) | GGCAGCAGGAAAGGCCCTTCCCCTAGTGCGGTGTGGGCAGGGAGGAAGCAGGCTGGAGGGCAGA |
| RPL13_NM_033251_r1_24 | /5Biosg/AGATGATACCTGAACCCTGACTTCCCGTCAAGTCGGCTGGTAGGAGGGCCCCCGGA |
| (SEQ ID NO: 828) | ACACGCAGATCAGAGTGCTTTTGTCCCACAGTAAAAACAGCAAGGCGGTGTGATGGGGCACAGA |
| RPL13_NM_033251_r1_25 | /5Biosg/TAGATTAAGCAAACGGAGAGAACGTAAGGTTAGCAACACAGCCCTGGAGACTGTGA |
| (SEQ ID NO: 829) | CTTTCAGTGAAATGAGGAGGGGACTCTGGCAGGTTCAGTGCAGACTACCCCGCCCCAGGTGCAC |
| RPL13_NM_033251_r1_26 | /5Biosg/CTCACCAAACTCACAATGTCCCAGCGCAACCTAGAAAAATGACCTCTTGCTAAGAG |
| (SEQ ID NO: 830) | CACCGGCCAACAGCAGGGCCCCAGACAACAGAGAAAGCCCATCCTCTCCTGTTAGGGCTCTTAA |
| RPL13_NM_033251_r1_27 | /5Biosg/GCAGAAGCCTTCCTCAGCCCCAGGAAGGGCAGAGCCGCACTAGCTGCATTTCAGGT |
| (SEQ ID NO: 831) | AGGGGAGCAGCAGGCAGCAGGCAGCCCGGCCCAGCCCAGCCCAGCCCAGCCCACATCCATGACC |
| RPL13_NM_033251_r1_28 | /5Biosg/AGAGTCTTTCTTGGAGCAGACAGGCCCGTACCTGGATCAGGAGATCTTGCTCCAGC |
| (SEQ ID NO: 832) | TACAAGGACTGAGTGTCAGCAGGGAGGCTGCCTCACCCACCTGCCCAGCCCCCCAGCTATCCTG |
| RPL13_NM_033251_r1_29 | /5Biosg/GGGCACGGCCCACACTCAGGCACTTGTCCCCACCAGCGGGGTATCCTGGGCCGACA |
| (SEQ ID NO: 833) | GGTGCTGAATCAGGCGGTCCCCAGGGACTAAGTTAAAACAAAGGGCCCCTTTGCATCTGGTGGC |
| RPL13_NM_033251_r1_30 | /5Biosg/CCTGGCACAGAGGGCTGGTTCCAGCCCGGGTTCCACAGCGCCCCCTAGGTCAGGCC |
| (SEQ ID NO: 834) | CACACATCACAACTGAAAAGCCACTTCCTAGTCAGGACGGCCCCACTCAGGGATGGCCACACTC |
| RPL13_NM0333251_r1_31 | /5Biosg/ACGAAATCAAGGTGTTCCCAGAGCCGCTTTGCCTGAGGTGCGGTGGGGGCCCTTCC |
| (SEQ ID NO: 835) | TCTCAGCTCCCAGTGACCCACGCCATGGCAGCTGCCCCTCAGGGCCGGCGGAACCTGTCTGCGG |
| RPL13_NM_033251_r1_32 | /5Biosg/TGAGGAGCCTGGGCGCACCTGCACTTCCTCCACCGGGCAGCAGACCACAAGCTCGT |
| (SEQ ID NO: 836) | GGCACACAGGTCAGTTTCTCAGGCTTCACAGCTTGGCTGCCCTCCCTGGGACGGCTCACATGGA |
| RPL13_NM_033251_r1_33 | /5Biosg/TGCCGTAAAACCAACGCTTCTACAAAAGGCCACGGGCACCACAGAGCCAGCCCGTG |
| (SEQ ID NO: 837) | CTCCCCGGGGGCGACAGGCTGAATAGGAAATGCTCACACCCCGAAGGAATCCTGCAAAACGGAA |
| RPL13_NM_033251_r1_34 | /5Biosg/GCCTCCTGAGACTATCCCCGATCAGTTCTGCAGGCGCCGGAGCGCGCCTGGCCCCA |
| (SEQ ID NO: 838) | TGACCCCCGTGGGCACTGCTGGCAAAGCCCCACCCACTGTGGGAGGCTGCCCCAGAGATGAACC |
| RPL13_NM_033251_r1_35 | /5Biosg/GTGAGAAACGGTGCATCACCGAGGCAGGCGACTCGTGCCTCCTGAGACTATCCCCG |
| (SEQ ID NO: 839) | ATCACCGGGGCAGGAGACTAGCGCCTTCTGAGACTATCCCCGATCACCGGGGCACGTGACTAGC |
| RPL13_NM_033251_r1_36 | /5Biosg/AACTGAAATTCCTAATGTCTAGCCAAAAGAAAAAGCAAAATAAACAATACATGGAG |
| (SEQ ID NO: 840) | GAAAATTGTGTGCAGCTCCTATCCGCACACGTCTTCCCCTTTAGCTCGGGCGAGAGCAGCCGGT |
| RPL13_NM_033251_r1_37 | /5Biosg/TGGGCTCATGCCATCCTCCCCCCTCAGCCTCCCAAAGTTTTGAGACTATAGGCGTG |
| (SEQ ID NO: 841) | AGCGACCTCACCCAGCCCTGCATGATAATTTTAAAATAGAAAAGGAAAAATACAACTTGGGAAA |
| RPL13_NM_033251_r1_38 | /5Biosg/TCCAGGGTCGAAACTACTGCATAATAGTTTTGTTTTTTTTTTTCCTGAGATAGGGT |
| (SEQ ID NO: 842) | CTCGCTCTGTCATGCAGGCTGGAGGACAGTGGAGCGATCTCGGCTCACTGCAGCCTTAAACTCC |
| RPL13_NM_033251_r1_39 | /5Biosg/AAATGATTGGCAATTACATATATTTTTAACAAAGCAACTACAGGATAACCAGATTC |
| (SEQ ID NO: 843) | CAAATGAAACTACGCCTGGATGGGCGAGGTACACGTGTTACAACTCAAAGATGCACACTCGTCT |
| NM_003973_RPL14_r1_1 | /5Biosg/AAAGGAGACATAGGCCACCCGGCCAACCTCCACGAAGCGCCTGAACACCATGTTGG |
| (SEQ ID NO: 844) | CGGCGTTAGGCGAGAAGGAAGAAGACCCGCCCAACCCTGCGCATGTGTAGAAGAACGCACCGCC |
| NM_003973_RPL14_r1_2 | /5Biosg/TGAAAGGCATGGCCTGTCTCCTCACTTGAGTGCAAGGTCCATCGACCAAAGCCCTG |
| (SEQ ID NO: 845) | TTCTGATCAATAACATCTACAATCGCGACCAATTTTCCGGCATGAGGTCCAAAGGAGACATAGG |
| NM_003973_RPL14_r1_3 | /5Biosg/CTGCCCATTTTGTATTGATGTCTGCCTTCTGCCAGGCTTGTCGGACATACTTCTGG |
| (SEQ ID NO: 846) | TGGGCACTGTGCGGAAACTTGAGGATGAAATCAGTGAGCTGCATGCACTTGAAAGGCATGGCCT |
| NM_003973_RPL14_r1_4 | /5Biosg/ATTATTCTGTTCCTCATTTTCTTTGCCTTCATAACTTTAAAACGATCAAAATCTGT |
| (SEQ ID NO: 847) | CATCTTGGCTTTCCTTTCTCTGGCTTCAATCTTCTTGGCCCATCGTGTGGCTGCCCATTTTGTA |
| NM_003973_RPL14_r1_5 | /5Biosg/AGCAGCAGCAGCAGCAGCAGCAGCAGCAGTACCCTTAGTACCAGGTGCTTTTTTGG |
| (SEQ ID NO: 848) | GAGAAGCTTTCAGGAGAGCTGCCTTTTGAAGCTTCTTAACTTCATTCTTGATTATTCTGTTCCT |
| NM_003973_RPL14_r1_6 | /5Biosg/CTTTTGGAGCAGGCGCTGCTTTCTGGCCTGTGGCTTTCTGGGCAGGAACCTTCTGG |
| (SEQ ID NO: 849) | GCTGGAGCCTTTTTACTCGCGGCGGTGATCTTTTTTGCTGGAACTTTAGCAGCAGCAGCAGCAG |
| NM_003973_RPL14_r1_7 | /5Biosg/GTCAAAAAGAACCTTTATTACTTTTTATGATTGCCACTTATGCTTTCTTGCCAGAT |
| (SEQ ID NO: 850) | GCCTTTGGAGCAGGTGCTTTCTGGGCTGGAGCTTTTTGACCCTTCTGAGCTTTTGGAGCAGGCG |
| NM_003973_RPL14_r1_8 | /5Biosg/TTTGAACTGATTATTTAATGTTTATTATAATCCTACTATCAATCTGCCTCCTAACT |
| (SEQ ID NO: 851) | CCAGCCTCAACAGGCTTTAAATCCAAAGGCTTAAATACATTTGTCAACAGGTCAAAAAGAACCT |
| RPL15_NM_002948_r1_1 | /5Biosg/TTGGCGGCGAGGTCTCGGCGGCTAAGCGAGCGTCGGCGACTGTCTCTCCGCGAGAG |
| (SEQ ID NO: 852) | GAGGCAAGTTGGGGTCCAGGCTCCAAAGCCGGTGGCCGCGTACCGCGGTGGAGCCGCTGTCTTT |
| RPL15_NM_002948_r1_2 | /5Biosg/GGCATCGCGCCTGGTGGCGGAGTTCTGCCGAGTGGGGCGCCGCGGCCGCTATTGTC |
| (SEQ ID NO: 853) | CCGCCCCCTGCTCCGCAAGATTCGAGCCTGAGCGGCCTGGGCGTCTCGAGAGGTGAGAGAGTTG |
| RPL15_NM_002948_r1_3 | /5Biosg/AGACGGAAAGGAAAGAGCTGTACTCCCACAATGCCTTTTGGGCTCCTCCCCCACCT |
| (SEQ ID NO: 854) | CAGCCACCCTCTTTCCGGTGGAAAGCTGCGGCCTCCCAGAGCGTCATGGGACATGTAGTTCCGG |
| RPL15_NM_002948_r1_4 | /5Biosg/CGGTACTGCCAGCAGCGGACCCTCAGAAGAAAGCGCATGACATCAGACTGCTTCTT |
| (SEQ ID NO: 855) | TCTCCATAGCTCCTGGATGTACTTGTATGCACCCATCTTGGCTTACCTGATGGCTGCCGCCAGA |
| RPL15_NM_002948_r1_5 | /5Biosg/CCACCACGGCGAACACGAATCCTATATATAACGTAACCTTGCTTGGCCTTGTAGCC |
| (SEQ ID NO: 856) | CAGTCGGGGCGCTTTATCAGGCCGGGTGGGGCGGGGAGCCCTGTGGAGAGCAGAGAGCTGGCGG |
| RPL15_NM_002948_r1_6 | /5Biosg/GTGGCGTCCAGCTCGCTCCTCTGCAACGGACTGAAGGCTTCGAGCAAACTTTAGCT |
| (SEQ ID NO: 857) | GGTTAACACCATGATGGACAGGCTTGCCGTAAGTTGCACCCTTAGGAACTGGGCGTTTTCGGCC |
| RPL15_NM_002948_r1_7 | /5Biosg/GGTGTCAGGATTTCTTCTGATAGCTTTATGGAATGGATCAATGAGGATAACCTCAA |
| (SEQ ID NO: 858) | AAAATTTGTATGTGGAATCTTCACCAACCCAGTAAGAATTCAGGACTCTCAGAGCCCCACAGTG |
| RPL15_NM_002948_r1_8 | /5Biosg/GAGAGCCACCAATAGTGTGGTGGAACTTGTGGCCCTTTCCAAGGCCACGGCTCTTT |
| (SEQ ID NO: 859) | CGGCCTGCAGATGTCAGCCCACGCATCTCCCTGTGCTTGTGGACTGGTTTGGTGATCCACTGGG |
| RPL15_NM_002948_r1_9 | /5Biosg/CTGTAAGCAGACATGACTGTCCTAAATTGTTTATTAAGTATGAATTTTACAAACTT |
| (SEQ ID NO: 860) | TACTTATATTAGCGGTAACGGTGGAGCTGGAGAGTATTGCGCCTTCTCCAAGCTGCCCGGCGAG |
| RPL15_NM_002948_r1_10 | /5Biosg/TTAGAAACAAGATACACCACCACTTATTGTCTTCAAACATTATTGCACTTTAACTT |
| (SEQ ID NO: 861) | TCTTAATTTGACAAAGCATTCAAGAAACATCTGCAGACTAGTTTTAACAGACAAATAACACCTG |
| RPL15_NM_002948_r1_11 | /5Biosg/TAGTTTCAGTTCTCTCCTTTTAAAGAATTTATTAAGCCTGTTATACCACACAGTAT |
| (SEQ ID NO: 862) | GTTTTATACACTGACATACAACTCCCTAATAAGATAAAGCAAAGACAAAAAAGTTTATCTTATT |
| RPL15_NM_002948_r1_12 | /5Biosg/GTCTACACCTGCCAAACTAAAAAAAACGCAGCCACTTGAAAATAAACCAGCAGAGC |
| (SEQ ID NO: 863) | ATTGCCATCACTCCGATAAAGCTGCAGGTTTCATCACATGCACCAGACAAATCTACAGGGCTAG |
| RPL15_NM_002948_r1_13 | /5Biosg/GAAGCCATGGCACTGAACCAGGAGCTTTCACCTTTTATGTGAATTTAAAATGGTAC |
| (SEQ ID NO: 864) | TGTGGAGGCTGAGGCAATTTTCTTCAGGCTAACCCAGATTTTCTAAAGCCCAACTTAAAAAGTC |
| RPL15_NM_002948_r1_14 | /5Biosg/CAGCTTCCCACCCTAGCATTCATTGCTAGCTTACCAATCTTAAGGCTAATAATTTT |
| (SEQ ID NO: 865) | ATCGCACATTCAACTTCAAAACACACCAGTGTTTACCATTACCACTAATCACTGAATGCCATGA |
| RPL15_NM_002948_r1_15 | /5Biosg/CCCCTAATGTGACTGAAGGGATTCATAATGATCACAATTAGCATTACGGTTAAGTA |
| (SEQ ID NO: 866) | TTTTAGGGTTGACGTCTAAGCTCACACTTGAAAGGTATTTATCTAATGGCCACTGGCTCACCAG |
| RPL15_NM_002948_r1_16 | /5Biosg/TGGAAACCATGCGCTTGTGACTTTTTCTGTAGCCTATGGGAGTGGACAGAGTGGGT |
| (SEQ ID NO: 867) | AACCCAAGATGTTTTTAAGACTGACTGGACTAAGAATGACGTACTTATAGCCAACTACTTTCCC |
| RPL15_NM_002948_r1_17 | /5Biosg/TGGTATACTCATAGTCAAAGTGGGTGCACAAGTGATGGTCCAACTTTGTGGAATGG |
| (SEQ ID NO: 868) | TAAGGATTTTTAGGGTTGTTTGGCCAGAACAAGAGAAATAACTGCAGAAAACACATATGGTTGG |
| RPL15_NM_002948_r1_18 | /5Biosg/AGTCAGAATTTAAAATTGGGCAATTCCTTGTCTACATTTTCTTTACACTCAAATTG |
| (SEQ ID NO: 869) | ACCCTGGACAACAGTAGTCTAGTCTCCTGGAAGACATGGTGCAAACAGAAATGCAATGTGGTGG |
| RPL15_NM_002948_r1_19 | /5Biosg/GAACTTTTATGAACTTGCTATGAGTACTTCCTGGAAATCAATTAACTGAGTCTTTT |
| (SEQ ID NO: 870) | GAAACCCCTAGAGAAGATAGGAGAAAATTGGTTCAGAACGAGCATTTAAATTAAGTCAGCAAAG |
| RPL15_NM_002948_r1_20 | /5Biosg/GAAGGTCCATTTGTTTTAGTTTAATAGACATTTATTCCTTCAGTTGAACAACCTCT |
| (SEQ ID NO: 871) | ACACAATTAAAATGTATGACATAAGATCTTTTCTTTTTTTGTGAAGAAATTTAGGTCTCAAGAA |
| RPL17_NM_000985_r1_1 | /5Biosg/ATCCAGTCAACCCTCAGGCTGCTTAGGGAAAGAGGAAGAGCGGTGGATTTTGGGAG |
| (SEQ ID NO: 872) | AACTCATGAGAACTTGACTTCTCGCGAGATTCGTAGCCGAAGAAACGAGATCTGAGGAGGCAGG |
| RPL17_NM_000985_r1_2 | /5Biosg/GGTGTCCTAAGAAATGCCATCATCGCAGCCATACTTTCCCCCACCAAGGGCTTGCT |
| (SEQ ID NO: 873) | TTCCCTTTTATCTTCCAGTAACTACAGCACTGCTTCCACAGAAGTAGCCACACGGGCCTCACCA |
| RPL17_NM_000985_r1_3 | /5Biosg/CACCTAGAGGAAAGTACAGTGTAATTCTGTCAATACGTACTTACAGTAACTCATTC |
| (SEQ ID NO: 874) | AGTATAAATATCAGATGATTCGAACAGCTGCTCAGAGAGTAGTTGTTTTCATTATTAATCCAAA |
| RPL17_NM_000985_r1_4 | /5Biosg/TTGATGGCCTGAGCAGTTTCACGAGTGTTCTTAAAGTGAACACGAAGATTGGAACC |
| (SEQ ID NO: 875) | TCTTGATTTGCATGATTTCGTGGGGTTCTCCGGGTCAAGTGAATAGCGAACCATTTTCACAGAT |
| RPL17_NM_000985_r1_5 | /5Biosg/CCCCATTGCTTGGCCTGCGCACACCTGCCAACTCCACCATTGTAACGTCGGAATGG |
| (SEQ ID NO: 876) | TACACACTGTTTCTGTAAAGTGACATCTTTCAGATACTTCGTGGCTTTTCGTATATGCATACCC |
| RPL17_NM_000985_r1_6 | /5Biosg/ATATGCTCAATGACCAGAGAATCTACATCTAAACCCTTAAGTTCAGCATTACTCTC |
| (SEQ ID NO: 877) | TGCGTTTTTAAGCATGTGCAGCAAAAATTCAGCACTCTTTTTGGGCCACCGACCTTGTGTCCAG |
| RPL17_NM_000985_r1_7 | /5Biosg/ACAATCTGTTCCTTTTCCGTAAGGATCATCTCAATGTGGCAGGGAGAGCTCATGTA |
| (SEQ ID NO: 878) | TGGGTTAATCCGACCATGAGCTCTGTAGGTCCGGCGGCGCATCTTAGGTGCTTTGTTCACTTGG |
| RPL17_NM_000985_r1_8 | /5Biosg/TTTTCCTTTTAATTACATTTATTTTAATGCTGAATTTACTCCCGTGCCATAAGTTT |
| (SEQ ID NO: 879) | TTGTTTCTTCAGTTTCTTCTGGGATATCTTTTTCTTCTGGGCAACCTCCTCTTCTGGTTTAGGA |
| RPL18_NM_000979_r1_1 | /5Biosg/TAGCCGGGCGATAACGGCAGAGAGCATAGAGCGCAGGAACAAGCGCAACGTCCAAG |
| (SEQ ID NO: 880) | AGGGAAGGGCCAGCACGTCGGGGGCCTCTCTGGCCCTACCCAGGCCGTGTTCTCGATAGCTTTC |
| RPL18_NM_000979_r1_2 | /5Biosg/GGGGTGGGCGGGGAAAGCCTCAAGCGAGTGGCGCCGCCGTGACGTCACATGGGTGC |
| (SEQ ID NO: 881) | GACGCGGCGCTCTACCACGATCCGGACTTCTCTGTGGTGAGTTGGACGACTGACTAGCCGGGCG |
| RPL18_NM_000979_r1_3 | /5Biosg/TCCTGGCTCTTGGGCTCCTTGCGCCGAACCTTTCGGTCCTTGTTATGGCGGATGTC |
| (SEQ ID NO: 882) | CACTCCCATGATGGCGCCTCCTGCTCGGCCAGGTCCGGAAAGAGAGAACGGGCTGGGGTGGGCG |
| RPL18_NM0_00979_r1_4 | /5Biosg/GCCGGTTGGTGCGACTCATAAACAACCTCTTCAACACAACCTGGTTGAATGTGGAG |
| (SEQ ID NO: 883) | TTGGTTCTTCTGGCCAGAAACCTGTATAACTTGACCAACAGCCTCAGGTAGATATCCTGGCTCT |
| RPL18_NM_000979_r1_5 | /5Biosg/TACCTCCTGAACCCGCACATCATCAGTTATGGTCCCCACAACCACGGCCGTCTTGT |
| (SEQ ID NO: 884) | TTTCCCGGCCAGGAAGCTTCATCTTCCGGATCATCCGGGAAAGGGACAGAGGCGGCCGGTTGGT |
| RPL18_NM_000979_r1_6 | /5Biosg/TTAGGGGAGTCCAGGGCCAGCTGGTCGAAAGTGAGGATCTTGCCCCCTGCCCTGAG |
| (SEQ ID NO: 885) | GATGCCGCTGCGGGCCCGGCTGGTCACGCGCAGTGCACATACCTTCAGTTTGGGTACCTCCTGA |
| RPL18_NM_000979_r1_7 | /5Biosg/AGCGGACGTAGGGTTTGGTGTGGCTGTGCCGGGTTCCTGGGGCCTTGCCGAAATGC |
| (SEQ ID NO: 886) | CGGTACACCTCTCGGCCCTTGCGAGGACCGGAGAGCAGGACAGTGCCACAGCCCTTAGGGGAGT |
| RPL18_NM_000979_r1_8 | /5Biosg/TTTGCACTGTCAGCAAAAATCTTTTTAATAAGAGAGTAGGATCCAGGGTTAGTTTT |
| (SEQ ID NO: 887) | TGTAGCCTCGGCTGGCCCGTCGGCCTCTGGCACGCTCGAACTTCCGGCCCTTGGAGCGGACGTA |
| RPL19_NM_000981_r1_1 | /5Biosg/TCTTGCCACAGCGGAGGACACTAGAGGCGAGCCTCTTCTGAAGCCTGAGCATACTC |
| (SEQ ID NO: 888) | ATGGCTGCGGCCGCAGCAGCGAAAGGAAAGAGCTCGCTCGGGCCCGGCTCCTCCCATTATCTGC |
| RPL19_NM_000981_r1_2 | /5Biosg/GCTTGCGGATGATCAGCCCATCTTTGATGAGCTTCCGGATCTGCTGACGGGAGTTG |
| (SEQ ID NO: 889) | GCATTGGCGATTTCATTGGTCTCATTGGGGTCTAACCAGACCTTCTTCTTGCCACAGCGGAGGA |
| RPL19_NM_000981_r1_3 | /5Biosg/CGGGCATTGGCTGTACCCTTCCGCTTACCTATGCCCATGTGCCTGCCCTTCCGGCG |
| (SEQ ID NO: 890) | GGCCAAGGTGTTTTTCCGGCATCGAGCCCGGGAATGGACCGTCACAGGCTTGCGGATGATCAGC |
| RPL19_NM_000981_r1_4 | /5Biosg/CTGTGATACATGTGGCGATCGATCTTCTTAGATTCACGGTATCTTCAGAGCAGCCG |
| (SEQ ID NO: 891) | GCGCAAAATCCTCATTCTCCTCATCCATGTGACCTTCTCTGGCATTCGGGCATTGGCTGTACCC |
| RPL19_NM_000981_r1_5 | /5Biosg/GCCAGGAGCTTCTTGCGGGCCTTGTCTGCCTTCAGCTTGTGGATGTGTTCCATGAG |
| (SEQ ID NO: 892) | AATCCGCTTGTTTTTGAACACATTCCCCTTCACCTTCAGGTACAGGCTGTGATACATGTGGCGA |
| RPL19_NM_000981_r1_6 | /5Biosg/CTTGGATAAAGTCTTGATGATCTCCTCCTTCTTGGCCTGGAGGCGCTCTTCACGGC |
| (SEQ ID NO: 893) | GCTTGCGTGCTTCCTTGGTCTTAGACCTGCGGGCCTCAGCCTGGTCAGCCAGGAGCTTCTTGCG |
| RPL19_NM_000981_r1_7 | /5Biosg/TTGCAGATTAAGGCTTGTTTTATTTTAATGGCTGATCTATGTAATCACAGAGGCCA |
| (SEQ ID NO: 894) | GTATGTACAGACAAAGTGGGAGGTTTTATTTCTTGGTCTCTTCCTCCTTGGATAAAGTCTTGAT |
| RPL21_NM_000982_r1_1 | /5Biosg/GGAACAACTCCATGTTTTCTAAAAGGCCTAGAGAACATATATCGGGTGCCTCTCCT |
| (SEQ ID NO: 895) | CTTTCCCTTTGTGTTCGTCATTTTGGCGAATTACTGGAAGATGGCGGTTCCGGCCGAAAGGAAA |
| RPL21_NM_000982_r1_2 | /5Biosg/CTCTTCCAGTTTTGCCATCGTAACACTTGTGGGGCATTCCTTTTTGAACAGTACCC |
| (SEQ ID NO: 896) | ATTCCCTTGATGTCTACAATATCACCTTTCTTATAGATTCGCATATATGTGGCCAAAGGAACAA |
| RPL21_NM_000982_r1_3 | /5Biosg/CTCTTAGAGTGCTTAATGTGCTCAATACGCACATTAATTCTCTTGGCAAGAATCTT |
| (SEQ ID NO: 897) | GCCCTTAACTTGTTTGTTTACAACAATGCCAACAGCATGCTGGGTAACATTGTAGACTCTTCCA |
| RPL21_NM_000982_r1_4 | /5Biosg/TGCTTCTCTGGGTGGAGCAGGCTGGCGCTTTAGTTGAACCCAGGTACCTTTCTCTT |
| (SEQ ID NO: 898) | TGGCTTCTTTCTTTTTCTGATCATTTTCCTTCACACGTTTCAGGAAGCTATCTCGGCTCTTAGA |
| RPL21_NM_000982_r1_5 | /5Biosg/TTTTTTGTAGCCCAGAGGTCCTTTATTTTTTTTTTTAACACCTATAATGCCATGAA |
| (SEQ ID NO: 899) | TTCATAGGGAATAGGTTCCAGCAGCTCAGGCTCCTTCCCATTGGTTCTCACAAAGTGTGCTTCT |
| RPL22_NM_000983_r1_1 | /5Biosg/CAATCAAGAGTGAACTTCAGAACTTGCTTCTTTTTTTTGCCCCCCTTCACCACAAG |
| (SEQ ID NO: 900) | CTTTTTCACAGGAGCCATGGCGGCAGCGGAGTTAGAAAGGGAGGTGAGCGAACTACGCAGACGC |
| RPL22_NM_000983_r1_2 | /5Biosg/CGATGGTCACCACCCCTCCACCAAGGTTCCCAGCTTTTCCGTTCACTTTGATCCTT |
| (SEQ ID NO: 901) | TCTTGCAAAAACTGCTCAAAATTGGCAGCATCCATGATTCCATCTTCTACAGGGTGGGTGCAAT |
| RPL22_NM_000983_r1_3 | /5Biosg/TACGCGCAACCAGTCACGTAGATTATTCTTCTTCAAATATTTTTTGGTGAGATATT |
| (SEQ ID NO: 902) | TCAAATACCTTTTGGAGAAAGGCACCTCGGATGTCACGGTGATCTTGCTCTTGCTCCTTTCGAT |
| RPL22_NM_000983_r1_4 | /5Biosg/AAGAACTCATACAAAATTTTCCAGATAAATGAAATTTAATCCTCGTCTTCCTCCTC |
| (SEQ ID NO: 903) | TTCTTCGTCCTGGTTAATCTGGAAGTAACGTAATTCGTAACTCTCTTTGCTGTTAGCAACTACG |
| RPL22_NM_000983_r1_5 | /5Biosg/AAATAAATGAGTGGCGAACCAAGGGAAGCCCTTTGACTATGATTTCCAATTTTCTG |
| (SEQ ID NO: 904) | TTCAATCCACACTGCAGAGATACAAGGATAAACCACCATTTTGGTTCCCAAGTTTTATTCAAGA |
| RPL22_NM_000983_r1_6 | /5Biosg/ACCCGCATCTGCCTCCCCAATGGCTGTCAGTTCGGTAAAGTCACCCTCTCCTTCTA |
| (SEQ ID NO: 905) | CTCTGGTATTACCACGAGAATTGAAATTTTTAAGCAGAAAAAAAAAGAAGTCAAGTTACAAATA |
| RPL22_NM_000983_r1_7 | /5Biosg/TTAATGAATGAGAGAAGCCCATTTGTATCCCTGAATCATTGAGAAAAGCAACAGAT |
| (SEQ ID NO: 906) | ACAACTGACAGTCACACTTTTTAAAATCAAACAGTCACTACCTTCAGCCCACACCTCCACACCC |
| RPL22_NM_000983_r1_8 | /5Biosg/TGCGTCAAGAGAAATTTGTAATCAAAAAATGAAAGTAAGACGAATGGCCCAGAAAC |
| (SEQ ID NO: 907) | CCGCATTTTATTGACAGTCATTTTCCCACAGAGAATCTTAGAAAGATGTCGCGTTTTCTTTTAA |
| RPL22_NM_000983_R1_9 | /5Biosg/GTTAATTTAGAACCTGGGACTTTTACAATCGATTCCCCAAACCCCTTTATGGCAGC |
| (SEQ ID NO: 908) | AACACTGAAGGAGCACAGTTTCTCTCTCTAGGAAGAAGAGGATTAGCAGACATAATTGTGTGCG |
| RPL22_NM_000983_r1_10 | /5Biosg/GGAGACCACATCTGGAAGCACTATTCCTACAGCTCCCTCTGCTGGAGACGAAAGTG |
| (SEQ ID NO: 909) | ACAGTTTGTTGCAAAGACCTGTAGAAACATTACTTACACGTTCATTTCTGTACACATTTAGTTA |
| RPL22_NM_000983_r1_11 | /5Biosg/GCACCTCCCACCTCCTTGGCATGGGAGGCTTCCCAGTCACTCCTCCTTCGTGTCCC |
| (SEQ ID NO: 910) | CTCTTTCAAGAACCTCCAGAGCTCTTGGCATCAGGGGCCCCCATTGCTGGGCCCCACACGGGAG |
| RPL22_NM_000983_r1_12 | /5Biosg/TGCATTTTTTTCCCAGCCTTGGTAGCCCCTTTCAGTTGTCTAGCCTCTGCACTGTG |
| (SEQ ID NO: 911) | GCACACCACTGACATTAAATCCAGTCATGGCCTAAGTGGCATGAGGCAGCTATTTCCAGGGCAC |
| RPL22_NM_000983_r1_13 | /5Biosg/AAAACATGAATTTTAGACACCGTAAATTCTAATGCAGACACTTTTGCATTACTGTT |
| (SEQ ID NO: 912) | TGAATTTCAGAAGGGCACCACAAGGCACCAGAGTCTTTCAAAGTCACTCACAGCAACAATTGCA |
| RPL22_NM_000983_r1_14 | /5Biosg/CAGAGCACCAAATGTTTAATGGGAGCCAAGGTAGGACTGAGCATTGAACTTCCAGC |
| (SEQ ID NO: 913) | TATGCAACTCGCAGGGCACAATTTCAAGTGTGGAAACCATCTGTAGGCAAGCTCTTTTAAAAAC |
| RPL22_NM_000983_r1_15 | /5Biosg/CAGAGAAGAAATATGCAAGCAATTCTGCTTCAAAGAAATTTGCATAGAAATGGAAA |
| (SEQ ID NO: 914) | AATGCCAGAGCCTTTAACACAAGTGAAATTGCAAAGCCTCAACACGTTCAACTCAATCCACAGA |
| RPL22_NM_000983_r1_16 | /5Biosg/AGTAGAATTTGTAAACTCAAGCCACAAACTTAGTTAATAATCATGGTTAAGGGACA |
| (SEQ ID NO: 915) | TTGCCAAAGAGCAACTGATGCCTCAGTGAAGTTTGAAAGAAACTCTGCTTTCTGTGACGGCAGA |
| RPL22_NM_000983_r1_17 | /5Biosg/TTTTCAAATCTGGGACTAGTTTCTTTTTTTCTTTTAACTGAAATGCCAACTTCAGC |
| (SEQ ID NO: 916) | CCAGGGTTTTTTCACAACCAAACTAAAAATGACTTACTACATGGGAACATCAATGCAACAAGTA |
| RPL22_NM_000983_r1_18 | /5Biosg/TTTTTTACAGTAAGAGTAGCCAGGTGTTAGCCACTTTAATAGAAAATATGATCAAA |
| (SEQ ID NO: 917) | ACTCGATTACAAGAGTTCAAAAAGACATAGAAAACCAGTGAGTTTCAATTTTATTACAAGTTTT |
| RPL23A_NM_000984_r1_1 | /5Biosg/GACACCTTTCAACACTGCCTTCTTGGCCTTTAAAGCCTTCGCTTTGGCTTCAGCTT |
| (SEQ ID NO: 918) | TAGGAGGGGCAGGAGCTTCCTTCTTCGCTTTCGGCGCCATCTTGTGAAAAGGGTCTCCAATGCT |
| RPL23A_NM_000984_r1_2 | /5Biosg/TCCTGGGAGCGCTCTTCCGAGGATATTTGGGCTGTCTCCGGAGTCGCAGTGTCTTC |
| (SEQ ID NO: 919) | GGCCGCCGGAAGGTGGGTGACGTGCGGATCTTCTTCTTTTTGTGGCTGTGGACACCTTTCAACA |
| RPL23A_NM_000984_r1_3 | /5Biosg/TTTAACATCCACAATGAACACAAGTGTGTTGTTGTCTTCTATCTTCTTCATGGCAG |
| (SEQ ID NO: 920) | ACTCAGTGGTCAGCGGAAACTTGATGATAGCATAGTGGTCAAGCTTGTTTCTCCTGGGAGCGCT |
| RPL23A_NM_000984_r1_4 | /5Biosg/GAACATATGCCTTCTTCTCTCCATCAGGCCGAATCAGGGTGTTGACCTTGGCCACA |
| (SEQ ID NO: 921) | TCAATGTCATACAGCTTCTTCACAGCCTGTTTAATCTGGTGCTTGTTGGCTTTAACATCCACAA |
| RPL23A_NM_000984_r1_5 | /5Biosg/TGAAAAGATATATATATATATATATTCAGAATTAGGCAGCTGGACTCAGTTTAGAT |
| (SEQ ID NO: 922) | GATCCCAATTTTGTTGGCAACATCCAAAGCATCGTAATCAGGAGCCAGTCGAACATATGCCTTC |
| RPL23A_NM_000984_r1_6 | /5Biosg/TTCAAAAGGATAAGTAGAACAGGAGTCTTGCCCAAGCCCTGTCATGTCAGTGTGTG |
| (SEQ ID NO: 923) | TGTGTGGCCTCCCAGCCCAACCAGAAATTGACAGACAGGCATGTATATGGTGAAAAGATATATA |
| RPL23A_NM_000984_r1_7 | /5Biosg/CTCAACCATGGAGGCAAGTTCTGAGTTCTGGTTTTCCTAGGACACTAACTCTAGTC |
| (SEQ ID NO: 924) | ACAAAGATCTCTGGAATGATCATACATGGTGGAGTGGCAGGGTGAGGTATTTCAAAAGGATAAG |
| RPL23A_NM_000984_r1_8 | /5Biosg/CCAATCCACACTCCCCTGAAATGGATGCTAACACCCTTCAAACCCTGGGCATTGGT |
| (SEQ ID NO: 925) | TTCATACACAGCCTCTTCCAGGGATAAAAGGGGTTCTTGTACAGCTTGTTACTCAACCATGGAG |
| RPL23A_NM_000984_r1_9 | /5Biosg/TCCTCTGAAGTCCTTGTCCTTTATTGGGCAAGGGGTGAGGGAAGCAGACAGACCGG |
| (SEQ ID NO: 926) | TTGGACATAGTAGATGGGTGTGTGAGGACAAAATGCTACCAGAGAGCCAGCCAATCCACACTCC |
| RPL23_NM_000978_r1_1 | /5Biosg/AGAGGACCCACCACGTCCTCGCTTCGACATCTTGAACGCCGGAAAAAAGAAAAAAG |
| (SEQ ID NO: 927) | GAAGTCGATTCAAAGGACACTGGGATATGAACTTTAACGCCCCGCCCACCCTCCTCACGTGGCC |
| RPL23_NM_000978_r1_2 | /5Biosg/GCCGTCCCTTGATCCCCTTCACGGAGATGATATACAGGTTTTTGGCTCCTGTGTTG |
| (SEQ ID NO: 928) | TCAGCACAATTGATTACAGCTCCTACCGGAAGACCCAAGGAAATCCGGAATTTCGCACCAGAGG |
| RPL23_NM_000978_r1_3 | /5Biosg/GACTTTCGTTGTCGAATGACCACTGCTGGATGTACCTTTTTTCTGAGCTCTGGTTT |
| (SEQ ID NO: 929) | GCCTTTCTTGACTGTGGCCATCACCATGTCACCCACACCAGCAGCGGGAAGTCTGTTCAGCCGT |
| RPL23_NM_000978_r1_4 | /5Biosg/ACACTCCTTTGCTACTGGTCCTGTAATGGCAGAACCTTTCATCTCGCCTTTATTGT |
| (SEQ ID NO: 930) | TCACTATGACTCCTGCATTATCTTCAAAATAAAGAAACACGCCATCTTTTCTACGGTATGACTT |
| RPL23_NM_000978_r1_5 | /5Biosg/TTTTGCAAACAAATACTTTTTAATGGGTTTAGTTTTTTTTTTTATTTTTTACAAAT |
| (SEQ ID NO: 931) | ATACTGGAGAATCATGCAATGCTGCCAGCATTGGATGCAATCCGGGGCCACAAGTCTGCACACT |
| RPL24_NM_000986_r1_1 | /5Biosg/CCGTCGGTCCTGGCGTAGCGCCTCCCGTGTCCGGGGTAGATCTTGTACCCGCTAAA |
| (SEQ ID NO: 932) | ACTGCACAGCTCGACCTTCATGGCGACAGCTCCACGGAAAGACAAAAGATGGCGAAAAGAAAGA |
| RPL24_NM_000986_r1_2 | /5Biosg/ACTGTCCCTTTTTGTGCTTCCTTCTGTAGAGGACAGTCCAGTTTATCTGCCGAGGA |
| (SEQ ID NO: 933) | TTCCTCTTGGAAAGGAAAGCCGACTCGCATTTCGCATTAAGAAACTGGAAAACCTTCCCGTCGG |
| RPL24_NM_000986_r1_3 | /5Biosg/CTAACTTCAGGTTTCTGATTCCTCTTGGCCATTATATCAGCAAGAGATGCACCAGT |
| (SEQ ID NO: 934) | AATGGCCCTCTGGAATTTGACTGCTCGGCGGGTTCTTTTCTTTTGAATTTCTTCCGACTGTCCC |
| RPL24_NM_000986_r1_4 | /5Biosg/AGGTGCTGCCTTTGTAGGTGCCTTAGCAGCAGCCATTGCAGTCTTTTTAGATGCTT |
| (SEQ ID NO: 935) | GCTTAGCCTTTTTTGCTTCCTTAGCAGCCCTGATAGCTTGTTCTCGTTGAGCCTTTCTAACTTC |
| RPL24_NM_000986_r1_5 | /5Biosg/TTTTTTTTCTAGAGTTATAATCCAATCTTTATTTAAAAATCTAATCTGCCAGTTTA |
| (SEQ ID NO: 936) | GCGTTTTCCACCAACTCGGGGAGCTGAAACTTTCACAGGCTTCACAATCTTTTGCTTAGGTGCT |
| RPL26_NM_000987_r1_1 | /5Biosg/AGTCACAAAGGGATTAAACTTCATTTTGGCCGCTCCCGCTTCGGTGATGGCCGCAA |
| (SEQ ID NO: 937) | AAGGGAAGAGAACTACACGCTGCTTCCGGTTCTGTAAGTTTACCAAAGATCTCGCGAGACCTAT |
| RPL26_NM_000987_r1_2 | /5Biosg/CATGGATCGCACGTTGTACTTCTGTCTCAGCTCTTTGGAAAGAGGGGAAGACATAA |
| (SEQ ID NO: 938) | TCTTCCTTCGAATGTGGGAAGGTGCATTGAAATGCCTTTTGCGATTCTTGCTTCGGTCGGAAGT |
| RPL26_NM_000987_r1_3 | /5Biosg/GCTGCACCCGTTCAATGTAGATAACATATTTCTTCCTGTAAACCTGGACTACTTTG |
| (SEQ ID NO: 939) | CCAATTTGCTGACCTTTATAGTGTCCACGTACAACCTGAACTTCATCATCCTTTCGGATGGGCA |
| RPL26_NM_000987_r1_4 | /5Biosg/CGAGATTTGGCTTTCCGTTCGAGGATCTTTTTGCGGTCTTTGTCCAGTTTTAGCCT |
| (SEQ ID NO: 940) | AGTGATAACCACCTTGCTGGGGTGAATGCCTACGTGGACAGTTGTGCCATTAGCCTTTTCCCGC |
| RPL26_NM_000987_r1_5 | /5Biosg/TTTTTTTTCAGGCTCTTTGTTTCAAGTTTTAATCAAAGCTTGTATATAAGATTACT |
| (SEQ ID NO: 941) | TTATTCCTGCATCTTCTCAATGGTTTCTTCCTTGTATTTGCCCTTTTCCTTTCCTACTTGGCGA |
| RPL27A_NM_000990_r1_1 | /5Biosg/AACTGTTGAAGAATCGGGGCTAGGTGTGCTGTACTGCACGCCGCCCGTGCTTGCTT |
| (SEQ ID NO: 942) | GAGCCAGGCCCTTTTCTGGCGCCTTGGTCAGGGAACCGCCTAAGCCGACCCTAAGGGCCTCGTT |
| RPL27A_NM_000990_r1_2 | /5Biosg/GGGATTGCGCGCCTGCTAGTCGCTTCCTCTTTCTGAGGGTGGTGATCCCCCATCAC |
| (SEQ ID NO: 943) | GGAGTGTCCTGGTGGCGGTGTACGGTTTCGGTGAGGCTCGTTGTGCTAGGCTCGGAGGGCGAGA |
| RPL27A_NM_000990_r1_3 | /5Biosg/CCCAGACGAAAAAGGAAGGCCTTCGCCAAGTCTCGCGAGGTATCGGCCACGGGGGC |
| (SEQ ID NO: 944) | GGGGCAAAACGCCTCACTTCCGGTCACAGAGGCCGGGCTTCGGGCGGGGCCTCCTGGTCTCGCG |
| RPL27A_NM_000990_r1_4 | /5Biosg/ACCACCAGCATTACCGCGGCCGCCGGGGTGCTTCCGGTGCTTGCCTATGCGGCCGT |
| (SEQ ID NO: 945) | GGCCGTGGCTCACGTGGCCCCTAAGTTTCCGGGTCTTCCTCAGTCTGGATGGCATGTTGGCAGC |
| RPL27A_NM_000990_r1_5 | /5Biosg/TTGTCAAGGTTGACAGTTGGGCAGAAGCTCTGGTTCCTCTTTAAGTGGTAATGCTT |
| (SEQ ID NO: 946) | CATACCAACTTTCCCAAAGTAGCCTGGGTGGTATTTGTCGAAGTTGATCCGGTGGTGATGCAGA |
| RPL27A_NM_000990_r1_6 | /5Biosg/GCTTTCCCTTTCCCAGAACTTTGTAGTAGCCCGATCGCACCACATCAATGATGGGA |
| (SEQ ID NO: 947) | GCAGCCCCAGTCTTGTTTTTAGCAGCATTCACCCGTGTCTGTTCACTGACCAAAGTCCACAATT |
| RPL27A_NM_000990_r1_7 | /5Biosg/TGAAACTCCCTCCATGTGGCTTCAAGCCACCAGGACACAGGCCCCCCCAACACTCT |
| (SEQ ID NO: 948) | TAATCTTCTCCTCAGCTCTTCTGCTGAAGAATTTGGCCTTCACGATGACAGGCTGCTTTGGGAG |
| RPL27A_NM_000990_r1_8 | /5Biosg/AGGACCCCATGTCTTCATACCCTAAGTACTAGAACCTGGGCTCCCAATGCACACAG |
| (SEQ ID NO: 949) | GATGTAGAGGTGCCACTGAAGAACCTACACTCACACCACAAGGAAAAAGTAGTTAGCATTTAAT |
| RPL27A_NM_000990_r1_9 | /5Biosg/ATACATGCAGGCCTTTCAGTGATCAACAGGTATTATTCTGACTTTATAAACAGAAA |
| (SEQ ID NO: 950) | TGTTTAACTGGCCAACGTCTGTGTTGGTCTTACGTTTACCATATATTTGAGGGAAGTCAGGAGA |
| RPL27A_NM_000990_r1_10 | /5Biosg/CTCATTCCTATCACACTGCCCCTCTTTAGAATGTGACCCATCAAAATGGAATCTTT |
| (SEQ ID NO: 951) | CCTTTTGAGCCCTGTTGAACAATTAGGGTACATTCTCTCATTCACTGTAAAATTCAGAGTACAA |
| RPL27A_NM_000990_r1_11 | /5Biosg/GCCGAGATCGTGCCACTGCACTCCAGCCTGGCAAGAGAGACTCCGTTTAAAAAAAA |
| (SEQ ID NO: 952) | AAAAAAGATGAGGGGGCCACGTGGAAAAACCTTGGGCTGAGAACTTAAGTCCTAAAGGACCATC |
| RPL27A_NM_000990_r1_12 | /5Biosg/CCCATCTCTACTGAAAATACAAAAATTAGCTGGGCATGGTGGTGGGCGCCTGTAAT |
| (SEQ ID NO: 953) | CCCAGTTAGTCAGGAAGCTGAGGCAGGAGAATCGCTTAACCTGGGAGGCGGAGGCTGCAGTGAG |
| RPL27A_NM_000990_r1_13 | /5Biosg/TTAAGGAGCCATGGCCGGGCGCAGTGGCTCACACTATAATCCCAGAACTTTTGGAG |
| (SEQ ID NO: 954) | GCCGATGTGGGCAGATCACTTGAGGTTAGGAGTTAGAGACCAGCATGGCCAACATGGTGAAACC |
| RPL27A_NM_000990_r1_14 | /5Biosg/TTTCAAACCCACTTTGACATACAGAAAAGCCCTGGCATACTGAAATTTCAGATTGC |
| (SEQ ID NO: 955) | TGGACCCCCATCTCGAGATTTTGATTCATCACATTCTGGATGTAACAATAATTTGGATCAAGAT |
| RPL27A_NM_000990_r1_15 | /5Biosg/GTCACCATCAGTTTTCCAATAGCAAGAGACACAAATCCTAGAAAAACTTATTTCAA |
| (SEQ ID NO: 956) | TTTCCAGCCTTCAGGATTTCCAAATTTTCCCGATACATTTCAGACTAGAAGAAAAATTAACTAT |
| RPL27A_NM_000990_r1_16 | /5Biosg/AGTTTTAGACTGTATTATTTTGGGTCAAAAAAAGCTCAAAATCACAGGAAGAACCA |
| (SEQ ID NO: 957) | TAAAACTGTAGCAAATTATAATTTACCGAGATCTATGAGGATCTTATTTGTAAACATGAATTGG |
| RPL27A_NM_000990_r1_17 | /5Biosg/TCCTGACAGATGTGTCCCATAGACAGCCATGGTTCTCGGCTTATTATGACCCCACA |
| (SEQ ID NO: 958) | GTGCACTAAGGAAACCTCCACTAACTAAAACCCAGGAGTCTAAGTGCCATCTTATTTGTCTATA |
| RPL27A_NM_000990_r1_18 | /5Biosg/TTTAAAATTGTCCAGCTGAATCGAGGTTTCACTACTTGTAATTCTAGCCTATTCTT |
| (SEQ ID NO: 959) | ATGTAAGCCAATTCAGTGCTTCTCTGTGCCTTCTATTTGATCCCCAACATCCTCTAAAGGTTGT |
| RPL27A_NM_000990_r1_19 | /5Biosg/GGCTAATTTTGTGTATTTAGTAGAGACAAGGTTTCTCCAGGTTGGTCAGGCTGGTC |
| (SEQ ID NO: 960) | TGGAGAGCCCTTATTTCTAACTTCTCCAGCACAACAGAAGATACAAGCCAAATGATACATTTGT |
| RPL27A_NM_000990_r1_20 | /5Biosg/TGTTGCCCAAGCTGGAGTGCAATGGCACGATCTCGGCTCACAGCAACCTCCGCCTC |
| (SEQ ID NO: 961) | CTGGAGATTCTCCTGGCTCAGCCTCCAAAGTAGCTGGGATTACAGGCATGTGCCACCACGCCTG |
| RPL27A_NM_000990_r1_21 | /5Biosg/ATGAACTACTGACCACTTATTGTGGGATCCTTGGATTCAGGTTCCATATTACAAGT |
| (SEQ ID NO: 962) | CCTCTATGGGCCCTAATGGAGAGCCCTTACTTTTTTTGGGGGGGGTGGACAGAGTTTCACTCTT |
| RPL27A_NM_000990_r1_22 | /5Biosg/TTTTAATCTACACAATCCTCAGGTAGGCACTTTCACTGAAATTTTAAAGATGAGGA |
| (SEQ ID NO: 963) | AATTAGGACTAAAAGTCACACAGGTATTGGGTGAAGACCAAATATTGAGTCTTTTAATTCATCA |
| RPL27A_NM_000990_r1_23 | /5Biosg/GTCTGAACCTAGGAGGCGGAGCTTGCAGTGATCTGAGATCGCACCACTGCACTCCA |
| (SEQ ID NO: 964) | GCCTGGGCGACAGAGCGAGACTCCGTCTCAAAACAAAACAAAAAATTAAGTGCACGGTTTCCAT |
| RPL27A_NM_000990_r1_24 | /5Biosg/CGAGACCATCCTGGCTAACACGGTGAAACCCTGTCTCTACTAAAAATGCAAAAAAT |
| (SEQ ID NO: 965) | TAGCCGGGCGCAGTGGCGGGCGCCTGTAGTCCCAGCTACTTGGGAAGCTGAGGCAGGAGAATGG |
| RPL27A_NM_000990_r1_25 | /5Biosg/TAATAGTTTGCACTTAAGTTACTAAGTCAAACACAAAAATTAAGTGCCTGGGCCGG |
| (SEQ ID NO: 966) | GCGCGGTGGCTCATGCCTGTAATCCCAGCACTTTGGGAGGCTGAGGCGGGCAGATCAGGAGATC |
| RPL27A_NM_000990_r1_26 | /5Biosg/CATAGCACTTCCCGTTTGCCTAACCTTCCCAAACCAGTTTCCTGAACCAGTATTCC |
| (SEQ ID NO: 967) | CTCTGCTTGGACCAATTTCCCACTGACAATCACATGAAGGCCAATTGACTCCATCTGCTCCCGT |
| RPL27A_NM_000990_r1_27 | /5Biosg/GTCCATTATGCCGTCTGAATCGTACCTTTTCCATACTCACTCCCATTTTCTTGGTT |
| (SEQ ID NO: 968) | CTGCCTTCAGCCTCCCATTGTAGCCGTCCTTTACAGCCCGGCCAGAGTAATCTTTCTCTCCACC |
| RPL27A_NM_000990_r1_28 | /5Biosg/ACAACTCCTGGGGAAGAAGGTAGCCATTAAGGGACTAGGGATCCTTCCCCCCAAAC |
| (SEQ ID NO: 969) | TCTACTTCCTTCCATGCCTTATCATTTTGTGGTCGGCCCACTACAATTCAGTCTCCAAGTCCCG |
| RPL27A_NM_000990_r1_29 | /5Biosg/TCTGTTCCTCATCACATGAAACAGTTTTTAGAACTGTTAGTAGCTGTAGCTTCTGG |
| (SEQ ID NO: 970) | TGAGTGAACAAGCCAGCAACGAAATCAGTGTTTCTTTCCCAGGCCAGGGGATCGGATGGCCTAA |
| RPL27A_NM_000990_r1_30 | /5Biosg/TATGAGCCTCCATGAACACAGCAGGCTATTCAAAGCTTGGCAGTCATGAAAATATT |
| (SEQ ID NO: 971) | TTAGTAAATGCGGGTTCAAGAAAGCACTGGAATCGGCGGACTTAGGGCTCAAAACTATTTTCGT |
| RPL27A_NM_000990_r1_31 | /5Biosg/ATCAAGTTGCACCTGCAGCTCCCACGTTTGAACGCAACTATATTTAAGGCATATAT |
| (SEQ ID NO: 972) | ACATTAGACCAGAAATTAGATCTTTTGCAATTCAAGCTTTAGCCAGCCACTAGAGATCGCCAGT |
| RPL27A_NM_000990_r1_32 | /5Biosg/GCTAGTTAGCCGCTAAGTAGGGTTTAGTAGTACATTTATTTTGATTGAAAGCTCGC |
| (SEQ ID NO: 973) | CTGATTTAGCTGCCCTTGACGAGCACTGCTTGTGCAAATTATGTGGCAGCCATTTGTCATAAAA |
| RPL27A_NM_000990_r1_33 | /5Biosg/CCACTGAGGTCGGGTCGTGCTGACTGATGAACGCGGCCGATGGAGCCACCAGGTGG |
| (SEQ ID NO: 974) | GCGCTGCACAGCTTCCGCCCGCGCTTCCGACTTGCAGTCCGTCCGTGGGCTGTCTGCTCTTGGG |
| RPL27A_NM_000990_r1_34 | /5Biosg/GGGCTTCTTCCGGTCTGGGACGCGGGGGAGTCATCGTCTCCGGAAGTAGAAGCTAC |
| (SEQ ID NO: 975) | TCACCGGAAGTGGTTCTCAGGTGCCGGGGTAAGACCCAAGGTCCGGTCTGTGTTGTGAGGACGC |
| RPL27A_NM_000990_r1_35 | /5Biosg/CGCCCAAGGACCAGGGGTGGAGTTGAACATCCCGCCTCTCCAGGGGAGTTATCTGC |
| (SEQ ID NO: 976) | GCTCGCGCACCACTGGCCGGCTCCGCCCTTGCCGGAAGTGGCCGAGCGAGGCCGGTCTCCGCCG |
| RPL27A_NM_000990_r1_36 | /5Biosg/GGAGGTTCCCATCTCGAAGATTTACGTAAAGGCCACGCCCACTGTTCTTAAACGCA |
| (SEQ ID NO: 977) | CCCTAAAGGGGCTCGAAGTGCGGCGAGGTTGGCCATTCCTCCGCTTCGAAGGGGACCCACGGCC |
| RPL27A_NM_000990_r1_37 | /5Biosg/ATTCCTTTTATGTTCTTCTAGAATATTTCCATCCGTGTACAAGCACTTTCTTTTTT |
| (SEQ ID NO: 978) | AGCCCAAAGTGCCCTTGAATGTCGCTGACGCTCATTACCTTTTAGACAATTGAGACAAATTCTG |
| RPL27A_NM_000990_r1_38 | /5Biosg/TTCTCAAGATACTTTCGCTCCATTTATATTGCCTCTCAATATGAAAATACGCATTG |
| (SEQ ID NO: 979) | GGTTGAACAAAAAACTGCACCAAAACATACTTCGTGCTCATTTCCCTAACCTCCTAAGAGGAAA |
| RPL27A_NM_000990_r1_39 | /5Biosg/ATTCCAAAACAAATTTATTTGCCTGTGGAGGTGGGGGTGTAATTTAAAAGGCCATG |
| (SEQ ID NO: 980) | AAAACTCTCGAAAGAGAAAATATTACTTTTCAGCAACGGCAAGTTCTGGTAGTTTTTTTTTTTT |
| RPL27A_NM_000990_r1_40 | /5Biosg/AGAGGAAAATAAAAATTGGGATGTAAAATAGAGCCAGGAATTAAATTTACGCTTTA |
| (SEQ ID NO: 981) | ACCTATATTCTTGCTAGGGGAGCTACACAAAATACGTTTCTAGAGCCAGATGATGTGGTATGCA |
| RPL27A_NM_000990_r1_41 | /5Biosg/TTCAGTTACATTTTTGTTAAAAACATCTTTTCGCATGTACACTTATAACCTACTTT |
| (SEQ ID NO: 982) | ATACCAAAATGACTTACAGGGATACACAAACTAGGTTATTTGAAACAACGTAAAGTGGGAATGA |
| RPL27_NM_000988_r1_1 | /5Biosg/GATGACAGCTTTGCGTCCGGAGTAGCGTCCAGCCAGGACAAGCACCACCTTCCCAG |
| (SEQ ID NO: 983) | GTTTCATGAACTTGCCCATTTCGGCAGCAACCACCCGGCCCTACCAGCAAAAAGGAAAGAAGGA |
| RPL27_NM_000988_r1_2 | /5Biosg/TCTCTTGGCGATCTTCTTCTTGCCCATGGCAGCTGTCACTTTGCGGGGGTAGCGGT |
| (SEQ ID NO: 984) | CAATTCCAGCCACCAGAGCATGGCTGTAGGGGCGATCTGAGGTGCCATCATCAATGTTCTTCAC |
| RPL27_NM_000988_r1_3 | /5Biosg/AGCAGGATCTCTGAAGACATCCTTATTGACGACAGTTTTGTCCAAGGGGATATCCA |
| (SEQ ID NO: 985) | CAGAGTACCTTGTGGGCATTAGGTGATTGTAGTTATACACTTTCACAAAAGATTTTATCTTTGA |
| RPL27_NM_000988_r1_4 | /5Biosg/TTTTTAATGATCAAAACAAAGCATCTAAAACCGCAGTTTCTGGAAGAACCACTTGT |
| (SEQ ID NO: 986) | TCTTGCCTGTCTTGTATCTCTCTTCAAACTTGACCTTGGCCTCCCGTCGGGCCTTGCGTTTAAG |
| RPL28_NM_000991_r1_1 | /5Biosg/GCTGTAGGTCTGCTTATTCCTCTTGATCAGGAAACTGGAGCAGTTCCGCACGACCA |
| (SEQ ID NO: 987) | TCCATTGCAGATGCGCAGACATGGCGGCGGCTCCCTTCGCAGCGGCGACCTGAGACGGAAAGAG |
| RPL28_NM_000991_r1_2 | /5Biosg/CTTAATGACCACCACGACACCTTTGCCGTCGGCTGCCGGCTCCACGCCCACAGTCT |
| (SEQ ID NO: 988) | TGCGGTGAATCAGTCCGTTGTAGCGGAAGGAATTGCGGGCCTTCAAGTTATTGGGCTCAGTGCT |
| RPL28_NM_000991_r1_3 | /5Biosg/GGGGCGGTACTTGTTCTTGCGGATCATGTGTCTGATGCTGCTGAGCGTGGCGCGAG |
| (SEQ ID NO: 989) | CATTCTTGTTGATGGTGGTCCGCACATAGGAGGTGGCAGGCTTCCGCTGGCCGGATCTCCGCTT |
| RPL28_NM_000991_r1_4 | /5Biosg/CTGGGGGCAGGGGGCTCAGGAGCTCTTGGTGGGGCGGGTCCGCTTCCTCTTCACCA |
| (SEQ ID NO: 990) | TCACAGGCTTCTGGCTGCGCAGGATGGCGCTGGCCCTGCGGATGGCTGCCATGCGCAGGTCGGG |
| RPL28_NM_000991_r1_5 | /5Biosg/TGCAGAGTTTTGATGACATGGCCTGAATGACAACACACAGGGCCAGAGCTCCCCAG |
| (SEQ ID NO: 991) | AGCGTTTCAAAAAGGGAGGCCCAGTCGAGGCAGGTGAGAAAGCCAGCTGACTTTATTGCTCTGG |
| RPL28_NM_000991_r1_6 | /5Biosg/GTCAGCAAACTTTTTCTGTGAAGACAGTGACCCTTGCCCTCACCAGGGACAAGGCA |
| (SEQ ID NO: 992) | GCTACCCCACCCCTCCTGCATGGCCAGCCATTGACATCACCTCCAGATGGACAAGGTGACATGC |
| RPL28_NM_000991_r1_7 | /5Biosg/CCCTCAGCCTGCACCCTTCTGGGCTAGAALAGGCCTTTCTTCTTCTGAATGGAAGC |
| (SEQ ID NO: 993) | CACTGCCCCAGCTGGGATTCTTCAGGCCACCTCACAATGGGACAGTAGGTCTCAATCACAAGTC |
| RPL28_NM_000991_r1_8 | /5Biosg/CCACTCCCTGGATGCAAAAGTCATCTCATCTTCCTCCCGTGATGTCTGTCGTCCCC |
| (SEQ ID NO: 994) | CAGGTCTTGGGAACACCCCAAGTCCCCAAACCGTGCTACAGCACCAGGGCCCAGGGCCCAGCCC |
| RPL28_NM_000991_r1_9 | /5Biosg/GGCTGTACCCTGGGGCTGCCATCTCCCTTACAGTTATAAGCCACCACCCACCAAGT |
| (SEQ ID NO: 995) | TGCCCATCTCAGAGATGAGGTTGCATCAAGTAAAGCCCATCCCCTCCAAATGTGGCTGCACCCA |
| RPL28_NM_000991_r1_10 | /5Biosg/AGCGGCCACTGAGATGAGACCTAAGTGACAACAGCCAGCTCAAAGAGACAGGGGAG |
| (SEQ ID NO: 996) | GGATGGGGACACCTGGAGGACGGGAGTAGGGGGACAATGCTAAGGCTGCTCAATGCCTGCTGGC |
| RPL28_NM_000991_r1_11 | /5Biosg/TAGGATGGTCTCAAACTCCTGGCCTCACAAGCTGTAATAAACACTAGGGAGCAAAC |
| (SEQ ID NO: 997) | AGGCCAAAGGAAACAAGAGGGCTGGGCAATCAGGAAAGCTTGGGTGACAGGGTGGCCCAGGAGC |
| RPL28_NM_000991_r1_12 | /5Biosg/CTCAAGTGCTCCGCTCACCTCAGCCTCTACTCAACCTCTGAGGAGCTGGGACCACA |
| (SEQ ID NO: 998) | GGCGGTGCACCACCACACCCAGCTAATTTTATTTTAGAGACGGTGTCTCATTATGTTGCCTAGG |
| RPL28_NM_000991_r1_13 | /5Biosg/TGTGAGGATCAACAGCAGGACTCTTTCCCCATCCAGGCCTCACTGTGTGTCATGGT |
| (SEQ ID NO: 999) | GTCTTCCAGGCTGGAGTGTAGTGGTGGGGCTCATGGGCTCACTGCAGCCTCATACTCTTGGCTC |
| RPL28_NM_000991_r1_14 | /5Biosg/AGGGAGCAGAGTCCTTCTTTGAATCGGGTACCCCAGGATGGCAGGAACCAGGGGTG |
| (SEQ ID NO: 1000) | CCATGGATGCTGGATCCTTGGTCCCTGGCAGTGGCTGACAGAGTTAGGTGCCCAGGAAACATGT |
| RPL28_NM_000991_r1_15 | /5Biosg/AGGGCAGTGGTCTGCCTCAAAACGCAGGGCTCCCAGCCCCGTACGACCCCAACCAA |
| (SEQ ID NO: 1001) | TGGCAAGTCGAGGCCCTGACAGTCCCCTTACTCTCAGTCAGAGCCGGGGGTGGTCTCAGACAGG |
| RPL28_NM_000991_r1_16 | /5Biosg/CCCAACTAAGGGAAGGACAAAGGAAATGGTAATTCACAGCAGACAGGGGCTGGCAA |
| (SEQ ID NO: 1002) | GTGGTCAGATCTGCTCTCCTTCGAGCTGGGCAAGACTGGAGCAGACAGGACTGAGGTCGGAAGG |
| RPL28_NM_000991_r1_17 | /5Biosg/AGTGAGGCCAGCTGGAGGTGGGCCTGAGTGGGATTGGGACAGTGCGGGGGGTCCTG |
| (SEQ ID NO: 1003) | AGCAGGTACCAAGTCACCCAACTCAGTTTTAAGGCAACACCTCTCAATCTGAGCTAATAGACCC |
| RPL28_NM_000991_r1_18 | /5Biosg/TGAGCAGTGAGCTCTCCTTATGAAGGCGGAGTCCCAGCGCAGTGAGGCCAGCCAGA |
| (SEQ ID NO: 1004) | GATGGGCCTGAGTGGGACTGGGACAGTGGGGGCTCCTGAGCAGGTACGAAGTCACCAGCGGAGT |
| RPL28_NM_000991_r1_19 | /5Biosg/AGACAAACATGAGACAGATAAACCTGCCAGCTTGTGGGCTGCAAATCAGTGCTGTG |
| (SEQ ID NO: 1005) | GAGGACAACTGAAGCAGGTGCCAGGGGAGAGGCCTCACGAGAAGGGGCCATCTACTAACGTGAG |
| RPL28_NM_000991_r1_20 | /5Biosg/AAGTCTCCAGTGAGCAAACTCTCAGGACCGGTGGGCTTTATACATTCAATATGCAC |
| (SEQ ID NO: 1006) | CATTCATCAAGCATCTCATTATATGCTGGTGTGCTAGACAAACCCCTTGCCCACCAGCACAAGA |
| RPL28_NM_000991_r1_21 | /5Biosg/ACTCGGGAGGCTGAGGCAGGAGAATCGCTTGAACCCAGGAGGTGGAGGCTGCAGTG |
| (SEQ ID NO: 1007) | AGCCAAGATCGCGCCATTGCACTCGCCAGCCTGGGCAACAGAGCGAGACTCCATCTCCAGAAAG |
| RPL28_NM_000991_r1_22 | /5Biosg/GGTGGGTAATTTGAAGTCAGGAGTTCCAAACCAGCCTGGGCAAAATGGTGAAACCC |
| (SEQ ID NO: 1008) | CATCTCTGCTAAAAATACAATACTGAGCTGGGTGTGGTGACACCCACCTGTAATCCCAGCTACT |
| RPL28_NM_000991_r1_23 | /5Biosg/GGCACAAGCCTCCAGGGCAGGCAAGGAGGTGGTTAAGGACAAGGCCTTCTGAGCAG |
| (SEQ ID NO: 1009) | CAGCAGCCACATCAGAAAGCCTCGAGCGCCTGTAATGCCAGCACTTTGGGAGGCTGAGGCAGGT |
| RPL28_NM_000991_r1_24 | /5Biosg/CAGAGGCCCACTAAGCTGGGCTGTGTTACGGATGAGACCAGGGGAGGCCACACAGC |
| (SEQ ID NO: 1010) | AGGGAGGATTCAGATAGGGATGGAGAAAGCCAGCAGGACTCCACAGGGGGTGGGGCCTAGAGGC |
| RPL28_NM_000991_r1_25 | /5Biosg/CATCTCCCTCCCTCCAGCCCTCTCCTGGTTCCTCCACCCCAACCCACCGATAGCAT |
| (SEQ ID NO: 1011) | TCAGATACCAGACGTGGCCTCCCCAGCCCAGCCACACAGACAGGCTGGCCACCCGCAGGAACAG |
| RPL28_NM_000991_r1_26 | /5Biosg/TGGAATAGATGGCCCCATCCAACATCTCTCTCTCCAAGATGAGGTCTAGAGGAGGG |
| (SEQ ID NO: 1012) | AAATGGTGTCTCCACGTTTGCTCAGCACACTGAGGACTCCAAACTCTGTGGGGCTGAGACCATC |
| RPL28_NM_000991_r1_27 | /5Biosg/TCTTTGGCTTTCAGCATTTTTACTATGGTGAGCCTGCCTGTTTTCTTAATATCATG |
| (SEQ ID NO: 1013) | TTTTTGGTTTAACTGGTTTTCCAATTTCCAGGCCAACAGACATGATTTGTGTGAATAAAGCTGG |
| RPL28_NM_000991_r1_28 | /5Biosg/CAGCACTCTGGATGTGTCCCCACTGCCTGGGCCTGGTTTCTGATGAGGGCAGCTAC |
| (SEQ ID NO: 1014) | TAACTTTATCAGGGACCTTCTGTATGTGACAAGACGCTTTTCTTTTGTTCTCCCAATTTTGTCT |
| RPL28_NM_000991_r1_29 | /5Biosg/GAGCTGGGACTGGAACCCAGGCAGTTTGATTCCTGGCTTCCGTGAACCATTAGCCC |
| (SEQ ID NO: 1015) | TGAGCTGGGAATGCTGCCGGGCATCACTCTTGGGGATAGGATGACCTGGGGGAGGTTCTTTCAG |
| RPL28_NM_000991_r1_30 | /5Biosg/CACCTGCCTTATGCAGGGCTGTGAAAGGCACTCAAAATGGCCTTTTACATATGGGA |
| (SEQ ID NO: 1016) | AACTGAGGCACGGTGCTACTAAACGAGCTGCCCAAGTCCCCACAGCTGGGCATAACTGGCAGAG |
| RPL28_NM_000991_r1_31 | /5Biosg/GGAGTCCCCAGCGCTCTGCACAACTTTGGGCTTGGCAGCCTTCCAGGGTGCGCCCT |
| (SEQ ID NO: 1017) | ACGCTCACTCACCATGCAGCTACTGGACTAAGAGCTGGAGAGACAGCAGTGAACACTGAGACAC |
| RPL28_NM_000991_r1_32 | /5Biosg/GGAACTTGCCCAAGGTTGCACAGCCACCGAGCAGCAGAGCCCTGACCCAGTAGATG |
| (SEQ ID NO: 1018) | CCAGCATGTCCTGTGAACAGAGGATGCCTCAGTCCCGAGTCTCTGCTGCTGTGGGGAGTCTGGA |
| RPL28_NM_000991_r1_33 | /5Biosg/GATTTGTGGGCACAGTCAAGCAGGTTTTGCACATTTAATCTTCTCATCAACCCTGA |
| (SEQ ID NO: 1019) | GTAGTAGGGTCTATCGACACGCATGTTACAGATGAGGGTACGAAGACACAGAGAGGTTGAGGAA |
| RPL28_NM_000991_r1_34 | /5Biosg/GAAACACGAGCATTAGTTTGTGTACAAATTAGTCATAGGCATGTGAAGACATCTGT |
| (SEQ ID NO: 1020) | GATCATGAGTCCATCTGATCAAAATATTTATAAAAAGTCATTAATTTATTCCTACAATCAGGAT |
| RPL28_NM_000991_r1_35 | /5Biosg/TATTCACAAACACGGGCAGGAGGCTGGGAAAGCTCACAGCCCCACAGGACGGCGGG |
| (SEQ ID NO: 1021) | GCAGACCTGGAGCCACCAGGCATTACTGCACATGGATCTGGCATGTCTTCCAGGTGCTTGGGAA |
| RPL28_NM_000991_r1_36 | /5Biosg/TTTTTAAAGTAGATGATTTTATTCTAGTAACTGGCTACAAGAGAAACAGGGGAATT |
| (SEQ ID NO: 1022) | GATACCATTCCTGGGCAGCGCCAAACAGCCTGGGCTGGAAATGCAGCTGAGGACAGAATGATAT |
| RPL29_NM_000992_r1_1 | /5Biosg/GTGGTGTGGTTCTTGGACTTGGCCATGTCTGCACCATAAGCCGCGGCTCCCGAAGC |
| (SEQ ID NO: 1023) | GCCTAGAACCGGAAGAGAAAGGGGCTGCGGTGCAGCACGGGAAATAGGGTCAACAACGCCGGAA |
| RPL29_NM_000992_r1_2 | /5Biosg/CTTGGCAAAGCGCATGTTCCTCAGCAACTTGGGGTCCACCCCCTTAAGAGATTCGT |
| (SEQ ID NO: 1024) | ATCTTTGTGATCGGGGTTTCTTGATACCATTTCTGTGCCATTTTCGGGACTGGTTGTGTGTGGT |
| RPL29_NM_000992_r1_3 | /5Biosg/GGGATCTTGGGCTTAACCTCCTTGGGCTTTACGAGGGCCTTGATAGCCTCGGCACG |
| (SEQ ID NO: 1025) | TGCACTCATGGCCTTGGCATTGTTGGCCTGCATCTTCTTTAGGCCCTTTTTGTTGTGCTTCTTG |
| RPL29_NM_000992_r1_4 | /5Biosg/CTTGGCCTTTGGCCGGCACAGCCTGAGCCCCTTGGCAATACGGGCACGAGCACGCT |
| (SEQ ID NO: 1026) | TCCCAAGCTTGGGGTGGGCAATGTAGGCAAGTCGATCGAGCTTGCGGCTGACACCCTTTGGGAT |
| RPL29_NM_000992_r1_5 | /5Biosg/AGAGATATCTACTCTGAAGCCTTTGTAGGGGCCTGGGTACGTTTGGGAGCCTGAGC |
| (SEQ ID NO: 1027) | TGGAACTGAAGCTGGGGCTGCAGCCTGGGCCTTGGTTTGATCCTTGGCCTTGGCCTTGGCCTTG |
| RPL29_NM_000992_r1_6 | /5Biosg/TTTCCTGCCTCAGGTTTATTTGTACAAATAGCACAGGAGGACCCCAGCCCCATGCA |
| (SEQ ID NO: 1028) | GATGGTAGCCCAGGGGCGGGGGTGGGGGGTCGCACCAGTCCTTCTGTCCTCATGTTGGCAGAGA |
| RPL30_NM_000989_r1_1 | /5Biosg/ACCATCTTCCTGCCTTAGCAGCGGGACGGCCCCCAACCAACAGCAGCCGCTAAGAT |
| (SEQ ID NO: 1029) | GGCCGGGGAACGAGAAAGGAAAGACTTCCCACAATGCAAAGCTCTTCACGGCAGAGCCGGAACT |
| RPL30_NM_000989_r1_2 | /5Biosg/GATCATCTTCAGAGTCTGCTTGTACCCCAGGACGTACTTCCCACTTTTCATAACGA |
| (SEQ ID NO: 1030) | GTTGGAGCCTAGAGTTGATCGACTCCAGCGACTTTTTCGTCTTCTTTGCGGCCACCATCTTCCT |
| RPL30_NM_000989_r1_3 | /5Biosg/GTAGTGATGGACACCAGTTTTAGCCAACATAGCATAGTACTCTATTTCAGATTTCC |
| (SEQ ID NO: 1031) | TCAAAGCTGGGCAGTTGTTAGCGAGAATGACCAATTTCGCTTTGCCTTGTCTGATCATCTTCAG |
| RPL30_NM_000989_r1_4 | /5Biosg/TTCTGGCATGCTTCTAATGATGTCAGAGTCACCTGGATCAATGATAGCCAGTGTGC |
| (SEQ ID NO: 1032) | ACACTCTGTAGTATTTTCCGCATGCTGTGCCCAGTTCAATATTATTGCCACTGTAGTGATGGAC |
| RPL30_NM_000989_r1_5 | /5Biosg/TAGATACAATGTTTTTAAAACAAGCAAATTTTATTAAAGGAAAATTTTGCAGGTTT |
| (SEQ ID NO: 1033) | AAGGTTTGCAGGTGAAATTTTGTAGGTGAAAAGGTTTACTTTTCACCAGTCTGTTCTGGCATGC |
| RPL31_NM_000993_r1_1 | /5Biosg/TTTCTTCTCGCCACCCTTCTTTGCGGGAGCCATTCTGCAGCGTCCAAGTTGGAAAG |
| (SEQ ID NO: 1034) | GAAGAGCGCGAAGGATTGTGGGAGAAGGAAAGCCGCAGTTGCAAGTACAACTTCCGGGTCGCCA |
| RPL31_NM_000993_r1_2 | /5Biosg/GAGGTGCACGCTTCTTGAAGCCCACTCCATGGATGCGCTTGTGAATGTTGATGGTG |
| (SEQ ID NO: 1035) | TATTCTCGGGTTACCACTTCGTTGATGGCAGAACGGCCCTTTTTCTTCTCGCCACCCTTCTTTG |
| RPL31_NM_000993_r1_3 | /5Biosg/TCCTTTGGCCCAGACAGCTTTGTTGAGCCTGGTGTCAATGCGCACATCTGGAGTTC |
| (SEQ ID NO: 1036) | CCATCTCCTTCATGGCAAATTTCCGAATCTCTTTGAGTGCCCGAGGTGCACGCTTCTTGAAGCC |
| RPL31_NM_000993_r1_4 | /5Biosg/ACATAGGTAACCAAAGTATATAGCTTATTTGGTGAATCTTCATCCTCATTACGTTT |
| (SEQ ID NO: 1037) | TCTGGACAGCCGCACACGGATTCGGTATGGCACATTCCTTATTCCTTTGGCCCAGACAGCTTTG |
| RPL31_NM_000993_r1_5 | /5Biosg/TTTTGAAGGCAATTTTATAACTTTATTTGATCTGACGATCAGCGATTAGTTCTCAT |
| (SEQ ID NO: 1038) | CCACATTGACTGTCTGTAGATTTTTGAAAGTGGTAACAGGTACATAGGTAACCAAAGTATATAG |
| RPL32_NM_000994_r1_1 | /5Biosg/TTCACAAGGGGTCTGAGGGCGGCCATGATGCCGAGAAGGAGATGGCTGCCACCTCC |
| (SEQ ID NO: 1039) | GTAGGCAGCGCCGAGGAAGAGAGAAGGGACGGTGGCGCGCAAGGGCTGATGGGTCGTAACCCCT |
| RPL32_NM_000994_r1_2 | /5Biosg/TCTTCTACGAACCCTGTTGTCAATGCCTCTGGGTTTCCGCCAGTTACGCTTAATTT |
| (SEQ ID NO: 1040) | TGACATATCGGTCTGACTGGTGCCGGATGAACTTCTTGGTTCTCTTTTTGACGATCTTGGGCTT |
| RPL32_NM_000994_r1_3 | /5Biosg/AGCACTTCCAGCTCCTTGACGTTGTGGACCAGGAACTTCCGGAAGCCACTGGGCAG |
| (SEQ ID NO: 1041) | CATGTGCTTTGTTTTTTTGTTGCTTCCATAACCAATGTTGGGCATCAAGATCTGGCCCTTGAAT |
| RPL32_NM_000994_r1_4 | /5Biosg/GGCATTGGGGTTGGTGACTCTGATGGCCAGTTGGGCAGCTCTTTCCACGATGGCTT |
| (SEQ ID NO: 1042) | TGCGGTTCTTGGAGGAAACATTGTGAGCGATCTCGGCACAGTAAGATTTGTTGCACATCAGCAG |
| RPL32_NM_000994_r1_5 | /5Biosg/GGCCAAGAAGCTGAAGACTTAAAATCAAGGAAGGAAGATGCCAGATGGCAGTTTTT |
| (SEQ ID NO: 1043) | ACATTTATTTAAACAGAAAACGTGCACATGAGCTGCCTACTCATTTTCTTCACTGCGCAGCCTG |
| RPL32_NM_000994_r1_6 | /5Biosg/TGTGCACTTGAGGCCACATTCCCCTGTTCAAGGACTGAGTGTCCTTTACAAATCCC |
| (SEQ ID NO: 1044) | CTCCAAATGGGAGATTCCAAAGGGGCTTAAGCAAAAGAACAATCTCTGTGGCAAACTAAGTTGG |
| RPL32_NM_000994_r1_7 | /5Biosg/ATGACTACTTGTGGCTTGGGAGCCACAAGCTTCTTCAAGTGTCTCAGAACCTACCT |
| (SEQ ID NO: 1045) | GGTGTGAGGGCCAAGTCTGTACCCCTCATACCCAGCCTCAACTGGAGATGACTAAGGCTAGTCT |
| RPL32_NM_000994_r1_8 | /5Biosg/CATCCCGTAAGAATCTGTGTAAGAAATTCATCTGGATATTTCCATGCATATTTTCT |
| (SEQ ID NO: 1046) | CAATTCTCACAAGCATCACATGGAATAACTTGTCACCTAACTTTACAAAAGCAAGGCTAAGAAT |
| RPL32_NM_000994_r1_9 | /5Biosg/GTGGGAGGGTTATAGGAGACTGAAAGTGCTTTTCCAGTTAACCGTGGTGGATTACC |
| (SEQ ID NO: 1047) | TGGAAGAGCAATTTGTACATCATCCTGTTCTTTTTGGACAGAAGTTACAGGATGCAATTTAGGC |
| RPL32_NM_000994_r1_10 | /5Biosg/AAAAAAAATCAGGACCACGTGTCACTAAATTCTGAGTACCAGTCAAGAGGCTCAAT |
| (SEQ ID NO: 1048) | TGTCACCAGTTAGGTCCCCGCCTTGGCAAACTGCTGTAACATTATACCTGAAGCAGCAGCTGGT |
| RPL32_NM_000994_r1_11 | /5Biosg/CTGTAGTCCCAGCTACTTGGAAGGCTGAGGTGGGAAGATCACCTGAGCCTGGGGAG |
| (SEQ ID NO: 1049) | GTCAAGGCTGTAGTCAGTGTGCCACTGCACTCCCAGCCTGGGTGAGAGCAAGACCCCGTCTCCA |
| RPL32_NM_000994_r1_12 | /5Biosg/GTACTTTGGGAGGCTGAAGCAGGATTGCTTGAGCTCAGGAGTTCGGGAGCCTAGGC |
| (SEQ ID NO: 1050) | AACATGGCTAGACCACCTCTACAAAAAAACTTCAAAAACTACCCAGGTTTGGAGGTGTGCATCT |
| RPL32_NM_000994_r1_13 | /5Biosg/TTTCTGCTAAGTTGACCAGGATGGTTACAGAAAAACATCCTGTAAGCATTTCTGTC |
| (SEQ ID NO: 1051) | TCATAAGTACCACATCCCATATCCCTCATGACCTATATACTACAGATGATGCCTGTAATCCCAG |
| RPL32_NM_000994_r1_14 | /5Biosg/TTTTTTTTTTGACCAGTTTGCATTATAAATATTTATGACTAGGTTTTGAACAGGAG |
| (SEQ ID NO: 1052) | ACAATCTGTAAGATTCCTGTCTAGACTAGAAGTAGAAAAGCTTTATTATACCCAGCGCAGCATT |
| RPL34_NM_000995_r1_1 | /5Biosg/GGGTTCGGGACAGCCTAGTTTTGTTAGAGGCTGTATTGTAGGAAAGCCTACGTCGG |
| (SEQ ID NO: 1053) | TATGTCAAACGCTGGACCATTCTGAGTGCCTGCAGACAACGTCCCCGGAAGAGGAAGAAAAAAG |
| RPL34_NM_000995_r1_2 | /5Biosg/CTTTAGGTCTTACAGCACGAACCCCTCGAAGTCTGCCTGGGCACACACCACATGCA |
| (SEQ ID NO: 1054) | GATTTTGGTGCTTTCCCAACCTTCTTGGTATAAAGGTAAACAATTCTATTACCAGGGGTTCGGG |
| RPL34_NM_000995_r1_3 | /5Biosg/TGCTCCTCGATAAGGAAAGCACGCTTGATCCTGTCACGAACACATTTAGCACACAT |
| (SEQ ID NO: 1055) | GGAACCACCATAGGCCCTGCTGACATGTTTCTTTGTTTTGGACAATCTCATAAGAACTTTAGGT |
| RPL34_NM_000995_r1_24 | /5Biosg/CTTTTTCTATTGGACAGCGTCTTTTCATTTTTATTACTCAAAAAAGTTTCATTTTT |
| (SEQ ID NO: 1056) | TTATTTAGCTTTCTGACTCTGTGCTTGTGCCTTCAACACTTTCACAACGATTTTCTGCTCCTCG |
| RPL34_NM_000995_r1_5 | /5Biosg/TCTCACCATAACAATCTCAAGATGCCAAGCAGACGTCACTGGATGCAAAACTGGAA |
| (SEQ ID NO: 1057) | AGAGATGCACACAACTGGCTCTCTCAAGCTGAGGTGAGGTAGCTCCAGCACACCAACTTTTTCT |
| RPL34_NM_000995_r1_6 | /5Biosg/AGTGATCATATACTGTAACTTGAGTAGTATCTAGGGATGTGCTGGTAGTATTTAAC |
| (SEQ ID NO: 1058) | CTCCAGCTCTCTGGGGGAAAACAGGATTTTGCAGCATTTGCTGAGGTGTAAATACTCTCACCAT |
| RPL34_NM_000995_r1_7 | /5Biosg/AAACTCTTTACAGAATCGAAGTATTAATAAGTACTTATAATAACAATCAAGTCATT |
| (SEQ ID NO: 1059) | ATCAAACATGTACCAGATATATATCAGAGCTCCTGGTACCAAGAGCATACTATATTAGTGATCA |
| RPL34_NM_000995_r1_8 | /5Biosg/CCCCTTTGCAAGATTTTTAATTCTTTTCCCTTCCATTCATACAGAGAACTGGTCAC |
| (SEQ ID NO: 1060) | ATAGAGAACTGGATTCAATAAAAGGCTCATTTTGGATTTTATAAAATCAAACCCTAAACTCTTT |
| RPL35_NM_007209_r1_1 | /5Biosg/AGGTCGTCCAGCTGTTTCAGCAGCTCCTCCTTCTTCTTCCCGCGAAGATCTCGAGC |
| (SEQ ID NO: 1061) | CTTGATCTTGGCCATTGCTGCACAAGCCGCCAACGCCGCCGCCCGCTCCGAGGGAAAGAGGAAG |
| RPL35_NM_007209_r1_2 | /5Biosg/TAATAACTGTGAGAACACGGGCAATGGATTTCCGGACGACTCGGATCTTAGAGAGC |
| (SEQ ID NO: 1062) | TTGGAGGCCGCACCGCCTGTCACTTTGGCGACGCGCAGCTGGGACAGCTCCACCTTCAGGTCGT |
| RPL35_NM_007209_r1_3 | /5Biosg/CCTCGTGCTTGTTGAGCCGGCGGCGCATGGCACGTGTCTTCTTAGGCCGCAGGTCC |
| (SEQ ID NO: 1063) | AGGGGCTTGTACTTCTTGCCCTTGTAGAATTTCCTGAGGTTTTCTTTCTGAGTCTGGTTAATAA |
| RPL35_NM_007209_r1_4 | /5Biosg/GCAGTCTCAGCCAGCTGTGCTTTATTGACAATGCGCCCCTCAGGCCTTGACCGCGT |
| (SEQ ID NO: 1064) | ACTTCCGCAGCGGGTACAGCCGCTCCTTCCGCTGCTGCTTCTTGGTCTTCAGGTTCTCCTCGTG |
| RPL36A_NM_021029_r1_1 | /5Biosg/AACCGCGAGGCAAACGCAAGTATATAGGAGGTTCCCGATCGCACTTCCTCATGGGA |
| (SEQ ID NO: 1065) | GTCGGTAGGAGCAATCATAGAGTGTAAGGCTCAGCGCAGCGCCCTCGGGCGGCTGAGAGGACTC |
| RPL36A_NM_021029_r1_2 | /5Biosg/CTGTGTCACTTTATGGGGTTGGTGCTTGCCACACTTCTTACAGAAAGTCCGGCGGG |
| (SEQ ID NO: 1066) | TTTTAGGGACGTTAACCATGCTTGCGTGAGCGCTATCGGCGCGGAAAGAAAGAAACCGCGAGGC |
| RPL36A_NM_021029_r1_3 | /5Biosg/TTTAGCCTTTTTCCGGAAAATCGGCTTAGTTTGCCCACCATAGCCACTCTGCTTCC |
| (SEQ ID NO: 1067) | TGTCATAACGCCGCTTTCCCTGGGCGTACAGAGAATCCTTGCCCTTCTTGTACTGTGTCACTTT |
| RPL36A_NM_021029_r1_4 | /5Biosg/TCTTATCTCCTCCCAGTTCAAAATGCTTGCATCTTTTAATAGCCAGCATTCTCTTA |
| (SEQ ID NO: 1068) | GATCTGCAGTTGGGCTCAACGCACTCAAGCCTTAGCACAATCTTCTTTGTAGTTTTAGCCTTTT |
| RPL36A_NM_021029_r1_5 | /5Biosg/CCCTCCCAAAAGATGAACAGCAAACAAATGAAGTGAACATAAACTCAAGATTTTAT |
| (SEQ ID NO: 1069) | TGTCTTCATAATAAAAGATGACACTTAGAACTGGATCACTTGGCCCTTTCTCTTCTTATCTCCT |
| RPL36A_NM_021029_r1_6 | /5Biosg/CAACATATGATGTGAACTCAACCAGTCAGCTTCAATGCAAAACAAGTAGTACCAGT |
| (SEQ ID NO: 1070) | AAGAGGCATAAATTTCCCCACAAGCGGAATTGTATTGATGGCTCTAGCTTATTCCCTCCCAAAA |
| RPL36A_NM_021029_r1_7 | /5Biosg/CCCTATTAGCCTGAAATGTGTCTACTCACATGATAACCAATAACCATTGCATAATG |
| (SEQ ID NO: 1071) | ACAAACTGCCTCATAAGGCCCCTAGTGATTGAAGTGCCAAATTAGAAAATTGCAACATATGATG |
| RPL36A_NM_021029_r1_8 | /5Biosg/GAAGAACAATGCCAAAATTCTTTATTAAATTAACTGTTATAACAAAGATCTGACAC |
| (SEQ ID NO: 1072) | TTAACTCTTAGCAAAGACATCTCATATTCACTATGAATGTGTTACTGACTTCTCCCTATTAGCC |
| RPL36_NM_033643_r1_1 | /5Biosg/CACTTTGTGGCCCTTGTTGAGGCCCACGGCCATAGGGTAGCGTAGGGCCATGGCTG |
| (SEQ ID NO: 1073) | CTGCTCTCCAGAGACGGCCGTGGCGGAAGGGCTGGCGCCGCGCGGAACTCCGGGATATCTACTC |
| RPL36_NM_033643_r1_2 | /5Biosg/CGGGGCAAAGCCACACACCTCCCGAATCATGTCCCGCACGAACTTGGTGTGTTTGG |
| (SEQ ID NO: 1074) | TCAGACGCCCGCGGCGTCGGCTGTGCCTGGGCTTGCTCACGTTCTTGGTCACTTTGTGGCCCTT |
| RPL36_NM_033643_r1_3 | /5Biosg/CCGCTTCCTCTTGGCGCGGATGTGCGTCCCCACCCTTTTCTTGATAAATTTGAGGG |
| (SEQ ID NO: 1075) | CCCGTTTGTCCTTGGAGACCTTCAGTAACTCCATGGCGCGCCGCTCGTACGGGGCAAAGCCACA |
| RPL36_NM_033643_r1_4 | /5Biosg/TTTTCTGTCAAGCTGTTCTTTATTTCAGGGAGAGGGCAGGGGAGGGGCTCAGTCTT |
| (SEQ ID NO: 1076) | TCTTGGCAGCAGCTTTCCTCATGGCGGCCAGTACGTTGCTCAGCTCCTCCCGCTTCCTCTTGGC |
| RPL37A_NM_000998_r1_1 | /5Biosg/GTATTTACCGACGATCCCGACTTTCTTGGTACGTTTGGCCATGTCGCCGCGACCTA |
| (SEQ ID NO: 1077) | GGTCCGAGCCCAGAAAGGAAGAGACGCGGTGCGCAGGCGCGGTATTAGGCGGACGAGCGGAAGT |
| RPL37A_NM_000998_r1_2 | /5Biosg/TCATCTTGGTTTTGCCACAGAAAGAGCAAGTGTACTTGGCGTGCTGGCTGATTTCA |
| (SEQ ID NO: 1078) | ATTTTCTTCACCATTTTCCGGAGGGAGGCCCCATAGCGGGTCCCGTATTTACCGACGATCCCGA |
| RPL37A_NM_000998_r1_3 | /5Biosg/GACTTTACCGTGACAGCGGAAGTGGTATTGTACGTCCAGGCACCGCCAGCCACTGT |
| (SEQ ID NO: 1079) | CTTCATGCAGGAACCACAGTGCCAGATCCCCACAGCTCGTCTCTTCATCTTGGTTTTGCCACAG |
| RPL37A_NM_000998_r1_4 | /5Biosg/TTTTGTTACATAAATTAACCCATTTATTATAGGCCAGTGATGTCTCAAAGAGTAGA |
| (SEQ ID NO: 1080) | GGAGCGTCTACTGGTCTTTCAACTCCTTCAGTCTTCTGATGGCGGACTTTACCGTGACAGCGGA |
| RPL37_NM_000997_r1_1 | /5Biosg/GATGACGTTCCCTTCGTCATCTCGCTTCTGCGGCCGAGACCAGAAAGACCGGAAGA |
| (SEQ ID NO: 1081) | GAAGGCACTTCCGCTATATCACGCTCAGAAGCTTCCGCCCAGGGAAGCACTTCCTGCGGGGCGC |
| RPL37_NM_000997_r1_2 | /5Biosg/CTTGCGCTTGGCAGGGTAGCCACATTTGCCACAGGTCGACTTCTGAAGGTGGTAGG |
| (SEQ ID NO: 1082) | CCTTAGAGCCACAGCGGCGGCACAACGTGTGCGTCTTATTGCGACGCTTTCCAAACGATGACGT |
| RPL37_NM_000997_r1_3 | /5Biosg/TTCCTTCACGGAATCCATGCCTGAATCTGCGGTATACAATTTTTAGGTGCCTCATT |
| (SEQ ID NO: 1083) | CGACCAGTTCCGGTGGTATTTCGTCTTTTAGCCTTGGCACTCCAGTTATACTTTCTCTTGCGCT |
| RPL37_NM_000997_r1_4 | /5Biosg/CAAAACCAGATATGTATTTTTTAAAACCAGAACATTTATTGCATGACTAATCGTTG |
| (SEQ ID NO: 1084) | ACATTCTTAAGATGAACTGGATGCTGCAACAGCTGCCCTCTTGGGTTTAGGTGTTGTTCCTTCA |
| RPL37_NM_000997_r1_5 | /5Biosg/AACATTCCAAAACAGTCACTTAACAAGTAAATCTGATATGAAGCTAGCCCAGTCCC |
| (SEQ ID NO: 1085) | TAAACCTACAGTATTTCACTGATACTACAAGCCTAATTGATTAAAAATACCTTACCAAAACCAG |
| RPL37_NM_000997_r1_6 | /5Biosg/GTGCTATTTTAATCTGCTATATTGTCTTCCAGTGCCCTCTGCTTCAAGGACTCCTG |
| (SEQ ID NO: 1086) | GAATTCTGCTTGTTTCTCATTGCCTTTAACCGTGTTACAAACCCAGTCCAAAAGTAAACATTCC |
| RPL37_NM_000997_r1_7 | /5Biosg/CCCCCTAGTCATGACAGCATAGCAGATGAACATGTTGCTAAAGGTAATTTAAACAT |
| (SEQ ID NO: 1087) | CATACTCTTTCAAATTTACTCAAACATCTAAAATACCCACCTATGCCACATGAGCTGTGCTATT |
| RPL37_NM_000997_r1_8 | /5Biosg/GTGAGCATTACAGATACTAGGATAAGATCATCTTTTGAAAAACAGAAGCCACCTAA |
| (SEQ ID NO: 1088) | ACTCCTACCCATACGTTTCCAGTGGTACTCCCAAGCTCTATGTGACTAATAATCATCCCCCTAG |
| RPL37_NM_000997_r1_9 | /5Biosg/AGCCCTACTTTTTCTCAAACCTATTTACATATTCTTAAGTTGACTCACACCTAGCA |
| (SEQ ID NO: 1089) | GGCTTTTTTCACTTAGCTAGCCACCTTACACACAGCCCACAAGTCAGGTGTGCCAAGTGAGCAT |
| RPL37_NM_000997_r1_10 | /5Biosg/GAGGGCTAAAAAAATTCGACCAAGTAGTCCCACATCATTGCCTTTAGTCTACAAAT |
| (SEQ ID NO: 1090) | CTCAGGGATTATACTTGATTTTCCTTTATTTCCTGCTCAATCTTTACTTGCACCCAAGCCCTAC |
| RPL37_NM_000997_r1_11 | /5Biosg/CATTTTAAAAAAACAAAAAAAACCCCCAACCCTGTCAATAAACCACGAAAACTAGG |
| (SEQ ID NO: 1091) | AAGGCACGACAGCAAGTTTCAGTGCTTTAACACAGAAACACCCAATTACCAAGTTGAGGGCTAA |
| RPL37_NM_000997_r1_12 | /5Biosg/TTAAATAGCATGACTTGGCTACATCACTATTTTATGTAGCTGTTCCAGCAATTCAT |
| (SEQ ID NO: 1092) | GTTCCCTTCAACTGCCCCTGCCCTCTCACTCAGCAGTCCAGTTGACTTTGTCCCTTCATTTTAA |
| RPL37_NM_000997_r1_13 | /5Biosg/CAATTAGCTAGCACATTATAAAGCCATTCCAGGTTACAAGTAAAAGTGATACCCTT |
| (SEQ ID NO: 1093) | CCAAGGCCAGGTTAAGAACCTCAGATGTTATTCTAAACACAGTGGAGAATTATAATTTAAATAG |
| RPL37_NM_000997_r1_14 | /5Biosg/TTTTGGGAGGAAGCCCAAGAGAGATTTAATCAGCAGGGTTTCTTTATAAGTGAATG |
| (SEQ ID NO: 1094) | TTCTGAAGCTGAGACCTGAAGGGTAAATTAGGAGTTAAAATACAAGATGAGAGTAGCAATTAGC |
| RPL38_NM_000999_r1_1 | /5Biosg/GGCGCGCTGGGCTCTGGCGCGGACCAGGACCTTTCTCACCCACGTATCACCCTAGA |
| (SEQ ID NO: 1095) | GACACTCACAGCAAGCAGCAACCGGGGAAAAGGACGAAAAAGAACGAGACTTCCGTTTCCGGGC |
| RPL38_NM_000999_r1_2 | /5Riosg/TTTTTCTTGATCTTGACAGATTTGGCATCCTTTCGTCGGGCTGTGAGCAGGAAGTC |
| (SEQ ID NO: 1096) | CTTGATTTCCTCAATTTTCCGAGGCATGGCGACGAGGCGCGCTGGGCTCTGGCGCGGACCAGGA |
| RPL38_NM_000999_r1_3 | /5Biosg/CTGCTTCAGTTTCTCTGCCTTCTCTTTGTCAGTGATGACCAGGGTGTAAAGGTATC |
| (SEQ ID NO: 1097) | TGCTGCATCGAACTTTAAACTTCACGTTGTCCTTATTTTTCTTGATCTTGACAGATTTGGCATC |
| RPL38_NM_000999_r1_4 | /5Biosg/TTTAGGATTTTTAGTATTTTAATATAATACAGTTCCAATCAGTGTGTCTGGTTCAT |
| (SEQ ID NO: 1098) | TTCAGTTCCTTCACTGCCAAACCGGGGGGCAGGGACTGCTTCAGTTTCTCTGCCTTCTCTTTGT |
| RPL39_NM_001000_r1_1 | /5Biosg/GGAATCGCTTAATCCTGAAAGTCTTGTGAGAAGACATGGCGAGCAGCGGAGTCAAG |
| (SEQ ID NO: 1099) | AACACACCACGATGGCGGAGAAAGGAAGAGGAGGGAAGCTGGCGGAAGAACGAGCTTAGAAGGC |
| RPL39_NM_001000_r1_2 | /5Biosg/CTTCTCCAATGTCTCCTTTTGGAGTTGTACCTGATTTTATTTCCAGTTTTCATCCG |
| (SEQ ID NO: 1100) | AATCCACTGGGGAATGGGACGATTTTGCTTTTGTTTCTTGGCCAGGAATCGCTTAATCCTGAAA |
| RPL39_NM_001000_r1_3 | /5Biosg/GTGACATTTTCAGCTTGATATGGTAACATGATCGTGACCTTCAGACAGCATAAATA |
| (SEQ ID NO: 1101) | TGTGTGCCATCTCATGTGCAATTCCTTATAGACCCAGCTTGGTTCTTCTCCAATGTCTCCTTTT |
| RPL39_NM_001000_r1_4 | /5Biosg/TTTAAAAAGAAAGGCCTTACATATTTATTACTGAATCCAGCCAACCAACGTGTTCA |
| (SEQ ID NO: 1102) | TAACAGATTCAGAGAGGAAAACACGTCGAAATCTCCAGATAGTGGTGACATTTTCAGCTTGATA |
| RPL3_NM_00967_r1_1 | /5Biosg/GCAGGAAGCCGAGGGACCCATGTCTGGGAGCGGAGAACTTTCTGTGAGACATCACG |
| (SEQ ID NO: 1103) | CCATCAAATCCCGCCGGTAGAGGCCGGTCGGCCTTACGGGTCCGCTATATAAAGCCAAATCTGG |
| RPL3_NM_00967_r1_2 | /5Biosg/GAGTCATGCCAGCCTTGTATCCCAGGAAGGCTGTGAGGTGGACCGGCTTGGACGGG |
| (SEQ ID NO: 1104) | TCATCCTTAGGGAAGCTCTTCACCTTCCCACGATGCCTGCTGCTGCGCTTCCGAGGCAGGAAGC |
| RPL3_NM_00967_r1_3 | /5Biosg/AGCCCACAATGCCCACAACCACCATGGGTGGTGTCTCTACAATGGTCACAGCCTCC |
| (SEQ ID NO: 1105) | ACCACCTCCTTCTTGTTCACCTTGGATCCCGGCCTGTCGACTTCCCGCACGATGTGAGTCATGC |
| RPL3_NM_00967_r1_4 | /5Biosg/CCTTCTTCTTAGATTTATGCCAATTCTTATAGAAACGCCTCTTGCATTCATCACTG |
| (SEQ ID NO: 1106) | ATGTGCTCAGCAAAGACAGTCTTGAAGGTCCGGAGGCCTCGAGGGGTTTCCACGTAGCCCACAA |
| RPL3_NM_00967_r1_5 | /5Biosg/TGTGGCTAATGACACGGATGACTTGGCAGTACTTCTTCATGCTGCTGAAGTCCTTC |
| (SEQ ID NO: 1107) | TCCAGCTGCTTCTTGCCATCCTCATCCTGCCATTTCTTGCAGTACTTGGTAAAGGCCTTCTTCT |
| RPL3_NM_00967_r1_6 | /5Biosg/GCTCAAGCCTCTCGCGGGCCCAGTCCAGCTTCTCGGCCACAGTGCCTCCGTTCACC |
| (SEQ ID NO: 1108) | TGGATCTCCATCAGGTGGGCCTTCTTCTGGCGCAGAGGAAGCAGGCGCATCTGGGTGTGGGCAA |
| RPL3_NM_00967_r1_7 | /5Biosg/CTTCTTGGTGTGCCAACGACTGGTGACCCCTTTGTAGCCTTTGCCCTTGGTCACCC |
| (SEQ ID NO: 1109) | CGATGACGTCGATCATCTCATCCTGCCCAAACACTTGGTTCACAGGTACCTGCTGCTCAAGCCT |
| RPL3_NM_00967_r1_8 | /5Biosg/GTGATGGTAGCCTTTCTGCCCAGCGCGTGCCACAGAGAAGGCTACACGAGCAGGAT |
| (SEQ ID NO: 1110) | GCCATGCCCCAATACAGGCCACCTTGCGCAGGCCTCGGTGGGTCTTGCGGGGCAGCTTCTTGGT |
| RPL3_NM_00967_r1_9 | /5Biosg/GCTCTTGTCAGATAGGTCATAGTCAGTGGAGGCATTGTTCTTGATCAGCTTGCCGT |
| (SEQ ID NO: 1111) | CCTTGATAAGGTAGCCCTGGCCAATCTTATAAATCTTCTTGTTGATCTCAGTGCGGTGATGGTA |
| RPL3_NM_00967_r1_10 | /5Biosg/GGACTTGCGGAGGGTGAGCACCCGCTTCTTGGTTCCCACCACACAGCCTTTCAGCA |
| (SEQ ID NO: 1112) | TGACAAAGTCATTGGTCACTTCACCATAGTGGACAAAGCCACCCAGAGGGTTGATGCTCTTGTC |
| RPL3_NM_00967_r1_11 | /5Biosg/TGCTTTCTTCTCCTCCATGGTCTGGAAGCGGCCATGGCCAAACTTGGAGGTGGTGT |
| (SEQ ID NO: 1113) | CAATGAACTTAAGGTCAATCTTCTCCAGAGCCCGCCGCTTCGTCTGCACCAGCAAGGACTTGCG |
| RPL3_NM_00967_r1_12 | /5Biosg/TTTGTCAGTGGAAAATAACTTTTATTGAGACCCCACCAACTGCAAAATCTGTTCCT |
| (SEQ ID NO: 1114) | GGCATTAAGCTCCTTCTTCCTTTGCAATTCGGTCTTTCTTCAGTGGTCCCATGAATGCTTTCTT |
| RPL41_NM_021104_r1_1 | /5Biosg/GCCTGCGCATTCGCTTCTTCCTCCACTTGGCTCTCATGGCGCAGAGGTTTCCAAAA |
| (SEQ ID NO: 1115) | AAATGGCGCTAAGGCCGAGAGAAAGGCGAAGTTCCGTCTACGGCTATTTAATGGAGCGCCGGGT |
| RPL41_NM_021104_r1_2 | /5Biosg/CTTGTACCAGCATCCCCAGCGTCTGGCATTCCATGTTTCTGCTCCTGTGGCCTCCA |
| (SEQ ID NO: 1116) | CGGTGCAACAAGCTAGCGGTTTACTTGGACCTCTGCCTCATCTTTCTTCTTTTGCGCTTCAGCC |
| RPL41_NM_021104_r1_3 | /5Biosg/AACATGGGCATTTTGTTTATGAGCAAGGTGGGTCTCAGAGGTGATCGGCGATCAGA |
| (SEQ ID NO: 1117) | GGGCGATGAAGTTCTAGATCCATTGAGACAAGCTCTAGACAGTAGCATGCAGTCCCACAACTTG |
| RPL41_NM_021104_r1_4 | /5Biosg/TTGATTTAATTCTTCAGCTAAAACAGCGGAAGAGGTGATTTATTATATGGTTGTTA |
| (SEQ ID NO: 1118) | CACTCGGCCACAAATAAACACAGAAATAGTCCAGAATGTCACAGGTCCAGGGCAGAGGACCAAC |
| RPL4_NM_000968_r1_1 | /5Biosg/CAGATGACTCCCCCTTTTCGGAGTACACCGATATCAGTGGGCGAGCACACGCCATG |
| (SEQ ID NO: 1119) | GCGGAGAGAGGAGACAGCCACGCTCCTCTCAGCCCGGCTGCTGCCACAGGAAAAGGAAGTGCTT |
| RPL4_NM_000968_r1_2 | /5Biosg/CTGCTAATTCACTGACAGCATAGGGCTGTCTGTTGTTTTTGCGCAAGTTGGTGTGA |
| (SEQ ID NO: 1120) | ACAAAGTTCACAATATCTGGTCGAATAGGAGCCTTGAATACAGCAGGCAAAGTGACATTTTTGC |
| RPL4_NM_000968_r1_3 | /5Biosg/CTCCACGACACATGTTTCCAAAAGCACCCTGGCCAGAGCGGTGAGTCCCACCACCT |
| (SEQ ID NO: 1121) | CGAACTCTGGGAATTCGAGCCACAGCTCTGCCAGTACCCCAAGACTCAGCACTAGTCTGATGAC |
| RPL4_NM_000968_r1_4 | /5Biosg/ACATGACCAGTGCTGGTAGGGCTGAGGCAGCCAGGGCAGAACAGATGGCGTATCGT |
| (SEQ ID NO: 1122) | TTTTGGGTTGTGTTCACTCTACGATGCCAACGGCGCCAGGTTTTGGTTGGTGCAAACATTCGGC |
| RPL4_NM_000968_r1_5 | /5Biosg/CATTCCAGGCTTTAAGTTTCTTAAGGAGCAAAACAGCTTCCTTGGTCTTCTTGTAG |
| (SEQ ID NO: 1123) | CCTTCAACTTTATCTTCAACTACCAAAGGAAGTTCAGGAACTTCCTCAATACGATGACCTTTAG |
| RPL4_NM_000968_r1_6 | /5Biosg/TGATGATACCATTATCCTCATTATAGATGATGCACGGGCCCCTGCGCTGGATACGG |
| (SEQ ID NO: 1124) | CGACGGTTTCTCATTTTGCCTTTGCCAGCTCTCATTCGCTGAGAGGCATAGACCTTTTTGATAT |
| RPL4_NM_000968_r1_7 | /5Biosg/TCCGGAAAGCACTTTCAGTCCAAATGCAGAAACGTCCCACATGCCCACCAGGAGCA |
| (SEQ ID NO: 1125) | AGCTTCAAAATGTTCAGCTTGCTTACATTAAGCAGAGTAATTCCAGGGATGTTTCTGAAGGCCT |
| RPL4_NM_000968_r1_8 | /5Biosg/GGATCTCTGGGCTTTTCAAGATTCTGCTAAGATCTGTATTAATCATCTTGTGCATG |
| (SEQ ID NO: 1126) | GGAAGATTGTAGTTACTCTTGAGGCAAGCGGCTTTACGCCAAGTGCCGTACAATTCATCTAACT |
| RPL4_NM_000968_r1_9 | /5Biosg/TGTTCCGGCGCATGGTCTTTGCATATGGGTTTAGCTTCAACATGATTCTCAAGTTT |
| (SEQ ID NO: 1127) | TTCAGTGGGTTCTTCTTTAGGACTCTGCGATGGATCTTCTTGCGTGGTGCTCGAAGGGCTCTTT |
| RPL4_NM_000968_r1_10 | /5Biosg/CTACCACAGGCTTCTTGCCTGCAACCGCCGCCTTCTCATCTGATTTGGCTTGTAGT |
| (SEQ ID NO: 1128) | GCCGCTGCTGCAGCAGCTGCCTTATCCACCCGGAGCTTGTGATTCCTGGCCTGGCGAAGAATGG |
| RPL4_NM_000968_r1_11 | /5Biosg/TAGGTTTCTTCTCTGCAGGCTTCTTTTCAGGGGCTGGTTTCTTGGTAGCTGCTGCC |
| (SEQ ID NO: 1129) | TTTTTTCCCACCAGAGGCTTCTTCTGCTTCTTAACACCAACACCAGCCTTCTTTCCTTTCTTAC |
| RPL4_NM_000968_r1_12 | /5Biosg/CATGTTTCTCACTGCCTGTATAATCAGGTCTTTATTCAAAAGAAGCTGTCCAAAAT |
| (SEQ ID NO: 1130) | GATTTGACCTTTATGGAATAATCAAATTTAAGAGTTTATGCAGCAGGCTTCTTCTCCTCTGTAG |
| RPL5_NM_000969_r1_1 | /5Biosg/CTTAAAGTAGGCCTTATTCTTAACAACTTTAACAAACCCCATCCTGCGGAACAGAG |
| (SEQ ID NO: 1131) | ACCGGCGTCCGCTGCTCGACAGAGACCTGCAGGCCCAGCGGCGCTAGGGGGTGGCAAAAGGGCC |
| RPL5_NM_000969_r1_2 | /5Biosg/TATCATCCTGTATTTGGGTGTGTTGTATTTATTTTTATCTTGTATCACCAAGCGTT |
| (SEQ ID NO: 1132) | TCCGAGCATAATAATCAGTTTTACCCTCTCGTCGTCTTCTAAATTTCACTTGGTATCTCTTAAA |
| RPL5_NM_000969_r1_3 | /5Biosg/CAACCTTCACACCATATTTTGGCAGTTCGTGTGCATACGCTGCGCAGACTATCATA |
| (SEQ ID NO: 1133) | TCCCCCTCTATACGGGCATAAGCAATCTGACAAATGATATCTCTGTTTGTCACACGAACTATCA |
| RPL5_NM_000969_r1_4 | /5Biosg/CATCACCAGTCACCTCCACTTGGCCTTCATAGATCTTGTCCATGCCAAACCTATTG |
| (SEQ ID NO: 1134) | AGAAGCCTGCGGGCCAGCAGCAGGCCAGTACAATATGCTGCAGGATAATTTGTCAGGCCAACCT |
| RPL5_NM_000969_r1_5 | /5Biosg/CAGCTCCCTTCAGGGCACCAAAAACTTTATTGCCAGTGGTAGTTCTGGCAAGGCCT |
| (SEQ ID NO: 1135) | GCATCCAAATAGCAGGTGAAGGCACCTGGCTGACCATCAATGCTTTCCACATTGTATTCATCAC |
| RPL5_NM_000969_r1_6 | /5Biosg/GCAACATTCTGGCCCATGATGTGCTTCCGATGTACTTCTGCATTAAATTCCTTGCT |
| (SEQ ID NO: 1136) | TTCAGAATCATAACCAGGCAATCGTTTGGTACTGTGAGGGATAGACAAGCCTCCATCCACAGCT |
| RPL5_NM_000969_r1_7 | /5Biosg/GCTTTCTTATACATCTCCTCCATCATGTCTGGAGTTACGCTGTTCTTTATGTATTG |
| (SEQ ID NO: 1137) | AGAGAACTGTTTCTTGTAAGCATCTTCATCTTCTTCCATTAAGTAGCGCATGTAATCTGCAACA |
| RPL5_NM_000969_r1_8 | /5Biosg/AGCTACCCGATCCTTCTTCTGAGCAAGGGACATTTTGGGACGGTTCCACCTCTTCT |
| (SEQ ID NO: 1138) | TTTTAACTTCTTTCTTGGGCTTCTTTTCATAGACTGGATTCTCTCGTATAGCAGCATGAGCTTT |
| RPL5_NM_000969_r1_9 | /5Biosg/TTTTTAGTTGCTGTTCATAAGTTTATTATCTATATCTGAAAAAATCATAGAAAATT |
| (SEQ ID NO: 1139) | GCTGGGTTTAGCTCTCAGCAGCCCGCTCCTGAGCTCTGAGGAAGCTTGCCTTCTTTTGAGCTAC |
| RPL6_NM_000970_r1_1 | /5Biosg/CATCAACCTTCTTGGCTTCGGGTTTCTTCTCTTTAGTATCTGGCTTCTCAACTTTT |
| (SEQ ID NO: 1140) | TCACCCGCCATCTTGCAAGATGGGAAAGAGAATTAAGGTCCCGGCTTCCGGTCTATAAGCAATC |
| RPL6_NM_000970_r1_2 | /5Biosg/ACATGGCAGATCGGGAATACCTGCCAATTCCTCTGACAAGGACAGGGTTGCGGCTG |
| (SEQ ID NO: 1141) | CAATGGGGCTTCCCCTTCTTGGGCTTTTTAGCTTTGAGGTTACCCTTTTTCACCTTGCCACCAG |
| RPL6_NM_000970_r1_3 | /5Biosg/CGCCGTTCTTGTCACCACCAACTGGTTTTGTAACAGTTGCGAGAACCTTCTCCTTC |
| (SEQ ID NO: 1142) | TTTTTCTTTTCAACCTTGGATTTAGCGGCTGAGTACTTCCTCTTGTACATGGCCTTTCTGGAAT |
| RPL6_NM_000970_r1_4 | /5Biosg/TGGCTCGCAGTTTTCTCACGTGCTGACTGAAGGGTTTTTTGCCGTGGCTCAACAGC |
| (SEQ ID NO: 1143) | TTTCGAGGCACATCTTCAGTAGGATAATATCTAGGCATTTTGCGAAGTTTAACCACCCGGGTAC |
| RPL6_NM_000970_r1_5 | /5Biosg/GATTGAGGACCAGAGGTCCAGTCACAAGTAATAAGCCACTAGCCAGCTGCTTCAGG |
| (SEQ ID NO: 1144) | AAAACCACCCTCTTGCCCCTGTGGCGTCCAGTGAGGATGATCAGAATGGTCCCGGGGGTAATGC |
| RPL6_NM_000970_r1_6 | /5Biosg/GCAGCTTCTTCTTCTTGAAGTAAGCATCAGTAAGATGTTTTGGGATTTTTACATTG |
| (SEQ ID NO: 1145) | CTGATATCGATTTTGGTTGAAGTGGCAATGACAAATTTCTGGTGTGTTCTTCGTAGAGGAACTC |
| RPL6_NM_000970_r1_7 | /5Biosg/CTTTGATTTTTGGTAAAATTTGTGAGTCCACAGCTTTCTGATCAATCTTGCGCTGC |
| (SEQ ID NO: 1146) | TCCGTAATCTCATATTTCTCTTTTTCTGTGTCGAAGATCTCACCTTCCTGGTGTCTGGGCTTCC |
| RPL6_NM_000970_r1_8 | /5Biosg/TTTTTTTGTAGTCAGCTATTTAATTAGGTTCTTAAGACATTTAGAACACCAATTTG |
| (SEQ ID NO: 1147) | TGAGGATAAATTCCATTCGTCAGAGCAAACACAGATCGCAGGTAGCCCTGGAGCTGAGGAATAG |
| RPL7A_NM_000972_r1_1 | /5Biosg/GGGATTCACCACTTTCTTAGCCTCCTGCTTCTTCACGACAGCTGGGGCCGGAGCCA |
| (SEQ ID NO: 1148) | CCTTCTTTCCCTTGGCCTTCTTTCCTTTCGGCATCTTGGGCGGCGGGAGGAGAGAGAAAGGAAA |
| RPL7A_NM_000972_r1_2 | /5Biosg/CTCTGCCGCTGCAACCTGATATAGCGGGGCCATTTCACAAAGCGGGTGAGGTCTCT |
| (SEQ ID NO: 1149) | TTTGGGCTGGATGTCCTGTCCAATGCCAAAATTCTTAGGCCTTTTCTCAAACAGGGGATTCACC |
| RPL7A_NM_000972_r1_3 | /5Biosg/GTCTGTACTTGTGGGCCAGCTTAAGCAGCTGAGTAGCTGTTTGGCGGTCCAGGGCC |
| (SEQ ID NO: 1150) | TGGGTGAACTGGTTAATCGCAGGAGGCACTTTCAGCCGCTTATAGAGGATGGCTCTCTGCCGCT |
| RPL7A_NM_000972_r1_4 | /5Biosg/AACTCCTGCTCGAAGGACAGGTGGTCTCTTCGTTGGGACGTCCCCTTTGCCAGCAG |
| (SEQ ID NO: 1151) | CCTTCTTCTCGGCCCGGGCCAACAGTCTCTGCTTCTTCTCTTGCTTTGTCTCTGGTCTGTACTT |
| RPL7A_NM_000972_r1_5 | /5Biosg/ATTTTACGACACAGGGCAGGCAAGAAGACAACCAGCTCGATGGGATCCACGTCGTG |
| (SEQ ID NO: 1152) | TGCAATCACCACCAGCTGAGCTTTCTTGTTCTCCACCAAGGTGGTGACGGTGTTAACTCCTGCT |
| RPL7A_NM_000972_r1_6 | /5Biosg/CGCCTTTGTCTTCCGAGTTCACCTGTGTGAAGGCGACAGTGGTGCAGGTCTTCCTG |
| (SEQ ID NO: 1153) | TGGACTAGACGTCCCAGTCTTGCCTTTCCCTTGATAATGCAGAAAGGGACCCCCATTTTACGAC |
| RPL7A_NM_000972_r1_7 | /5Biosg/GATACGAGCCACAGACTTAGGACCCAGGACATTGCCACCCCAGTGACGGCGGATCT |
| (SEQ ID NO: 1154) | CATCGTATCTGTCATTGTAATTGGTCCTGATAGCTTCCACCAGCTTAGCCAAAGCGCCTTTGTC |
| RPL7A_NM_000972_r1_8 | /5Biosg/GAAGGAAAATTTGTATTATTTCAATTATTTTTATGTACAGAAAACTCAACAGTGTA |
| (SEQ ID NO: 1155) | CATTTAACCCAGTTTAGTGGCAAGTTCTTTAGCCTTTGCCTTTTCGAGCTTGGCGATACGAGCC |
| RPL7_NM_000971_r1_1 | /5Biosg/AGGCGCTTGATCTTCAGCTCTGCGAAATTCCTTCGCTTTTTCTTAAGGGTTTCTGG |
| (SEQ ID NO: 1156) | CACAGCAGGAACCTCCTTCTTCTTCTCTTCTACACCCTCCATGGTTCCAGCCGGAAAAAGAGGA |
| RPL7_NM_000971_r1_2 | /5Biosg/CTCGCCATTCGAATTTCAGTTCTGTACATCTGCCTATATTCCTTGTGATAGTGCTT |
| (SEQ ID NO: 1157) | TGCTTTTTCATAGATAAGCTTCCTCCTTGCCTTTCGAAGCATCTTTTGGGCAAACTTCTTTCTC |
| RPL7_NM_000971_r1_3 | /5Biosg/CGAAGAAGCTGCAACACCTTTCGAACCTTTGGGCTCACTCCATTGATACCTCTGAT |
| (SEQ ID NO: 1158) | TCTGATGACAAACGCCAATTTGGGTTCTGCAGGTACATAGAAGTTGCCAGCTTTTCTTGCCATC |
| RPL7_NM_000971_r1_4 | /5Biosg/AGTTCATTTACTGACTTCAGATTGGGGTACCCCCATGCAATATATGGCTCTACAAT |
| (SEQ ID NO: 1159) | CCTCAGCATGTTAATCGAAGCCTTGTTGAGCTTCACAAAGGTTCCATTGAAGATTTGACGAAGG |
| RPL7_NM_000971_r1_5 | /5Biosg/TCATGAATCAAATCCTCCATGCAGATGATGCCGTATTTACCAAGAGATCGAGCAAT |
| (SEQ ID NO: 1160) | CAAAGCGTTATCTGTCAAAGCAATTCGCTTCTTATTGATTTTGCCATAACCACGCTTGTAGATT |
| RPL7_NM_000971_r1_6 | /5Biosg/GCATCTCCACCTTCTACAAAATGGGTGGTCTTTTTCTTCATTCCACCTCGTGGAGA |
| (SEQ ID NO: 1161) | AGACAATTTGAAGGGCCACAGGAAGTTATTTGCCTCTTTGAAGCGTTTTCCAACAGTATAGATC |
| RPL7_NM_000971_r1_7 | /5Biosg/TTTTATTTCAATTTGAGAGCAGGTACTGTTTATTAACCAACCAGCTTAGAAAAATA |
| (SEQ ID NO: 1162) | ATCATGGTAGACACCTTAGTTCATTCTTCTAATAAGCCTGTTGATCTGGTCCTCCCTGTTGCCA |
| RPL8_NM_000973_r1_1 | /5Biosg/CGCGCGGAACACAGACCCGGCGCCCTTCCTCTGTCCACGGATCACACGGCCCATGG |
| (SEQ ID NO: 1163) | CGACGGGTCCTGTTCACCAGCGCGGCCGAAAGAGGAAACACGGCGTCAGCGAGCGGCCTTATCT |
| RPL8_NM_000973_r1_2 | /5Biosg/GCGGCCCGGGTCGTGGATGATGTCCTTGACGATGCCCTTGATGTAGCCGTGCCGCT |
| (SEQ ID NO: 1164) | CAGCGAAATCCACGGCGCGCAGGCGCGCAGCGCCTTTACGGTGCTTCACGTGCGCGCGGAACAC |
| RPL8_NM_000973_r1_3 | /5Biosg/GCAATACACAAACTGGCCCGTGTGAATGCCCTCGGCGGCAATGAACAGCTCCGTCC |
| (SEQ ID NO: 1165) | GCTTCTTAAACCGATACGGATCCCGGAAGACCACCTTGGCGAGGGGCGCGCCGCGGCCCGGGTC |
| RPL8_NM_000973_r1_4 | /5Biosg/CAGCTTGCCACGGTCTCCAGGCTTCTCCTCCAGGCAGCACACGATTGTACCCTCAG |
| (SEQ ID NO: 1166) | GCATGGTGCCCACAGGGAGCACATTGCCAATGTTGAGCTGGGCCTTCTTGCCGCAATACACAAA |
| RPL8_NM_000973_r1_5 | /5Biosg/GTTGGCTGAGGAGATAACCTTCTTGGAGCCGGAGGGCAGCTTCACACGGGTCTTCT |
| (SEQ ID NO: 1167) | TGGTCTCAGGGTTGTGGGAGATAACGGTGGCATAGTTCCCTGATGCCCGGGCCAGCTTGCCACG |
| RPL8_NM_000973_r1_6 | /5Biosg/TCGTGGCCAGCAGTTCCTCTTTGCCTTATATTTGTGGTACGCCCGGCCAGCCTTCA |
| (SEQ ID NO: 1168) | AGATGGGTTTGTCAATTCGGCCACCTCCAGCCACCACACCAACCACAGCTCTGTTGGCTGAGGA |
| RPL8_NM_000973_r1_7 | /5Biosg/TTTGCGGCCAGCAGGGGCATCTCTGCGGATGGTGGAGGGCTTGCCGATGTGCTGGT |
| (SEQ ID NO: 1169) | GGTTGCCACCTCCAAAAGGATGCTCCACAGGATTCATGGCCACACCCCGTACTCGTGGCCAGCA |
| RPL8_NM_000973_r1_8 | /5Biosg/TTGGCATAAACACAAACTTTATTGAGGCCCTCAGCACTAGTTCTCTTTCTCCTGCA |
| (SEQ ID NO: 1170) | CAGTCTTGGTTCCCCGGAGACGTCCAGTCCGGCGGGCAGCAATGAGACCCACTTTGCGGCCAGC |
| RPL9_NM_000661_r1_1 | /5Biosg/TCAGAGTAATGTCGACATTTTCTGGAATGTCGACAGTCTGATTGCTGAGAATAGTC |
| (SEQ ID NO: 1171) | TTCATTCTCGCAGTAGACGCAGCAAAGAAAGAACGTCTGTCGTCATTACGTACTTGTATCGCGT |
| RPL9_NM_000661_r1_2 | /5Biosg/GGAGCCTCTTTTTTTTCTTTCCAAGAAGGCTGAGTTCAACATTGATGTGATTGAAG |
| (SEQ ID NO: 1172) | TCCCTCCGCAGGGTTCCTCTGGGGCCCTTCACGATAACTGTGCGTCCCTTCAGAGTAATGTCGA |
| RPL9_NM_000661_r1_3 | /5Biosg/TGTAACGGAAGCCCAGTGTAACACCCTTGATCATGTTCTGTACATGACTACAAATA |
| (SEQ ID NO: 1173) | GTCCGAACGGTAGCCAGTTCCTTTCTGTTACCCCACCATTTGTCAACCCGGAGCCTCTTTTTTT |
| RPL9_NM_000661_r1_4 | /5Biosg/CCCTGCGGATATATTTTTCACCCAAGAAATTTCGGATTTCAACAAGAGACCCATTC |
| (SEQ ID NO: 1174) | TCCTGGATAACAACGTTGATGGGGAAGTGAGCATACACAGACCTCATCTTGTAACGGAAGCCCA |
| RPL9_NM_000661_r1_5 | /5Biosg/TCAAAGCCGCTGAATTTGAAACAAGCTCAATGTCATTTCCTTCAAGGATTAATTCA |
| (SEQ ID NO: 1175) | TCTTTCTGGGCTTGAGATACTGAACAAGCAACACCTGGTCTCATCCGAACCCTGCGGATATATT |
| RPL9_NM_000661_r1_6 | /5Biosg/TTAGATCTTATTCATCAGCCTGCTGAACAGTTCCTTTTTCAGAGACATAGATACCA |
| (SEQ ID NO: 1176) | TCCAAAAATTTCCTGATATCCTTGTTTTTAACTGTTGTGGCTTGCTGAATCAAAGCCGCTGAAT |
| RPL9_NM_000661_r1_7 | /5Biosg/TTTAAGAAGAAGGTCCAAATCAATAGGTCTTTTATTGCATCATTTAAATATCACAA |
| (SEQ ID NO: 1177) | GTAGGTCTTAAGTGTCATCTGGCATCTTCTTTCTGTAGCCAGGTAACTCTTAGATCTTATTCAT |
| RPLP0_NM_001002_r1_1 | /5Biosg/GGACGCCTGGCGAGAGAAGGGCCTCGCGCCCGCGCGTGCCTTTTATAATGCGAACA |
| (SEQ ID NO: 1178) | AAGTAGCCAATCAGAAACCGCGGATAGCGCTCCTGTCTATTGGCTGCGCCATCGCCCGTCAGAC |
| RPLP0_NM_001002_r1_2 | /5Biosg/TGATCTTAAGGAAGTAGTTGGACTTCCAGGTCGCCCTGTCTTCCCTGGGCATCACG |
| (SEQ ID NO: 1179) | GCGGTGCGTCAGGGATTGCCACGCAGGGTTTAAAGACGATGTCACTTCCACGAGGACGCCTGGC |
| RPLP0_NM_001002_r1_3 | /5Biosg/AGCACCACAGCCTTCCCGCGAAGGGACATGCGGATCTGCTGCATCTGCTTGGAGCC |
| (SEQ ID NO: 1180) | CACATTGTCTGCTCCCACAATGAAACATTTCGGATAATCATCCAATAGTTGGATGATCTTAAGG |
| RPLP0_NM_001000_r1_4 | /5Biosg/GAACACAAAGCCCACATTCCCCCGGATATGAGGCAGCAGTTTCTCCAGAGCTGGGT |
| (SEQ ID NO: 1181) | TGTTTTCCAGGTGCCCTCGGATGGCCTTGCGCATCATGGTGTTCTTGCCCATCAGCACCACAGC |
| RPLP0_NM_001002_r1_5 | /5Biosg/TCTGGGCTGGCACAGTGACTTCACATGGGGCAATGGCACCAGCACGGGCAGCAGCT |
| (SEQ ID NO: 1182) | GGCACCTTATTGGCCAGCAACATGTCCCTGATCTCAGTGAGGTCCTCCTTGGTGAACACAAAGC |
| RPLP0_NM_001002_r1_6 | /5Biosg/GTCTTGATCAGCTGCACATCACTCAGGATTTCAATGGTGCCCCTGGAGATTTTAGT |
| (SEQ ID NO: 1183) | GGTGATACCTAAAGCCTGGAAAAAGGAGGTCTTCTCGGGCCCGAGACCAGTGTTCTGGGCTGGC |
| RPLP0_NM_001002_r1_7 | /5Biosg/GTAGATGCTGCCATTGTCGAACACCTGCTGGATGACCAGCCCAAAGGAGAAGGGGG |
| (SEQ ID NO: 1184) | AGATGTTGAGCATGTTCACCAGCGTGGCTTCGCTGGCTCCCACTTTGTCTCCAGTCTTGATCAG |
| RPLP0_NM_001002_r1_8 | /5Biosg/ATGCAACAGTTGGGTAGCCAATCTGCAGACAGACACTGGCAACATTGCGGACACCC |
| (SEQ ID NO: 1185) | TCCAGGAAGCGAGAATGCAGAGTTTCCTCTGTGATATCAAGCACTTCAGGGTTGTAGATGCTGC |
| RPLP0_NM_001002_r1_9 | /5Biosg/GGATCAGCCAAGAAGGCCTTGACCTTTTCAGCAAGTGGGAAGGTGTAATCCGTCTC |
| (SEQ ID NO: 1186) | CACAGACAAGGCCAGGACTCGTTTGTACCCGTTGATGATAGAATGGGGTACTGATGCAACAGTT |
| RPLP0_NM_001002_r1_10 | /5Biosg/GTCCGACTCCTCCGACTCTTCCTTGGCTTCAACCTTAGCTGGGGCTGCAGCAGCAG |
| (SEQ ID NO: 1187) | CAGGAGCAGCTGTGGTGGCAGCAGCCACAGGGGCAGCAGCCACAAAGGCAGATGGATCAGCCAA |
| RPLP0_NM_001002_r1_11 | /5Biosg/TTTTTTACTTTTTAAAGAAGTAAGCCTTTATTTCCTTGTTTTGCAAATAAAACTGG |
| (SEQ ID NO: 1188) | CTAAGTTGGTTGCTTTTTGGTGATTAGTCAAAGAGACCAAATCCCATATCCTCGTCCGACTCCT |
| RPLP1_NM_001003_r1_1 | /5Biosg/GAGTGTAGGGCTGGCGCTGCCGGACGCGGTGCTAGTCGCCGGATGAAGTGAGGGCC |
| (SEQ ID NO: 1189) | TCACCCCAACGCAGCCTTAGCTTCCTCGGAAGGACCGAGCACCTTGGCGGCAGCTGAGGAAAGG |
| RPLP1_NM_001003_r1_2 | /5Biosg/GCATTGATCTTATCCTCCGTGACTGTCACCTCATCGTCGTGCAGAATGAGGGCCGA |
| (SEQ ID NO: 1190) | GTAGATGCAGGCGAGCTCGGAGACAGAGGCCATGGCGCGGGCGAGTGTAGGGCTGGCGCTGCCG |
| RPLP1_NM_001003_r1_3 | /5Biosg/CATTGCAGATGAGGCTCCCAATGTTGACGTTGGCCAGGGCCTTTGCAAACAAGCCA |
| (SEQ ID NO: 1191) | GGCCAAAAAGGCTCAACATTTACACCGGCTGCTTTAATGAGGGCATTGATCTTATCCTCCGTGA |
| RPLP1_NM_001003_r1_4 | /5Biosg/TTCCACTTTCTTCTCCTCAGCTGGAGCAGCAGCAGTGGAGGGGGCAGGACCTCCTG |
| (SEQ ID NO: 1192) | CTGGTGCAGCACCAGCTGCTGGAGCAGGTCCACCGGCCCCTACATTGCAGATGAGGCTCCCAAT |
| RPLP1_NM_001003_r1_5 | /5Biosg/AAAGTTCAGCTTTTTATTGAACATGTTATAAAAGAGGTTTAGTCAAAAAGACCAAA |
| (SEQ ID NO: 1193) | GCCCATGTCATCATCAGACTCCTCGGATTCTTCTTTCTTTGCTTCCACTTTCTTCTCCTCAGCT |
| RPLP2_NM_01004_r1_1 | /5Biosg/GGCGACGTAGCGCATCGCGGCGGCGTCTGCGGCGGAGGCGGCGGAGAAGTCTCACG |
| (SEQ ID NO: 1194) | CGTGCGACCTCGGTGGCGACAGGGAGGAAAAGGAAGGCGCCGACGCAAGAGGCGGGGTTAAACC |
| RPLP2_NM_01004_r1_2 | /5Biosg/CGTCGTCCGCCTCGATACCCACGCTGTCCAAGATCTTCTTGATGTCCTTGGCGCTG |
| (SEQ ID NO: 1195) | GGGGAGGAGTTGCCCCCTAGGGCAGCCAGCAGGTAGGAGGCGACGTAGCGCATCGCGGCGGCGT |
| RPLP2_NM_01004_r1_3 | /5Biosg/CAGCAGGTACACTGGCAAGCTTGCCAATACCCTGGGCAATGACGTCTTCAATGTTT |
| (SEQ ID NO: 1196) | TTTCCATTCAGCTCACTGATAACCTTGTTGAGCCGGTCGTCGTCCGCCTCGATACCCACGCTGT |
| RPLP2_NM_01004_r1_4 | /5Biosg/TTCTTCTCATCTTTCTTCTCCTCTGCTGCAGCAGGGGCAGAACCAGCAGCAGGGGC |
| (SEQ ID NO: 1197) | TGCAGAGCCTGGGGCAGCAGAGACGGCTACAGCCCCACCAGCAGGTACACTGGCAAGCTTGCCA |
| RPLP2_NM_01004_r1_5 | /5Biosg/TTGAGATGTGTAAAAAGGCTTTATTTGCAGGGGAGCAGGAATTTAATCAAAAAGGC |
| (SEQ ID NO: 1198) | CAAATCCCATGTCATCATCTGACTCTTCAGACTCCTCCTTCTTCTCATCTTTCTTCTCCTCTGC |
| RPS10_NM_001014_r1_1 | /5Biosg/ATCCGGTTCTTCTTAGGCATCAACATCTCTGCGGCTGCAGGGTCCGGTACCGGGGC |
| (SEQ ID NO: 1199) | TGGAAAGGAAGGAGCATGCGCGGTGCTGCGTCTCTTCCGGGCTGGCGTGGACCCGCCCCCCGCC |
| RPS10_NM_001014_r1_2 | /5Biosg/TGAAGGTTGGGCACATTCTTGTCTGCCAGCTCCGGGTGCTTAGGCATGTGGACATC |
| (SEQ ID NO: 1200) | CTTCTTGGCCACCATGACTCCCTCCTTAAAAAGGAGTTCATAAATGGCAATCCGGTTCTTCTTA |
| RPS10_NM_001014_r1_3 | /5Biosg/GATACTGGATACCCTCATTGGTAAGGTACCAGTAGAAATGTCTCCAGGCAAACTGT |
| (SEQ ID NO: 1201) | TCCTTCACGTAGCCTCGGGACTTGAGAGACTGCATGGCCTTCATGACATGAAGGTTGGGCACAT |
| RPS10_NM_001014_r1_4 | /5Biosg/GTCGCTCACCCTCCAGACCTTTAGGCCGAGGCCTGCCAGTCTCTGGACGGCTACGG |
| (SEQ ID NO: 1202) | CGTAGGGTGGCAGGCACAATCTCCGGGGGCAGATGAAGGTAATCACGGAGATACTGGATACCCT |
| RPS10_NM_001014_r1_5 | /5Biosg/GGTTGCTGACCCAGCCCCAGCCTCGGCTTTCTTGTCGGCACCAGGTGGCACAGCAC |
| (SEQ ID NO: 1203) | TCCGTCTGTAGGTATCTCTGTCAGCTTCCCCTCTTGTGAGTCTCGCAGGTCGCTCACCCTCCAG |
| RPS10_NM_001014_r1_6 | /5Biosg/TAAGGTTTTTTGGCTGTAAGTTTATTCAATGCAAAAGAATCCTCTCCAATTTTACT |
| (SEQ ID NO: 1204) | GAGGTGGCTGACCACGTCCACGACCAAATCCGCCTCTAAACTGGAATTCGGTTGCTGACCCAGC |
| RPS11_NM_001015_r1_1 | /5Biosg/AGGCACGCTCAGTCTGAATGTCCGCCATCTTCCCGGCCGCCTGAAAAAAAGAAAGG |
| (SEQ ID NO: 1205) | GGCAGCAGCGTCCGGGTTTCCTTATCGATTACGCAGGGGCTGGAGAGGAAGTGACGTAAGGAGA |
| RPS11_NM_001015_r1_2 | /5Biosg/TGAAGCCCAGACCGATGTTCTTGTAGTACCGCGGGAGCTTCTCCTTGCCAGTTTCT |
| (SEQ ID NO: 1206) | CCCAGCAGGACCCTCTTCTTGTTTTGAAAGATGGTCGGCTGCTTTTGGTAGGCACGCTCAGTCT |
| RPS11_NM_001015_r1_3 | /5Biosg/TCTTGGTCACCACGCCAGAGAGGATCCGCCCTCGAATGGACACATTACCAGTGAAG |
| (SEQ ID NO: 1207) | GGGCATTTCTTGTCAATGTAGGTGCCCTCAATAGCCTCCTTGGGTGTCTTGAAGCCCAGACCGA |
| RPS11_NM_001015_r1_4 | /5Biosg/GGGGACAGGTGTACAGACATGTTCTTGTGGCGCTTCTCGAAGCGGTTGTACTTGCG |
| (SEQ ID NO: 1208) | GATGTAGTGCAGATAGTCTCGGCGGATGACAATGGTCCTCTGCATCTTCATCTTGGTCACCACG |
| RPS11_NM_001015_r1_5 | /5Biosg/CCGGCAGCCTTGGTGACCTTGAGCACGTTGAAGCGCACTGTCTTGCTCAGAGGCCG |
| (SEQ ID NO: 1209) | GCACTCGCCCACTGTGACGATGTCACCGATCTGGACGTCCCTGAAGCAGGGGGACAGGTGTACA |
| RPS11_NM_001015_r1_6 | /5Biosg/CGCGGAAGATCCCAAGTCTGGCCTGGGAATGAGAAAATAACTTTATTTCATTGTGG |
| (SEQ ID NO: 1210) | GGAGCGGGCCGATGTCCAGCCTCAGAACTTCTGGAACTGCTTCTTGGTGCCGGCAGCCTTGGTG |
| RPS12_NM_001016_r1_1 | /5Biosg/TCCATTACACCTCCAGCAGCAATGCCTTCCTCGGCCATGGCGGTGGGTTACGGGTG |
| (SEQ ID NO: 1211) | AAGTTGAATCTTGAACGCACCCAAGCCTCCGCCTCCGCGCGACTCGGCGGCGGCAGGGAAAGAG |
| RPS12_NM_001016_r1_2 | /5Biosg/CGCTTGTCTAAGGCTTTGGCAGCTTCGCGAATTCCACGTGCTAGGCCATCGTGGAT |
| (SEQ ID NO: 1212) | GAGGGCAGTCTTCAGAACCTCTTGTAAAGCAGTATTAACGTCCATTACACCTCCAGCAGCAATG |
| RPS12_NM_001016_r1_3 | /5Biosg/AATTAGGTTGATTTGGTGTTCAGCACAAAGGGCCTCCACCAACTTGACATACATAG |
| (SEQ ID NO: 1213) | GCTCATCACAGTTGGATGCAAGCACACAAAGATGGGCTTGGCGCTTGTCTAAGGCTTTGGCAGC |
| RPS12_NM_001016_r1_4 | /5Biosg/TACACAACTGCAACCAACCACTTTACGGGGTTTCCCCTCTCTGTCAATTTTACAAA |
| (SEQ ID NO: 1214) | GGCCTACCCATTCTCCTAGTTTCTTGTTGTCATCAACCTTAATTAGGTTGATTTGGTGTTCAGC |
| RPS12_NM_001016_r1_5 | /5Biosg/TTTGTGAGCCAAAGATTTATTTCTTCATTTCTTGCATTTGAAATACTCTTCAATGA |
| (SEQ ID NO: 1215) | CATCCTTGGCCTGAGACTCCTTGCCATAGTCCTTAACTACTACACAACTGCAACCAACCACTTT |
| RPS13_NM_001017_r1_1 | /5Biosg/ATGTCAACTTCAACCAAGTGGGGACGCTGCGTCGATAGGGTAAAGCCGACTGGGAC |
| (SEQ ID NO: 1216) | AGGCCCTTCCCGGGAGCATGCATGCGACCCATGATGGCGGCGATCAGGCAACGAAAGGAGAGCG |
| RPS13_NM_001017_r1_2 | /5Biosg/GTACTTGTGCAACACCATGTGAATCTCTCAGGATTACACCGATCTGTGAAGGAGTA |
| (SEQ ID NO: 1217) | AGGCCCTTCTTGGCCAGTTTGTAAATCTGCTCCTTCACGTCGTCAGATGTCAACTTCAACCAAG |
| RPS13_NM_001017_r1_3 | /5Biosg/ACAGCAACTGCTTTCTTAATTAAATGGTAGAGATCTTCAGGAAGATCAGGAGCAAG |
| (SEQ ID NO: 1218) | TCCCTTAGACTTAAGAATTCTTAAAATTTTATTGCCTGTCACAAAACGTACTTGTGCAACACCA |
| RPS13_NM_001017_r1_4 | /5Biosg/TTGGTCTTATAATATCGAGCCAAACGGTGAATCCGGCTCTCTATTAGAATCAGACG |
| (SEQ ID NO: 1219) | GAATTTAGCATCCTTATCCTTTCTGTTCCTCTCAAGATGCTTTCGAACAGCAACTGCTTTCTTA |
| RPS13_NM_001017_r1_5 | /5Biosg/TAGTTAAACAATCATTTTATTGCTTGAGTACACAGACAAATTTATGCGACCAGGGC |
| (SEQ ID NO: 1220) | AGAGGCTGTAGATGATTCATATTTCCAATTGGGAGGGAGGACTCGCTTGGTCTTATAATATCGA |
| RPS14_NM_005617_r1_1 | /5Biosg/CCACCTGAGGTCCGAGGCTGATGACCTGTTCTTCCTTCTTTTCCTTCCCCTTTCGA |
| (SEQ ID NO: 1221) | GGTGCCATTTCTGCACGTCGTCTCCAGACTCCACACCGGAAAGAGAGAGTGGGAGGGGGCGGAG |
| RPS14_NM_005617_r1_2 | /5Biosg/CCACCAGTCACACGGCAGATGGTTTCCTTGCCAGAAAGATCAGTGACATGGACAAA |
| (SEQ ID NO: 1222) | AGTGTCATTGAAGGATGCAAAGATATGGCAGACACCAAATACATTCTCTCCTTCAGCCACCTGA |
| RPS14_NM_005617_r1_3 | /5Biosg/TTTGATGTGTAGGGCGGTGATACCCAGCTCCTTGCACCTCTGGGCCACATCCTGGG |
| (SEQ ID NO: 1223) | CAGCCAACATAGCAGCATATGGTGAGGATTCATCTCGGTCTGCCTTTACCTTCATCCCACCAGT |
| RPS14_NM_005617_r1_4 | /5Biosg/GGGTGACATCCTCAATCCGCCCGATCTTCATACCCGAGCGGGCAAGGGCTCTGAGG |
| (SEQ ID NO: 1224) | GCCGACTGGGCCCCAGGTCCAGGGGTCTTGGTCCTATTTCCTCCTGTGGCCCGGAGTTTGATGT |
| RPS14_NM_005617_r1_5 | /5Biosg/TGAAACAGTTTACATGAAGGCAATTTATTAACAGAAAATATTTTGAGGAATCTTGT |
| (SEQ ID NO: 1225) | TCACAGACGGCGACCACGGCGACCCCCCTTCCTGCGAGTGCTGTCAGAGGGGATGGGGGTGACA |
| RPS15A_NM_001019_r1_1 | /5Biosg/TGCCAGGACATTCATGCGCACCATTGTGGCTTAGATTGCAGGATGGCGCCGATGGC |
| (SEQ ID NO: 1226) | AGACGGATGAAATTGGAGCTCTCAGAGAGTGCTACTCACCGGCGCGGAAAGATGGCGGAAAGAG |
| RPS15A_NM_001019_r1_2 | /5Biosg/GCTTCATCATCACAGTGAGAAACCGGACGATGACTTTGGAGCACGGCCTAATAAGC |
| (SEQ ID NO: 1227) | ACCTGGCGTTTGCCTCTCTTTTCGGCATTGTTGATACTCTTGAGAGCATCTGCCAGGACATTCA |
| RPS15A_NM_001019_r1_3 | /5Biosg/AATCTGGGGCTGATCACCCCACACTTGTTTAGCCTGCCTGTGAGGTTCACAACAAT |
| (SEQ ID NO: 1228) | TTTCCCAGCTCTGTGGTCATCAATGATTTCAAATTCGCCAATGTAACCATGCTTCATCATCACA |
| RPS15A_NM_001019_r1_4 | /5Biosg/TTCATGGTCCATGATGCCAGCTGAGGTTGTCAGTACAATGAAACCAAACTGGCGGG |
| (SEQ ID NO: 1229) | ATGGAAGCAGATTATTCTGCCATTTTTCCAGGTCTTTGAGTTGCACGTCAAATCTGGGGCTGAT |
| RPS15A_NM_001019_r1_5 | /5Biosg/TTGGAAGCACCAGAGTCCATGAGGCATTTTATTTGTAAATATATGTATTACATCCC |
| (SEQ ID NO: 1230) | TAGAAAAAGAATCCCAGGATTTTCCCTCCTGTGTGTTTTCGTCTTGCTTCTTCATGGTCCATGA |
| RPS15_NM_001018_r1_1 | /5Biosg/CGCCGCGGTAGGTGAACTTGCGCAAGGTCCGCTTCTTCTTCTGCTCTACTTCTGCC |
| (SEQ ID NO: 1231) | ATCTTGCCGGATCCTCAGAAGAGATCGCTTTGGTCCGCGCCTGCGCAGTTATCGCGAGACTGCC |
| RPS15_NM_001018_r1_2 | /5Biosg/TGCTGCTTCCGCCGCAGGCCCCGGTTCAGCCGCCGCCGCTGGCGCGCACTGTACAG |
| (SEQ ID NO: 1232) | CTGCATCAGCTGCTCGTAGGACATGTCCAGCAGCTGGTCGAGGTCCACGCCGCGGTAGGTGAAC |
| RPS15_NM_001013_r1_3 | /5Biosg/TCGGGTAGGATGATCATGTCCCGCAGGTGCGTCTTCACCACTTCCGGCTTCTCCAT |
| (SEQ ID NO: 1233) | GGGCGGCGCCTCCTTCTTGGCCTTGCGCAGGCGCTTCAGCAGGGAGTGCTGCTTCCGCCGCAGG |
| RPS15_NM_001018_r1_4 | /5Biosg/GGTGATGGAGAACTCGCCCAGGTAGTGGCCGATCATCTCGGGCTTGATCTCCACCT |
| (SEQ ID NO: 1234) | GGTTGAAGGTCTTGCCGTTGTAGACGCCCACCATGCTGCCCACCATCTCGGGTAGGATGATCAT |
| RPS15_NM_001018_r1_5 | /5Biosg/GGAGTCATGTGCGCCTTTATTAGCTGAGCCATTACTTGAGAGGGATGAAGCGGGAG |
| (SEQ ID NO: 1235) | GAGTGGGTGGCCCCGATGCCGGGCCGGCCATGCTTTACGGGCTTGTAGGTGATGGAGAACTCGC |
| RPS16_NM_001020_r1_1 | /5Biosg/AGACCTGCACAGACTGCAGCGGGCCCTTGGACGGCATGGCTCCGAGCGTGGACTAG |
| (SEQ ID NO: 1236) | ACAACCTCACCGCGCGGCGCCGCAACCGGAAAAGGAAAGCTAGGGGCCACCCTGGCCGCTTTTC |
| RPS16_NM_001020_r1_2 | /5Biosg/TCATCTTCTTCCGGATTTGGCGGACCTGTTGGTGCTGAGCATAAGAGGTCTTCCGT |
| (SEQ ID NO: 1237) | TTGATGAGACCATTGCCGCGTTTGCAGTGCGCCACAGCTGTCGCTGTCTTCTTGCGTCCGAAGA |
| RPS16_NM_001020_r1_3 | /5Biosg/GCTTTGGAGATGGACTGACGGATAGCATAAATCTGGGCCACGTGACCACCACCCTT |
| (SEQ ID NO: 1238) | TACACGGACACGGATGTCTACACCAGCAAATCGCTCCTTGCCGAGAAGCAGAACTGGCTCCAGC |
| RPS16_NM_001020_r1_4 | /5Biosg/TTTGGACTCGCAGCGACGAGGGTCAGCTACCAGCAGGGTCCGGTCATACTGGATGA |
| (SEQ ID NO: 1239) | GGATGTCTTTGATCTCCTTCTTGGAAGCCTCATCCACATATTTCTGGTAATAGGCCACCAGGGC |
| RPS16_NM_001020_r1_5 | /5Biosg/TTCTTGAAACTTTAAAATCCCTCAAAAACTGTTTATTATACAAGTGAGTTTTGAGT |
| (SEQ ID NO: 1240) | CACGATGGGCTTATCGGTAGGATTTCTGGTAGCGAGCGCGGGCACCAGGGCCTCCAAACTTTTT |
| RPS17_NM_001021_r1_1 | /5Biosg/TCGTGTGGAAGTCGTTGCCCAGGCGCGTGTAGTACTTTTCTATGATGACCCGGGCC |
| (SEQ ID NO: 1241) | GCCTTCTTCACGGTTTTGGTGCGAACGCGGCCCATGTTGGCGGGTCCTTGGTAAAAGAGGAAAC |
| RPS17_NM_001021_r1_2 | /5Biosg/GAATTCGCTTCATCAGATGCGTGACATAACCTGCTATCTTGTTGCGGAGCTTTTTG |
| (SEQ ID NO: 1242) | CTGGGGATAATGGCGATCTCCTCGCACACGCGCTTGTTCGTGTGGAAGTCGTTGCCCAGGCGCG |
| RPS17_NM_001021_r1_3 | /5Biosg/ATCCAAGGCTGAGACCTCAGGAACATAATTGTCTCTCCTTTCTCTCTCCTCCTCCT |
| (SEQ ID NO: 1243) | GCAGCTTGATGGAGATACCTCTTACTGGGCCTCTCTGAATTCGCTTCATCAGATGCGTGACATA |
| RPS17_NM_001021_r1_4 | /5Biosg/GTAGGCTGAGTGACCTGAAGGTTGGACAGACTGCCGAAGTCCAAAAGCTTCAGCAT |
| (SEQ ID NO: 1244) | TTCCTTAGTGTCAGGATCTACTTCAATAATCTCCTGATCCAAGGCTGAGACCTCAGGAACATAA |
| RPS17_NM_001021_r1_5 | /5Biosg/TTTTTGCTGTTGTCCCAGATTTATTGAAAATAATACAGCACTACAGTAAAAATTCA |
| (SEQ ID NO: 1245) | AACAGGTCCCCGAGGCGTTTTGAAATTCATCCCAACTGTAGGCTGAGTGACCTGAAGGTTGGAC |
| RPS18_NM_022551_r1_1 | /5Biosg/TTTCCGCCGCCCATCGATGTTGGTGTTGAGTACTCGCAAAATATGCTGGAACTTTT |
| (SEQ ID NO: 1246) | CAGGGATCACTAGAGACATGGCTGCAGCACAAGCGGCGGCGTGTAGGCCTCCTGTGGAAGAGAG |
| RPS18_NM_022551_r1_2 | /5Biosg/CAGTGAGTTCTCCCGCCCTCTTGGTGAGGTCAATGTCTGCTTTCCTCAACACCACA |
| (SEQ ID NO: 1247) | TGAGCATATCTTCGGCCCACACCCTTAATGGCAGTGATGGCAAAGGCTATTTTCCGCCGCCCAT |
| RPS18_NM_022551_r1_3 | /5Biosg/CTGTATTTTCCATCCTTTACATCCTTCTGTCTGTTCAAGAACCAGTCTGGGATCTT |
| (SEQ ID NO: 1248) | GTACTGGCGTGGATTCTGCATAATGGTGATCACACGTTCCACCTCATCCTCAGTGAGTTCTCCC |
| RPS18_NM_022551_r1_4 | /5Biosg/GACACGAAGGCCCCAGAAGTGACGCAGCCCTCTATGGGCCCGAATCTTCTTCAGTC |
| (SEQ ID NO: 1249) | GCTCCAGGTCTTCACGGAGCTTGTTGTCCAGACCATTGGCTAGGACCTGGCTGTATTTTCCATC |
| RPS18_NM_022551_r1_5 | /5Biosg/TTGTATATAAACTATTTATTAACAGACAAGGCCTACAGACTTATTTCTTCTTGGAC |
| (SEQ ID NO: 1250) | ACACCCACGGTGCGGCCACGGCGGCCAGTGGTCTTGGTGTGCTGGCCTCGGACACGAAGGCCCC |
| RPS19_NM_001022_r1_1 | /5Biosg/CCGGAGTGTAATAGAGAGAGGATAGAGAGCTCCTGTTCGGAGCTGGGGGAACTTGG |
| (SEQ ID NO: 1251) | CTTCGTTTGCGTCGTTCGTGGCTGGAAGGAACAGTGGTGGAGAATACTATGATGGCGAAAGTAC |
| RPS19_NM_001022_r1_2 | /5Biosg/GTCCCCGCCTAGAGGGGAGGGTGTCTAGTGAGGGGTGGAGAGGTAAAGGGGAGGGC |
| (SEQ ID NO: 1252) | CAAGGGGTCGCGCGTGGAGGCCTGGGTTTCCTCCCGCGTTTCCTTCTCCCGGAGTGTAATAGAG |
| RPS19_NM_001022_r1_3 | /5Biosg/GGTTTCCCGGGCGCACAAGTCGGGCGTAGGGTCTCGCGAGAGTTCCGAAAGCTCGC |
| (SEQ ID NO: 1253) | GAGAGCGAGGGTAGACGCTGAGGCTCCGCCTCTCTCAGGGCGAAAGTTCGTCCCCGCCTAGAGG |
| RPS19_NM_001022_r1_4 | /5Biosg/AGGCTGCCAGAGCTCTGACGAACTCCTGCTGGTTCACGTCTTTTACAGTAACTCCA |
| (SEQ ID NO: 1254) | GGCATCGTGCGGCCTCCGCGCTGCCAGCCAGGGGAAAGGGAACGACGGGGTTTCCCGGGCGCAC |
| RPS19_NM_001022_r1_5 | /5Biosg/CGTGTAGAACCAGTTCTCATCGTAGGGAGCAAGCTCTTTGTGCTTGGCCAGCTTGA |
| (SEQ ID NO: 1255) | CGGTATCCACCCATTCGGGGACTTTCAGCTTCCCGGACTTTTTGAGGAAGGCTGCCAGAGCTCT |
| RPS19_NM_001022_r1_6 | /5Biosg/CTGGGCATGACGCCGTTTCTCTGACGTCCCCCATAGATCTTGGTCATGGAGCCAAC |
| (SEQ ID NO: 1256) | CCCAGCGCCACCCCGGAGGTACAGGTGCCGCGCTGTGGAAGCAGCTCGCGTGTAGAACCAGTTC |
| RPS19_NM_001022_r1_7 | /5Biosg/GGTGTCAGTTTGCGGCCGCCATCTTGGTCCTTTTCCACCATTTTCAGCCCCTCCAG |
| (SEQ ID NO: 1257) | GGCTTGGAGGACCCGGCGGGCCACACTCTTGGAGCCTCGGCTGAAGTGGCTGGGCATGACGCCG |
| RPS19_NM_001022_r1_8 | /5Biosg/TTTACGAATGAGGCAATTTATTAACCCAGCATGGTTTGTTCTAATGCTTCTTGTTG |
| (SEQ ID NO: 1258) | GCAGCTGCCACCTGTCCGGCGATTCTGTCCAGATCTCTTTGTCCCTGAGGTGTCAGTTTGCGGC |
| RPS20_NM_001023_r1_1 | /5Biosg/GGACCAAAAATCCTCAGCCCTTACGACCGCGTCTTCCTCAAAAAGAAAGGGGTGGG |
| (SEQ ID NO: 1259) | ACTTGAGCAACGCTTATATAGCACGCTTGCATCCGGGTCCCACACGCCCCGGAACAGGAAATAT |
| RPS20_NM_001023_r1_2 | /5Biosg/CTCCGGCTCCACGGGTGTTTTTCCGGTATCCTTAAAAGCCATGGCTGTTGCGCGCG |
| (SEQ ID NO: 1260) | GGCTTCCTGACCGACTTGTTCCTCGGCGAGAGCGAACAGCGGTGAGTCAGGAGCAGGAGCGTGC |
| RPS20_NM_001023_r1_3 | /5Biosg/AACTGGTCCTTTCACTTTGAGATTCTTTTCTTTTGCGCCTCTTATCAAGTCAGCAC |
| (SEQ ID NO: 1261) | ACACCTTTTCCAAGGATTTTACGTTGCGGCTTGTTAGGGTGATTCGAATTCGGTGAATTGCCAC |
| RPS20_NM_001023_r1_4 | /5Biosg/AGGACTGTGCAAGTCAATGAGTCGCTTGTGAATTCTCATCTGGAAACGATCCCACG |
| (SEQ ID NO: 1262) | TCTTAGAACCTTCACCACAAGGAGTTTTTCTTGTAGTGATTCTCAAAGTCTTGGTAGGCATTCG |
| RPS20_NM_001023_r1_5 | /5Biosg/CAGAAGTTAACAACTGGTCATCAATTTATTAAAATAGTTGACTTAAGCATCTGCAA |
| (SEQ ID NO: 1263) | TGGTGACTTCCACCTCAACTCCTGGCTCAATACTGATGGAAGTAATCTGCTTAACAATCTCAGA |
| RPS20_NM_001023_r1_6 | /5Biosg/AACGACAACGAAAACAGGATAGACCACTCTAAGATACCCATATATTCCACCTGAAA |
| (SEQ ID NO: 1264) | AGTGGAAGTCTCATAGTACACCTTTATATTCCTAAAATCTGCCAGTATTCTGAATAAAAACCAA |
| RPS20_NM_001023_r1_7 | /5Biosg/TCCCCATACCAATCAGAATTTATCTAGAGTATGCCAAAGTTATAAACTGCCAGTGT |
| (SEQ ID NO: 1265) | CCAATAAAACCAGTCAGGTCTCACATATTATGTAGTTAACGGAATCCAGTTTTCAAATGACTCA |
| RPS21_NM_001024_r1_1 | /5Biosg/GTCCACGAACTCGCCGGCGTCGTTCTGCATTTCGAGGCTGGGCTGCGCCTGCTGCC |
| (SEQ ID NO: 1266) | ACCACACCGCGCGCGAGAGAGAAAGGAAGCAGCTAGTCACCCGGCAGAGATATCGGCGGGCAAG |
| RPS21_NM_001024_r1_2 | /5Biosg/CTTGTCAACCTCGGCCACGTTCATCTGGATGGATGCGTGGTCCTTGGCACCGATGA |
| (SEQ ID NO: 1267) | TGCGATTGCTAGCGGAGCATTTCCGCGGCACGTACAGGTCCACGAACTCGCCGGCGTCGTTCTG |
| RPS21_NM_001024_r1_3 | /5Biosg/CCAATCGGAGAATGGAATCATCTGACTCACCCATCCTACGAATGGCCCCGCAGATA |
| (SEQ ID NO: 1268) | GCATAAGTTTTAAACTGGCCATTAAACCTGCCTGTGACCTTGTCAACCTCGGCCACGTTCATCT |
| RPS21_NM_001024_r1_4 | /5Biosg/TTTTTAGGTTTTCATTATTTATTTATGACAAATATTCCACATCTGTGATTCTCTCC |
| (SEQ ID NO: 1269) | AGTCAAAAGTTCTTTGAGACGATGCCATCGGCCTIGGCCAATCGGAGAATGGAATCATCTGACT |
| RPS23_NM_001025_r1_1 | /5Biosg/AGTACGAAGTCCACGACACTTGCCCATCCTGTCGGCGCCACGGGCCTGAGCGAAAG |
| (SEQ ID NO: 1270) | AGAGAAGCACCGCAGGAAGGAGCCACAAGCCACCGCGAGCAAGCCCCGCCCAGCCAAGGACCCC |
| RPS23_NM_001025_r1_2 | /5Biosg/CTTTTGCATGAGAAGCACCTCCAAAAGGGTTGGCCTTTAGGGCTGTGCCCAAATGA |
| (SEQ ID NO: 1271) | GCTTTCTTATACTGTTTATCATGCCACTTCTGGTCTCGTCGGTGACTACGGAGCTTCCTAGCAG |
| RPS23_NM_001025_r1_3 | /5Biosg/GTCATTGGGTACAAAGGCTGTGATTTTCTTGCCATTCTTGATCAGCTGGACCCTTA |
| (SEQ ID NO: 1272) | CACACTTCCTAATGGCAGAATTTGGCTGTTTGGCTTCAACTCCTACTTTTTCCAGCACGATTCC |
| RPS23_NM_001025_r1_4 | /5Biosg/CATTGGCTACTTTGACAACCTTAAAGCGGACTCCAGGAATATCACCAACAGCATGA |
| (SEQ ID NO: 1273) | CCTTTGCGACCAAATCCAGCAACCAGAACTTCATCATTTTCCTCAATAAAGTTCAAGCAACCGT |
| RPS23_NM_001025_r1_5 | /5Biosg/GGACAGTAAGATACAAACATTTTTTGGCATATGAAAATTTATTACTACAGTGTTTT |
| (SEQ ID NO: 1274) | CACCATTAATATTTATGATCTTGGTCTTTCGTTCTTGCCTTTGTATAGGGCCAAAAGAGAAACA |
| RPS23_NM_001025_r1_6 | /5Biosg/ACAACCGTGTCTTATTGTCTCCTAAAATTGGTACAGGTCCTGCGTTTGCTGGTTTA |
| (SEQ ID NO: 1275) | GGATAAAAAAAATAAGGGGGGGTGGTGGTGGTAATGAACATGATCTTCGTGGTGAGAACAGGGG |
| RPS23_NM_001025_r1_7 | /5Biosg/TTCTTAAAGTAATACTACAATACTGCACATTTATTCCAGATCTATCCCATTTTCTT |
| (SEQ ID NO: 1276) | ATTCCACAGGATGAGGGAAACTGTGCCAGGTTACAAATGTTATTAACTCTAGAGAATCCTGAAA |
| RPS23_NM_001025_r1_8 | /5Biosg/TAATCTATGTGCAATGAAATTTGGTAACTGAAGTGTTGCACCCGATATGGAAAATA |
| (SEQ ID NO: 1277) | CCAAGAATAAAAAAGAAAGTATCTCACTAGAATTTCAGTGAGTTTTTGAAGTTCCCTTAAAGTT |
| RPS23_NM_001025_r1_9 | /5Biosg/CAGATCCTGACACCTTTAAATAATAAACAGAGTGGGGAGCAATTTTGAGTTTACTA |
| (SEQ ID NO: 1278) | TTTATCATCTTGGGCCCCTGTAAGATGGATACAAGAATGTGCATTTCCAAGGGTACCTAAGATA |
| RPS23_NM_001025_r1_10 | /5Biosg/ACTGAAAACATCAGTCTTGTCGTATACCTTATCTCCCTTGCCTGGCATATACTTCC |
| (SEQ ID NO: 1279) | ATCAACTAAATGATCCAATGCCTGTATTCTCCTAAACACATTAATGTAGACACATTACAACACA |
| RPS23_NM_001025_r1_11 | /5Biosg/GTACTCAATACCTAGCTTAAATTATCAAAACACAACCAATCTTGTCTCTACACTAA |
| (SEQ ID NO: 1280) | ACCACTTCATTTGCTGACTAGTCTTTGAAGACATGAAGGTCTCTGACAACCTCATGAGAAGATA |
| RPS23_NM_001025_r1_12 | /5Biosg/TGTTAACAGGAGTTTCTTACATCAGATTTAAAGCAGAAGGCTCACAGCTTCATTTC |
| (SEQ ID NO: 1281) | AACCATGCCACTGTATCCATGAAAACTCTGAAACAAGTATTTGTGGACAGCAAAATCCACATGT |
| RPS23_NM_001025_r1_13 | /5Biosg/AAAGGTTCTGCTCTTGATCCTACGGAAAGTGGGCAACCAAACCAATTGTTTTCCAA |
| (SEQ ID NO: 1282) | AGATCCTAAAGAATGTAAAGCTAGGCTTTGATCAAAGGGCTAATTAACCCATACTTACTATTTG |
| RPS23_NM_001025_r1_14 | /5Biosg/TGAGGATGACCAACTGAGACCAGTGTTGCTGGAGCACAAAGTGCCAAGGAGAGAAA |
| (SEQ ID NO: 1283) | TGGCAGGCTAAGGCTGGAATATGCAGGTATGAAGCGCAACCTGGGTTCTTCTGTGCCATGTGAA |
| RPS23_NM_001025_r1_15 | /5Biosg/TCAAGAGATAAACCGAAGTGTGCTGGAAAACACACGAGCTCTTTAGTAAGAGCAGG |
| (SEQ ID NO: 1284) | CGACATGCAAGGAACCATAGTAACAGGAACAGAGGTCCTGAGGCTGGATATGGAACCCAAGTTT |
| RPS23_NM_001025_r1_16 | /5Biosg/TGTTGAGCCCCTGGGAACAGCTTAGCCAAATACTGACTGCAGCTACACGTTAGGAA |
| (SEQ ID NO: 1285) | ACACTGGTACAAACCAACAGGCAGCCTTTCCGCCCTTGCGGAGGGAGAGACTAGCATCATCATC |
| RPS23_NM_001025_r1_17 | /5Biosg/GCCTGGAATCGCGCTGCAACCGCGGCCTCTTTCCCTGGTTCTGGCCAGGTGAGCGA |
| (SEQ ID NO: 1286) | GGCCTAGCAATCTCTCGCGCATCACAAAGGATTGGGTCAGTTAAGGCTCATCCGAAAGCATCTG |
| RPS23_NM_001025_r1_18 | /5Biosg/CCAGGCCACATCCCTGGGCGCCCGCGCTGTCCCACTCCCTTCCTGGTGCGCCCCGC |
| (SEQ ID NO: 1287) | CTCGCGGTCACAAGGGCCCCACCGCCCGGGGAGAGCTGCCCACCCCGCGCCTGACGCCCAGGGC |
| RPS23_NM_001025_r1_19 | /5Biosg/AGCCCCTGGGCCTCAGGTAGGACTGCTTAGCAGTGAAAAGGGCGTCCTGGGTGCAG |
| (SEQ ID NO: 1288) | CCAACAGGGTACAGCTGCAAGCCATTGTTGAAGGTGTCTTGACGTATCAGAGAAGGCAGGTAAC |
| RPS23_NM_001025_r1_20 | /5Biosg/ACACAAGGCAGTGGGGAAAGGGGCTGGGGGGCCACACAGAGAAAGGAGAAGGCACT |
| (SEQ ID NO: 1289) | AAGATCAGGCTCAGCAGCTTCTGGATCTGAGGCGCCGGAGATTATTTATGGGTCAATCTGGCAG |
| RPS23_NM_001025_r1_21 | /5Biosg/GTTTTAAGATAAATCCGTTGGCGATACTTCTGAATGTCTATTTATGGATTCCCTAT |
| (SEQ ID NO: 1290) | CTTATTTTATTGATGTGTATTTTTGTCTCTCCTCCATGGATACATGAAGGAAAGGGCCTTGGAC |
| RPS23_NM_001025_r1_22 | /5Biosg/TAGATATATGATCCATTTTGAGTTAAGTTTGTATTACATGTAAGGTTGGGGTTCCT |
| (SEQ ID NO: 1291) | TTGTTTTGGTCTTTGACAAATGGAAGTCCACAACATTTATTGAAACTCTTCTTCGTCCATTGGT |
| RPS23_NM_001025_r1_23 | /5Biosg/CCAATTTATCTATTTTTTCTTTTACGGCATGTGCTTTTGGTGTAATGTCTAAAAAC |
| (SEQ ID NO: 1292) | TATACCTACGCCTAGGTTGTAAAGATAGTCTGTTTTCAGTTTTATAGCTTTCCATTTTACATTT |
| RPS23_NM_001025_r1_24 | /5Biosg/CTATTAATTTCTTCCCAACTCAGCCTCCCAAAGTGCTGGGATTACAGGCATGAACC |
| (SEQ ID NO: 1293) | ACAGCGCCCAGCTTAACAGGATTTTTCAGAGAGAAAAAGTTTTTTAATTTTAATGAAATCCACC |
| RPS23_NM_001025_r1_25 | /5Biosg/TTTTCTTAAAACAGGTACTGAGTATAAAACAATATAAAACAATATGAGAAGGTCTC |
| (SEQ ID NO: 1294) | TCTCTTCCCTCAAGAGTGTATGACTTTGCTGCCCAGGATGATCTTGAACTCCTGGCCTCAAGCT |
| RPS23_NM_001025_r1_26 | /5Biosg/TAACATCAGGTTCLAAGTCGTAAGTTGATTTTTAACTACTAGGTTTAGGCCAGGCA |
| (SEQ ID NO: 1295) | GGCCCAGGGCTGGTTTCAGGCCTGGTGCCGGGCTGCCTGTCTTTGATTTCACTTCCTTGTTTTT |
| RPS23_NM_001025_r1_27 | /5Biosg/ATGAAAGGGTCGTGATTGATTGAGCAATCAAGGGGATATGTGACAGGGGTTTCATG |
| (SEQ ID NO: 1296) | CACTGGTACAGAACACAACAGGGAGTTTCACAATTTTTTTATACAATGCTTGGAATCTACGGAT |
| RPS23_NM_001025_r1_28 | /5Biosg/TGTAGAAGGAAGGGCTTTATTCAGCTGGGAGCATCGGCAAGCTACAGCCTTAAAAT |
| (SEQ ID NO: 1297) | CTGAGCTCCTCAAGTGCACAATTTCTGTCCCTTTTAAGGGCTCACAACACTAAAGATTTCACAT |
| RPS24_NM_001026_r1_1 | /5Biosg/GCCAAGGAGGAAAAGAGAACCACGATTCGCCGGCCAATCATATCAACGCGCACGGA |
| (SEQ ID NO: 1298) | AGGACCCCGGGAAGAGCCGGCGTGGGGGCGTGGGCGCGCCACGAGGCTGACCGCGCCGATGCCT |
| RPS24_NM_001026_r1_2 | /5Biosg/GACATCAATGACCATTTGTTTCGTCTGAAGTAGTCGGTTGGTCATGAACTTTCTAG |
| (SEQ ID NO: 1299) | TGCGGATAGTTACGGTGTCGTTCATGATGGCGATCTATCTTCAGACAGCCAAGGAGGAAAAGAG |
| RPS24_NM_001026_r1_3 | /5Biosg/TTCTGAATCCAAAAACAAAGATGACATCCGGTGTGGTCTTGTACATTTTGGCTAGT |
| (SEQ ID NO: 1300) | TTTTCCGGAATTTCTGTCTTAGGCACTGTCGCGTTCCCGGGGTGAAGGACATCAATGACCATTT |
| RPS24_NM_001026_r1_4 | /5Biosg/GGCCATGTCTTGCAAGTCTATGTTTGGGTTCATTTTTCTTTGCATAATCCAGGGAA |
| (SEQ ID NO: 1301) | TCATAAATCATGCCAAAGCCAGTTGTCTTGCCACCACCAAAATGAGTTCTGAATCCAAATACAA |
| RPS24_NM_001026_r1_5 | /5Biosg/TTTTTGCCAGCACCAACATTGGCCTTTGCAGTCCCCCTGACTTTCTTCATTCTGTT |
| (SEQ ID NO: 1302) | CTTGCGTTCCTTTCGTTGCTTTCTTGAGGTCTTTTTCTTCTCATACAGGCCATGTCTTGCAAGT |
| RPS24_NM_001026_r1_6 | /5Biosg/TTTCAAACAACCAGACACATGAAGTTTTTAGTTTATTAATGTTCTTGCGAAAAATC |
| (SEQ ID NO: 1303) | CACAGTGGCCACAGCTAACATCATTGCAGCACCTTTACTCCTTCGGCTTTTTGCCAGCACCAAC |
| RPS25_NM_001028_r1_1 | /5Biosg/GTCTTTCTTGGCCGACTTTCCAGCGTCCTTCTTCTTCTTGTCGTCCTTAGGCGGCA |
| (SEQ ID NO: 1304) | TTGCGAAGCTCGGAGAATAGCAGCAGACACCGCAGCCTCGTCAAGATGTCGGACAAAAAGGAAG |
| RPS25_NM_001028_r1_2 | /5Biosg/AAACAAGACTAAGTTATTGAGCTTGTCCCGAACTTTGCCTTTGGACCACTTCTTCT |
| (SEQ ID NO: 1305) | TTTTGGCCTTGCCCCCGGATTTGTTCACTGGGTCTTTGTCTTTCTTGGCCGACTTTCCAGCGTC |
| RPS25_NM_001028_r1_3 | /5Biosg/CCTCGAATCTTCAGTCTCTCAGAGACCACAGCTGGGGTTATAAGTTTATAGTTGGG |
| (SEQ ID NO: 1306) | AACTTCCTTACAGAGTTTATCATAGGTAGCTTTGTCAAACAAGACTAAGTTATTGAGCTTGTCC |
| RPS25_NM_001028_r1_4 | /5Biosg/TTCTGGTGTAAATTACTTGAGCTCTGTGCTTTGAAACCAGTTTGATAAGTCCTTTA |
| (SEQ ID NO: 1307) | CTAAGGAGCTCCTGAAGGGCTGCCCTGGCCAGGGAGCCTCGAATCTTCAGTCTCTCAGAGACCA |
| RPS25_NM_001028_r1_5 | /5Biosg/TGATTTAATAAAGTTTTATTTTTCCAAATGTACAGCTGGTTGGACCTATTCATGCA |
| (SEQ ID NO: 1308) | TCTTCACCAGCAGCTGGAGCATCTCCACCCTTGGTATTTCTGGTGTAAATTACTTGAGCTCTGT |
| RPS26_NM_001029_r1_1 | /5Biosg/CAGCATTTCTGCTGAAATCTAGGGTGGAAATGCGTTCCTAGTGTTTATTTATCCAT |
| (SEQ ID NO: 1309) | GGAACAAAGTGCACATGGGACTATTTAGCCGGATGGCGGAAGAAAATCGAGGTTATGTGTCTCC |
| RPS26_NM_001029_r1_2 | /5Biosg/CCTGGCAGGGCCCTCCTATATAGCATGAGATCCCTACGCGGACACCGGGAGAATCC |
| (SEQ ID NO: 1310) | TTAGGAGACGGGCTTCAAGAACGGCAACTCACTTTACTCAAATATTCCTTTACATTCAGCATTT |
| RPS26_NM_001029_r1_3 | /5Biosg/CACAGTTAGTGCAGCGAATAGGCTGCACGTGGCCGCGGCCCTTTTTGGCACGACCA |
| (SEQ ID NO: 1311) | TTGTTCCTTCTTTTCTTTGTCATCTTGGAGGCACGGACCGGAGAGAGGAGACGGTGCCTGGCAG |
| RPS26_NM_001029_r1_4 | /5Biosg/AGCACATAGGCATCGAAGACGCTCGCTTCAGAAATGTCCCTGACTGCTGCGGCCTC |
| (SEQ ID NO: 1312) | CACTATGTTTCGAATGACGAATTTCTTAATGGCCTTGTCCTTGGGCACGCATCGGGCACAGTTA |
| RPS26_NM_001029_r1_5 | /5Biosg/AAATCGGGGTGGGGGTGTTCGGTCCTTGCGGGCTTCACGAGATCGATTCCTGACTA |
| (SEQ ID NO: 1313) | CTTTGCTGTGAATTGCACAACTCACACAGTAATGTAGCTTCACATACAGCTTGGGAAGCACATA |
| RPS26_NM_001029_r1_6 | /5Biosg/AAGTACAATTTCCATTTTATTTTTCTCCAGAGAATAGCCTGTCTTCAGTCTTTAAG |
| (SEQ ID NO: 1314) | AACTCAGCTCCTTACATGGGCTTTGGTGGGGGACGTGGGGCAGCACCCGCAGGTCTAAATCGGG |
| RPS27_NM_001030_r1_1 | /5Biosg/TTGGGGCTCTGCACCAGGCGTTTCTTCTTGTGTTTCCTCTTCTCCTCTTCTGGAGA |
| (SEQ ID NO: 1315) | GGGATGAAGGAGATCCTTTGCGAGAGGCATGTTCTCGTGTGCGTAGGTCGTCACCGCCGGAAAG |
| RPS27_NM_001030_r1_2 | /5Biosg/CTGTAGGCTGGCAGAGGACAGTGGAGCAGCCAACACACAAAACTACCGTTTGTGCA |
| (SEQ ID NO: 1316) | TGGCTAAAGACCGTGGTGATTTTATAGCATCCTGGGCATTTCACATCCATGAAGTAGGAATTGG |
| RPS27_NM_001030_r1_3 | /5Biosg/TCAGGATTTATCCAAAATGTGTTTATTGAGATGGTTTCCCACTCATCTTGACTCAG |
| (SEQ ID NO: 1317) | AGTGCTTTTAGTGCTGCTTCCTCCTGAAGGAACATCCTTCTGTAAGCCTTGCTTTTCCTCCTGT |
| RPS28_NM_001031_r1_1 | /5Biosg/ACCTGCGTGCACTGTCCCTGAGAACCGGTCCTGCCCAGGACCTTGGTGACCCTGGC |
| (SEQ ID NO: 1318) | CAGCTTGATAGGCTGCACACGGCTGGTGTCCATGATGGCGGCGCGGCGGCGGTCTGGCGGAGAG |
| RPS28_NM_001031_r1_2 | /5Biosg/AAAAGGGTGAGCACGTCGCCCTCGCGCACGGGGCCTTTTACATTGCGGATGATGGA |
| (SEQ ID NO: 1319) | TCGGCTCGTGTCGTCCATGAATTCCACGCGCACCTGCGTGCACTGTCCCTGAGTACCGGTCCTG |
| RPS28_NM_001031_r1_3 | /5Biosg/TCCCATCGGCCAAGTGGTCGAACCCGACATCCAAGACCCAGCGAGCAGCCAAGCTC |
| (SEQ ID NO: 1320) | AGCGCAACCTCCGGGCTTCTCGCTCTGACTCCAAAAGGGTGAGCACGTCGCCCTCGCGCACGGG |
| RPS28_NM_001031_r1_4 | /5Biosg/TTAACTTGAAACACAAACGCTTTATTTAAAGGAGCATCTCAGTTACGTGTGGCGGA |
| (SEQ ID NO: 1321) | CAAAAAAAAGGAGCAGACTGTGACAGACCATTCCCATCGGCCAAGTGGTCGAACCCGACATCCA |
| RPS29_NM_001032_r1_1 | /5Biosg/CCCTGGCCGAATTTTCGCGGGTGGCTCCAGTACAGCTGCTGGTGACCCATCTTGCT |
| (SEQ ID NO: 1322) | CTCAGCAGTGCAACGAGGTAAAAGGAAGAAGCTGGCCCACGCATGCGCTCTTCAAATTTTTGAG |
| RPS29_NM_001032_r1_2 | /5Biosg/TAATGAAACCGATATCCTTCGCGTACTGACGGAAACACTGGCGGCACATATTGAGG |
| (SEQ ID NO: 1323) | CCATATTTCCGGATCAGACCGTGCCGGTTTGAACAGACACGACAAGAGCGAGAACCCTGGCCGA |
| RPS29_NM_001032_r1_3 | /5Biosg/TTTTTGAAGGGTTTTTTCAAATGTTTATTTTATATACAAAGAATTATCATGGTTTT |
| (SEQ ID NO: 1324) | TCATTGAGTAGATGCCCCGGATAATCCTCTGAAGGAAGAGCATTTAGTCCAACTTAATGAAACC |
| RPS2_NM_002952_r1_1 | /5Biosg/TGCCACTGCCGAAACCTCCGCGGAAGCCACCGCGGTTCCCCATCCCAGGGCCACCA |
| (SEQ ID NO: 1325) | GGGCCCCCGGGCCCCCCCGCTGCACCGGCGTCATCCGCCATTTGGTGTTTTGTCGGAAAAGAAG |
| RPS2_NM_002952_r1_2 | /5Biosg/CCTTGACCAACCGGCCCAACTTGGTGACGGGCATCCACTCCTTATCCTCGGCCTTG |
| (SEQ ID NO 1326) | CCTCCGCGAGCTCCGCGGCCTCGGCCCCGGCCCCGTCCACGGCCGCGACCCCGGCCCCGGATGC |
| RPS2_NM_002952_r1_3 | /5Biosg/GGCATAATCTTCAAAACCTCATCCTTGAGAGAGGCCCCCAGGAAGAAATCAATGAT |
| (SEQ ID NO: 1327) | CTCTGATTCCTTAATAGGCAGGGAGAAGAGATAGATCTCCTCCAGGGACTTGATCTTCATGTCC |
| RPS2_NM_002952_r1_4 | /5Biosg/GCGGTGGCCACCTCCTTGGAGCACTTAACACCCAGACCGACGTGGCCATTGTAGTC |
| (SEQ ID NO: 1328) | CCCGATAGCAACAAATGCCTTGAACCTGGTGCGCTGGCCGGCACGGGTCTGCTTCTGCACTGGC |
| RPS2_NM_002952_r1_5 | /5Biosg/GCCGCAGCGGCCTGTCACCTTGCAAGGGACAGTGTGGGGCTTGCCGATCTTGTTCC |
| (SEQ ID NO: 1329) | CCCAGTAGCCTCTGCGCACGGGGACGATGGAGAGCTTGGCCAGGATGATGGCCCCACGGATGGC |
| RPS2_NM_002952_r1_6 | /5Biosg/GCCCCGGGCTGAGGTGTAGCAGTCATCGATACCAGCCATCATGAGCAGCTTCTTAG |
| (SEQ ID NO: 1330) | GCACAGGTGCGGAGACGATGCCAGTGCCCCTGGGTGCAGGGATGAGGCGTACCAGCACAGAGCC |
| RPS2_NM_002952_r1_7 | /5Biosg/CCTGATAGGGAGACTTGGTGAATACAGTCTCCTTCCAGAGGTCGGGGGTCAGGTAG |
| (SEQ ID NO: 1331) | CTGTAGGTCTTAGAAATGGCATCAAAGGTGGCCTTGGCGAAGTTGCCCAGGGTGGCAGTGCAGC |
| RPS2_NM_002952_r1_8 | /5Biosg/CACGCTTAATTCACTTTATTTTTCTTGTATAAAAACCCTATGTTGTAGCCACAGCT |
| (SEQ ID NO: 1332) | GGAGCCTGAGTCCGCTGCACGGAGACTCTGGTGTGGGTCTTGACGAGGTGGTCAGTGAACTCCT |
| RPS3A_NM_001006_r1_1 | /5Biosg/TGCCGCCTTTCGTAAGGCGCTTGTTCTTGCCAACCGCCATGGTGCTGGTCAGAGAG |
| (SEQ ID NO: 1333) | CCAAAAGGGCGGAAGTGGGAGTCGCGCGAGAACTTAGGCGTACGGGGGCGGGGCGCGCCGTCTA |
| RPS3A_NM_001006_r1_2 | /5Biosg/GGTCCTGGTGACGAGCGTCTTTCCAATATTTCTTATATTGAACATAGCAGGTGCTT |
| (SEQ ID NO: 1334) | TCACATCATACCAATCTTTCTTAGAAAATGGATCAACCACTTTCTTCTTGGCTCCCTTTTTGCC |
| RPS3A_NM_001006_r1_3 | /5Biosg/ACATCTTCAGTAATCAGCTTGAATTTTCTAAATGCAACTTCATCATTCTGCAAATC |
| (SEQ ID NO: 1335) | AGCAAGACTCACTTCAAACACACGACCCTTGAGACCATCAGATGCAATTTTGGTTCCTTGGGTC |
| RPS3A_NM_001006_r1_4 | /5Biosg/TAGTCTTGACATCAACGTGAGCTTCAATCATTGTCTGCCATTTTTTGACCATGGAA |
| (SEQ ID NO: 1336) | CACATTTTGTCACGGGTAAGATCCATGCCATGGAAGTTAGTCAGGCAGTTTTTACCCTGAACAT |
| RPS3A_NM_001006_r1_5 | /5Biosg/TCATCTTCTTCCGGATTTGGCGGACCTGTTGGTGCTGAGCATAAGAGGTCTTCCGT |
| (SEQ ID NO: 1337) | ATCTGATTGTTGCGTTTTTTAGTAAAACCAACACAGAACAGACGAAGCAAGTAACCATCGGTAG |
| RPS3A_NM_001006_r1_6 | /5Biosg/GAGAGGATAAATAGATTGGCAAGCCTTTTCTATGTCTTTTCCAATGCTGTCTGGAA |
| (SEQ ID NO: 1338) | TCAATTTATTGACCACTTCTTTCAAGTCATTTGTCTGCACCTCTCGGGTCATGATTTCCATCAT |
| RPS3A_NM_001006_r1_7 | /5Biosg/GTCTCGTCCCCAGTGGCTTTTCCAGAACTACTGCCTTCACCATGAAGCTCCATGAG |
| (SEQ ID NO: 1339) | CTTTCCCAATTCAAACTTGGGCTTCTTCAGCATTTTTACTTTTCTAACGAAGACATCATGGAGA |
| RPS3A_NM_001006_r1_8 | /5Biosg/TCAGAAGCAAACCATCACAAATAGCACTTTTTATTTGCCACTATTTGAAGTCTGAA |
| (SEQ ID NO: 1340) | CTTTAAACAGATTCTTGGACTGGTGGTTCATATCCATCAGCTCGTTCAACTTTAGCACCTGTCT |
| RPS3_NM_001005_r1_1 | /5Biosg/CAGTTCAGCTTTGAAGATGCCATCAGCGACAAACTTCCTCTTCTTGGATATTTGCA |
| (SEQ ID NO: 1341) | CTGCCATCTTGCCGCCGCGCTCCGCTGAAAGGAAAGGAAGTGGCTCGCGGGCGGAAGTGAGTAT |
| RPS3_NM_001005_r1_2 | /5Biosg/CCAAGAACATTCTGTGTTCTGGTGGCTAAGATAATGATTTCTGTCCTGGTTGGTGT |
| (SEQ ID NO: 1342) | AACTCGCACCTCAACTCCAGAGTAGCCATCTTCAGCCAGCTCCCGAGTAAGAAACTCATTCAGT |
| RPS3_NM_001005_r1_3 | /5Biosg/TGGCACACAGACCTCTAGTGGCCACCTTTTCAGCATAAAGCTCTACACTGCCCTCT |
| (SEQ ID NO: 1343) | GGAAAGCCAAACCTCTTCTGAACTACAGCAGTCAGTTCCCGAATCCGCCGGCCCTTCTCACCAA |
| RPS3_NM_001005_r1_4 | /5Biosg/CACAACCTCGCAGCCTTTGGCCCCACTCTCCATGATGAACCGCAGCACACCATAGC |
| (SEQ ID NO: 1344) | AGGCCCTCCGCACAGCAAGCCCTCCTAGGAGTTTGTAACGCAGAGACTCTGCCTGGGCAATGGC |
| RPS3_NM_001005_r1_5 | /5Biosg/AACACGTGGCGCACAGCAGTGTCAACGTAGTAGTTAACAGGGTCTCCGCTGTGGAT |
| (SEQ ID NO: 1345) | CATCAGGCCATCCACAAACTTCATGGATTLAGCCCTCTGTCCTCGGAGTTTCCCAGACACCACA |
| RPS3_NM_001005_r1_6 | /5Biosg/TCTTTGGGTTCCACAATGCTCACGTGGTCAGGCAGGGGCTTCTTAGGGCCAATCTT |
| (SEQ ID NO: 1346) | ACCAGTTGGGTCCCAGGGCAGCATGATCTTCACCTTGATGCCCAGCACACCCTGTCTGAGCAAC |
| RPS3_NM_001005_r1_7 | /5Biosg/CCAGAATACAGCTGCCAAGGAGACCCTGTTATGCTGTGGGGACTGGCTGGGGCATG |
| (SEQ ID NO: 1347) | GCAGGCGGCTCTGGCTTCCCACCCTTCTGTTCTGAGATGGGGGTGGTGGGCAGAATCTCATCTT |
| RPS3_NM_001005_r1_8 | /5Biosg/TATGTAATGCTCCAGAGGCCAACATGCCCCTCAGTCTGAATACTGAACAGTCTAGT |
| (SEQ ID NO: 1348) | CAGACCTTGTGTCTTTGTACAAAATTTTATTAAAGGTCTTTAGAGAGCAACATCCAGACTCCAG |
| RPS3_NM_001005_r1_9 | /5Biosg/CCTTGCTTGCTATCAGCCTTCTCTATCACACAAATATTATTTCTCTGGTTGGCTCT |
| (SEQ ID NO: 1349) | GCTTGGGTCTTACTGTTCTGGCCCTTGCAAATACCACAAGTATGGTTAAAACCAAGAAGATATG |
| RPS3_NM_001005_r1_10 | /5Biosg/CATTTATATTCACTTTCTATGTGCAACGTGGATCGCCTTATGCATAGGAAAAACTC |
| (SEQ ID NO: 1350) | AAACATATCCCTCTTCTAAACCTACAATCCTGAACTACAGGACAACGAATCAAGGTGCTGCCTT |
| RPS3_NM_001005_r1_11 | /5Biosg/GAGCCTTATGTGCCTTCAGTGGTGCAAGCAAATTTCCTTTACACTTTAGAGAGGTT |
| (SEQ ID NO: 1351) | GATTAACGAGTACATATGTAAAGAACACTTAGAGCACAGCATGACACAAAATATAATGGCCATT |
| RPS3_NM_001005_r1_12 | /5Biosg/TCCAGAACATAATAGCCTGTAGTACCAATGACTGGTTCCATGATCCCCTAAGAGAA |
| (SEQ ID NO: 1352) | CACAACTTAGGAATGTGGATTCTAATGATAGCTTTATACTGCTTAGGCAAATTTACTTCTGAGC |
| RPS3_NM_001005_r1_13 | /5Biosg/CTAGTTTTGCCTGAAAAAATAGTGCCGAATTTCTTGTGAAGTACAGACACTGGTAG |
| (SEQ ID NO: 1353) | ALAATACTTCAAAACATTGATAGATAAAATCAGAGACAATTTAAATGTTCTTCACAGTTCTCCA |
| RPS3_NM_001005_r1_14 | /5Biosg/GGCTGCATCACCAGGAAGCAGCCACCTGATAACAGCTTTCCCTGGAAGTCCCCTCT |
| (SEQ ID NO: 1354) | GTGATCAACCAGCATCTGATTTAACAACAGTCTCATGATTTTCAAGCCAACCTGTCCCTCACTA |
| RPS3_NM_001005_r1_15 | /5Biosg/TTACCATGTGCCAAGCAATATAAACACCATGTGCAGCTCAGTCTAATTGAGAAGAA |
| (SEQ ID NO: 1355) | AACCAAATCATCACAGCACCTGCTAAGCCATCTGTGGAGCCAGGGTTATCTCATCAGCCAGGCT |
| RPS3_NM_001005_r1_16 | /5Biosg/CACTATAGAAGTGTACACTTAAATGGTTAAGATGACCAATTTATGAGTCAAAAAGG |
| (SEQ ID NO: 1356) | AAGGGGGGGTCAGATACTTGTCAGTGTACGCCCTACATAATTACCTCAAATATTAAGCCCTTAC |
| RPS3_NM_001005_r1_17 | /5Biosg/TAAGAAAAATAAGTCTACATGTTCAATACACACACAACCATCCTTTTTGATTATTT |
| (SEQ ID NO: 1357) | TTGACCCAAAGTTGGTTGAATCCACAGATGCAAATCCCTTGGACAGAGGGCTAACTCTGTCACT |
| RPS3_NM_001005_r1_18 | /5Biosg/TTTTAGACTTTCAATCTTCACTGGATTACTTTTAATACCTAATGTACATGCTACAT |
| (SEQ ID NO: 1358) | AGCTGTTACAACTGCAGTGTACAAGAAAATAAGTCACAAGAAAATATAGTATTTTGAAAATAAG |
| RPS4X_NM_001007_r1_1 | /5Biosg/ATGCTTCTTGGGACCACGAGCCATGGCTGCGTTAGGCAAGGAAAGAGGACCTCCGT |
| (SEQ ID NO: 1359) | CTTCCGGTGCGCGTAGAAATTGGGGCTGGAGACTGCGCGCGCCGGATTTTCAGCTGCTCCGCCC |
| RPS4X_NM_001007_r1_2 | /5Biosg/AGGAAAATGATGAGGGGGAGACACTCTCTCAACTTGTGGGGACCGGTGGATGGACG |
| (SEQ ID NO: 1360) | AGGAGCAAACACACCGGTCAATTTATCCAGCATCCAATGCTTTGGAGCTGCCACCCGCTTCAGA |
| RPS4X_NM_001007_r1_3 | /5Biosg/TCCATGAATCCAGCAGGGTAGGTTATATCAGTTCGGACCTTGCCATCGATTTTAAT |
| (SEQ ID NO: 1361) | CAACCGCTGCATGCAAATCTTCTTTACTTCATCTCCTGTCAGGGCATACTTAAGTCTGTTCCTC |
| RPS4X_NM_001007_r1_4 | /5Biosg/TTCTCACTTTGCACAACTTGTACTTGGCCTCCTCAGGTGTAATACGATGTACAGCA |
| (SEQ ID NO: 1362) | AAGCGACCCTTGGTGTCATAGATCAGACGGAAATTCTCTCCCGTCTTGTCAATGCTGATGACAT |
| RPS4X_NM_001007_r1_5 | /5Biosg/TGCCAGTCTCCAAATCAATCTGAATGGTATCATTCACCTTGATGAGGGGATCGGGG |
| (SEQ ID NO: 1363) | TAGCGGATGGTGCGGGCATCATGAGTCACCAGATGAGGGATTCCTTTTGTGCCCACAAAGATCT |
| RPS4X_NM_001007_r1_6 | /5Biosg/CACGTCAAAAGATCCAGGGTGCCTCTCTCTGTTGGTGATCACACCAATTCTTCCTA |
| (SEQ ID NO: 1364) | GGTTAGCACCTCCAGTCACCATACACAGGTTACCAGTGTCGAACTTGATGAAATCAGTAATCTT |
| RPS4X_NM_001007_r1_7 | /5Biosg/GGTGAGGCGGATACCCTTTCCTCGGGCAAGAGAAATCCATGGTTTGTTGCCCTTGC |
| (SEQ ID NO: 1365) | CAATAACAAAAATGTTGGAAAGTCGAGTGGCAAAGCTGTTGCCATTGGCATCTTTCACGTGAAC |
| RPS4X_NM_001007_r1_8 | /5Biosg/TGCTATTAATCCTGCCACAATATTTTTAATTACGTACAAAGATCTGACATGTCACC |
| (SEQ ID NO: 1366) | CAGGGACCCATTTCACCCACTGCTCTGTTTGGCCGCCAGTCTTTTGTCTCTCTCTTCAGCAATG |
| RPS4Y1_NM_001008_r1_1 | /5Biosg/ACACCCGTTAGTTTGTCAAGCATCCAATGCTTCGGCGCTGCAACACGCTTTAAGTG |
| (SEQ ID NO: 1367) | CTTCTTGGGGCCCCGGGCCATGGCGAAACTCTGCGACGGAAGAGAATCTGTTCTTTTCCGGTGC |
| RPS4Y1_NM_001008_r1_2 | /5Biosg/TTCTTTACCTCATCTCCAGTCAACGCATACTTGAGTCTATTCCTGAGGAAGACGAT |
| (SEQ ID NO: 1368) | CAGAGGAAGACATTCCCTCAGCTTGTGGGGACCTGTCGATGGACGAGGTGCAAATACACCCGTT |
| RPS4Y1_NM_001008_r1_3 | /5Biosg/GCGGAAATGTTCACCTGTCTTCTCGATGCTGATGACATCCATGAATCCAGCAGGGT |
| (SEQ ID NO: 1369) | ATGTGACATCCACTCGAACCTTGCCATCAATTTTGATGAAACGTTGCATACATATCTTCTTTAC |
| RPS4Y1_NM_001008_r1_4 | /5Biosg/AGGGATTCCCTTCACTCCCACAGTAATCTTCCTCACTTTGCACAACTTGTACTTTG |
| (SEQ ID NO: 1370) | CCTCTTCCACTGTGATGCGGTGAACAGCAAAACGGCCCTTGGTGTCATAGACCAGGCGGAAATG |
| RPS4Y1_NM_001008_r1_5 | /5Biosg/ATTTGATAAAGTTGATTATCTTGCCAGTCCCTAAATCAATCTGCACAGTATCGTTC |
| (SEQ ID NO: 1371) | ACCTTGATGACAGGATCTGGGTAGCGGATGGTTCGAGCATCATGAGTCACCAGGTGAGGGATTC |
| RPS4Y1_NM_001008_r1_6 | /5Biosg/CCTTCACATGCACCACATCAAAAGAACCAGGATGTCTTTCCCTGTTGGTGATCACA |
| (SEQ ID NO: 1372) | CCAACACGACCGAGGTTGGCTCCACCAATCACCATACACAAATTGCCTGTATCAAATTTGATAA |
| RPS4Y1_NM_001008_r1_7 | /5Biosg/GCAACAGTAAGTCGAATGCCCTTTCCCCTGGGCAGGGAAATCCAAGGTTTATTGCC |
| (SEQ ID NO: 1373) | ATTGCCAATGACAAAAATTGTTGGAAAGCCTCGTGGCAAGCTGTTGCCATTGGCATCCTTCACA |
| RPS4Y1_NM_001008_r1_8 | /5Biosg/TTTTTAAGAAACGAGAATTCACTGTTTATTTGTGCAAAGAAAAAAAGATATGCTGC |
| (SEQ ID NO: 1374) | TACTGCAATTTAGCCACTGCTCTGTTTGGTGGCCAGCCTCTTATCTCTCTCTTCAGCAACAGTA |
| RPS5_NM_001009_r1_1 | /5Biosg/GTCTGGGGTCTCTGCCACCGCTGGTGCTGCTGTCTCCCACTCGGTCATCCTGAGAA |
| (SEQ ID NO: 1375) | CACAGCCTGAGCGTCTCTGTCACTCGGCGTAGACCACGCGCCGCCCTGGTACAGACAGCAAGAG |
| RPS5_NM_001009_r1_2 | /5Biosg/TGAGGCAGGTACTTGGCATACTTCTCCTTCACTGCAATGTAATCCTGCAGGGAAAT |
| (SEQ ID NO: 1376) | GTCATTGATCTGCACATCATCGGTGCTCCACTTCCCAAAGAGCTTGATGTCTGGGGTCTCTGCC |
| RPS5_NM_001009_r1_3 | /5Biosg/TCTTGCCGTTGTTGCGGCCGTGCATCATCATGGAGTTAGTGAGGCGCTCCACAATG |
| (SEQ ID NO: 1377) | GGACACTGAGCTTTGCGGAAGCGTTTGGCGGCATACCGCCCTGCACTGTGAGGCAGGTACTTGG |
| RPS5_NM_001009_r1_4 | /5Biosg/ACCACTGTTGATGATGGCGTTCACCAGGACCTGCAGAGGGTTCTCGCCTGTGAGCA |
| (SEQ ID NO: 1378) | GGTGTATGATCTCGAAGGCATGCTTGACGATGCGCACAGTCATGAGCTTCTTGCCGTTGTTGCG |
| RPS5_NM_001009_r1_5 | /5Biosg/CACAGCAGCCAGATGGCCTGGTTCACACGGCGCAGGGGGGACACATCCACAGCCTG |
| (SEQ ID NO: 1379) | TCGTCTCACAGTCCCGGCGCGCCCAATGCGTGTGGAGTCCTCCCGGGGACCACTGTTGATGATG |
| RPS5_NM_001009_r1_6 | /5Biosg/TGGCATAGGAGTTCGAGGAGCCCTTGGCAGCATTGATGAGCTCATCTGCCAGGCAC |
| (SEQ ID NO: 1380) | TCAGCAATGGTCTTAATGTTCCGGAAGGCAGCCTCACGAGCGCCTGTGCACAGCAGCCAGATGG |
| RPS5_NM_001009_r1_7 | /5Biosg/TTTTGGCTGGGACTGCCCCAAAGGGCAGACAGGTTTATTGGGCAGCAGCTGGGAAA |
| SEQ ID NO: 1381) | ATCAGCGGTTGGACTTGGCCACACGCTCCAGCTCGTCCTTCTTCTTAATGGCATAGGAGTTCGA |
| RPS6_NM_001010_r1_1 | /5Biosg/AGTACGAAGTTTGCG7TCATCGTCCACTTCAATGAGTTTCTGGCAGCCAGTGGCTG |
| SEQ ID NO: 1382) | GGAAGGAGATGTTCAGCTTCATCTTGAAGCAGCTGAACGCCTCCGAGGCGCCACGGAAAAGAGG |
| RPS6_NM_001010_r1_2 | /5Biosg/CCTGCTTCATGGGGAAACCTTGTTTGTCGTTCCCACCACTGATTCGGACCACATAA |
| SEQ ID NO: 1383) | CCCTTCCATTCTTCACCCAGAGCGTCAGCAGCAACTTCTGTGGCCATACGCTTCTCATAGAAAG |
| RPS6_NM_001010_r1_3 | /5Biosg/TTTGCATCCACAATGCAACCACGAACTGATTTTCTCTTTCTTTCTCCAGTTCTCCT |
| SEQ ID NO: 1384) | TGGTCTGTAACAGGAATGCCCCTTACTCAGTAGCAGGCGGACACGGCCATGGGTCAAGACACCC |
| RPS6_NM_001010_r1_4 | /5Biosg/AAAGTTTGCGGATTCTGCTAGCTCTTTTGGGGCCCAGGCGGCGAGGCACTGTAGTA |
| SEQ ID NO: 1385) | TCAGTCAGTCCAGGAATATCCTTCTCTCCTTTTTTTACAATAACCAAGTTGAGAACGCTCAGAT |
| RPS6_NM_001010_r1_5 | /5Biosg/ACACGTGGAGTAACAAGACGCTGAATCTTGGGTGCTTTGGTCCTAGGTTTCTTACC |
| SEQ ID NO: 1386) | TTCTTTATTTAAGGGCTTTCTTACAACATACTGGCGGACATCATCTTCTTTAGAGAGATTGAAA |
| RPS6_NM_001010_r1_6 | /5Biosg/CTTCTCCTTAGCCTCCTTCATTCTCTTGGCCAAAAGTTTAGCATATTCTGCAGCCT |
| SEQ ID NO: 1387) | CTTCTTTATTTTTCTTGGTACGCTGCTTCTTCAGAGCAATACGCCGCCGTTTGTGCTGCAGGAC |
| RPS6_NM_001010_r1_7 | /5Biosg/CAGAGTCTGATCTTATTTATTTGTTACTCAAAAAATCTTATTTCTGACTGGATTCA |
| SEQ ID NO: 1388) | GACTTAGAAGTAGAAGCTCGCAGAGAGGAAAGTCTGCGTCTCTTCGCAATTTGTTCCTGGCGCT |
| RPS7_NM_001011_r1_1 | /5Biosg/CTCGAACTGAACATGGCTTTCTCCTGGGAGAACTTGCCGAGCGCCGGCTTAGGAAG |
| SEQ ID NO: 1389) | AGACCCAAATCTCGCGAGAGCACGTCAAAATCCGGCGTCCGAAGGCAAGAGGCGGAAACAGCGC |
| RPS7_NM_001011_r1_2 | /5Biosg/TGAGCCTTGAGGTCCGAGTTCATCTCCAGCTCCAGAAGAGCCTGGGAGATGCCGGA |
| (SEQ ID NO: 1390) | CTCGAACTCGTCCGGCTTCTCGCCATTGGGCTTCACGATCTTGGCGCTCGAACTGAACATGGCT |
| RPS7_NM_001011_r1_3 | /5Biosg/GGAAAGATTTCAGTTGAGGAACGGGAACAAAGATTATGATAGCTTTCCGACCACCA |
| (SEQ ID NO: 1391) | CCAACTTCAATTTCCTTAGCTGCCGTAATATTCAGCTCCCTGAGCTGAGCCTTGAGGTCCGAGT |
| RPS7_NM_001011_r1_4 | /5Biosg/TTCGAGTTGGCTTAGGCAGAATTCTCCTCTGAGCGATAAAGACGACATGCTTCCCA |
| (SEQ ID NO: 1392) | CTGAACTTTTTCTCCAATTCGCGTACTAGCCGGACTTGGATTTTCTGGAAAGATTTCAGTTGAG |
| RPS7_NM_001011_r1_5 | /5Biosg/CCACAATTTCGCTTGGGAAGACCAAGTCCTCAAGGATGGCATCGTGCACAGCTGTC |
| (SEQ ID NO: 1393) | AGAGTACGGCTCCTGGGACGCTTTTGCTTATTTTTTGTACGGCTTTTTCGAGTTGGCTTAGGCA |
| RPS7_NM_001011_r1_6 | /5Biosg/AGAAAAAGTTTCAACCTTGTGTTCCACATTGTTCTGCTGTGCTTTGTCCAAATGAA |
| (SEQ ID NO: 1394) | CCTTTATGAGCCGGCTGCCATCTAGTTTGACGCGGATTCTCTTGCCCACAATTTCGCTTGGGAA |
| RPS7_NM_001011_r1_7 | /5Biosg/TTTACTGTGAATATATACTTTTTATTTAGTCATTTTTGTTTACAATTGAAACTCTG |
| (SEQ ID NO: 1395) | GGAATTCAAAATTAACATCCTTGCCCGTGAGCTTCTTATAGACACCAGAAAAAGTTTCAACCTT |
| RPS8_NM_001012_r1_1 | /5Biosg/CTGGGCGCCCCAACTCATACTTCCGCTTCTTGTGGTAGGGCTTTCTCTTGCCCCCG |
| (SEQ ID NO: 1396) | GTTTTGCGGCGCTTGTGCCAGTTGTCCCGAGAGATGCCCATCGCTCGGCGCTGGCTGGAAAGAG |
| RPS8_NM_001012_r1_2 | /5Biosg/AACACTCTGAGCCCCAGGAGAAATTCCCCACGTCCAACCTCAGGGCACGGTATTTC |
| (SEQ ID NO: 1397) | TTGTTACCTCCCCGCACACGGACTGTGTGGATGCGGCGGGGGCCAATCTTGGTGTTGGCAGCTG |
| RPS8_NM_001012_r1_3 | /5Biosg/GTCGGTACGGTGTGCTGTCGATGAGCACGATGCAATTCTTCACCAGGGTCTTGGTA |
| (SEQ ID NO: 1398) | CGAACCAGCTCGTTATTAGATGCATTGTAGACAACATCGATGATCCTTGTTTTACGAGTACAAC |
| RPS8_NM_001012_r1_4 | /5Biosg/CATATTTCTTCTGAATTTTTTTAGATCGTTTTTTGTTTAAAATCTCTTCTTCCTCA |
| (SEQ ID NO: 1399) | GGAGTCAGCTTGGCTCCCTTCTTGCGGCCCAGGGGCAGCGCATAGTGGGACTCGTACCACTGTC |
| RPS8_NM_001012_r1_5 | /5Biosg/CATAGCCATCTGCTCGGCCACACTGTCCCGGCCTTGAAGCGATGCACGCAAGAAGC |
| (SEQ ID NO: 1400) | TTGCCCTGCTGGAACTGCTCCTCCAGGAGACTGCTGATTTTGGCATTCTTTTTCCTTTCATCAT |
| RPS8_NM_001012_r1_6 | /5Biosg/TGTGGGAACAAAACAATAAACACCTTTATTACATGGGTGAAGACAAAACAAGGATT |
| (SEQ ID NO: 1401) | TATTTGCCTTTGCGGGCCTTGATTTTCCTAAGATAGAACTCCAACTCTTTGCCCTCTAGCACAT |
| RPS9_NM_001013_r1_1 | /5Biosg/TCTCGAAGGGTCTCCGCGGGGTCACATAAGTTTTGCGACAAACCCAGCTCCGGGCC |
| (SEQ ID NO: 1402) | ACTGGCATGTTGGCTCCGCTTCCCCGTCTGCGCCTAAGCAAACCACCCGGTCACTGAGAAAGAG |
| RPS9_NM_001013_r1_2 | /5Biosg/CAGCAGTTCCCGGGCGGCCTTGCGGATCTTGGCCAGGGTAAATTTGACCCTCCAGA |
| (SEQ ID NO: 1403) | CCTCACGTTTGTTCCGGAGCCCATACTCGCCGATCAGCTTCAGCTCTTGGTCGAGACGAGATTT |
| RPS9_NM_001013_r1_3 | /5Biosg/TCAGGCCCAGGATGTAATCCAGCTTCATCTTGCCCTCATCCAGCACCCCAATGCGG |
| (SEQ ID NO: 1404) | ACCAGCCGCCGCAGCAGGGCGTTGCCTTCGAACAGACGCCGTGGGTCCTTCTCATCAAGCGTCA |
| RPS9_NM_001013_r1_4 | /5Biosg/CTGCTTGCGGACCCTGATATGGCGCTGGCGGATCAGCACGCGAGCGTGGTGGATGG |
| (SEQ ID NO: 1405) | ACTTGGCCAAGCCCAGCTTGAAGACCTGGGTCTGCAGGCGTCTCTCTAAGAAATCCTCTATCTT |
| RPS9_NM_001013_r1_5 | /5Biosg/TCTTGGCATTCTTCCTCTTCACGCGGCCCGGGCGGCCACCCCCGTAGGGAGAGCGC |
| (SEQ ID NO: 1406) | AGAGAGAAGTCGATGTGCTTCTGGGAATCCAGGCGGACAATGAAGGACGGGATGTTCACCACCT |
| RPS9_NM_001013_r1_6 | /5Biosg/TTGTAAAGCGCTGATCCTGTTTATTTGGCAGGAAAACGAGACAATCCAGCAGCCCA |
| (SEQ ID NO: 1407) | GGAGGGACAGGTGGACTTAATCCTCCTCCTCGTCGTCTCCAGCCCCAGCCCCACCCTGGCCCTT |
| RPSA_NM_002995_r1_1 | /5Biosg/TCCTTCATTTGCAGGACATCAAGGGCTCCGGACATTGTGAAAATTTCCCTTTAAGT |
| (SEQ ID NO: 1408) | TACGACGGGAATCCAGAACAACGCCGTATGGACCCCTCTGCAGGTAGCACGGAAAAGACAGGCG |
| RPSA_NM_002995_r1_2 | /5Biosg/TTTATGATATAGATGCCATCACTTTTCCTTTTATAGATGTACTGTTCCATCTGGAA |
| (SEQ ID NO: 1409) | GTCAAGATTGGTGCCACCTAAGTGGGTTCCTGCTGCAAGGAACTTAAGGACATCCTCCTCCTTC |
| RPSA_NM_002995_r1_3 | /5Biosg/CAGCACAGCCCTCTGGCCAGTATTCCTGGAGGATATAACACTGACATCAGCAGGGT |
| (SEQ ID NO: 1410) | TTTCAATGGCAACAATTGCACGAGCTGCCAGCAGAAGCTTCTCCCAGGTCCTCTTGAGATTTAT |
| RPSA_NM_002995_r1_4 | /5Biosg/GTCAGTAACCACAAGAAGCCGTGGCTCCCGGAAGGCTGCCTGGATCTGGTTAGTGA |
| (SEQ ID NO: 1411) | AGGTTCCAGGAGTGAAGCGGCCAGCAATTGGAGTGGCTCCAGTGGCAGCAGCAAACTTCAGCAC |
| RPSA_NM_002995_r1_5 | /5Biosg/TGTTGCATGGGATGGCAATGTCCACATAGCGCAGAGGAGAATCTGTGTTACACAGC |
| (SEQ ID NO: 1412) | GCAATGGTAGGTAGGTTAACATAAGATGCCTCCGTGAGAGGCTGGTGGTCAGCCCTGGGGTCAG |
| RPSA_NM_002995_r1_6 | /5Biosg/AGTACAGATCAGGCATGACCTCCCATGGGTGTTCACGGGAAATGGTGCCACGCATG |
| (SEQ ID NO: 1413) | CGCAGAACTTCCCGAGCCAGCATCCACCACATCAAACCCACTGAGTGAGCTCCCTTGTTGTTGC |
| RPSA_NM_002995_r1_7 | /5Biosg/GTAGCAGTGAACTCAGGAGCGGGAGCAGTCCATTCACCCTGAAATTCCTCCTTGGT |
| (SEQ ID NO: 1414) | CACTGCCTTCTCAGCAGCAGCCTGCTCTTCTTTTTCAATCTCTTCAGGATCTCTGTAGAAGTAC |
| RPSA_NM_002995_r1_8 | /5Biosg/GCTGCAGACCAGTCTTCCGTGGCAGGCTGAGCGCTCCAGTCTTCAGTAGGGAATTG |
| (SEQ ID NO: 1415) | CTGAATAGGCACAGAGGGCACCTGTACACCTTCAGACCAGTCTGCAACCTCAGGCTGAGTAGCA |
| RPSA_NM_002995_r1_9 | /5Biosg/AACTGATGTTTATTTTCCATCAACCATTTTTCCATGCTGCTTAAGAGCCTATGCAA |
| (SEQ ID NO: 1416) | GAACAGCTTAAGACCAGTCAGTGGTTGCTCCTACCCATTCAGTGGCCTGAGCAGTGGGAGCTGC |
| RPSA_NM_002995_r1_10 | /5Biosg/TTTTTTAGAAGTACAACTCATGTCGAGACATGTATTTATTATTATGCTTTGATATG |
| (SEQ ID NO: 1417) | CAATCCCAGGTATTCTGATCTGGAGTAAAAAGCAAACTAAATGAAGACAACTTTTAGAAACTGA |

APPENDIX 9
Human globin mRNA sequences

LOCUS NM_000558 576 bp mRNA linear PRI 18 JAN. 2014
DEFINITION Homo sapiens hemoglobin, alpha 1 (HBA1), mRNA.
(SEQ ID NO: 1418)

| | |
|---|---|
| 1 | actcttctgg tccccacaga ctcagagaga acccaccatg gtgctgtctc ctgccgacaa |
| 61 | gaccaacgtc aaggccgcct ggggtaaggt cggcgcgcac gctggcgagt atggtgcgga |
| 121 | ggccctggag aggatgttcc tgtccttccc caccaccaag acctacttcc cgcacttcga |
| 181 | cctgagccac ggctctgccc aggttaagag ccacgacaag aaagtggccg acgcgctgac |
| 241 | caacgccgtg gcgcacgtgg acgacatacc caacacgctg tccgccctga gcgacctgca |
| 301 | cgcgcacaag cttcgggtgg acccggtcaa cttcaagctc ctaagccact gcctgctggt |
| 361 | gaccctggcc gcccacctcc ccgccgagtt cacccctgcg gtgcacgcct ccctggacaa |
| 421 | gttcctggct tctgtgagca ccgtgctgac ctccaaatac cgttaagctg gagcctcggt |
| 481 | ggccatgctt cttgcccctt gggcctcccc ccagcccctc ctccccttcc tgcacccgta |
| 541 | cccccgtggt ctttgaataa agtctgagtg ggcggc |

LOCUS NM_000517 622 bp mRNA linear PRI 18 JAN. 2014
DEFINITION Homo sapiens hemoglobin, alpha 2 (HBA2), mRNA.
(SEQ ID NO: 1419)

| | |
|---|---|
| 1 | cataaaccct ggcgcgctcg cgggccggca ctcttctggt ccccacagac tcagagagaa |
| 61 | cccaccatgg tgctgtctcc tgccgacaag accaacgtca agaccgcctg gggtaaggtc |
| 121 | ggcgcgcacg ctggcgagta tggtgcggag gccctggaga gaatgttcct gtccttcccc |
| 181 | accaccaaga cctacttccc gcacttcgac ctgagccacg gctctgccca ggttaagggc |
| 241 | cacggcaaga aggtggccga cgcgctgacc aacgccgtgg cgcacgtgga cgacatgccc |
| 301 | aacgcgctgt ccgccctgag cgacctgcac gcgcacaagc ttcgggtgga cccagtcaac |
| 361 | ttcaagctcc taagccactg cctgctggtg accctggcca cccacctccc cgccgagttc |
| 421 | acccctgcgg tgcacgcctc cctggacaag ttcctggctt ctgtgagcac cgtgctgacc |
| 481 | tccaaatacc gttaagctgg agcctcggta gccgttcctc ctgcccgctg ggcctcccaa |
| 541 | cgggccctcc tcccctcctt gcaccggccc ttcctggtct ttgaataaag tctgagtggg |
| 601 | cagc |

LOCUS NM_000518 626 bp mRNA linear PRI 18 JAN. 2014
DEFINITION Homo sapiens hemoglobin, beta (HBB), mRNA.
(SEQ ID NO: 1420)

| | |
|---|---|
| 1 | acatttgctt ctgacacaac tgtgttcact agcaacctca aacagacacc atggtgcatc |
| 61 | tgactcctga ggagaagtct gccgttactg ccctgtgggg caaggtgaac gtggatgaag |
| 121 | ttagtggtga ggccctgagc aggctgctgg tagtctaccc ttggacccag aggttatttg |
| 181 | agtcctttga ggatctgtcc actcctgatg ctgttatggg caaccctaag gtgaaggctc |
| 241 | atggcaagaa agtgctcggt gcctttagtg atggcctggc tcacctggac aacctcaagg |
| 301 | gcacctttgc cacactgagt gagctgcact gtgacaagct gcacgtggat cctgagaact |
| 361 | tcaggctcct gggcaacgtg ctggtctgtg tgctggccca tcactttggc aaagaattca |
| 421 | ccccaccagt gcaagctgcc tatcagaaag tggtgactgg tgtggctaat gccctggccc |
| 481 | acaagtatca ctaagctcgc tttcttgctg tccaatttct attaaaggtt cctttgttcc |
| 541 | ctaagtccaa ctactaaact aggggatatt atgaagggcc ttgagcatct ggattctgcc |
| 601 | taataaaaaa catttatttt cattgc |

LOCUS NM_000519 774 bp mRNA linear PRI 10 JAN. 2014
DEFINITION Homo sapiens hemoglobin, delta (HBD), mRNA.
(SEQ ID NO: 1421)

| | |
|---|---|
| 1 | agggcaagtt aagggaatag tggaatgaag gttcattttt cattctcaca aactaatgaa |
| 61 | accctgctta tcttaaacca acctgctcac tggagcaggg aggacaggac cagcataaaa |
| 121 | ggcagggcag agtcgactgt tgcttacact ttcttctgac ataacagtgt tcactagcaa |
| 181 | cctcaaacag acaccatggt gcatctgact cctgaggaga agactgctgt caatgccctg |
| 241 | tggggcaaag tgaacgtgga tgcagttgat ggtgaagccc tgagcagatt actggtggtc |
| 301 | tacccttgga cccagaggtt ctttgagtcc tttggggatc tgtcctctcc tgatgctgtt |
| 361 | atgggcaacc ctaaggtgaa ggctcatggc aagaaggtgc taggtgcctt tagtgatggc |
| 421 | ctggctcacc tggacaacct caagggcact ttttctcagc tgagtgagct gcactgtgac |
| 481 | aagctgcacg tggatcctga gaacttcagg ctcttgggca atgtgctggt gtgtgtgctg |
| 541 | gcccgcaact ttggcaagga attcacccca caaatgcagg ctgcctatca gaaggtggtg |
| 601 | gctggtgtgg ctaatgccct agctcacaag taccattgag atcctggact gtttcctgat |
| 661 | aaccataaga agaccctatt tccctagatt ctattttctg aacttgggaa cacaatgcct |
| 721 | acttcaaggg tatggcttct gcctaataaa gaatgttcag ctcaacttcc tgat |

LOCUS NM_000559 584 bp mRNA, linear PRI 10 JAN. 2014
DEFINITION Homo sapiens hemoglobin, gamma A (HBG1), mRNA.
(SEQ ID NO: 1422)

| | |
|---|---|
| 1 | acactcgctt ctggaacatc tgagattatc aataagctcc tagtccagac gccatgggtc |
| 61 | atttcacaga ggaggacaag gctactatca caagcctgtg gggcaaggtg aatgtggaag |
| 121 | atgctggagg agaaaccctg ggaaggctcc tggttgtcta cccatggacc cagaggttct |
| 181 | ttgacagctt tggcaacctg tcctctgcct ctgccatcat gggcaacccc aaagtcaagg |
| 241 | cacatggcaa gaaggtgctg acttccttgg gagatgccac aaagcacctg gatgatctca |
| 301 | agggcacctt tgcccagctg agtgaactac actgtaacaa gctgcatgtg gatcctgaaa |
| 361 | acttcaagct cctgggaaat gtgctggtga ccgttttggc aatccatttc ggcaaagaat |
| 421 | tcacccctga ggtgcaggct tcctggcaga agatggtgac tgcagtggcc agtgccctgt |
| 481 | cctccagata ccactgagct cactgcccat gattcagagc tttcaaggat aggctttatt |
| 541 | ctgcaagcaa tacaaataat aaatctattc tgctgagaga tcac |

LOCUS NM_000184 583 bp mRNA linear PRI 10 JAN. 2014
DEFINITION Homo sapiens hemoglobin, gamma G (HBG2), mRNA.
(SEQ ID NO: 1423)

| | |
|---|---|
| 1 | acactcgctt ctggaacgtc tgaggttatc aataagctcc tagtccagac gccatgggtc |
| 61 | atttcacaga ggaagacaag actactatca caagcctgtg ggacaaggtg aatgtggaag |
| 121 | atgctggagg agaaaccctg ggaaggctcc tggttgtcta cccatggacc cagaggttct |
| 181 | ttgacagctt tggcaacctg tcctctgcct ctgccatcat gggcaacccc aaagtcaagg |
| 241 | cacatggcaa gaaggtgctg acttccttgg gagatgccat aaagcacctg gatgatctca |
| 301 | agggcacctt tgcccagctg agtgaactgc actgtgacaa gctgcatgtg gatcctgaga |
| 361 | acttcaagct cctgggaaat gtgctggtga ccgttttggc aatccatttc ggcaaagaat |
| 421 | tcacccctga ggtgcaggct tcctggcaga agatggtgac tggagtggcc agtgccctgt |
| 481 | cctccagata ccactgagct cactgcccat gatgcagagc tttcaaggat aggctttatt |
| 541 | ctgcaagcaa tcaaataata aatctattct gctaagagat cac |

APPENDIX 10
DNA globin mRNA Capture Probes/Baits
(Note: /5Biosg/ = 5′-biotin)

| Name | Sequence |
|---|---|
| NM_000558_HS_HBA1_r1_1 | /5Biosg/TCCGCACCATACTCGCCAGCGTGCGCGCCGACCTTACCCCAGGCGGCCTTGACGTT |
| (SEQ ID NO: 1424) | GGTCTTGTCGGCAGGAGACAGCACCATGGTGGGTTCTCTCTGAGTCTGTGGGGACCAGAAGAGT |
| NM_000558_HS_HBA1_r1_2 | /5Biosg/GCGTCGGCCACCTTCTTGCCGTGGCCCTTAACCTGGGCAGAGCCGTGGCTCAGGTC |
| (SEQ ID NO: 1425) | GAAGTGCGGGAAGTAGGTCTTGGTGGTGGGGAAGGACAGGAACATCCTCTCCAGGGCCTCCGCA |
| NM_000558_HS_HBA1_r1_3 | /5Biosg/TGGCTTAGGAGCTTGAAGTTGACCGGGTCCACCCGAAGCTTGTGCGCGTGCAGGTC |
| (SEQ ID NO: 1426) | GCTCAGGGCGGACAGCGCGTTGGGCATGTCGTCCACGTGCGCCACGGCGTTGGTCAGCGCGTCG |
| NM_000558_HS_HBA1_r1_4 | /5Biosg/CGGTATTTGGAGGTCAGCACGGTGCTCACAGAAGCCAGGAACTTGTCCAGGGAGGC |
| (SEQ ID NO: 1427) | GTGCACCGCAGGGGTGAACTCGGCGGGGAGGTGGGCGGCCAGGGTCACCAGCAGGCAGTGGCTT |
| NM_000558_HS_HBA1_r1_5 | /5Biosg/GCCGCCCACTCAGACTTTATTCAAAGACCACGGGGGTACGGGTGCAGGAAGGGGAG |
| (SEQ ID NO: 1428) | GAGGGGCTGGGGGGAGGCCCAAGGGGCAAGAAGCATGGCCACCGAGGCTCCAGCTTAACGGTAT |
| NM_000517_HS_HBA2_r1_1 | /5Biosg/GACCTTACCCCAGGCGGCCTTGACGTTGGTCTTGTCGGCAGGAGACAGCACCATGG |
| (SEQ ID NO: 1429) | TGGGTTCTCTCTGAGTCTGTGGGGACCAGAAGAGTGCCGGCCCGCGAGCGCGCCAGGGTTTATG |
| NM_000517_HS_HBA2_r1_2 | /5Biosg/GGCTCAGGTCGAAGTGCGGGAAGTAGGTCTTGGTGGTGGGGAAGGACAGGAACATC |
| (SEQ ID NO: 1430) | CTCTCCAGGGCCTCCGCACCATACTCGCCAGCGTGCGCGCCGACCTTACCCCAGGCGGCCTTGA |
| NM_000517_HS_HBA2_r1_3 | /5Biosg/GCGGACAGCGCGTTGGGCATGTCGTCCACGTGCGCCACGGCGTTGGTCAGCGCGTC |
| (SEQ ID NO: 1431) | GGCCACCTTCTTGCCGTGGCCCTTAACCTGGGCAGAGCCGTGGCTCAGGTCGAAGTGCGGGAAG |
| NM_000517_HS_HBA2_r1_4 | /5Biosg/GGGAGGTGGGCGGCCAGGGTCACCAGCAGGCAGTGGCTTAGGAGCTTGAAGTTGAC |
| (SEQ ID NO: 1432) | CGGGTCCACCCGAAGCTTGTGCGCGTGCAGGTCGCTCAGGGCGGACAGCGCGTTGGGCATGTCG |
| NM_000517_HS_HBA2_r1_5 | /5Biosg/CGAGGCTCCAGCTTAACGGTATTTGGAGGTCAGCACGGTGCTCACAGAAGCCAGGA |
| (SEQ ID NO: 1433) | ACTTGTCCAGGGAGGCGTGCACCGCAGGGGTGAACTCGGCGGGGAGGTGGGCGGCCAGGGTCAC |
| NM_000517_HS_HBA2_r1_6 | /5Biosg/GCTGCCCACTCAGACTTTATTCAAAGACCAGGAAGGGCCGGTGCAAGGAGGGGAGG |
| (SEQ ID NO: 1434) | AGGGCCCGTTGGGAGGCCCAGCGGGCAGGAGGAACGGCTACCGAGGCTCCAGCTTAACGGTATT |
| NM_000518_HS_HBB_r1_1 | /5Biosg/CTTCATCCACGTTCACCTTGCCCCACAGGGCAGTAACGGCAGACTTCTCCTCAGGA |
| (SEQ ID NO: 1435) | GTCAGATGCACCATGGTGTCTGTTTGAGGTTGCTAGTGAACACAGTTGTGTCAGAAGCAAATGT |
| NM_000518_HS_HBB_r1_2 | /5Biosg/GCCCATAACAGCATCAGGAGTGGACAGATCCCCAAAGGACTCAAAGAACCTCTGGG |
| (SEQ ID NO: 1436) | TCCAAGGGTAGACCACCAGCAGCCTGCCCAGGGCCTCACCACCAACTTCATCCACGTTCACCTT |
| NM_000518_HS_HBB_r1_3 | /5Biosg/TCACTCAGTGTGGCAAAGGTGCCCTTGAGGTTGTCCAGGTGAGCCAGGCCATCACT |
| (SEQ ID NO: 1437) | AAAGGCACCGAGCACTTTCTTGCCATGAGCCTTCACCTTAGGGTTGCCCATAACAGCATCAGGA |
| NM_000518_HS_HBB_r1_4 | /5Biosg/GGGGTGAATTCTTTGCCAAAGTGATGGGCCAGCACACAGACCAGCACGTTGCCCAG |
| (SEQ ID NO: 1438) | GAGCCTGAAGTTCTCAGGATCCACGTGCAGCTTGTCACAGTGCAGCTCACTCAGTGTGGCAAAG |
| NM_000518_HS_HBB_r1_5 | /5Biosg/TTAATAGAAATTGGACAGCAAGAAAGCGAGCTTAGTGATACTTGTGGGCCAGGGCA |
| (SEQ ID NO: 1439) | TTAGCCACACCAGCCACCACTTTCTGATAGGCAGCCTGCACTGGTGGGGTGAATTCTTTGCCAA |
| NM_000518_HS_HBB_r1_6 | /5Biosg/GCAATGAAAATAAATGTTTTTTATTAGGCAGAATCCAGATGCTCAAGGCCCTTCAT |
| (SEQ ID NO: 1440) | AATATCCCCCAGTTTAGTAGTTGGACTTAGGGAACAAAGGAACCTTTAATAGAAATTGGACAGC |
| NM_000519_HS_HBD_r1_1 | /5Biosg/TTTTATGCTGGTCCTGTCCTCCCTGCTCCAGTGAGCAGGTTGGTTTAAGATAAGCA |
| (SEQ ID NO: 1441) | GGGTTTCATTAGTTTGTGAGAATGAAAAATGAACCTTCATTCCACTATTCCCTTAACTTGCCCT |
| NM_000519_HS_HBD_r1_2 | /5Biosg/CAGCAGTCTTCTCCTCAGGAGTCAGATGCACCATGGTGTCTGTTTGAGGTTGCTAG |
| (SEQ ID NO: 1442) | TGAACACTGTTATGTCAGAAGAAAGTGTAAGCAACAGTCGACTCTGCCCTGCCTTTTATGCTGG |
| NM_000519_HS_HBD_r1_3 | /5Biosg/TCCCCAAAGGACTCAAAGAACCTCTGGGTCCAAGGGTAGACCACCAGTAATCTGCC |
| (SEQ ID NO: 1443) | CAGGGCCTCACCACCAACTGCATCCACGTTCACTTTGCCCCACAGGGCATTGACAGCAGTCTTC |
| NM_000519_HS_HBD_r1_4 | /5Biosg/GCCCTTGAGGTTGTCCAGGTGAGCCAGGCCATCACTAAAGGCACCTAGCACCTTCT |
| (SEQ ID NO: 1444) | TGCCATGAGCCTTCACCTTAGGGTTGCCCATAACAGCATCAGGAGAGGACAGATCCCCAAAGGA |
| NM_000519_HS_HBD_r1_5 | /5Biosg/TGCCAAAGTTGCGGGCCAGCACACACACCAGCACATTGCCCAAGAGCCTGAAGTTC |
| (SEQ ID NO: 1445) | TCAGGATCCACGTGCAGCTTGTCACAGTGCAGCTCACTCAGCTGAGAAAAAGTGCCCTTGAGGT |
| NM_000519_HS_HBD_r1_6 | /5Biosg/TGGTTATCAGGAAACAGTCCAGGATCTCAATGGTACTTGTGAGCCAGGGCATTAGC |
| (SEQ ID NO: 1446) | CACACCAGCCACCACCTTCTGATAGGCAGCCTGCATTTGTGGGGTGAATTCCTTGCCAAAGTTG |
| NM_000519_HS_HBD_r1_7 | /5Biosg/ATCAGGAAGTTGAGCTGAACATTCTTTATTAGGCAGAAGCCATACCCTTGAAGTAG |
| (SEQ ID NO: 1447) | GCATTGTGTTCCCAAGTTCAGAAAATAGAATCTAGGGAAATAGGGTCTTCTTATGGTTATCAGG |
| NM_000184_HS_HBG2_r1_1 | /5Biosg/CTTCCACATTCACCTTGCCCCACAGGCTTGTGATAGTAGCCTTGTCCTCCTCTGTG |
| (SEQ ID NO: 1448) | AAATGACCCATGGCGTCTGGACTAGGAGCTTATTGATAACCTCAGACGTTCCAGAAGCGAGTGT |
| NM_000184_HS_HBG2_r1_2 | /5Biosg/GACTTTGGGGTTGCCCATGATGGCAGAGGCAGAGGACAGGTTGCCAAAGCTGTCAA |
| (SEQ ID NO: 1449) | AGAACCTCTGGGTCCATGGGTAGACAACCAGGAGCCTTCCCAGGGTTTCTCCTCCAGCATCTTC |
| NM_000184_HS_HBG2_r1_3 | /5Biosg/CCACATGCAGCTTGTCACAGTGCAGTTCACTCAGCTGGGCAAAGGTGCCCTTGAGA |
| (SEQ ID NO: 1450) | TCATCCAGGTGCTTTATGGCATCTCCCAAGGAAGTCAGCACCTTCTTGCCATGTGCCTTGACTT |
| NM_000184_HS_HBG2_r1_4 | /5Biosg/CACTCCAGTCACCATCTTCTGCCAGGAAGCCTGCACCTCAGGGGTGAATTCTTTGC |
| (SEQ ID NO: 1451) | CGAAATGGATTGCCAAAACGGTCACCAGCACATTTCCCAGGAGCTTGAAGTTCTCAGGATCCAC |
| NM_000184_HS_HBG2_r1_5 | /5Biosg/GTGATCTCTTAGCAGAATAGATTTATTATTTGATTGCTTGCAGAATAAAGCCTATC |
| (SEQ ID NO: 1452) | CTTGAAAGCTCTGCATCATGGGCAGTGAGCTCAGTGGTATCTGGAGGACAGGGCACTGGCCACT |

What is claimed is:

1. A method of selectively removing a undesired RNA target from a population of RNA molecules, comprising:
(a) contacting the population of RNA molecules with a DNA oligonucleotide comprising a bait to form a mixture; and
(b) isolating the undesired RNA target from the mixture.

2. The method of claim 1, wherein the step of contacting the population of RNA molecules with a DNA oligonucleotide comprising a bait comprises incubating the mixture in an appropriate buffer at a temperature sufficient to form a bait:undesired RNA target complex.

3. The method of claim 1, wherein the step of isolating the undesired RNA target comprises:
(i) forming a bait:undesired RNA target complex; and
(ii) separating the bait:undesired RNA target complex from the mixture.

4. The method of claim 1, wherein the bait comprises a sequence having substantial sequence complementarity to a sequence within the undesired RNA target.

5. The method of claim 1, wherein the bait includes a covalent modification to enable selection of the bait:undesired RNA target complex.

6. The method of claim 5, wherein the covalent modification is a biotinylated group.

7. The method of claim 6, wherein the bait:undesired RNA target complex is contacted with a solid support coupled to avidin or streptavidin.

8. The method of claim 7, wherein the solid support comprises magnetic particles.

9. The method of claim 3, wherein the bait:undesired RNA target complex is separated from the mixture by immobilization on magnetic particles and the magnetic particles are subsequently removed from the desired RNA.

10. The method of claim 9, wherein the magnetic particles are removed from the desired RNA by attraction to a magnet.

11. The method of claim 10 wherein the magnet used to attract the magnetic particles is inserted into the vessel containing the desired RNA.

12. The method of claim 1, wherein the undesired RNA target comprises a highly abundant RNA in the population of RNA molecules.

13. The method of claim 12, wherein the highly abundant RNA is selected from an rRNA, a mRNA encoding a ribosomal protein and a mRNA encoding a globin protein.

14. The method of claim 12, wherein the highly abundant RNA comprises rRNA.

15. A method of performing massively parallel sequencing of RNA from a sample, comprising:
(a) contacting a complex population of total RNA with a plurality of DNA oligonucleotides comprising baits to form a mixture, wherein at least one member of the plurality of DNA oligonucleotides comprising baits has substantial sequence complementarity to a sequence within at least one species of an undesired RNA target;
(b) isolating the at least one species of an undesired RNA target from the mixture to form a depleted population of total RNA;
(c) preparing a cDNA library of the depleted population of total RNA; and
(d) sequencing the cDNA library of the depleted library population of total RNA.

16. The method of claim 15, wherein the step of isolating the at least one species of an undesired RNA target from the mixture to form the depleted population of total RNA comprises:
(i) forming a plurality of hybrid complexes between the at least one species of an undesired RNA target and a plurality of oligonucleotides as baits; and
(ii) separating the plurality of hybrid complexes from the mixture.

17. The method of claim 16, wherein each member of the plurality of oligonucleotides as baits comprises a covalent modification.

18. The method of claim 17, wherein the covalent modification comprises a biotinylated group.

19. The method of claim 16, wherein the step of separating the plurality of hybrid complexes from the mixture comprises contacting the mixture comprising the plurality of hybrid complexes with a solid support coupled to avidin or streptavidin.

20. The method of claim 16 wherein at least one member of the plurality of oligonucleotides as baits comprises a biotinylated group at the 3′ end and at the 5′ end of the oligonucleotide.

21. The method of claim 20 wherein the plurality of oligonucleotides are between 45 and 80 bases long.

22. The method of claim 15, wherein the undesired RNA target comprises a highly abundant RNA in the complex population of total RNA.

23. The method of claim 22, wherein the highly abundant RNA is selected from an rRNA, a mRNA encoding a ribosomal protein and a mRNA encoding a globin protein.

24. The method of claim 15, wherein the step of preparing the cDNA library of the depleted population of total RNA comprises:
fragmenting the depleted population of total RNA to form a depleted population of fragmented RNA; and
converting the depleted population of fragmented desired RNA to form double-stranded cDNA,

25. A kit comprising a capture reagent for use in a selection method of an undesired RNA, wherein the capture reagent comprises a plurality of DNA bait oligonucleotides, wherein each member of the plurality of DNA bait oligonucleotides is prepared individually by a synthetic chemical process.

26. The kit of claim 25, wherein the undesired RNA comprises a highly abundant RNA in a total RNA sample.

27. The kit of claim 26, wherein the highly abundant RNA is selected from rRNA, a mRNA encoding a ribosomal protein and a mRNA encoding a globin protein.

28. The kit of claim 26, wherein the highly abundant RNA comprises rRNA.

29. The kit of claim 25, wherein the plurality of DNA bait oligonucleotides comprises at least two members selected from a group consisting of SEQ ID NOS: 17-645, 725-1417, 1424-1452, and 1454-1456.
